(19) United States
(12) Patent Application Publication
(10) Pub. No.: US 2004/0096894 A1
(43) Pub. Date: May 20, 2004

(54) Crystal structures of P- selectin, P- and E-selectin complexes, and uses thereof

(76) Inventors: William Stuart Somers, Cambridge, MA (US); Jin Tang, Canton, MA (US); Raymond Camphausen, Arlington, MA (US); Jasbir Seehra, Lexington, MA (US)

Correspondence Address: Craig J. Arnold, Amster, Rothstein & Ebenstein, 90 Park Avenue, New York, NY, 10016, US

(21) Appl. No.: 09/859,722

(22) Filed: May 17, 2001

Related U.S. Application Data
(60) Provisional application No. 60/205,875, filed on May 19, 2000.

(51) Int. Cl. C12Q 1/68; G01N 33/53; C07K 14/47; C07K 14/415

(52) U.S. Cl. 435/7.1; 530/370; 530/395; 536/53

(57) ABSTRACT

The present invention relates to the crystal and three dimensional structures of the lectin and EGF-like (LE) domains of P-selectin, the crystal and three dimensional structures of P-selectin LE and E-selectin LE each complexed with SLe^{X}, as well as the crystal and three dimensional structure of P-selectin LE complexed with a functional PSGL-1 peptide modified by both tyrosine sulfation and SLe^{X}. The present invention also provides methods for identifying agents which activate or inhibitor each of the foregoing structures. In addition, the present invention provides agents identified by such methods.

This application claims the benefit of U.S. Provisional Application No. 60/205,875 filed May 19, 2000.

### FIELD OF THE INVENTION

The present invention relates to the crystal and three dimensional structures of the lectin and EGF-like (LE) domains of P-selectin, the crystal and three dimensional structures of P-selectin LE and E-selectin LE each complexed with SLe^{X}, as well as the crystal and three dimensional structure of P-selectin LE complexed with a functional PSGL-1 peptide modified by both tyrosine sulfation and SLe^{X}. These structures are critical for the design and selection of agents that interfere with the cellular rolling of leukocytes in the inflammation process.

### BACKGROUND OF THE INVENTION

The selectins are a family of cell-surface glycoproteins responsible for early adhesion events in the recruitment of leukocytes into sites of inflammation and their emigration into lymphatic tissues (reviewed in (Kansas, 1996) and (Vestweber and Blanks, 1999)). As part of a multistep process (Springer, 1994), selectins promote the initial attachment (tethering) and subsequent rolling of leukocytes over vessel walls where they become activated as a consequence of exposure to locally produced chemokines. Firm adhesion of the leukocytes mediated by integrins precedes their extravasation into the underlying tissue. P-selectin (CD62P) and E-selectin (CD62E) are induced on the surface of vascular endothelium in response to inflammatory stimuli. P-selectin, also expressed by activated platelets, is translocated within minutes from intracellular stores to the cell-surface following induction by inflammatory mediators. E-selectin is transcriptionally regulated and appears within a few hours of activation of the vascular endothelium. L-selectin (CD62L), a third member of the selectin family is expressed constitutively on leukocytes. In addition to its role in inflammation, L-selectin mediates the attachment of lymphocytes to specialized high endothelial venules in the course of their migration from the blood to lymphoid tissues.

The selectins share a number of structural and functional properties. They consist of a highly homologous N-terminal calcium-dependent (C-type, (Drickamer, 1988)) lectin domain, an epidermal growth factor (EGF)-like domain, variable numbers of complement regulatory-like units, a transmembrane domain, and an intracellular region. It is generally accepted that selectin binding is mediated predominantly through weak protein-carbohydrate interactions between the lectin domain and glycan ligands on apposing cells. A number of diverse glycan structures have been described as able to support and/or to inhibit selectin binding. However, an epitope displayed by the sialyl Lewis^{X} (SLe^{X}, NeuNAc2,3Gal1,4 [Fuc1,3] GlcNAc) tetrasaccharide and related structures appears to be a physiologically relevant recognition component common to all three selectins (Foxall et al., 1992). Additional factors for recognition have been suggested by the isolation of specific glycoprotein counterreceptors of apparent high affinity (or avidity) yet of diverse structure. These include GlyCAM-1 (Lasky et al., 1992), MAdCAM-1 (Berg et al., 1993), CD34 (Baumheuter et al., 1993), ESL-1 (Levinovitz et al., 1993), and PSGL-1 (Moore et al., 1992); (Sako et al., 1993). However, there is only limited evidence that any of these heavily glycosylated proteins are in fact essential raising the possibility that it is the cell- or tissue-specific glycosylation capabilities (i.e., ability to produce SLe^{X}-like glycans), and not the expression of specific glycoproteins, which ultimately confers selectin reactivity. The clear exception is PSGL-1, a mucin-like homodimeric glycoprotein expressed by virtually all subsets of leukocytes (reviewed in (Yang et al., 1999) and (McEver and Cummings, 1997)). While it was anticipated that P-selectin recognition would be dependent upon SLe^{X}-like modifications of glycans within a mucin-like region of PSGL-1, an essential binding epitope was localized to the anionic, N-terminal portion of the polypeptide backbone well outside of the mucin domain (Pouyani and Seed, 1995; Sako et al., 1995; Wilkins et al., 1995). Numerous studies have demonstrated that P-selectin mediated binding and in vivo inflammatory responses can be greatly diminished by targeting this polypeptide determinant within PSGL-1. (McEver and Cummings, 1997; Yang et al., 1999). Moreover, recent studies of mice rendered genetically deficient in PSGL-1 have been described that show significant defects in P-selectin mediated leukocyte rolling and inflammatory recruitment (Yang et al., 1999). Therefore, PSGL-1 represents the single example of a selectin counterreceptor for which both polypeptide and SLe^{X}-modified glycan components are required for physiologically relevant binding.

In light of their unique function as mediators of cell attachment and rolling under the influence of shear stresses encountered within the vasculature, considerable efforts have been directed towards characterization of the underlying biophysical and molecular bases of selectin interactions. The selectins associate and dissociate with their ligands with rapid binding kinetics (Alon et al., 1997; Alon et al., 1995) and it is this property which appears responsible in part for their ability to mediate transient tethers and the cellular rolling phenomenon. Other factors including the mechanical (Alon et al., 1995; Puri et al., 1998) and unique structural properties (Chen et al., 1997) of selectin interactions also appear to be involved, but these remain incompletely characterized owing to a lack of high-resolution molecular structures of the selectins complexed with physiological ligands. Significant efforts have been made to overcome this limitation but to date these remain incomplete. An X-ray crystal structure of an E-selectin construct containing the lectin and EGF domains (lec/EGF) has been previously described (Graves et al., 1994) and, combined with site-directed mutagenesis studies (Kansas, 1996), suggested a putative SLe^{X} binding site localized to the lectin domain. Models for SLe^{X}binding to the E-selectin crystal structure have been proposed (Graves et al., 1994; Kogan et al., 1995; Poppe et al., 1997) based upon molecular docking of the free or bound solution structures of SLe^{X} ((Poppe et al., 1997) and references therein) using the X-ray crystal structure of the homologous rat serum mannose-binding protein (MBP-A) bound to oligomannose (Weis et al., 1992) as a guide. However, structures of E-selectin or other selecting, complexed with SLe^{X}-like carbohydrate ligands have not been determined confirming these hypotheses. In fact, prior to the present invention, such a crystal could not be obtained due to the blocking effect of high concentrations of calcium used to form the selectin crystals.

To date, crystal structures of MBP-A mutated to include E-selectin residues (the K3 mutant) co-complexed with SLe^{X} and related glycans (Ng and Weis, 1997) represent the only direct information of how selectins might bind their ligands. Collectively, these models and experimentally determined structures support an SLe^{X} binding motif in which two hydroxyl groups of the Fuc moiety ligate the lectin domain-bound calcium and additional binding interactions are perhaps mediated by the hydroxyl groups of Gal and the carboxylate moiety of NeuNAc. However, the models and MBP-A K3 mutant/SLe^{X} crystal structure differ in the orientation of SLe^{X} within the binding site and in the identity of molecular contacts.

An understanding of the structural basis for the high-affinity P-selectin/PSGL-1 interaction, is also incomplete and complicated by the observation that recognition is based upon both carbohydrate and polypeptide components. Mutagenesis studies have focused on the N-terminus of PSGL-1 and have demonstrated that P-selectin recognizes simultaneously one or more tyrosine sulfated residues within an anionic region of the PSGL-1 polypeptide and an SLe^{X}-containing O-glycan potentially localized to this region (Pouyani and Seed, 1995; Ramachandran et al., 1999; Sako et al., 1995; Wilkins et al., 1995). While the putative SLe^{X} binding site is anticipated to be similar between selectins and perhaps involved in the binding of the SLe^{X} component of PSGL-1 by P-selectin, the identity and structural basis of the P-selectin domain mediating the interaction with the PSGL-1 polypeptide are unknown. L-selectin has also been shown to recognize PSGL-1 (Kansas, 1996; Vestweber and Blanks, 1999) in the context of neutrophil-neutrophil interactions perhaps important for the amplification of leukocyte recruitment to sites of inflammation. This interaction is also affected by mutations within the N-terminus of the PSGL-1 polypeptide (Ramachandran et al., 1999) suggesting that P- and L-selectin have common binding requirements. In contrast, E-selectin binding to PSGL-1 or to other SLe^{X}-presenting counterreceptors has not been demonstrated to require polypeptide components.

### SUMMARY OF THE INVENTION

The present invention provides a crystal of lectin and EGF-like (LE) domains of P-selectin (“P-selectin LE”), as well as the three dimensional structure of P-selectin LE as derived by x-ray diffraction data of the P-selectin LE crystal. Specifically, the three dimensional structure of P-selectin LE is defined by the structural coordinates shown in FIG. 2, ± a root mean square deviation x-x from the backbone atoms of the amino acids of not more than 1.5 Å. The structural coordinates of the three dimensional structure of P-selectin LE are useful for a number of applications, including, but not limited to, the visualization, identification and characterization of various active sites of P-selectin LE, including the site in which SLe^{X} binds. The active site structures may then be used to design agents which interact with P-selectin LE, as well as P-selectin LE complexed with SLe^{X}, PSGL-1, or related molecules.

The present invention also provides a crystal of P-selectin LE complexed with SLe^{X}, as well as the three dimensional structures of P-selectin LE and SLe^{X} as derived by x-ray diffraction data of the P-selectin LE: SLe^{X} crystal. Specifically, the three dimensional structures of P-selectin LE and SLe^{X} are defined by the structural coordinates shown in FIG. 3, ± a root mean square deviation from the backbone atoms of the amino acids of not more than 1.5 Å. The structural coordinates of P-selectin LE and SLe^{X} are useful for a number of applications, including, but not limited to, the visualization, identification and characterization of various active sites of P-selectin, SLe^{X} and the P-selectin LE: SLe^{X} complex, including the SLe^{X} binding site. The active site structures may then be used to design various agents which interact with P-selectin LE, SLe^{X}, as well as P-selectin LE complexed with SLe^{X}, PSGL-1, or related molecules.

The present invention still further provides a crystal of lectin and EGF (LE) domains of E-selectin (“E-selectin LE”) complexed with SLe^{X}, as well as the three dimensional structures of E-selectin LE and SLe^{X} as derived by x-ray diffraction data of the E-selectin LE: SLe^{X} crystal. Specifically, the three dimensional structures of E-selectin LE and SLe^{X} are defined by the structural coordinates shown in FIG. 4, ± a root mean square deviation from the backbone atoms of the amino acids of not more than 1.5 Å. The structural coordinates of the three dimensional structures of E-selectin LE and SLe^{X} are useful for a number of applications, including, but not limited to, the visualization, identification and characterization of various active sites of E-selectin LE, SLe^{X} and the E-selectin LE: SLe^{X} complex, including the SLe^{X} binding site. The active site structures may then be used to design various agents which interact with E-selectin LE, SLe^{X}, as well as E-selectin LE complexed with SLe^{X}, PSGL-1 or related molecules.

Still further, the present invention provides a crystal structure of P-selectin LE complexed with a functional PSGL-1 peptide modified by both tyrosine sulfation and SLe^{X}, as well as the three dimensional structures of P-selectin LE and the PSGL-1 peptide as derived by x-ray diffraction data of the P-selectin LE: PSGL-1 peptide crystal. Specifically, the three dimensional structures of P-selectin LE and the PSGL-1 peptide are defined by the structural coordinates shown in FIG. 5, ± a root mean square deviation from the backbone atoms of the amino acids of not more than 1.5 Å. The structural coordinates of P-selectin LE and the PSGL-1 peptide are useful for a number of applications, including, but not limited to, the visualization, identification and characterization of various active sites of P-selectin LE, the PSGL-1 peptide and the P-selectin LE: PSGL-1 complex, including the SLe^{X} and PSGL-1 binding sites. The active site structures may then be used to design various agents which interact with P-selectin LE, PSGL-1, as well as P-selectin LE complexed with SLe^{X}, PSGL-1, or related molecules.

The present invention is also directed to an active site of an SLe^{X}binding protein or peptide, and preferably the SLe^{X} binding site of P-selectin LE, comprising the relative structural coordinates of amino acid residues TYR48, GLU80, ASN82, GLU92, TYR94, PRO98, SER99, ASN105, ASP106, GLU107 and bound calcium according to FIG. 3, ± a root mean square deviation from the backbone atoms of said amino acids of not more than 1.5 Å. Alternatively, the active site may include, in addition to the structural coordinates define above, the relative structural coordinates of amino acid residues TYR44, SER46, SER47, ALA77, ASP78, ASN79, PRO81, ASN83, ARG85, GLU88, CYS90, ILE93, LYS96, SER97, ALA100, TRP104, HIS108, LYS111 and LYS113 according to FIG. 3, ± a root mean square deviation from the backbone atoms of the amino acids of not more than 1.5 Å. The SLe^{X} active site may correspond to the configuration of the P-selectin LE in its state of association with an agent, preferably, SLe^{X}, or in its unbound state.

The present invention is further directed to an active site of an SLe^{X} binding protein or peptide, and preferably the SLe^{X} binding site of E-selectin LE, comprising the relative structural coordinates of amino acid residues TYR48, GLU80, ASN82, ASN83, GLU92, TYR94, ARG97, GLU98, ASN105, ASP106, GLU107 and bound calcium according to FIG. 4, ± a root mean square deviation from the backbone atoms of said amino acids of not more than 1.5 Å. Alternatively, the active site may include, in addition to the structural coordinates define above, the relative structural coordinates of amino acid residues TYR44, SER45, PRO46, SER47, ALA77, PRO78, GLY79, PRO81, GLU88, CYS90, LYS99, ASP100, TRP104, ARG108, LYS111 and LYS113 according to FIG. 4, ± a root mean square deviation from the backbone atoms of the amino acids of not more than 1.5 Å. The SLe^{X} active site may correspond to the configuration of the E-selectin LE in its state of association with an agent, preferably, SLe^{X}, or in its unbound state.

Still further, the present invention provides an active site of a PSGL-1 binding protein or peptide, and preferably the PSGL-1 binding site of P-selectin LE, comprising the relative structural coordinates of amino acid residues ALA9, TYR45, SER46, SER47, TYR48, GLU80, ASN82, LYS84, ARG85, GLU88, GLU92, TYR94, PRO98, SER99, ASN105, ASP106, GLU107, HIS108, LEU110, LYS 111, LYS 112, LYS 113, HIS114 and bound strontium according to FIG. 5, ± a root mean square deviation from the backbone atoms of said amino acids of not more than 1.5 Å. Alternatively, the active site may include, in addition to the structural coordinates define above, the relative structural coordinates of amino acid residues SER6, THR7, LYS8, TYR10, SER11, TYR44, TYR49, TRP50, ALA77, ASP78, ASN79, PRO81, ASN83, ASN86, ASN87, CYS90, ILE93, ILE95, LYS96, SER97, ALA100, TRP104 and CYS109 according to FIG. 5, ± a root mean square deviation from the backbone atoms of the amino acids of not more than 1.5 Å. The PSGL-1 binding site may correspond to the configuration of the P-selectin LE in its state of association with an agent, preferably, PSGL-1 or a PSGL-1 peptide, or in its unbound state.

In addition, the present invention provides a method for identifying an agent that interacts with P-selectin LE, comprising the steps of: (a) generating a three dimensional model of P-selectin LE using the relative structural coordinates according to FIGS. 2, 3 or 5, ± a root mean square deviation from the backbone atoms of said amino acids of not more than 1.5 Å; and (b) employing said three-dimensional structure to design or select an agent.

In addition, the present invention provides a method for identifying an activator or inhibitor of a molecule or molecular complex comprising an SLe^{X} binding site, comprising the steps of: (a) generating a three dimensional model of said molecule or molecular complex comprising an SLe^{X}binding site using (i) the relative structural coordinates according to FIG. 3 of residues TYR48, GLU80, ASN82, GLU92, TYR94, PRO98, SER99, ASN105, ASP106, GLU107 and bound calcium, ± a root mean square deviation from the backbone atoms of said amino acids of not more than 1.5 Å, or (ii) the relative structural coordinates according to FIG. 4 of amino acid residues TYR48, GLU80, ASN82, GLU92, TYR94, ARG97, GLU98, ASN105, ASP106, GLU107 and bound calcium, ± a root mean square deviation from the backbone atoms of said amino acids of not more than 1.5 Å; and (b) selecting or designing a candidate activator or inhibitor by performing computer fitting analysis with the three dimensional model generated in step (a). In another embodiment, the relative structural coordinates according to FIG. 3 further comprises amino acid residues TYR44, SER46, SER47, ALA77, ASP78, ASN79, PRO81, ASN83, ARG85, GLU88, CYS90, ILE93, LYS96, SER97, ALA100, TRP104, HIS108, LYS111 and LYS113, ± a root mean square deviation from the backbone atoms of said amino acids of not more than 1.5 Å. In yet another embodiment, the relative structural coordinates according to FIG. 4 further comprises the amino acid residues TYR44, SER45, PRO46, SER47, ALA77, PRO78, GLY79, PRO81, GLU88, CYS90, LYS99, ASP100, TRP104, ARG108, LYS111 and LYS113, ± a root mean square deviation from the backbone atoms of said amino acids of not more than 1.5 Å.

The present invention still further provides a method for identifying an activator or inhibitor of a molecule or molecular complex comprising a PSGL-1 binding site, comprising the steps of: (a) generating a three dimensional model of said molecule or molecular complex comprising a PSGL-1 binding site using the relative structural coordinates according to FIG. 5 of amino acid residues ALA9, TYR45, SER46, SER47, TYR48, GLU80, ASN82, LYS84, ARG85, GLU88, GLU92, TYR94, PRO98, SER99, ASN105, ASP106, GLU107, HIS108, LEU110, LYS111, LYS112, LYS113, HIS114 and bound strontium, ± a root mean square deviation from the backbone atoms of said amino acids of not more than 1.5 Å; and (b) selecting or designing a candidate activator or inhibitor by performing computer fitting analysis with the three dimensional model generated in step (a). In another embodiment, the relative structural coordinates according to FIG. 5 further comprises amino acid residues SER6, THR7, LYS8, TYR10, SER11, TYR44, TYR49, TRP50, ALA77, ASP78, ASN79, PRO81, ASN83, ASN86, ASN87, CYS90, ILE93, ILE95, LYS96, SER97, ALA100, TRP104 and CYS109, ± a root mean square deviation from the backbone atoms of said amino acids of not more than 1.5 Å.

In addition, the present invention provides a method for identifying an agent that interacts with SLe^{X}, comprising the steps of: (a) generating a three dimensional model of SLe^{X} using the relative structural coordinates according to FIG. 3 or 4, ± a root mean square deviation from the backbone atoms of said amino acids of not more than 1.5 Å; and (b) employing said three-dimensional structure to design or select an agent that interacts with SLe^{X}.

Still further, the present invention provides a method for identifying an agent that interacts with PSGL-1, comprising the steps of: (a) generating a three dimensional model of a PSGL-1 peptide using the relative structural coordinates according to FIG. 5, ± a root mean square deviation from the backbone atoms of said amino acids of not more than 1.5 Å; and (b) employing said three-dimensional structure to design or select an agent that interacts with PSGL-1.

The present invention also provides agents, activators or inhibitors identified using the foregoing methods. Small molecules or other agents which inhibit or otherwise interfere with the selectin-mediated cellular rolling of leukocytes over vascular tissue may be useful in the treatment of diseases involving abnormal inflammatory responses such as asthma and psoriasis.

Finally, the present invention provides a method for obtaining a crystallized complex of an E-selectin type molecule and a compound that coordinates calcium. The method comprises the steps of: (a) contacting a crystallized E-selectin type molecule with a compound that coordinates calcium in the presence of calcium ions and PEG to form a crystallized complex of the E-selectin type molecule and the compound that coordinates calcium; and (b) contacting the crystallized complex in the presence of a reduced concentration of calcium ions, and sufficient concentrations of PEG and an ionic salt to obtain a final crystallized complex, that upon cooling, is suitable for elucidating the three dimensional structures of the E-selectin type molecule and the compound that coordinates calcium by x-ray diffraction of the final crystallized complex.

Additional objects of the present invention will be apparent from the description which follows.

### BRIEF DESCRIPTION OF THE FIGURES

FIGS. 1A, 1B and 1C depict the resolution and BIAcore affinity analysis of the PSGL-1 19ek peptides.

FIG. 1A: Profile of the PSGL-1 19ek peptides resolved by anion-exchange chromatography. See text for definition of peak labels. Inset. Structure of the major PSGL-1 19ek peptide SGP-3.<Q denotes cyclization of the N-terminal Gln residue to pyroglutamate and SO₃ represents sulfation of Tyr residues. The over line in the numbered peptide sequence indicates residues of non-PSGL-1 origin that are associated with the enterokinase linker region.

FIGS. 1B-1 and 1B-2: Representative BIAcore sensorgrams of solution-phase P-LE binding to immobilized PSGL-1 constructs. P-LE (at 800 nM concentration) was injected over sPSGL (FIGS. 1B-1) and SGP-3 (FIGS. 1B-2). Binding signals of P-LE injected other less modified forms of SGP-3 (not shown) were identical to these results with regards to binding kinetics. P-LE binding to sPSGL and SGP-3 was inhibited by co-injection with solution-phase SGP-3 (dotted line) but not by a synthetic peptide containing no tyrosine sulfation or glycosylation (dashed line), both at 20 μM concentration. All curves reflect specific binding produced by subtracting non-specific binding from total binding.

FIGS. 1C-1-1C-6: Binding affinity determinations of solution-phase P-LE reacted with immobilized sPSGL and purified 19ek peptides by BIAcore analysis. First row, left-to-right: sPSGL (FIGS. 1C-1), SGP-3 (FIGS. 1C-2) and SGP-2 (FIGS. 1C-3). Second row, left-to-right: SGP-1 (FIGS. 1C-4), GP-1 (FIGS. 1C-5) and SP-1 (FIGS. 1C-6). P-LE at the indicated concentrations was reacted with immobilized ligands (to determine the total binding signal) and against control cells containing no ligand (to determine non-specific binding). The equilibrium responses (Req) at each concentration of P-LE are shown. At each P-LE concentration tested, specific binding signals (squares) were determined by subtracting non-specific responses (triangles) from total binding signals (diamonds). K_{D}s and standard deviations (shown) were determined by line-fitting of specific binding curves using BIAevaluation software (BIAcore) and are the products of three-to-ten separate experiments. K_{D} determinations using line-fitting agreed well with values determined by linear regression analysis of Scatchard plots (not shown).

FIG. 2 provides the atomic structural coordinates for P-selectin LE as derived by X-ray diffraction of a P-selectin LE crystal. “Atom type” refers to the atom whose coordinates are being measured. “Residue” refers to the type or residue of which each measure atom is a part—i.e., amino acid, cofactor, ligand or solvent. The “x, y and z” coordinates indicate the Cartesian coordinates of each measured atom's location in the unit cell (Å). “Occ” indicates the occupancy factor. “B” indicates the “B-value”, which is a measure of how mobile the atom is in the atomic structure (Å²). “MOL” indicates the segment identification used to identify each molecule in the crystal. Under “MOL”, “MOLA”, “MOLB”, “MOLC” and “MOLD” refers to each molecule of P-selectin LE, “SOLV” refers to water molecules, “MPDS” refers to MPD molecules and “CALS” refers to calcium ions. Due to disordered structures, Lys17 (MOLA), Lys17 (MOLC) and Asn57 (MOLC) of P-selectin are represented as alanines.

FIG. 3 provides the atomic structural coordinates for P-selectin LE and SLe^{X} as derived by X-ray diffraction of a P-selectin LE: SLe^{X} crystal. Figure headings are as noted for FIG. 2, except that under “MOL”, “A”, “B”, “C” and “D” refers to each molecule of P-selectin LE, and SLe^{X}, MDP molecules and calcium ions are not labeled under “MOL”. However, SLe^{X}, MDP molecules and calcium ions are identified under “Residue”. Due to disordered structures, Lys17 (MOLA), Lys17 (MOLC) and Asn57 (MOLC) of P-selectin are represented as alanines.

FIG. 4 provides the atomic structural coordinates for E-selectin LE and SLe^{X} as derived by X-ray diffraction of a P-selectin LE: SLe^{X} crystal. Figure headings are as noted for FIG. 2, except that under “MOL”, “SOLV” refers to water molecules, and E-selectin LE, SLe^{X} and calcium are not labeled under “MOL”. However, E-selectin LE, SLe^{X} and calcium are identified under “Residue”.

FIG. 5 provides the atomic structural coordinates for P-selectin LE and PSGL-1 peptide as derived by X-ray diffraction of a P-selectin:PSGL-1 peptide crystal. Figure headings are as noted for FIG. 2, except that FIG. 5 does not include a “MOL” heading. However, each molecule of P-selectin LE, PSGL-1 peptide, water and MPD are identified under “Residue”. Due to disordered structures, Asn57 (MOLA), Lys58 (MOLA), Asn71 (MOLA), Arg22 (MOLB), Asn57 (MOLB), Lys58 (MOLB), Glu72 (MOLB), Met125 (MOLB) and Arg157 (MOLB) of P-selectin are represented as alanines.

FIGS. 6A, 6B and 6C provide the amino acid sequences of P-selectin (FIG. 6A), E-selectin (FIG. 6B) and PSGL-1 (FIG. 6C). The segments of the sequences used to make the constructs in the crystals are underlined.

### DETAILED DESCRIPTION OF THE INVENTION

As used herein, the following terms and phrases shall have the meanings set forth below:

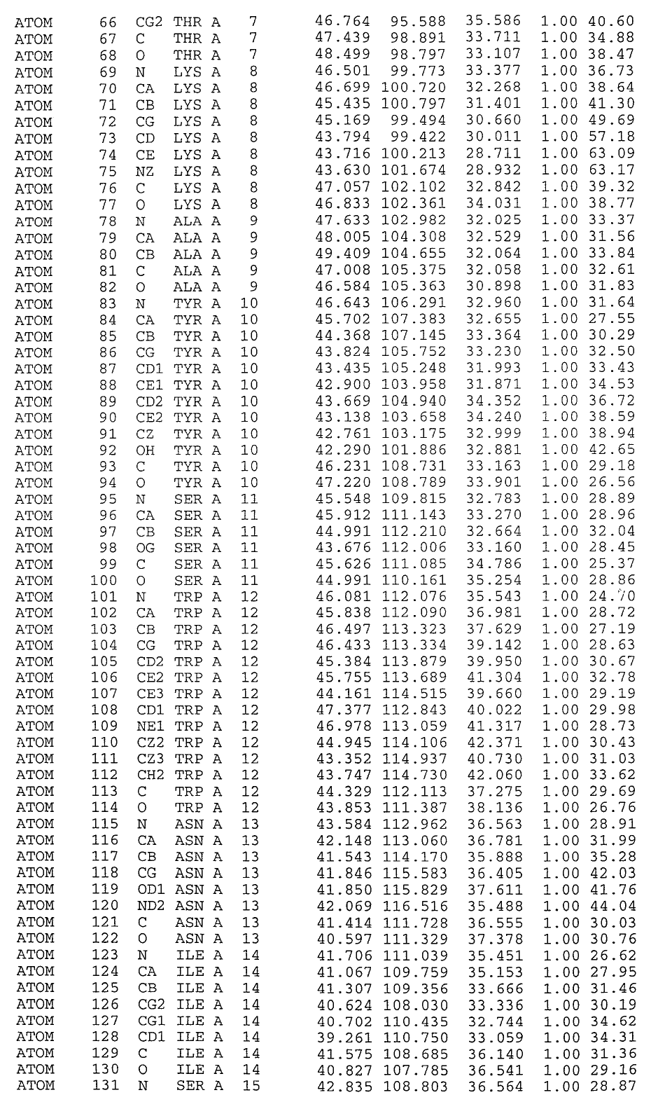

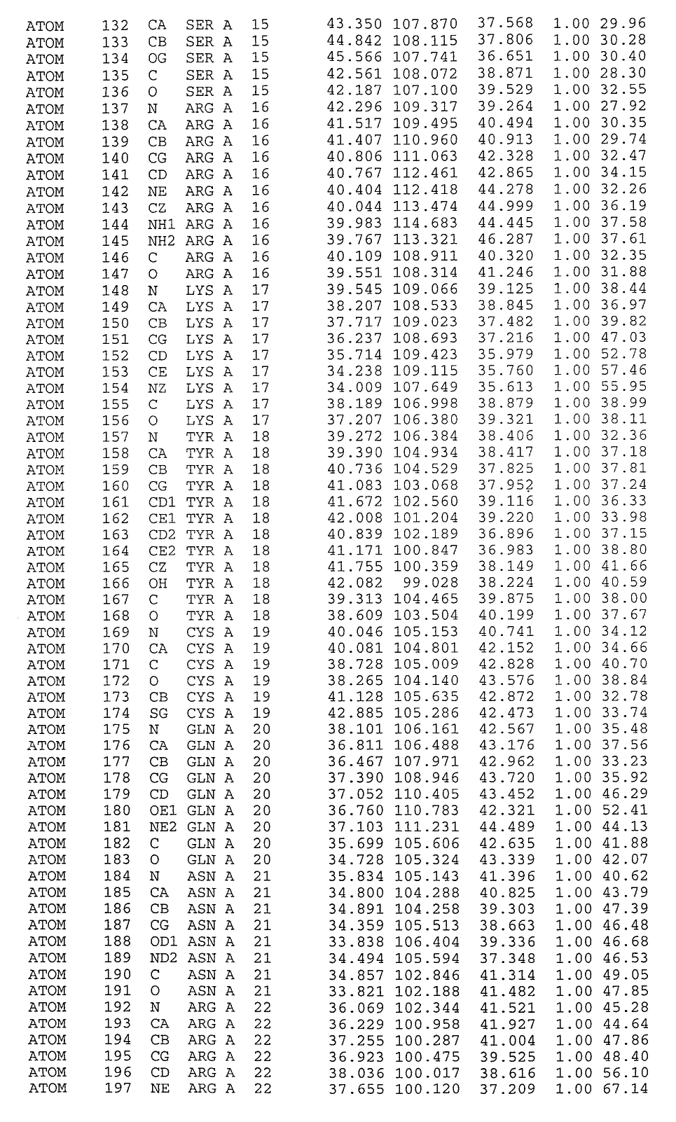

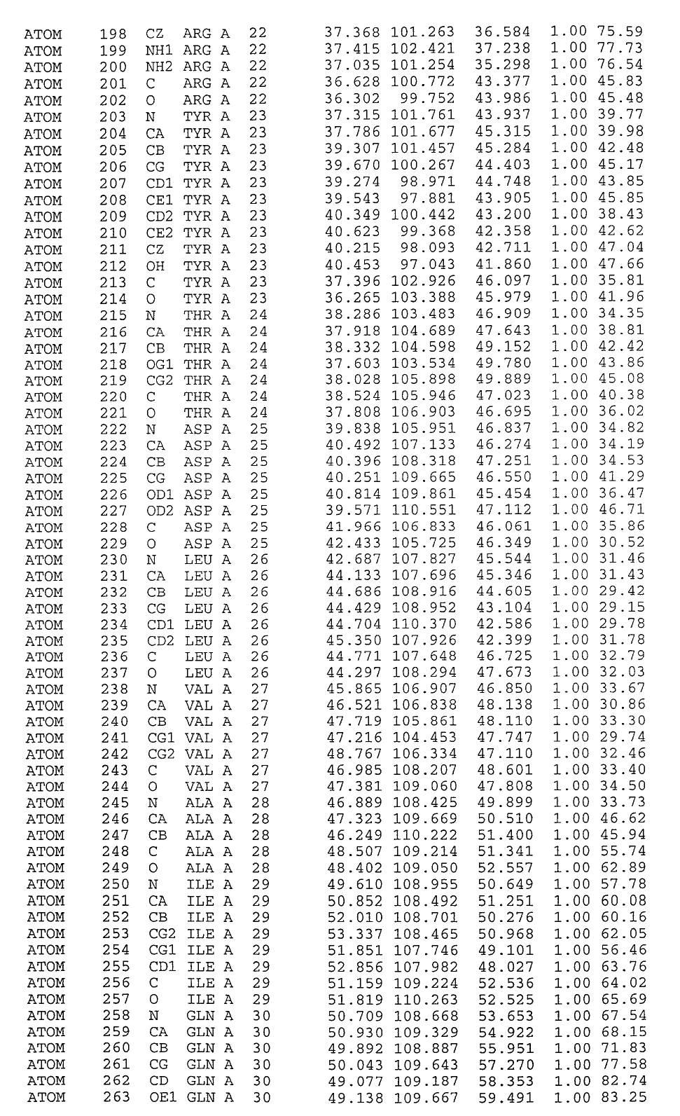

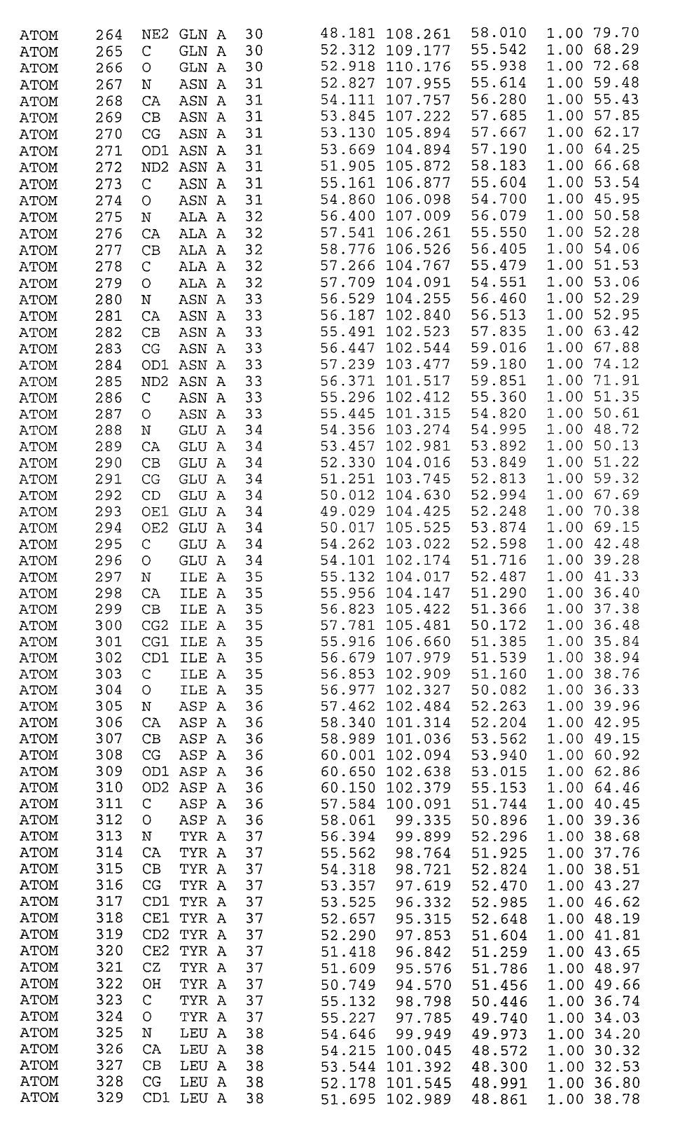

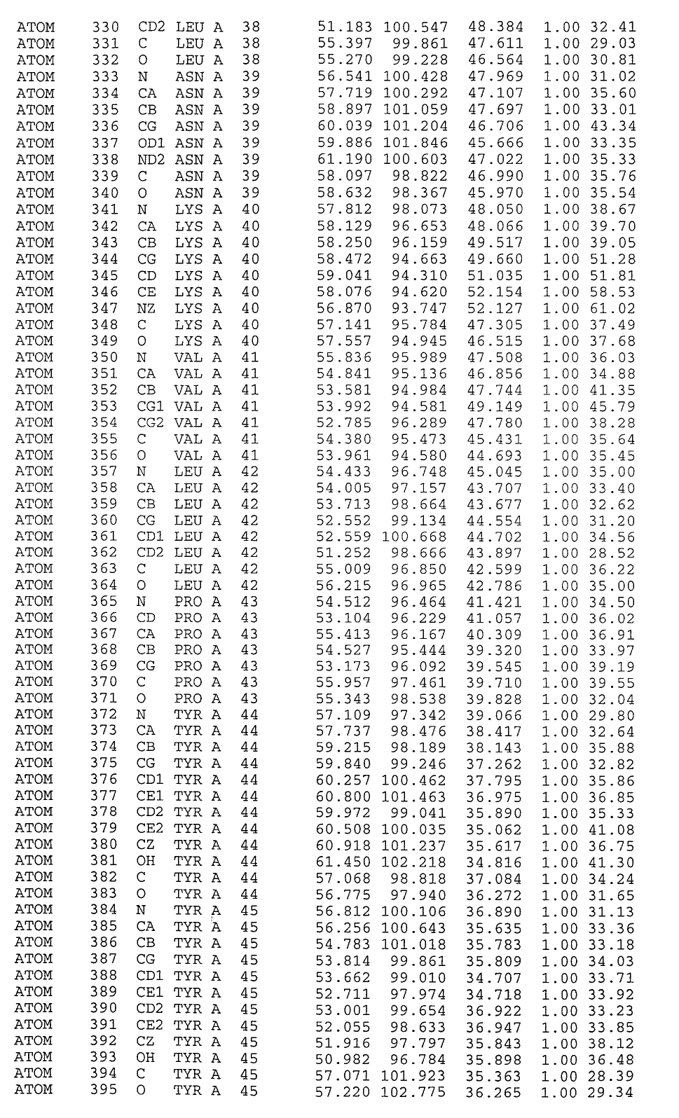

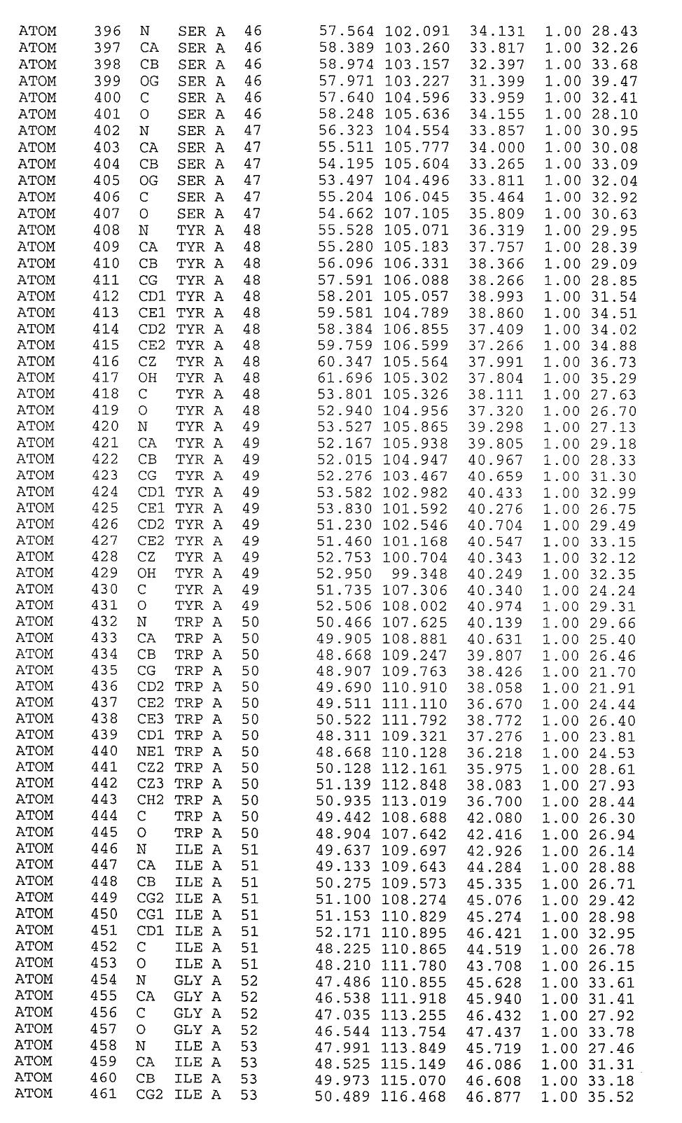

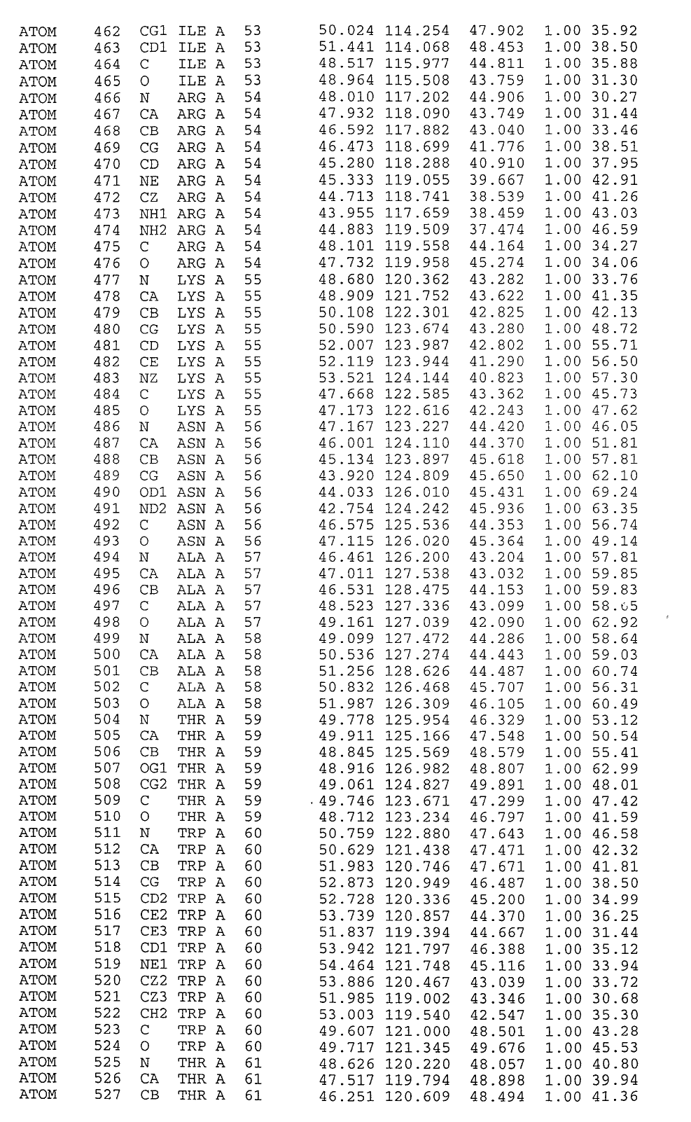

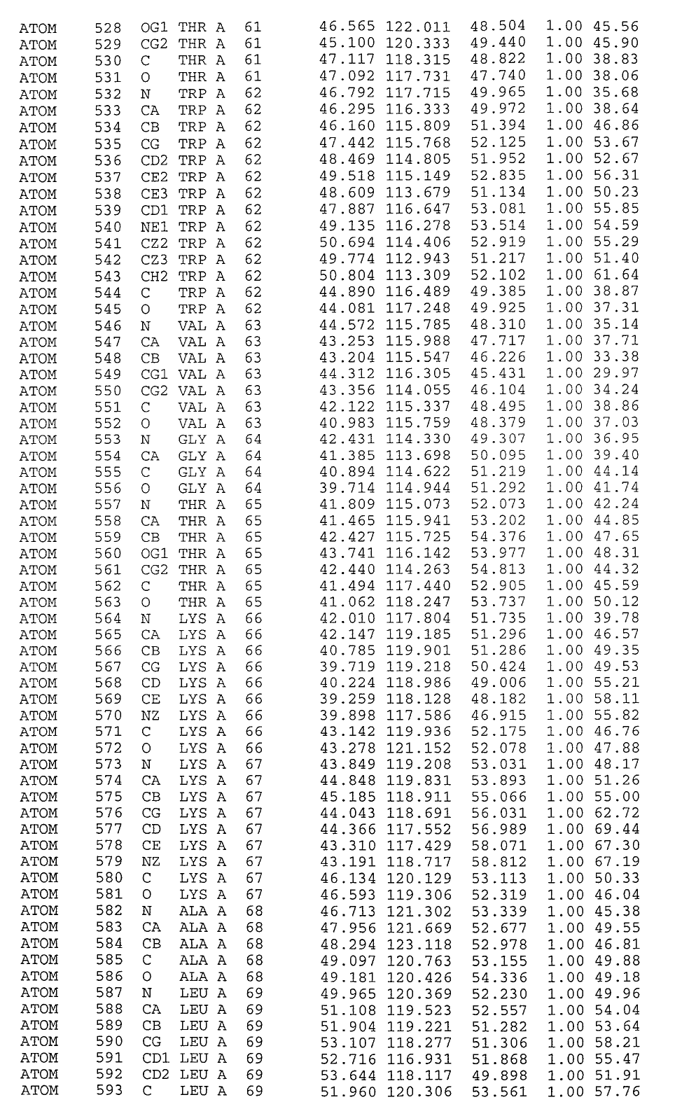

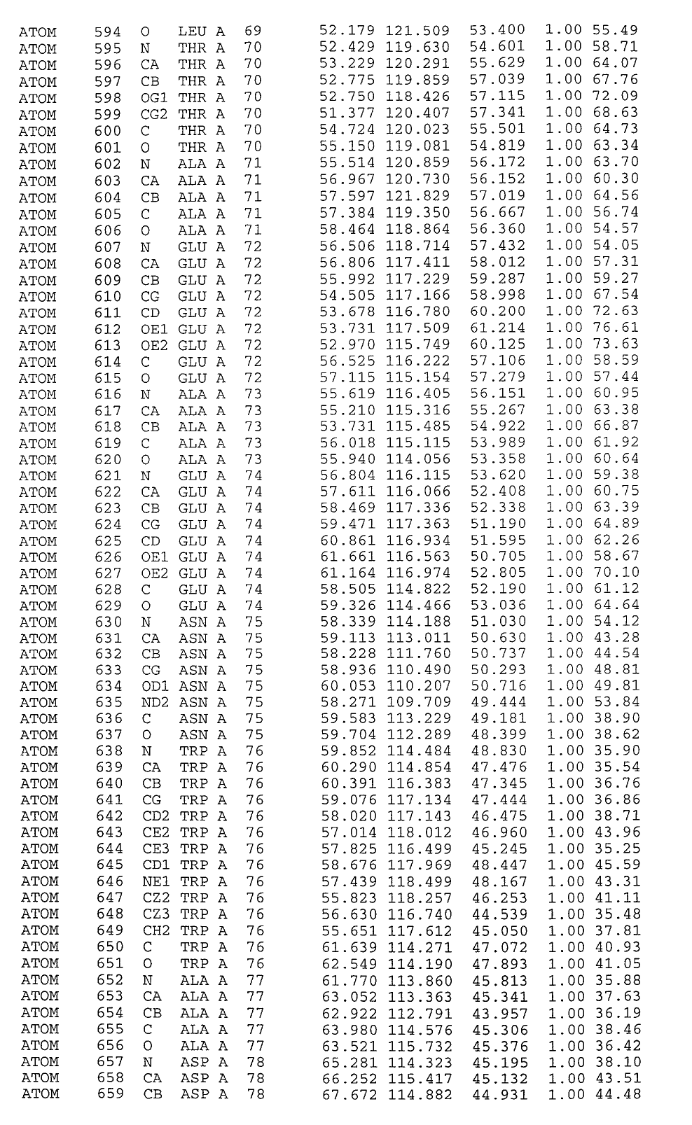

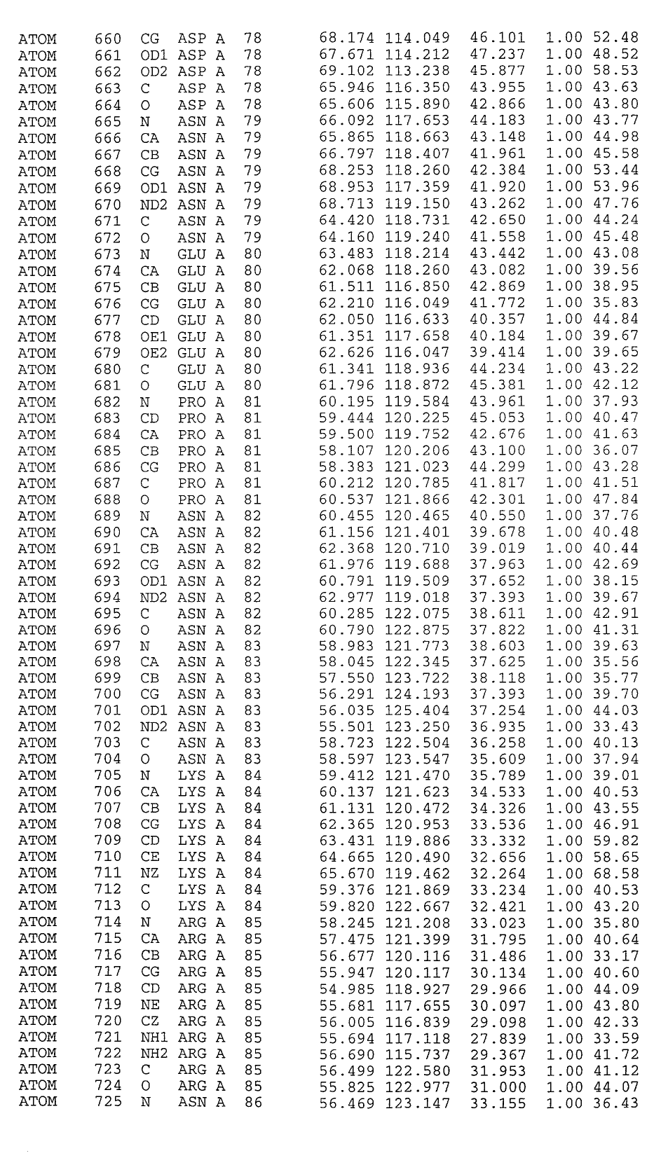

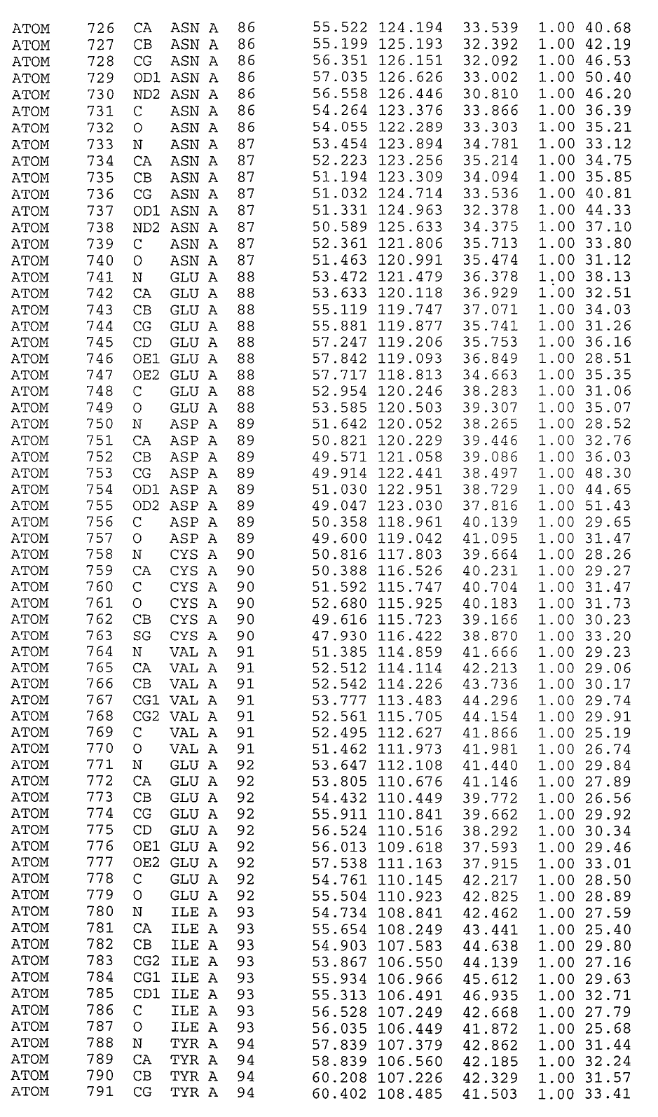

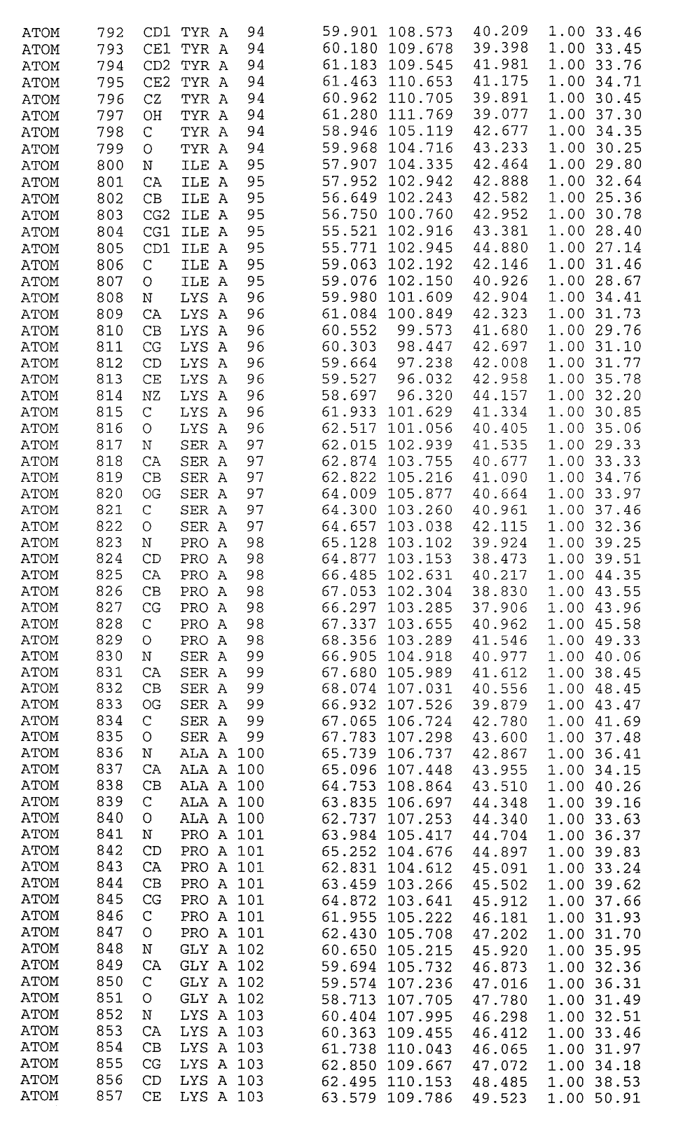

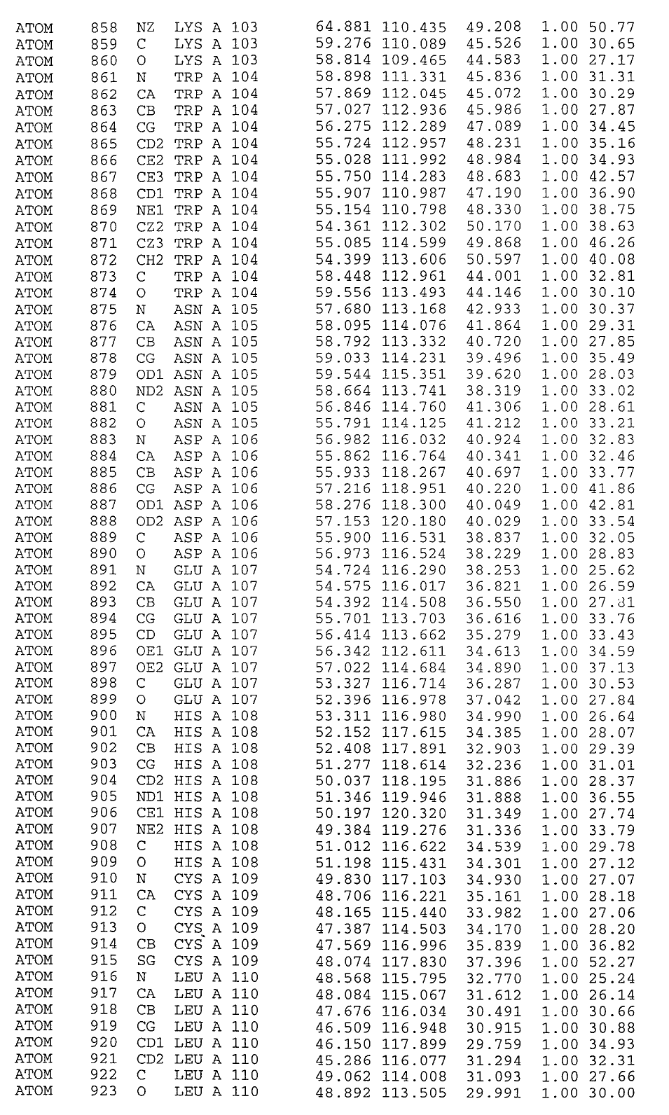

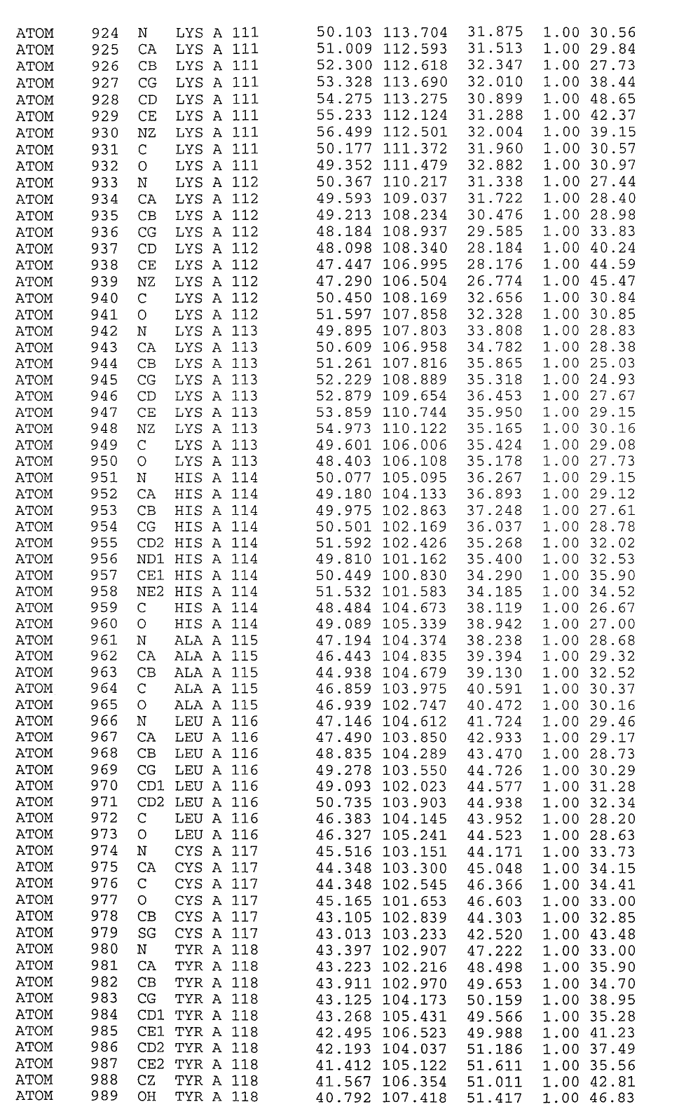

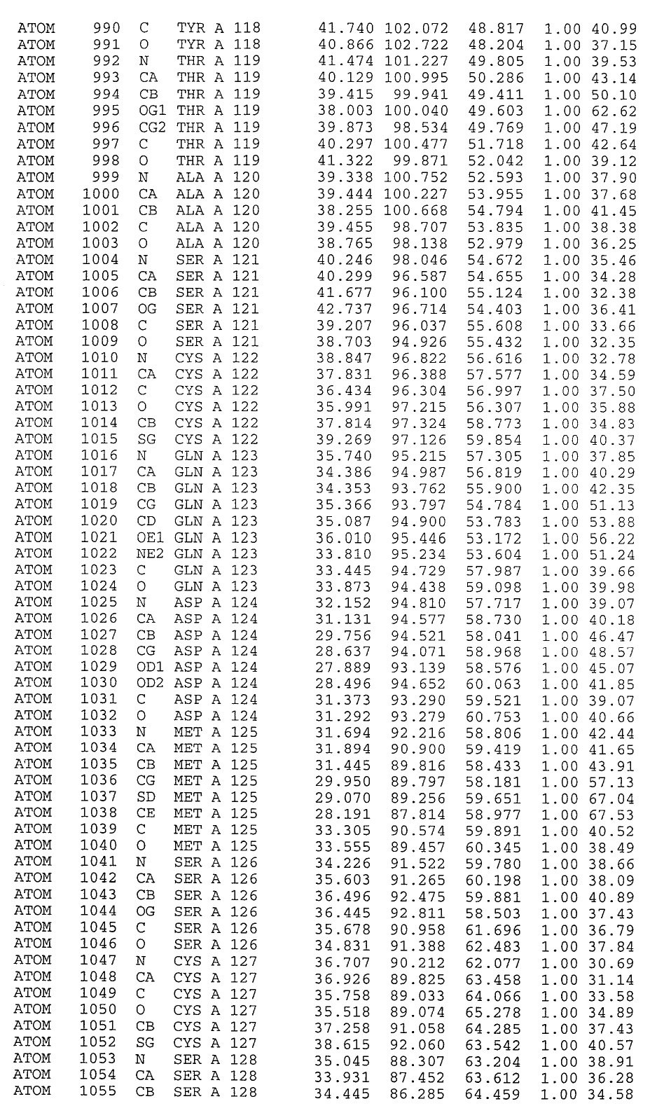

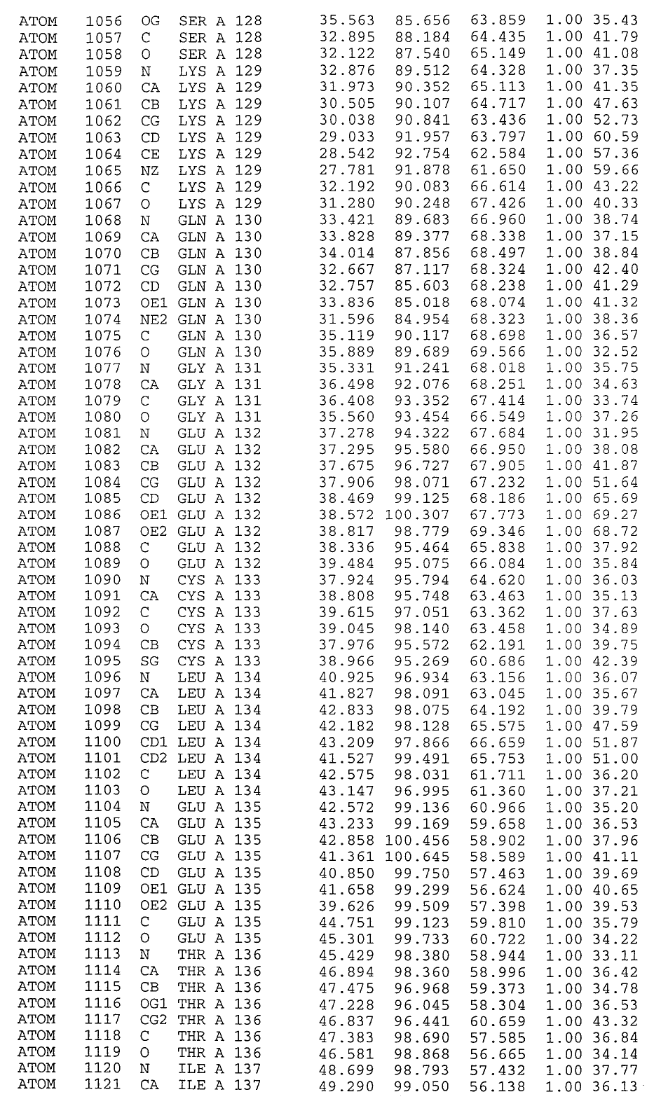

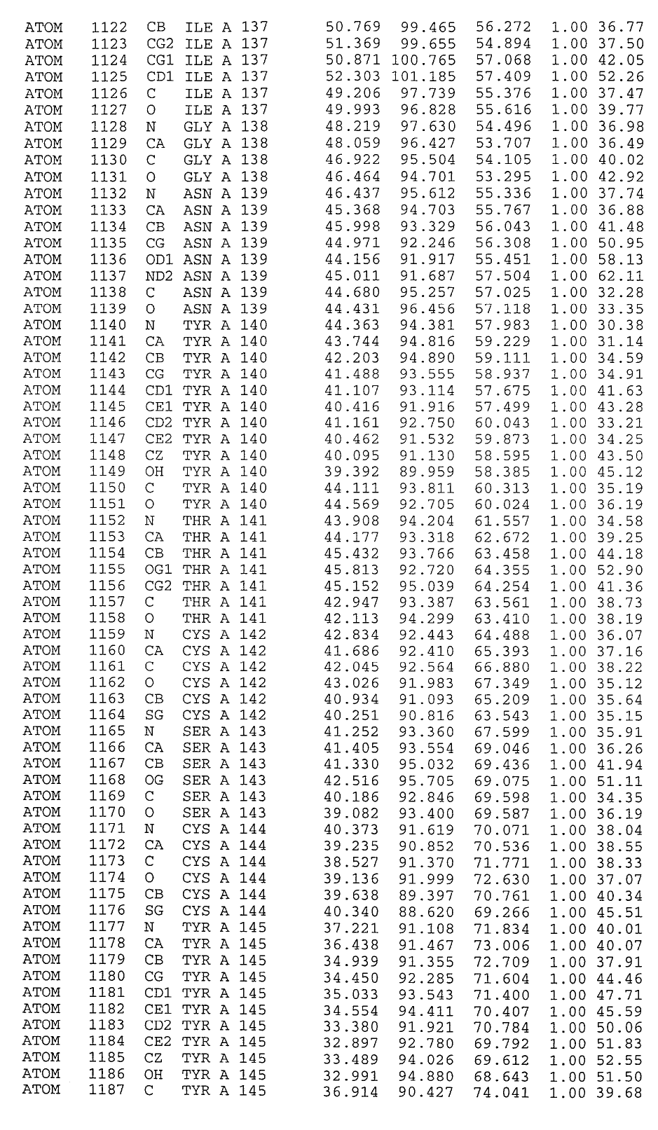

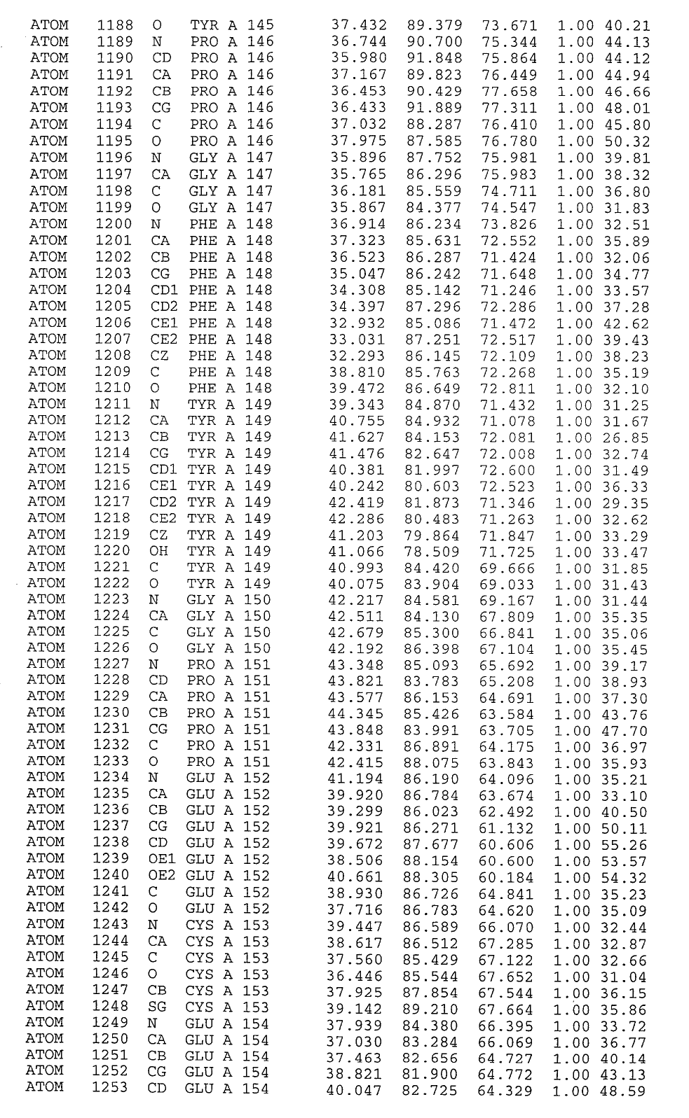

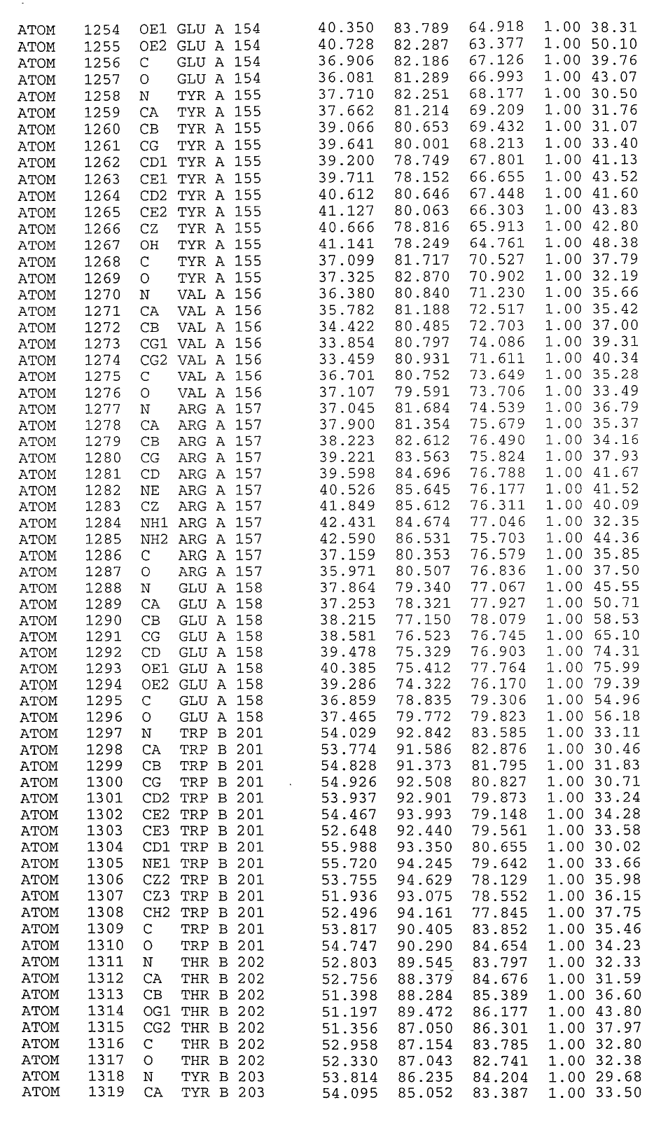

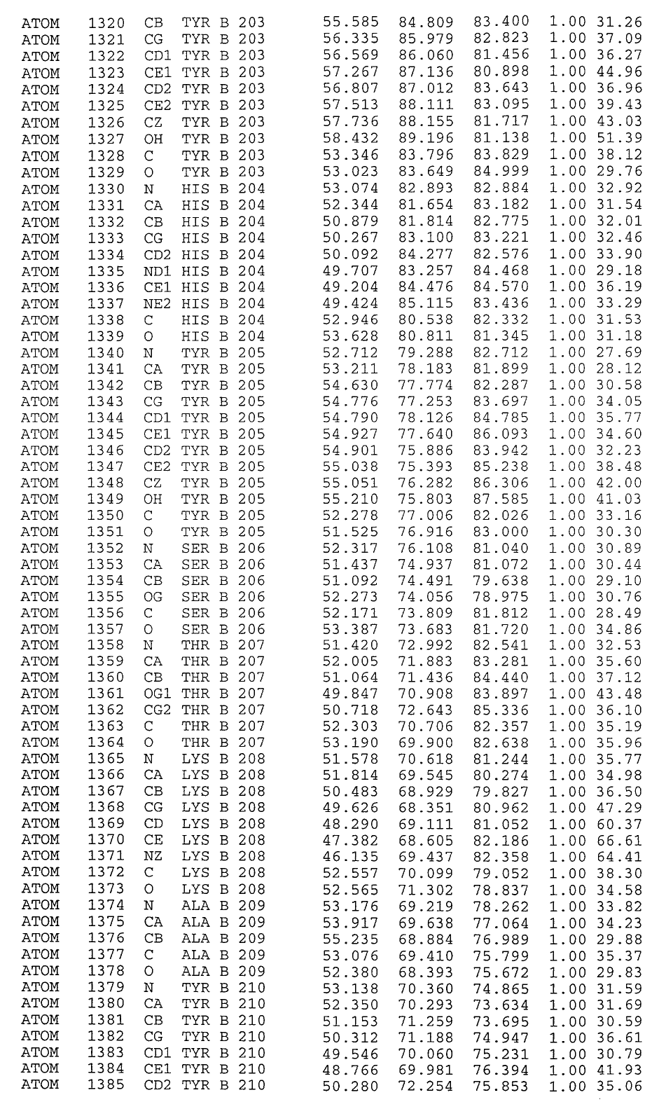

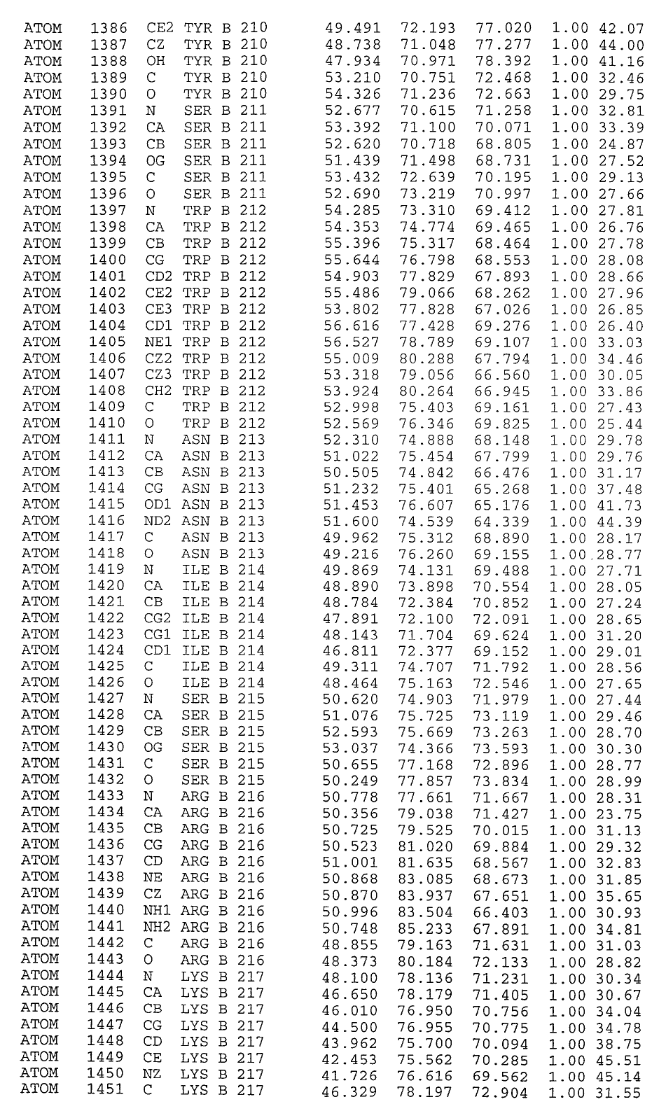

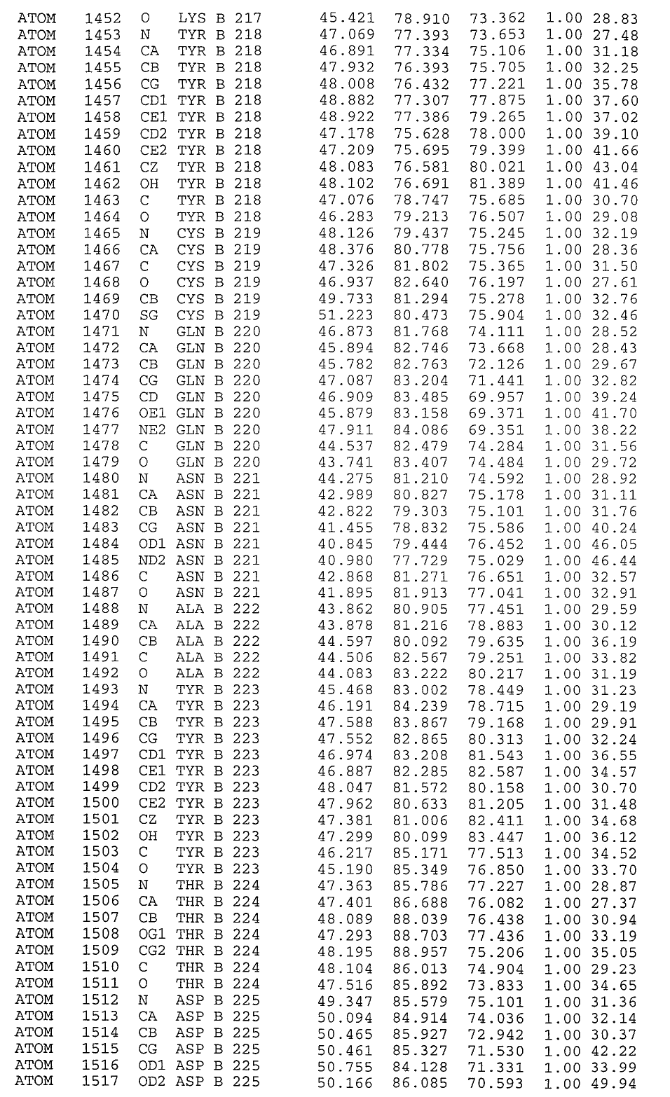

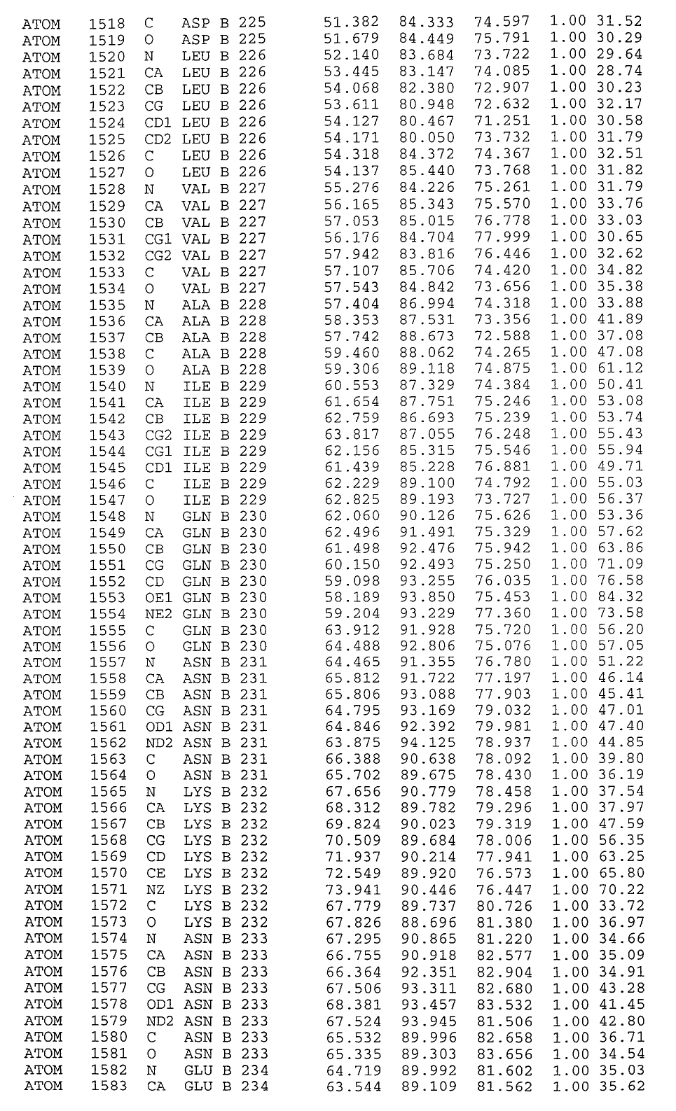

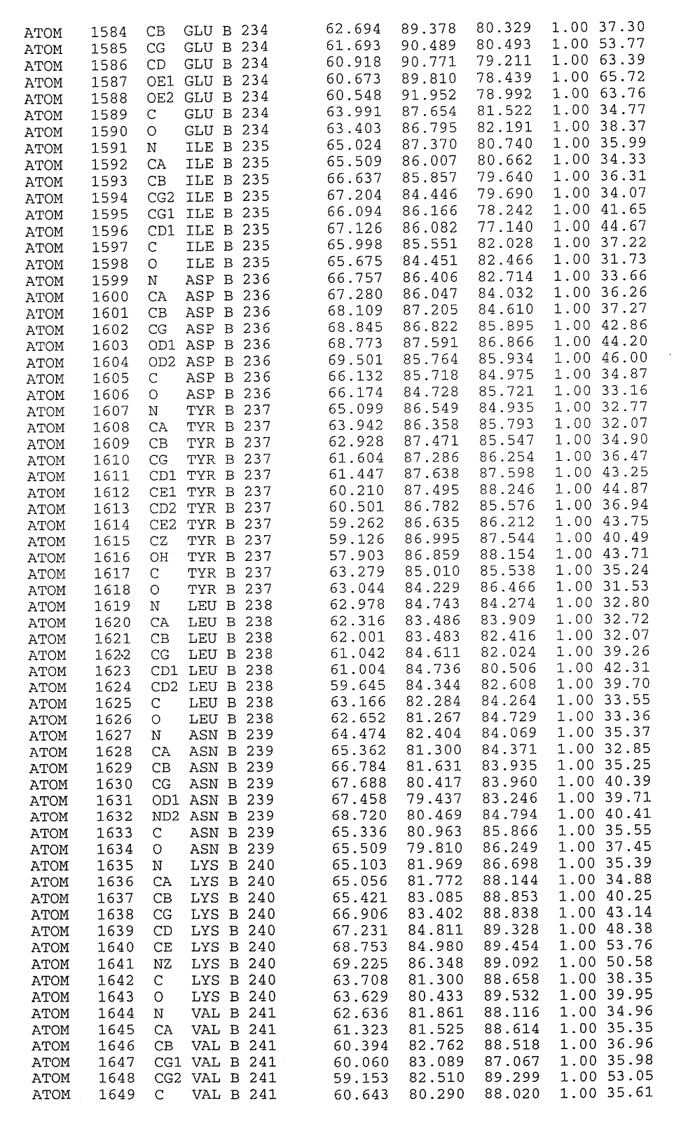

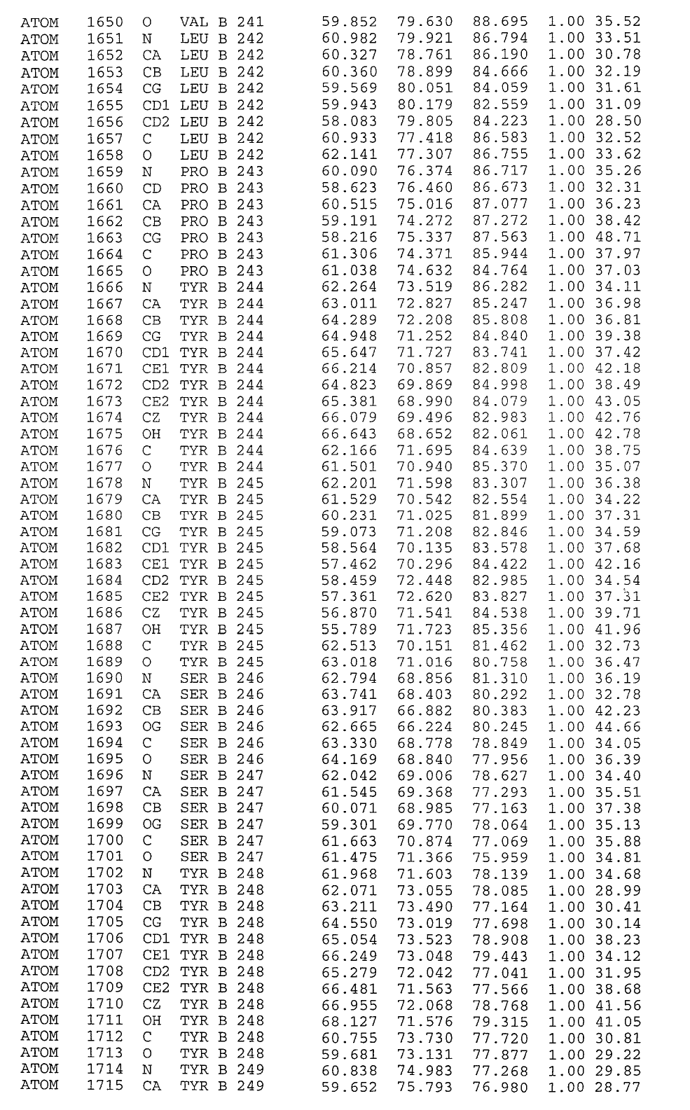

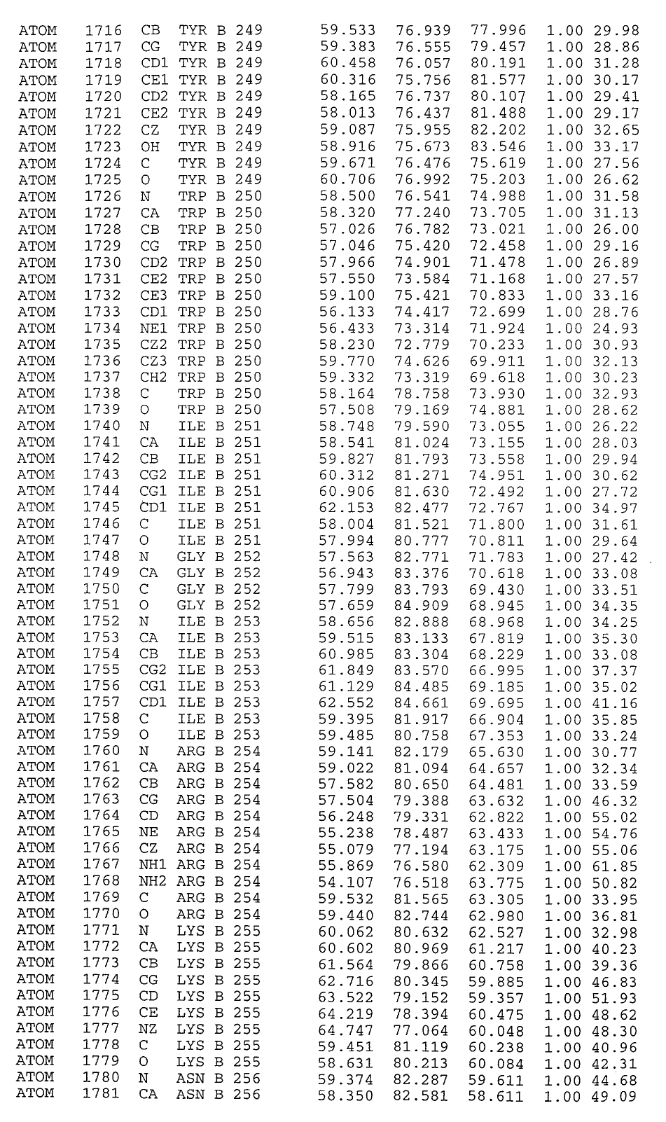

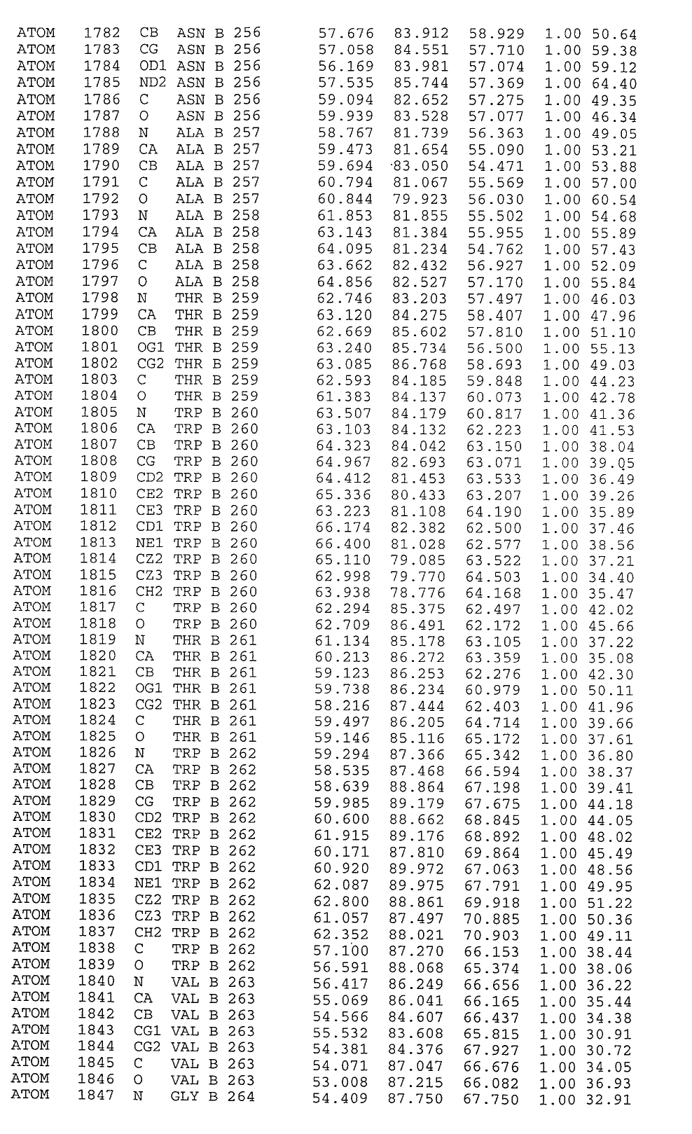

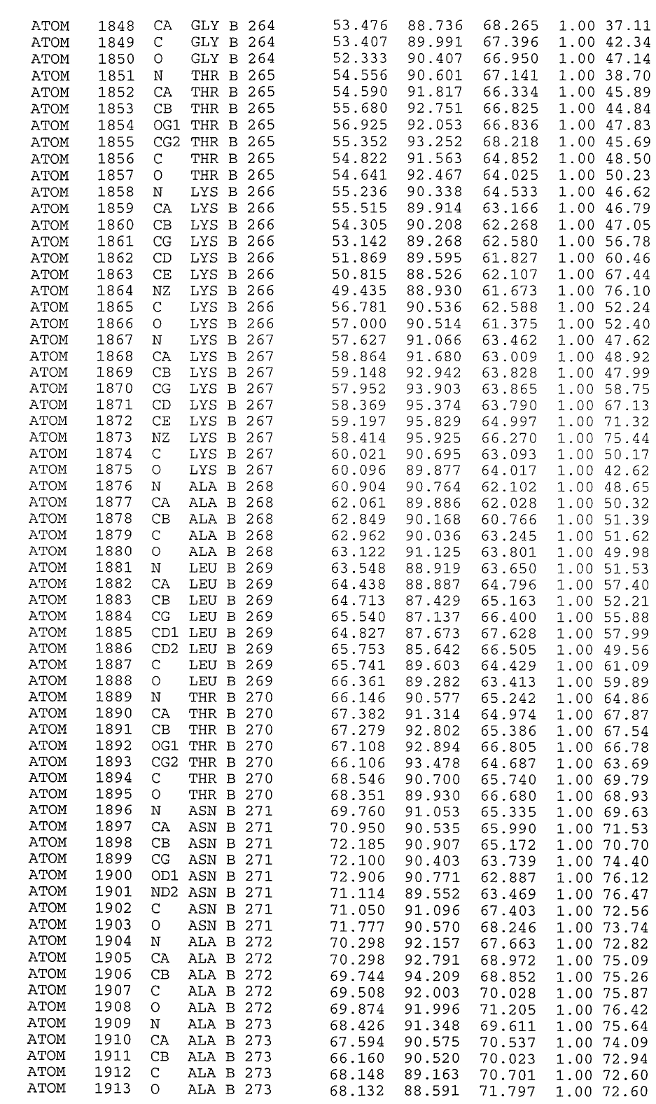

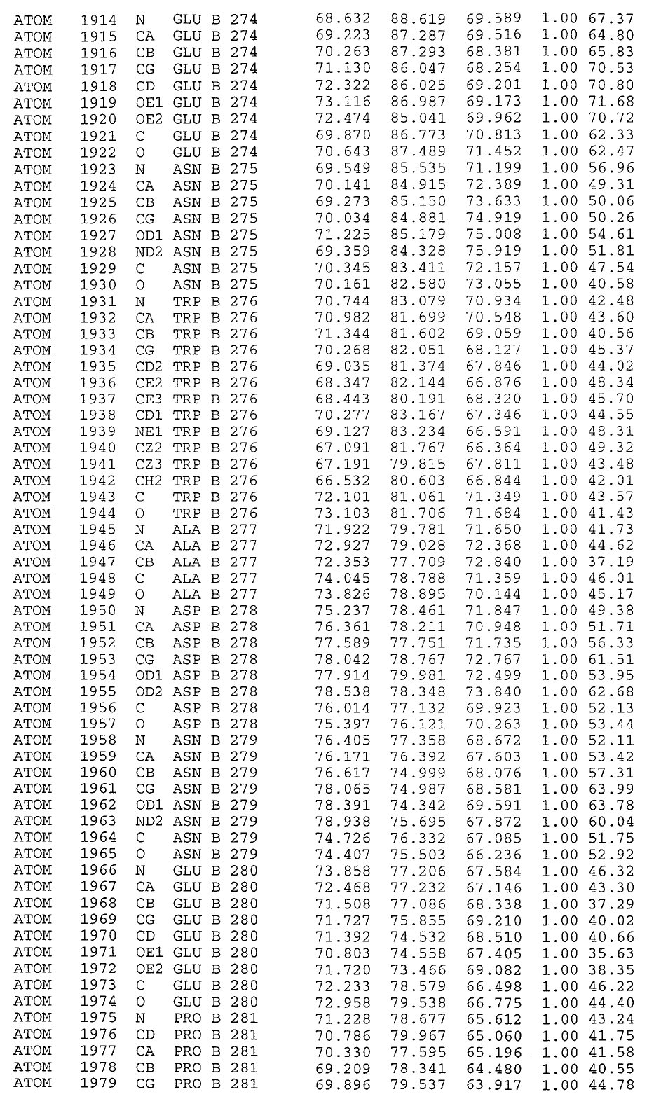

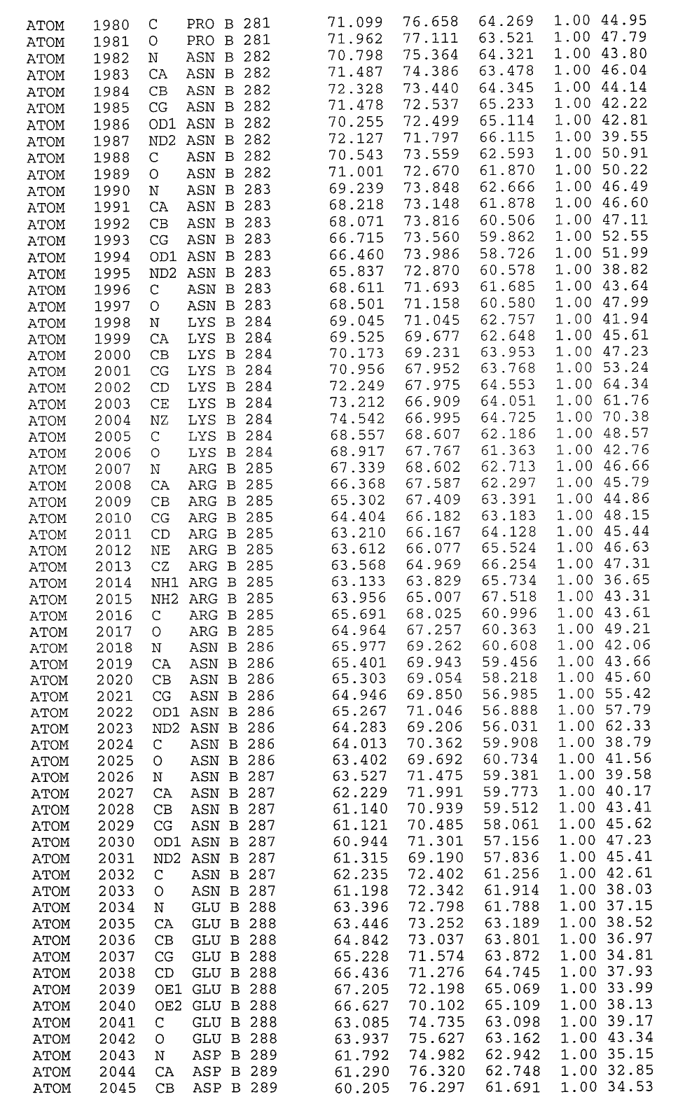

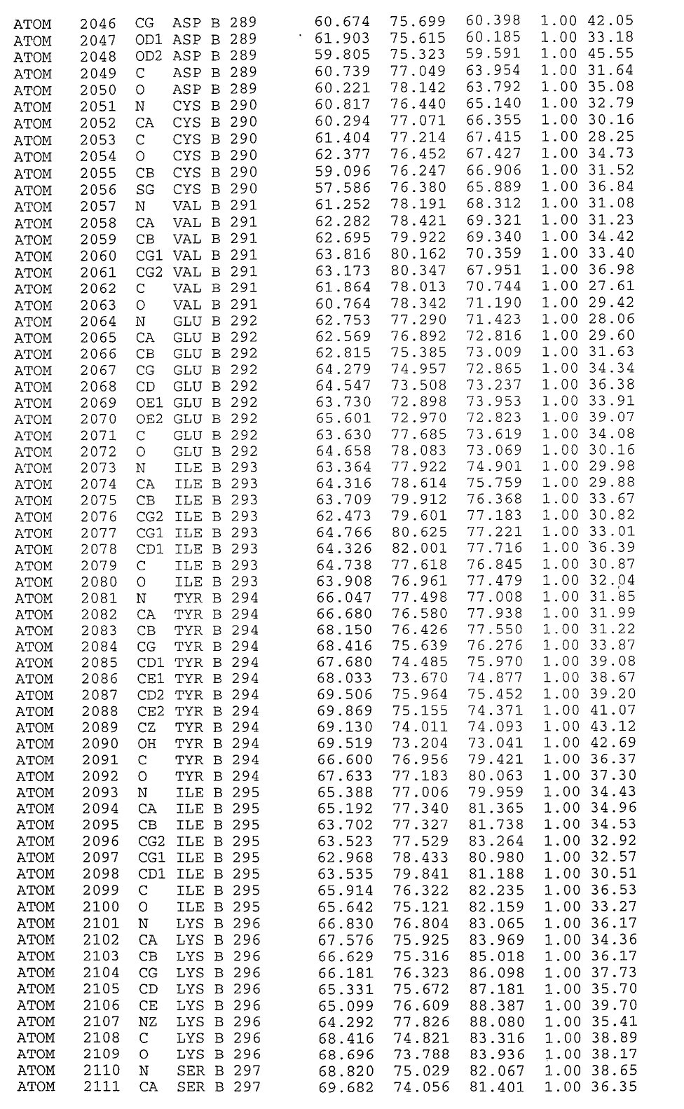

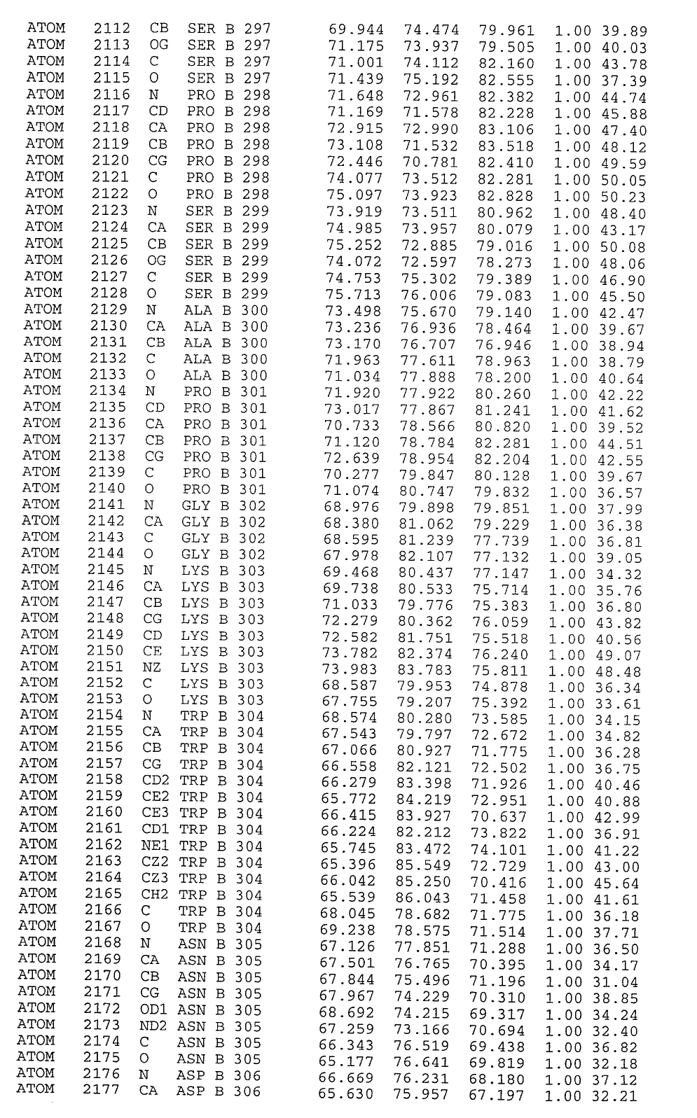

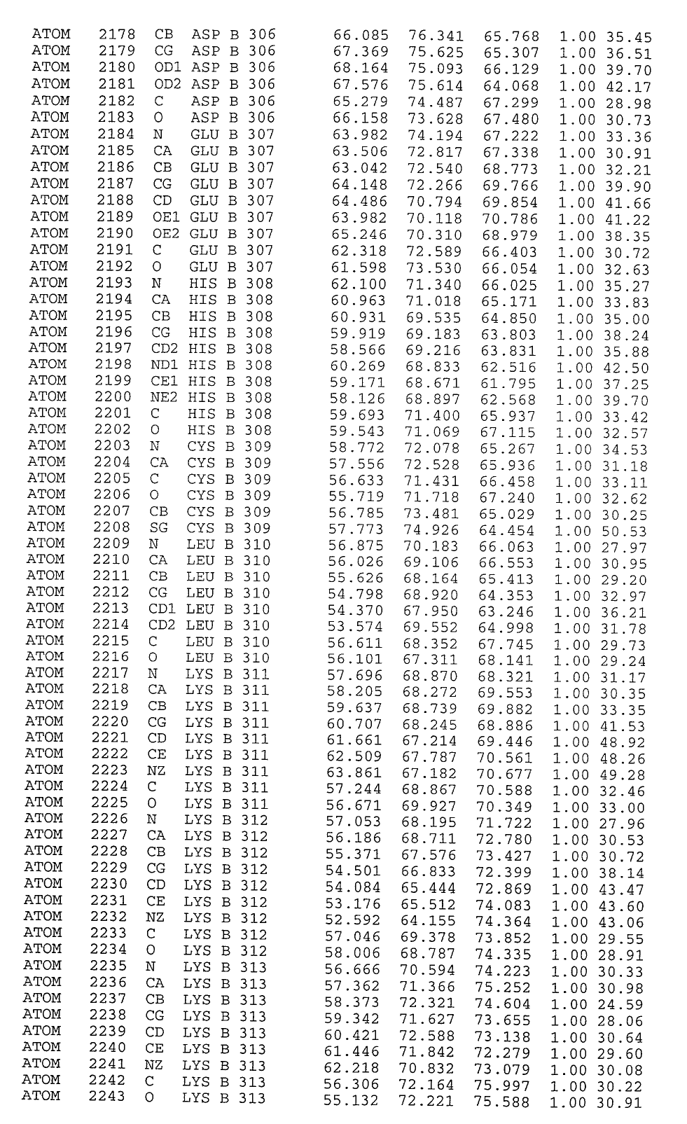

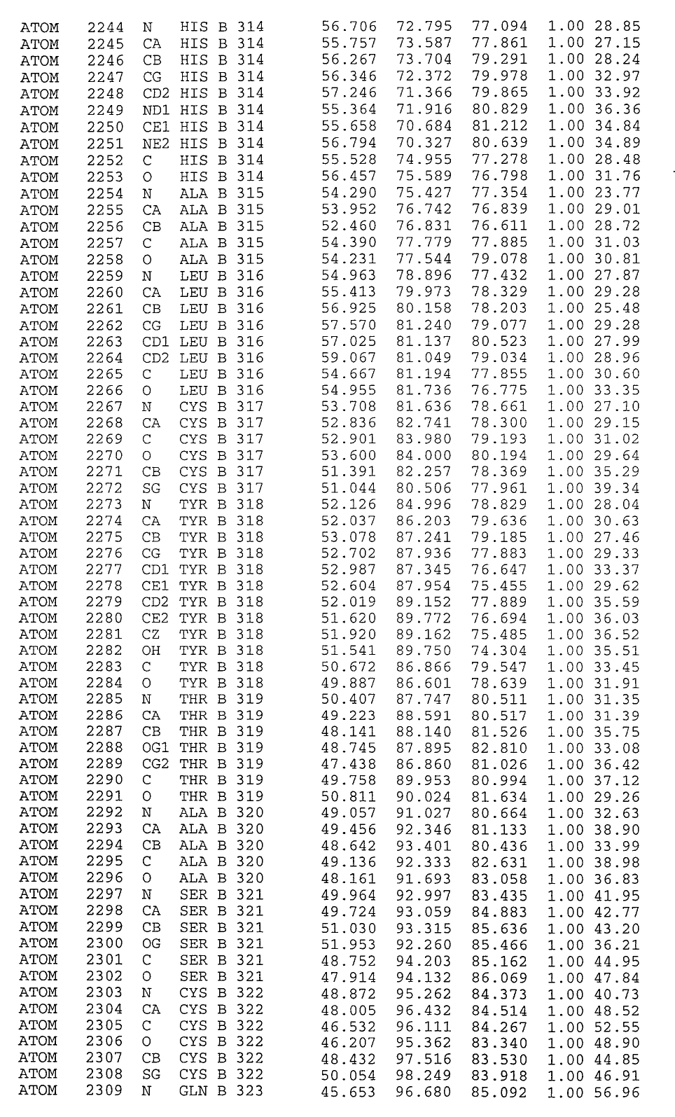

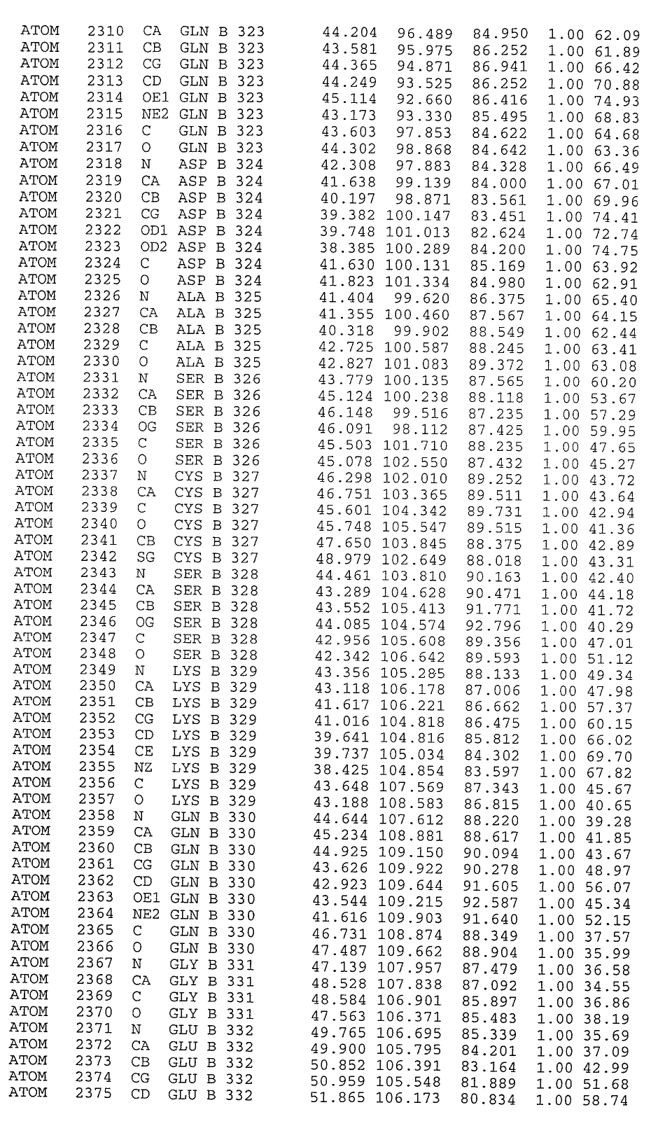

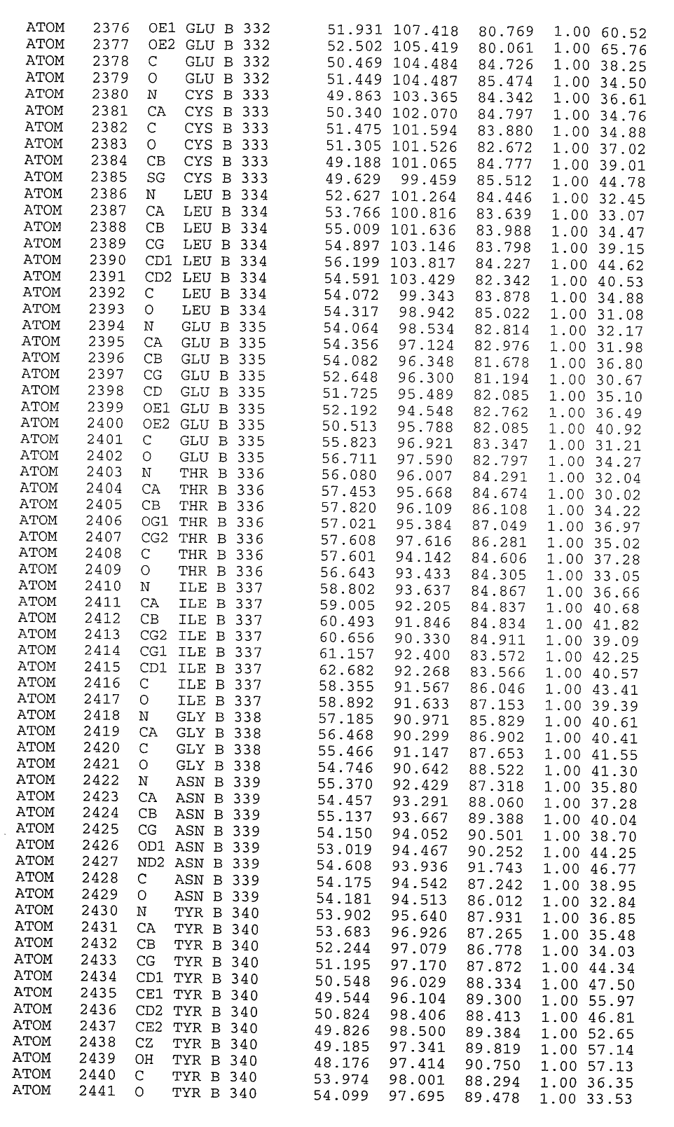

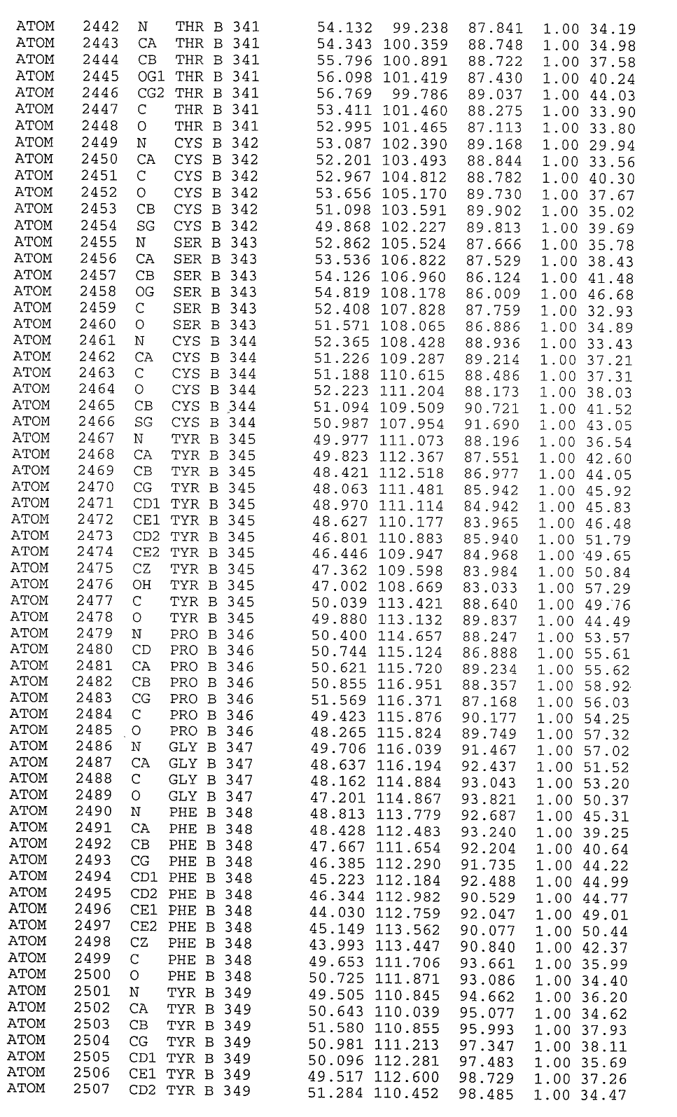

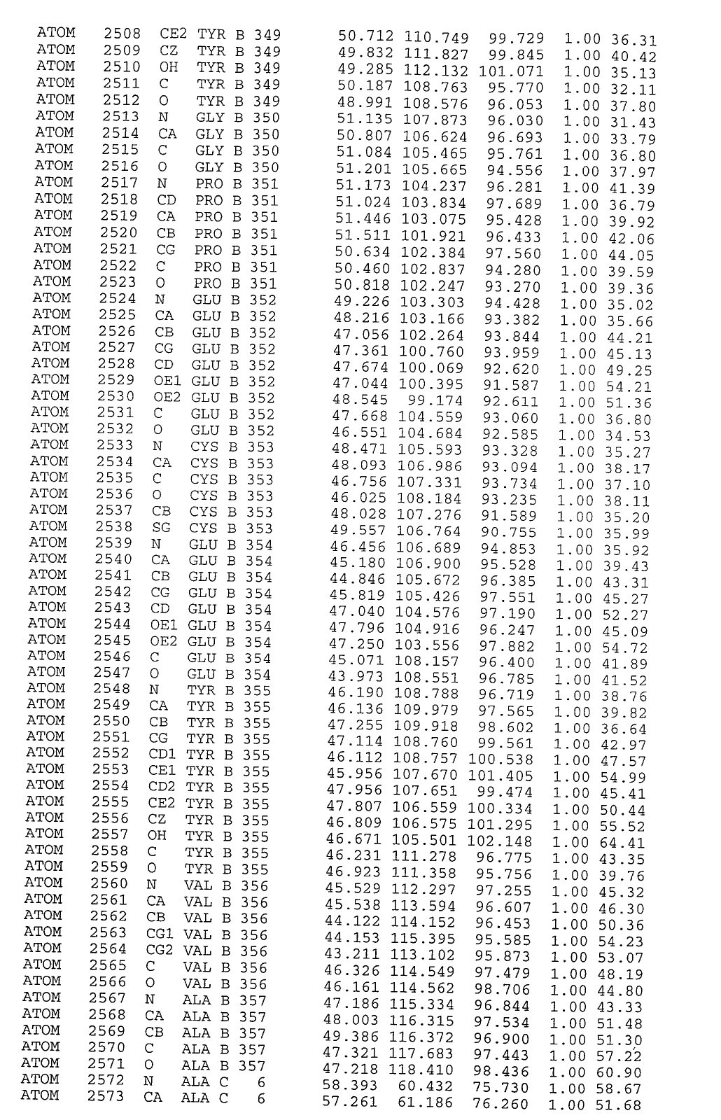

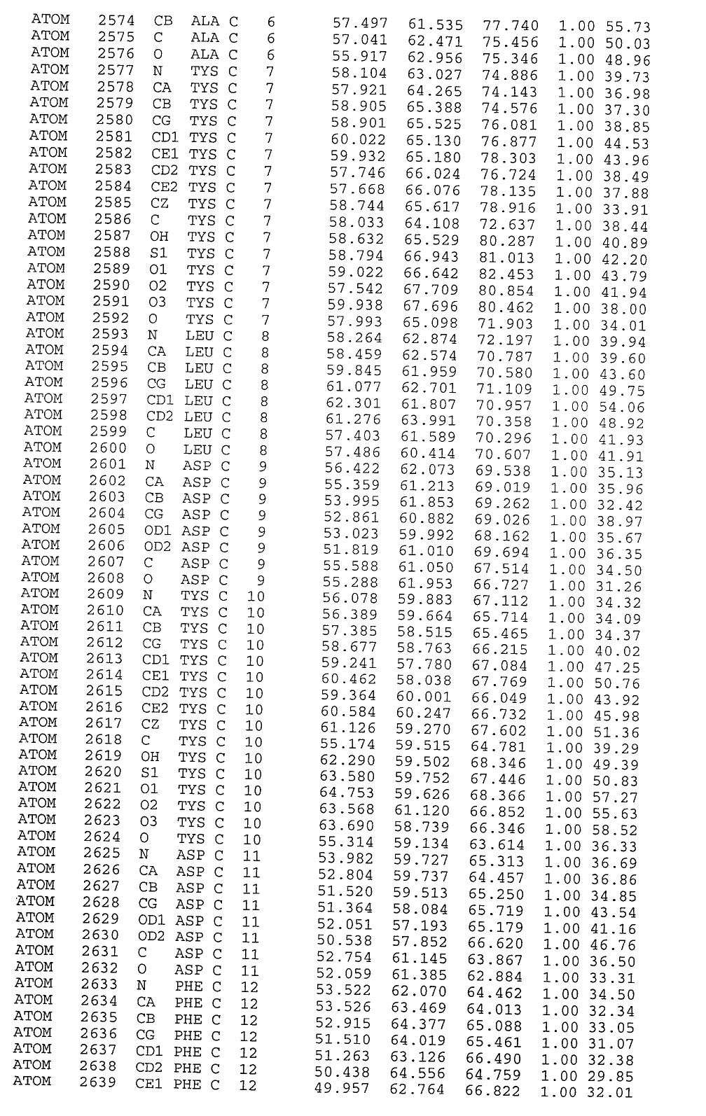

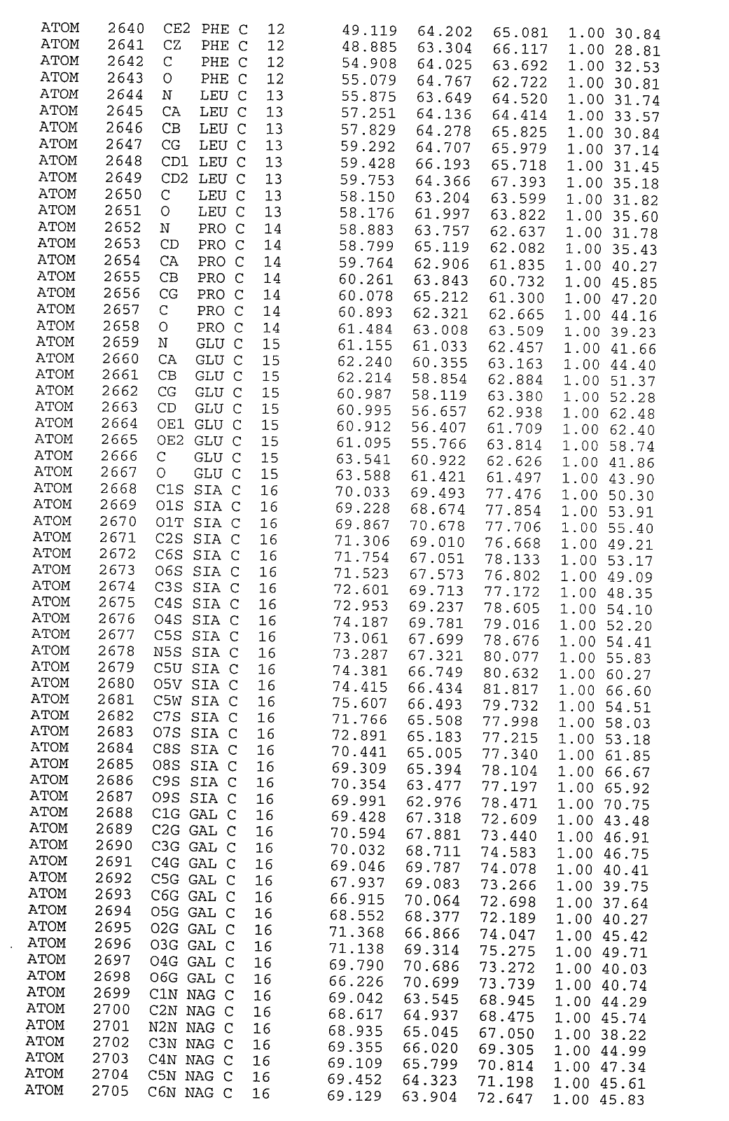

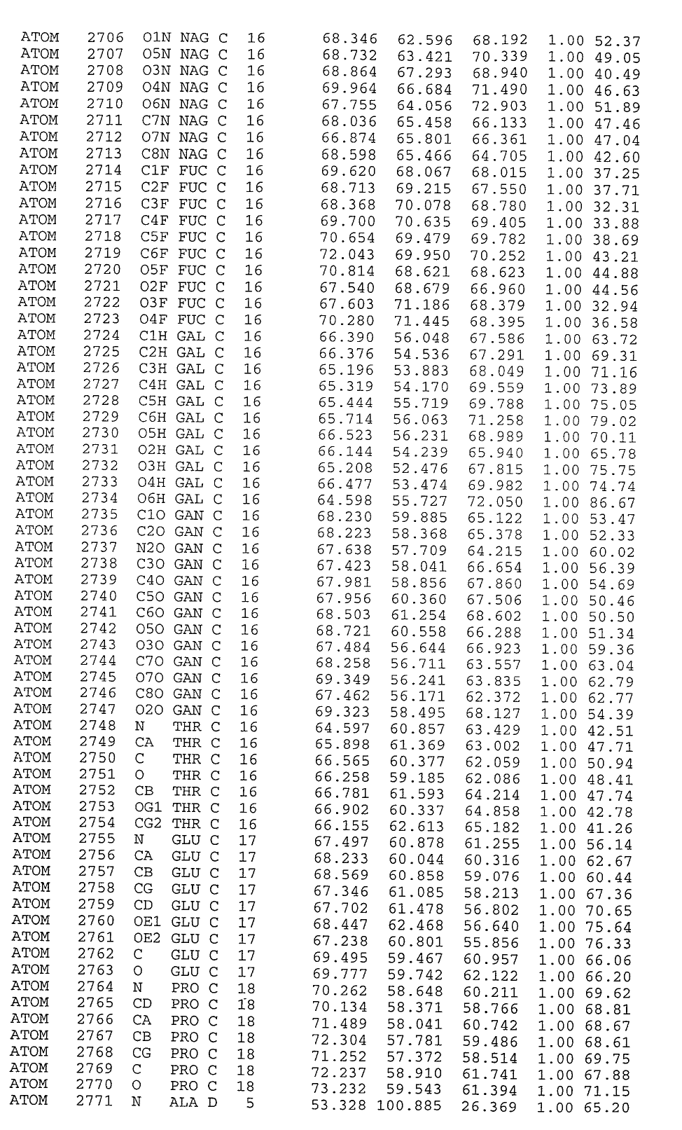

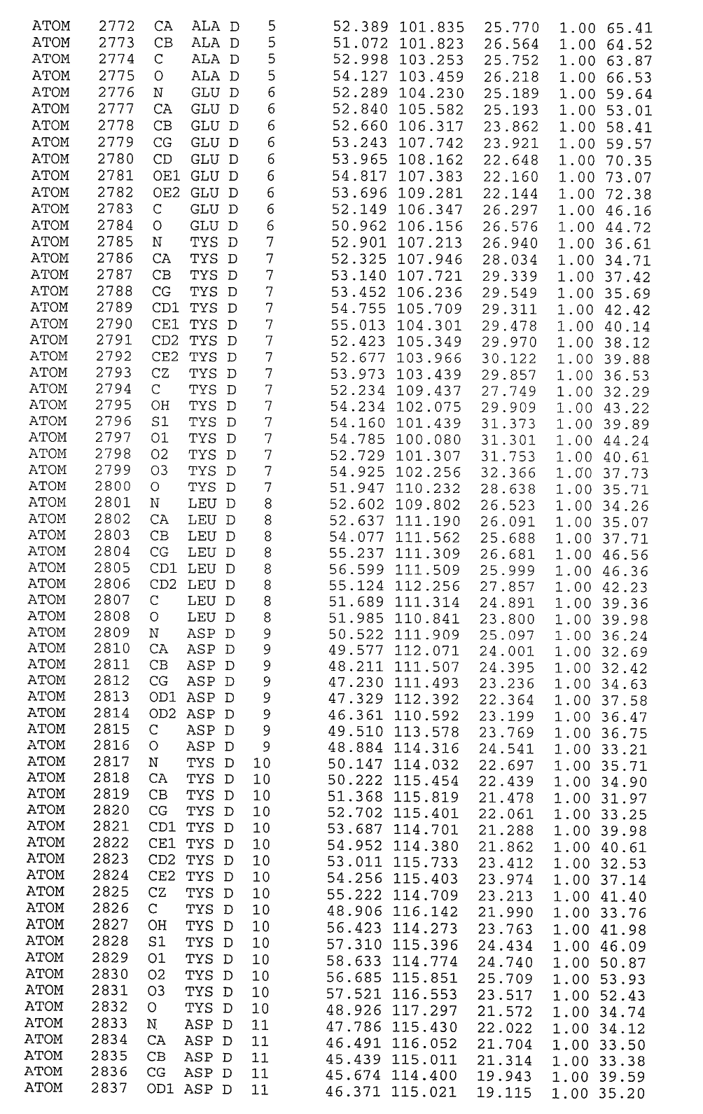

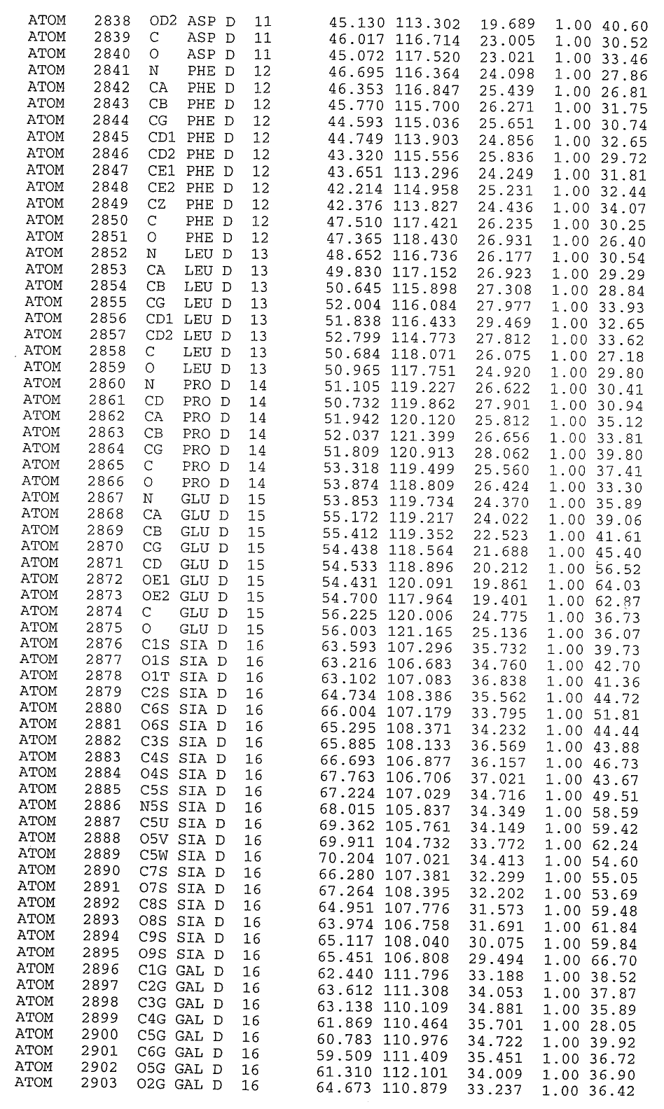

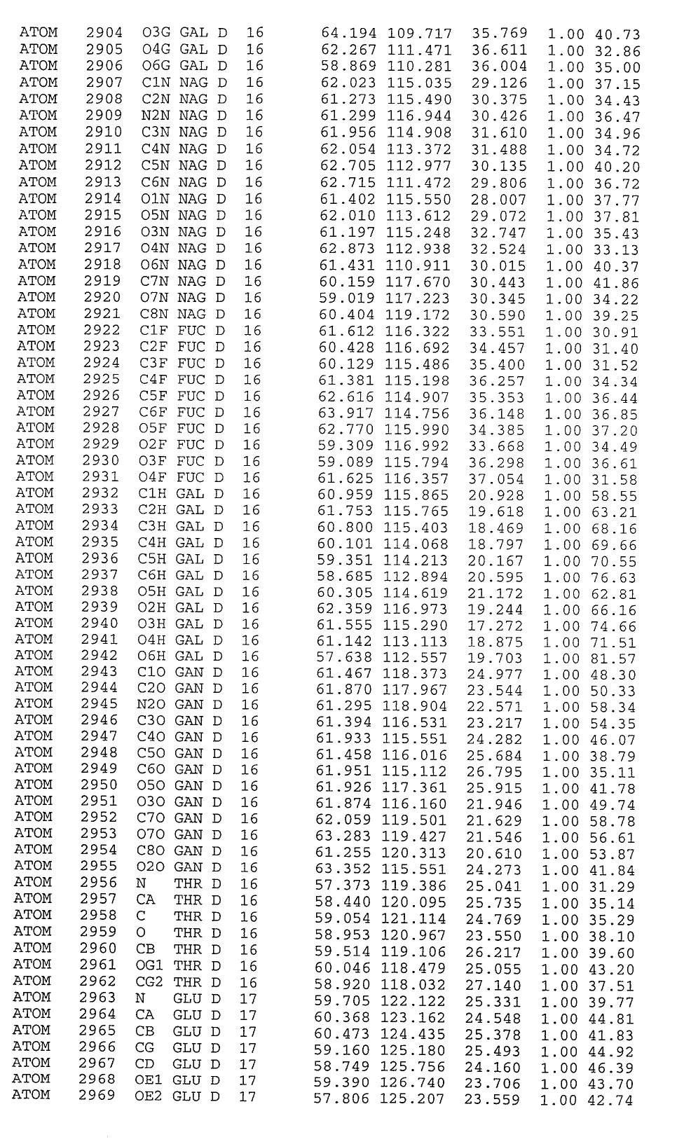

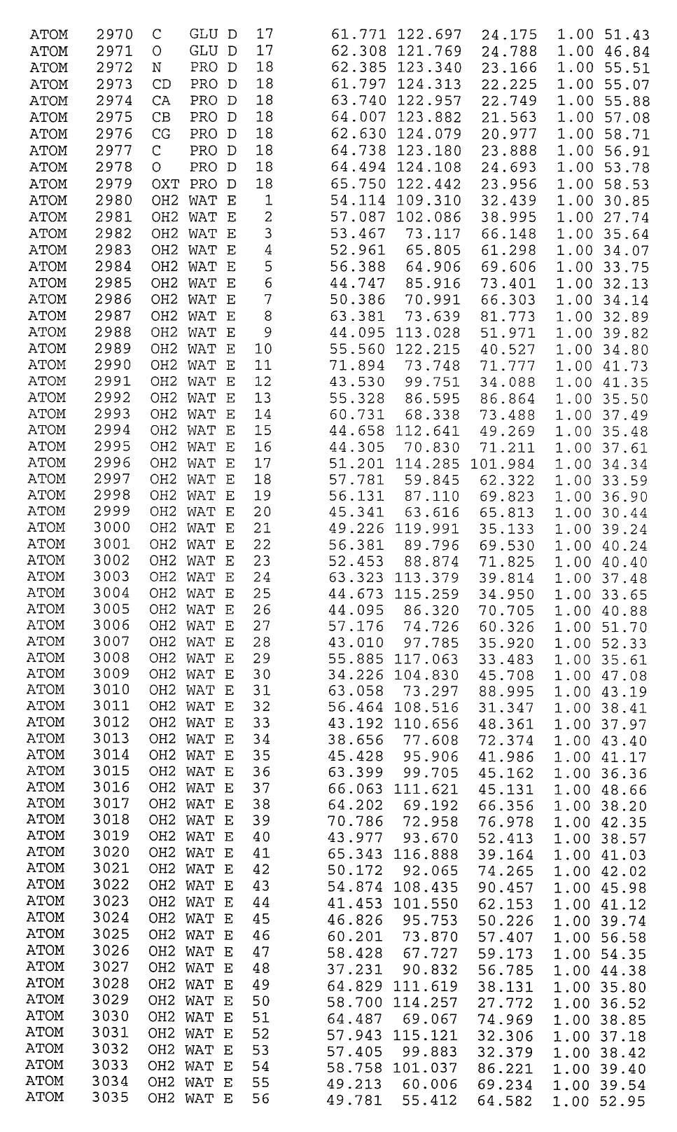

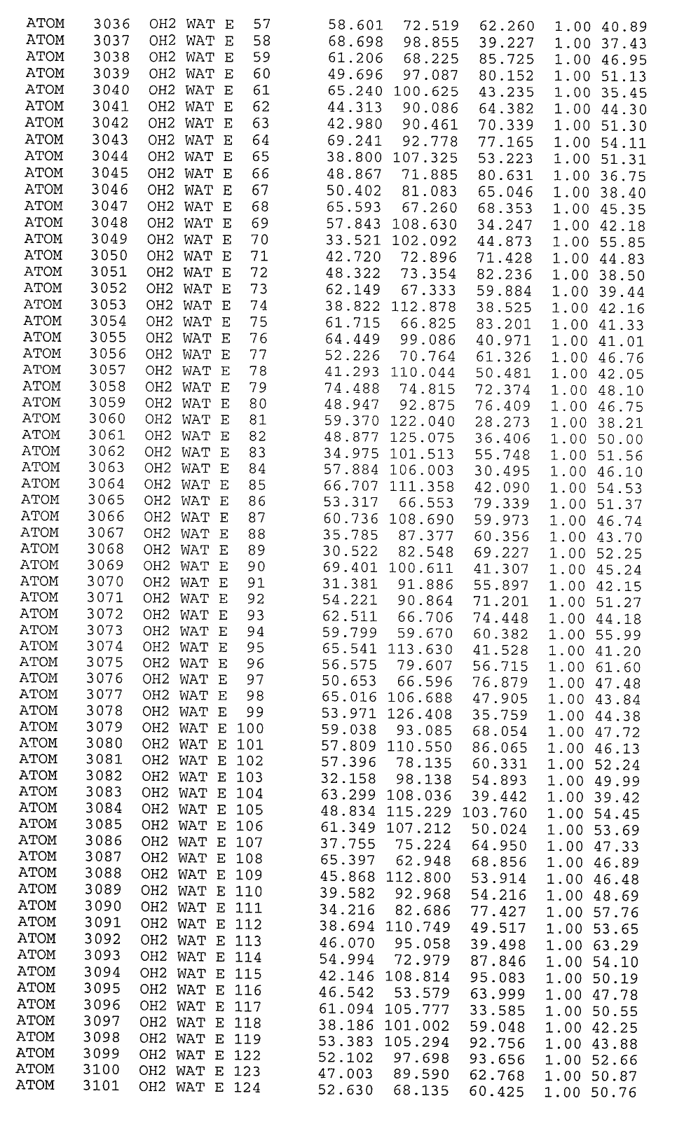

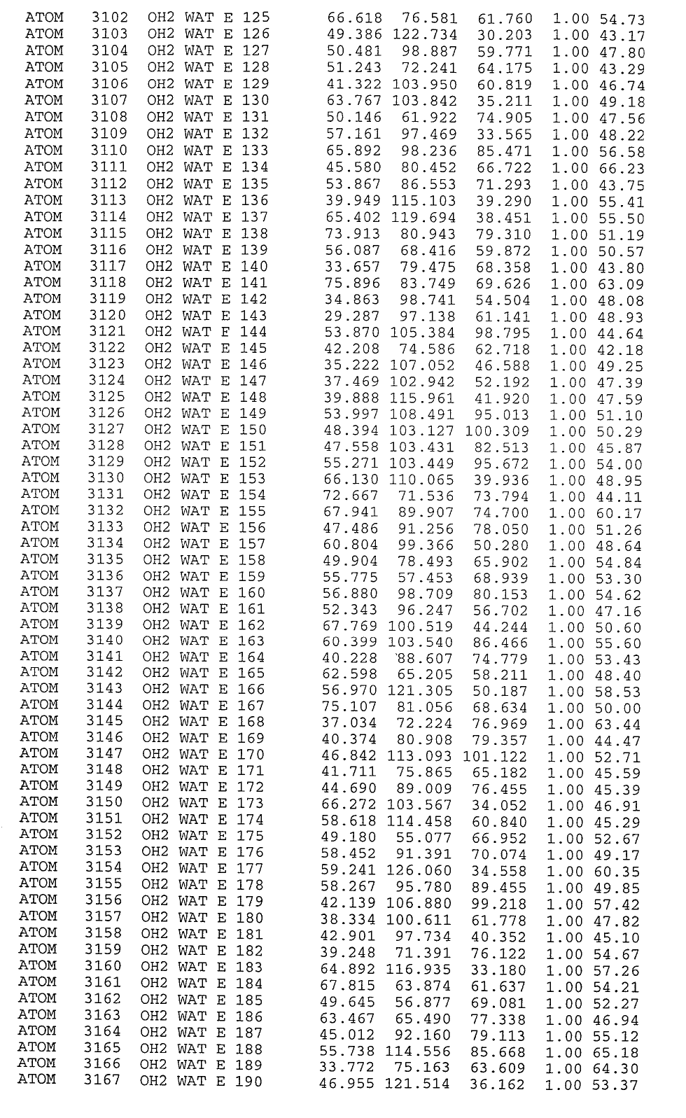

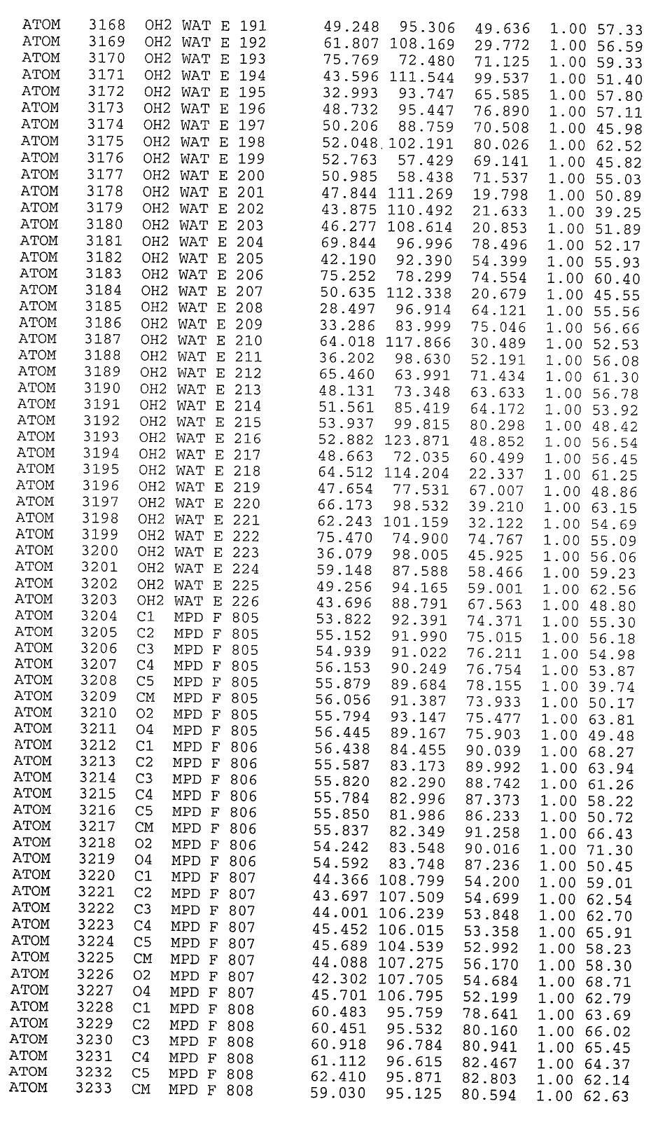

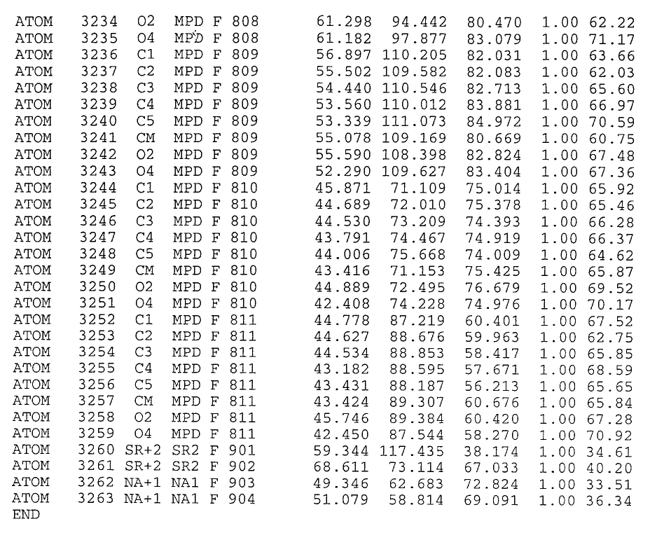

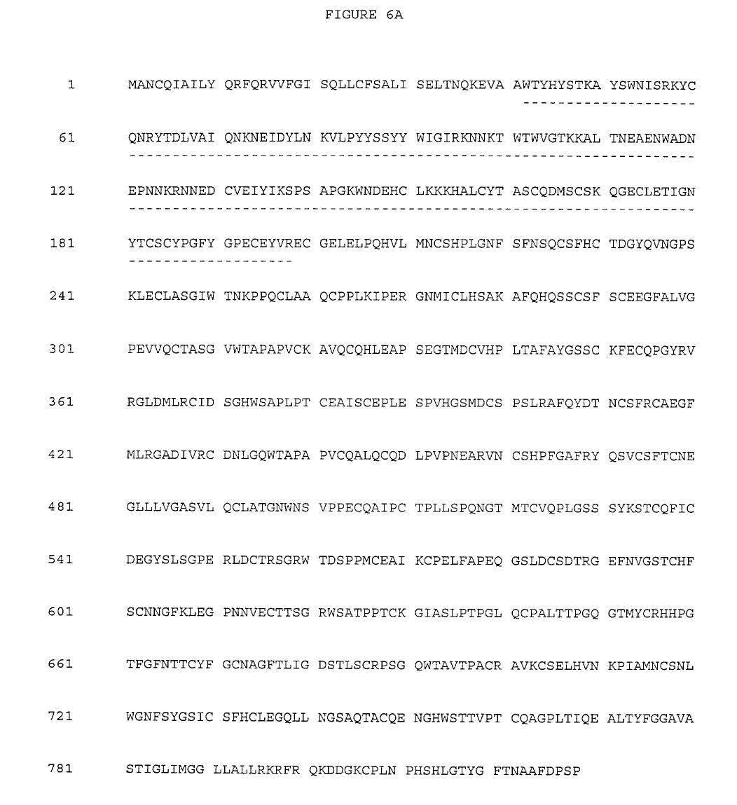

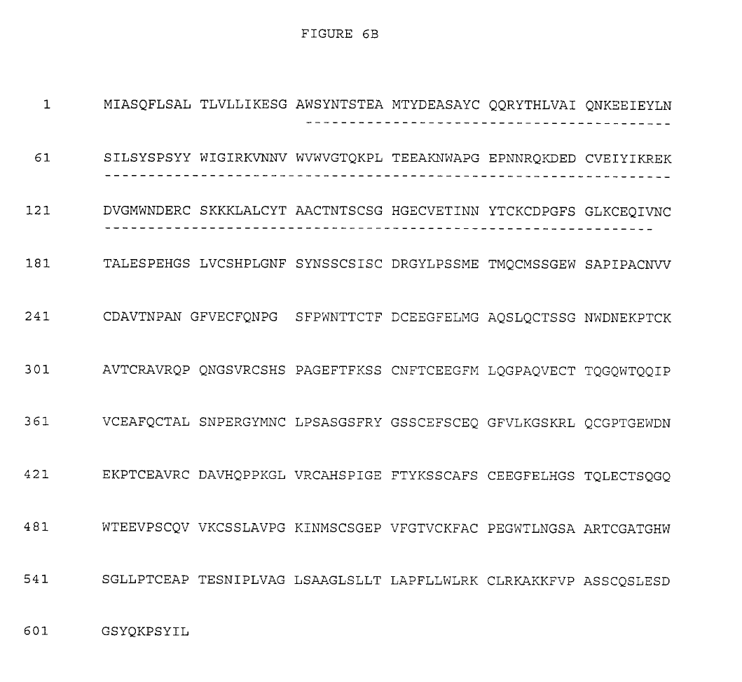

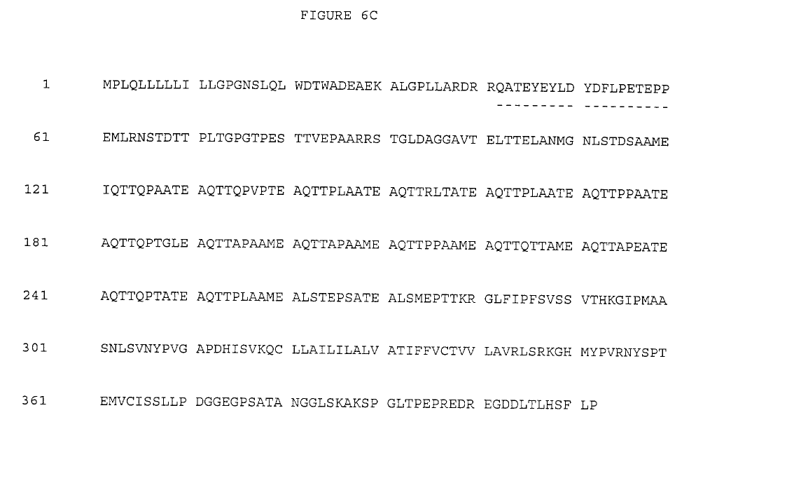

Unless otherwise noted, “selectins” are a family of cell-surface glycoproteins responsible for early adhesion events in the recruitment of leukocytes into sites of inflammation and their emigration into lymphatic tissues, and include P-selectin, E-selectin and L-selectin, and analogues thereof having selectin activity. P-selectin preferably has the amino acid sequence depicted in FIG. 6A, including conservative substitions. E-selection preferably has the amino acid sequence in FIG. 6B, including conservative substitutions. An “E-selectin type molecule” includes the entire E-selectin molecule as well as portions thereof, such as the lectin and/or epidermal growth factor (EGF)-like domains, and preferably is “E-selectin LE” as defined below.

The “LE domains” represent the lectin and epidermal growth factor (EGF)-like domains of selectin, and as used herein, “P-selectin LE” represents the lectin and EGF-like domains of P-selectin, while “E-selectin LE” represents the lectin and EGF-like domains of E-selectin. The amino acid sequences for P-selectin LE and E-selectin LE are shown (underlined) in FIGS. 6A and 6B, respectively, and include conservative substitutions.

“SLe^{X}” represents sialyl Lewis^{X} (SLe^{X}, NeuNAc2,3Gal1,4[Fuc1,3] GlcNAc) tetrasaccharide, and includes “SLe^{X} analogues” having similar structure and activity as SLe^{X}. An “SLe^{X} binding protein or peptide” is a protein or peptide that binds SLe^{X} and has an SLe^{X} binding site, and includes but is not limited to P-selectin, E-selectin, P-selectin LE and E-selectin LE. A “molecule or molecular complex comprising an SLe^{X} binding site” includes (i) P-selectin, E-selectin, P-selectin LE, E-selectin LE, (ii) complexes of P-selectin, E-selectin, P-selectin LE, or E-selectin LE with SLe^{X}, (iii) complexes of P-selectin, E-selectin, P-selectin LE, or E-selectin LE with other molecules, and (iv) other molecules or molecular complexes having an SLe^{X} binding site.

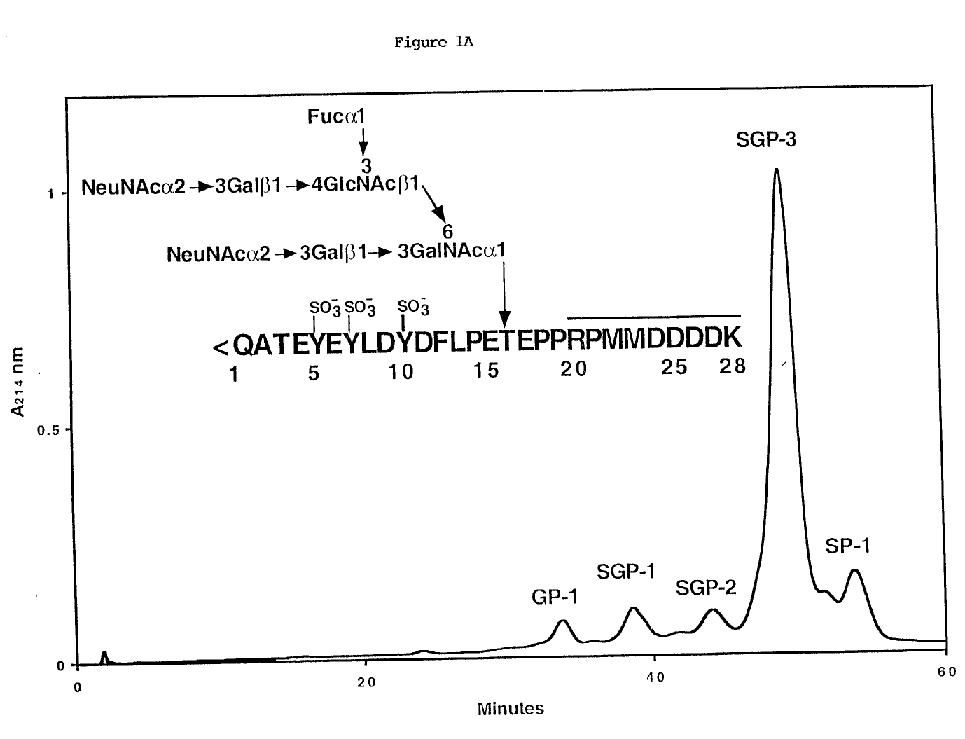

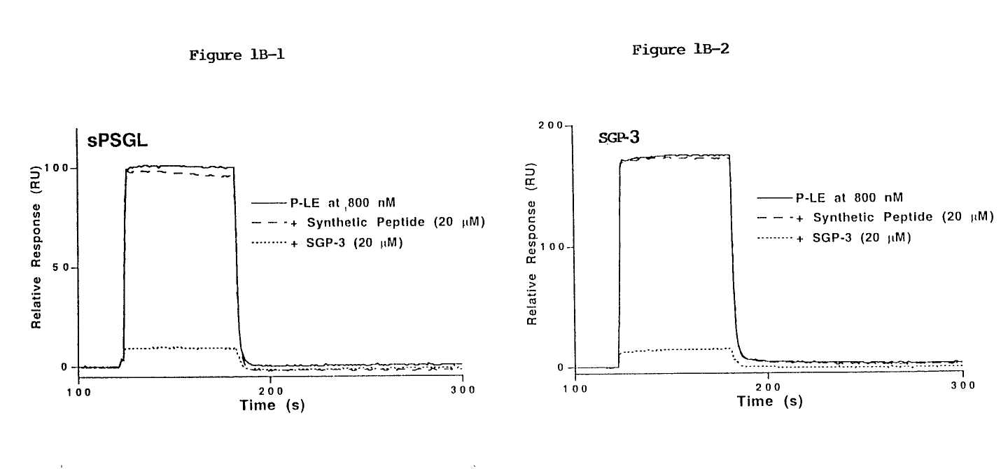

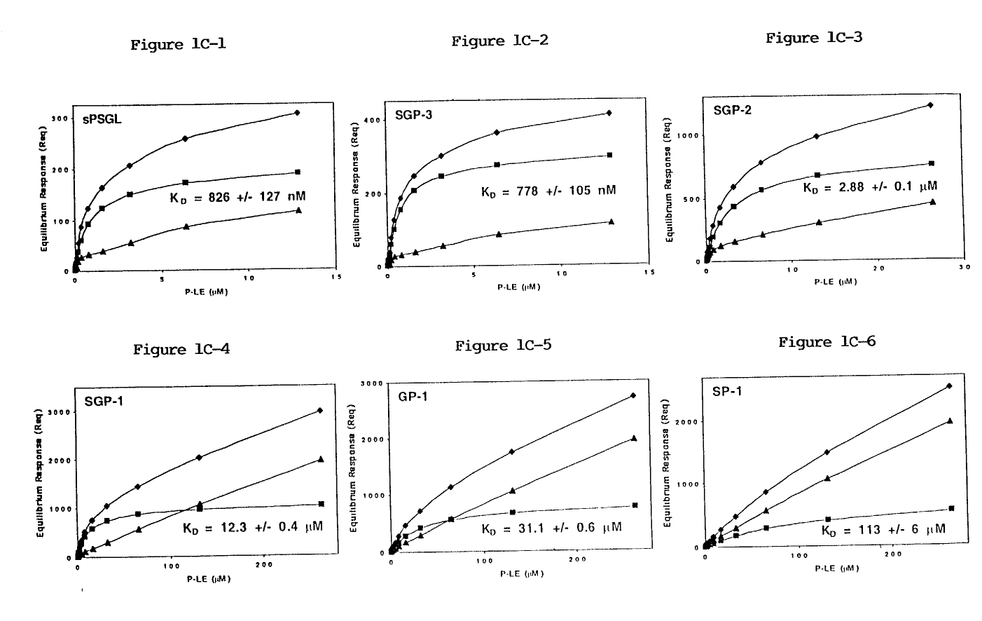

“PSGL-1” is a molecule having PSGL-1 activity, and includes a “PSGL-1 peptide” as defined below. The amino acid sequence of PSGL-1 is depicted in FIG. 6C, and includes conservative substitutions thereof. “PSGL-1 peptide” is a peptide modified by tyrosine sulfation and SLe^{X}, and includes the peptide structure depicted in FIG. 1A (denoted as SGP-3), including conservative substitutions thereof. A “PSGL-1 binding protein or peptide” is a protein or peptide that binds to PSGL-1 and has a PSGL-1 binding site, and includes but is not limited to P-selectin, E-selectin, P-selectin LE and E-selectin LE. A “molecule or molecular complex comprising a PSGL-1 binding site” includes (i) P-selectin, E-selectin, P-selectin LE, E-selectin LE, (ii) complexes of P-selectin, E-selectin, P-selectin LE, or E-selectin LE with PSGL-1, (iii) complexes of P-selectin, E-selectin, P-selectin LE, or E-selectin LE with other molecules, and (iv) other molecules or molecular complexes having a PSGL-1 binding site.

Unless otherwise indicated, “protein” or “molecule” shall include a protein, protein domain, polypeptide or peptide.

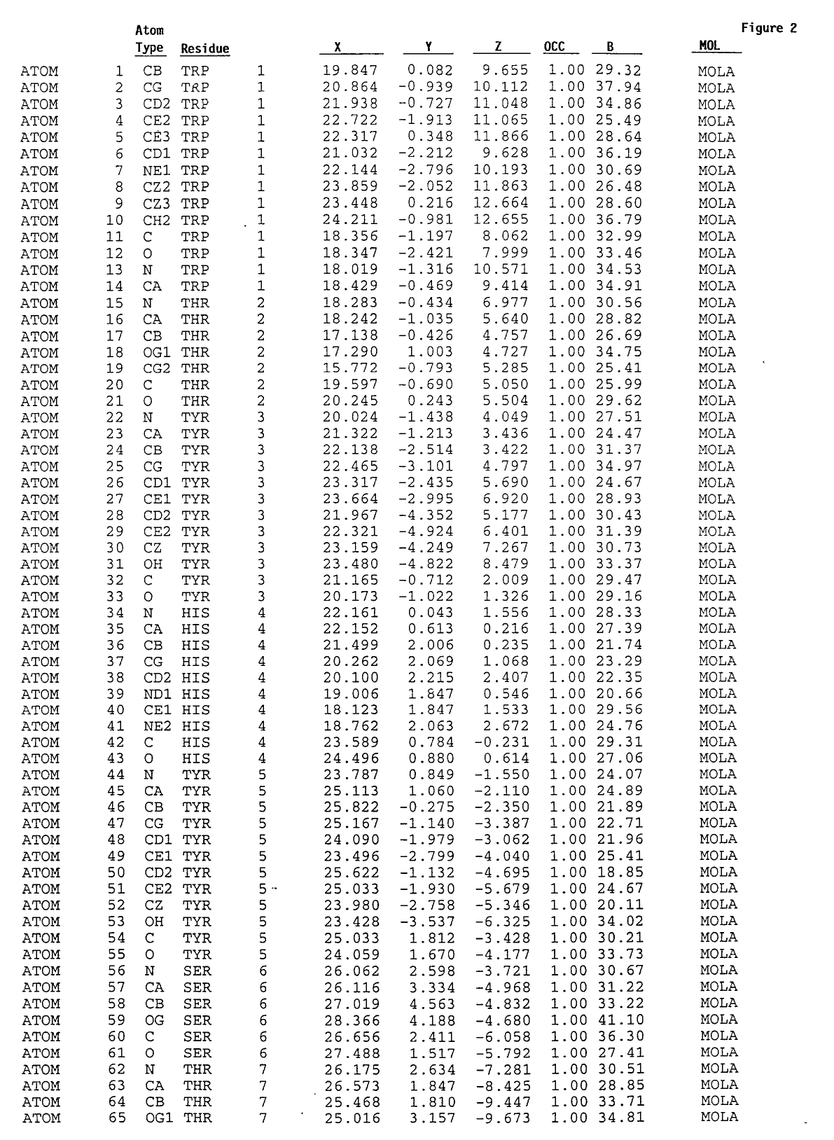

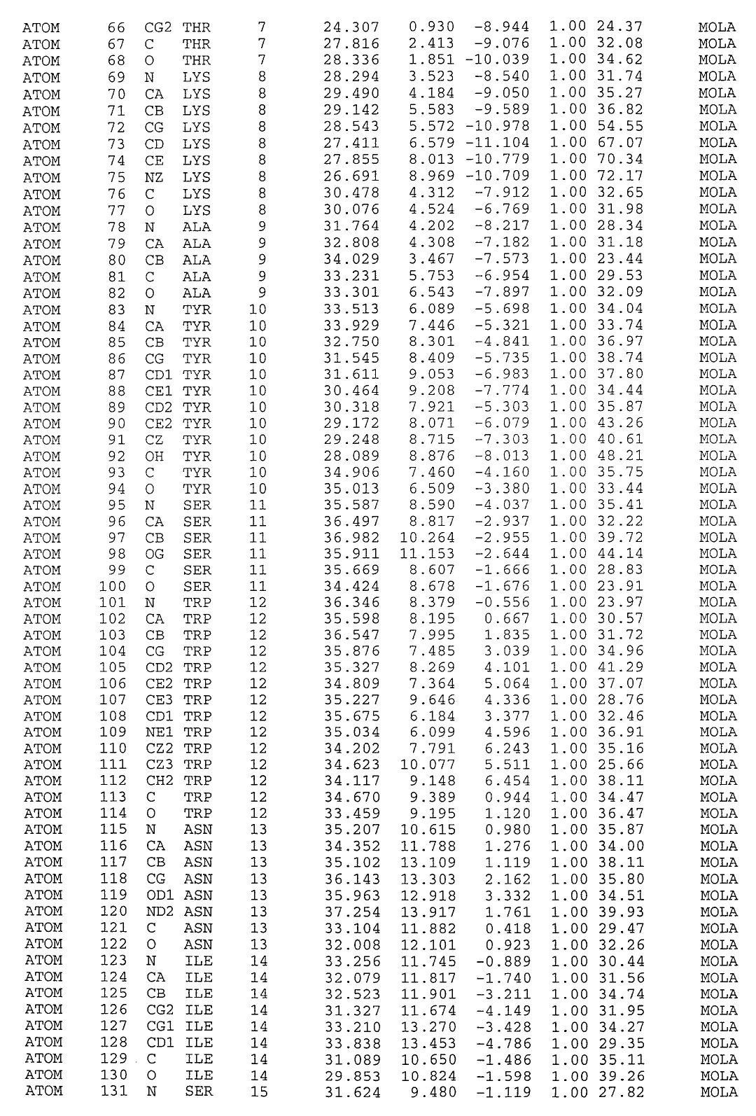

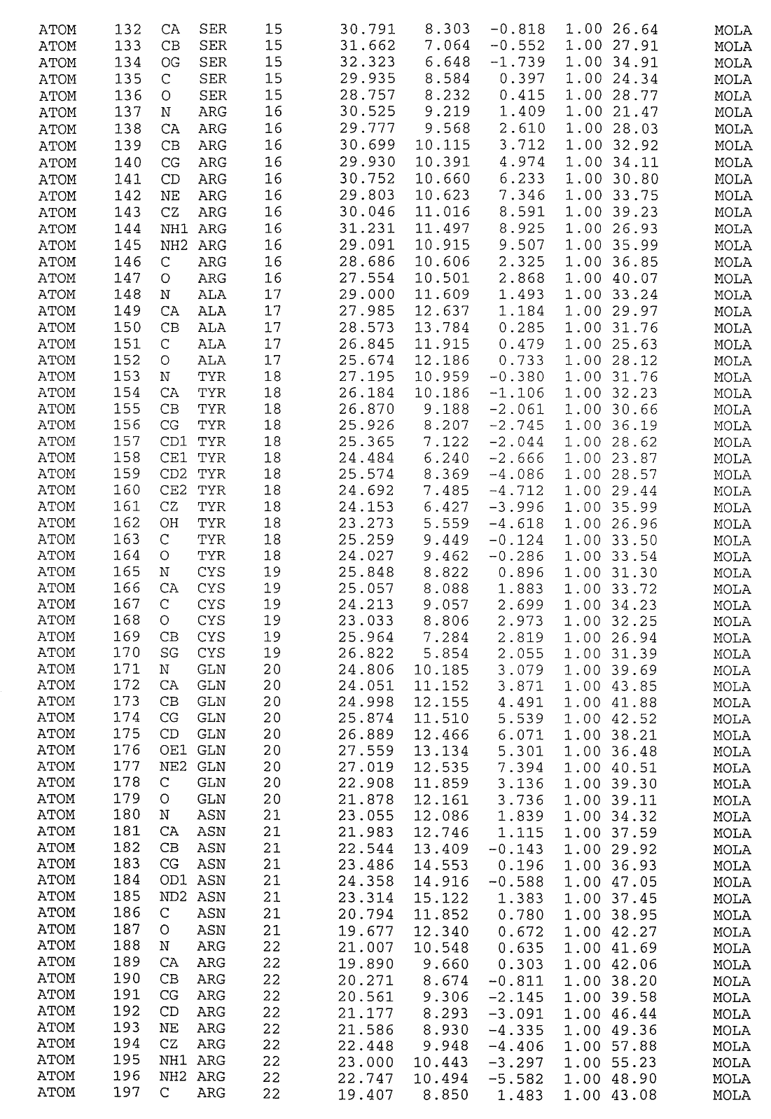

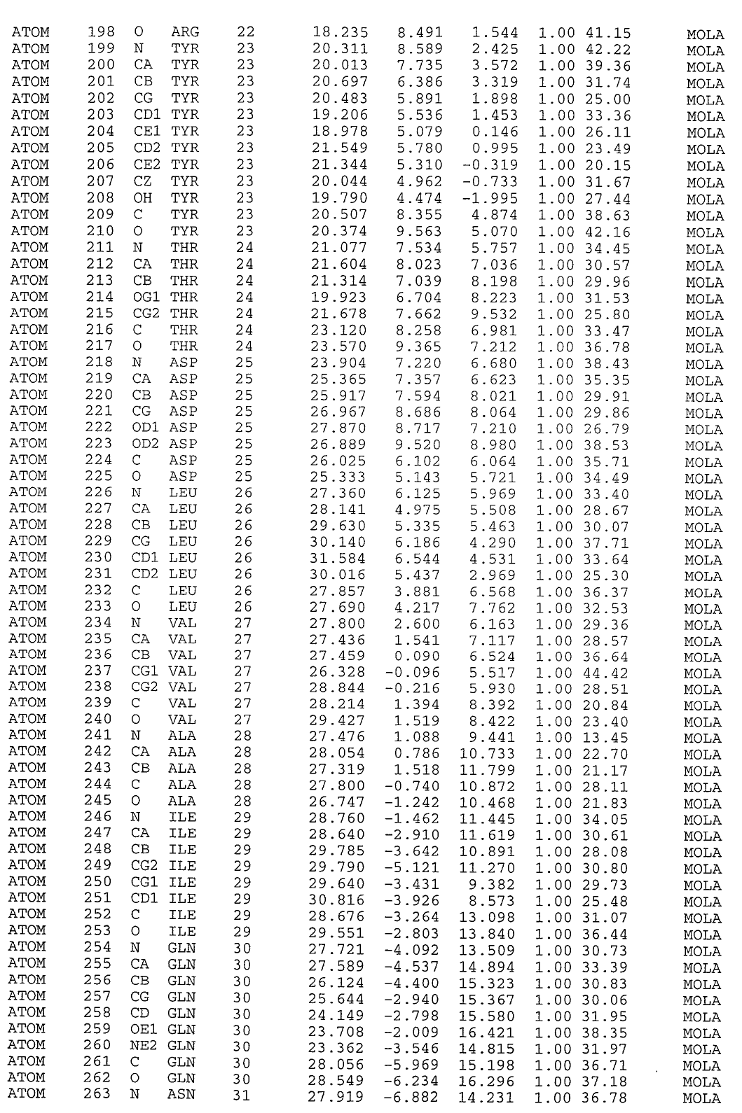

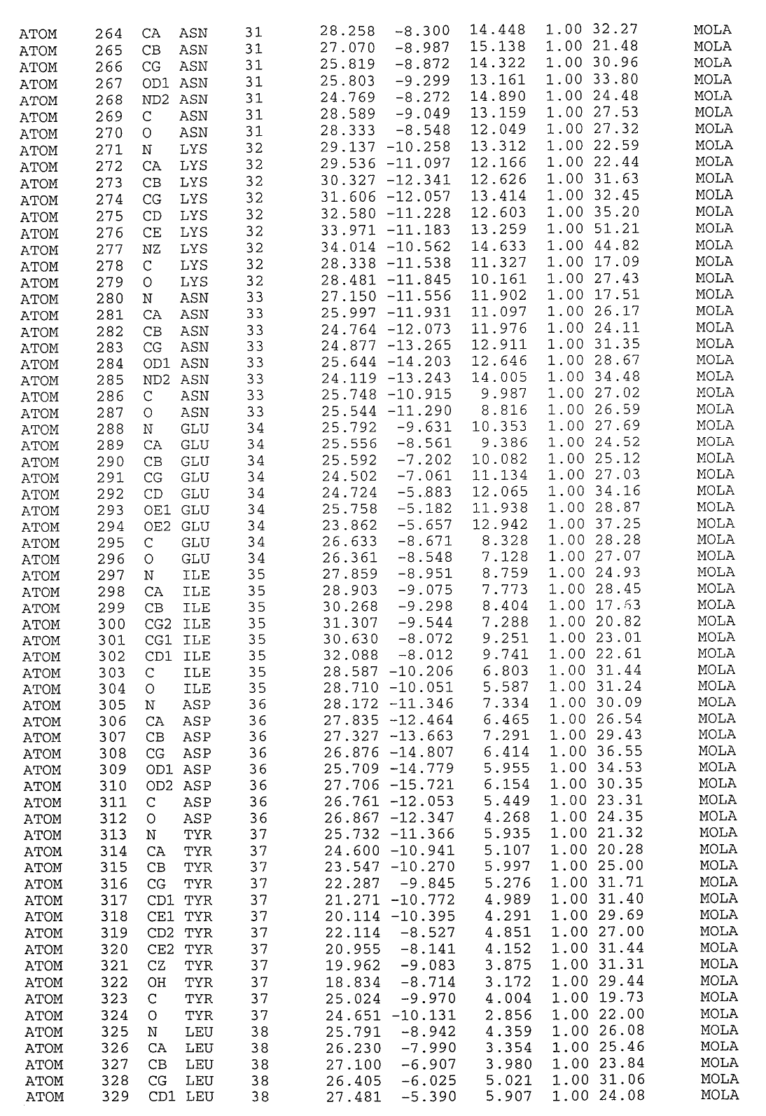

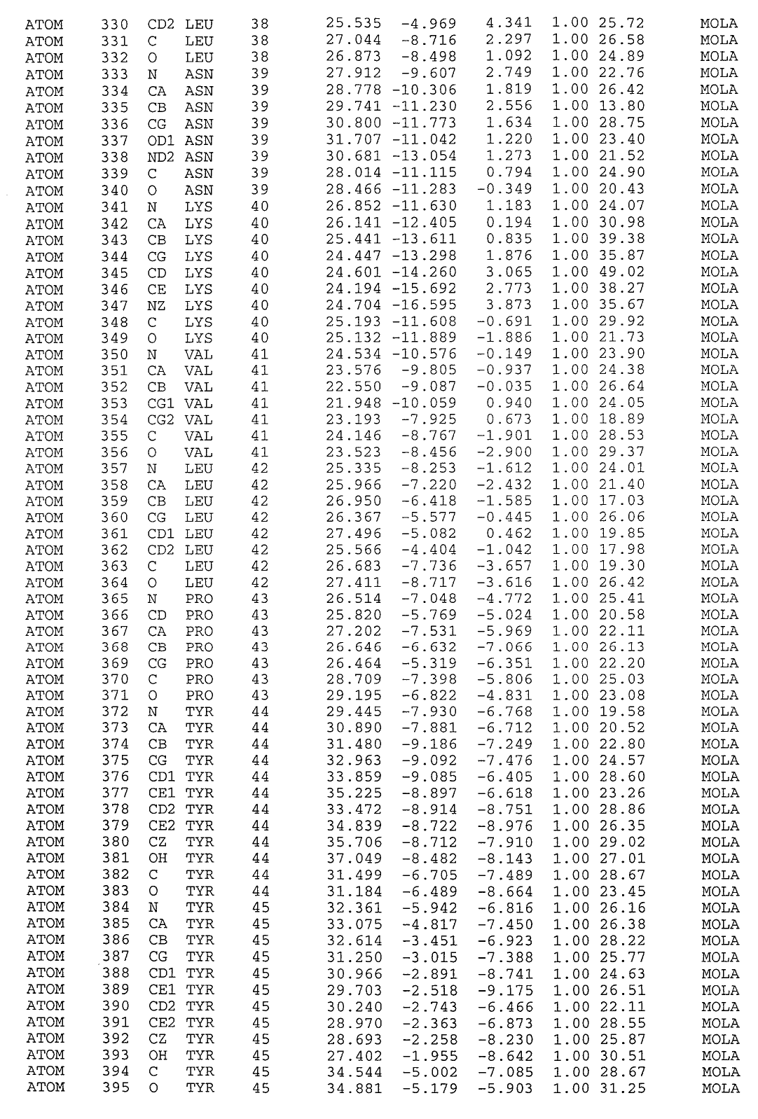

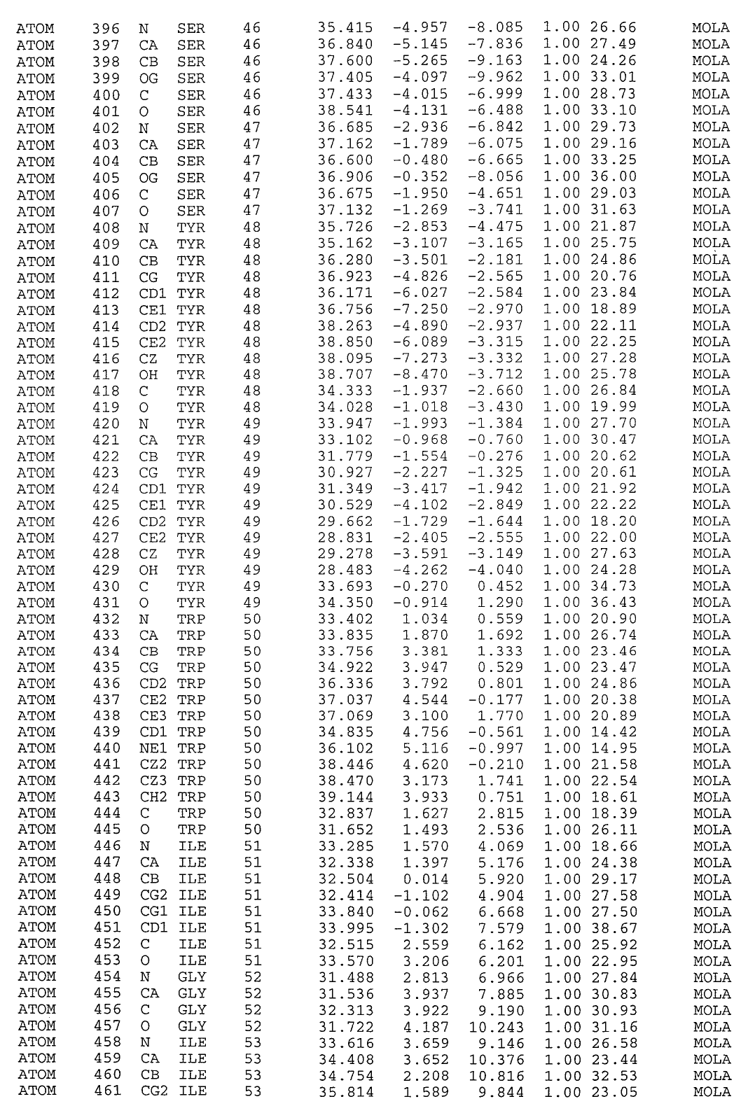

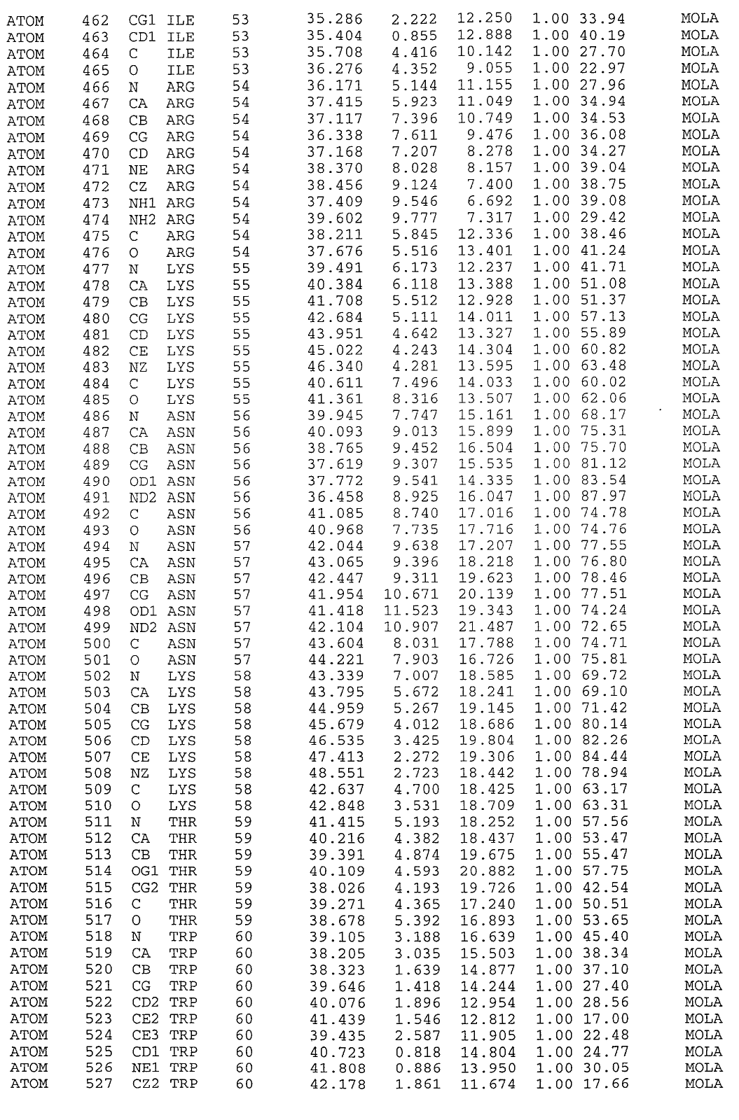

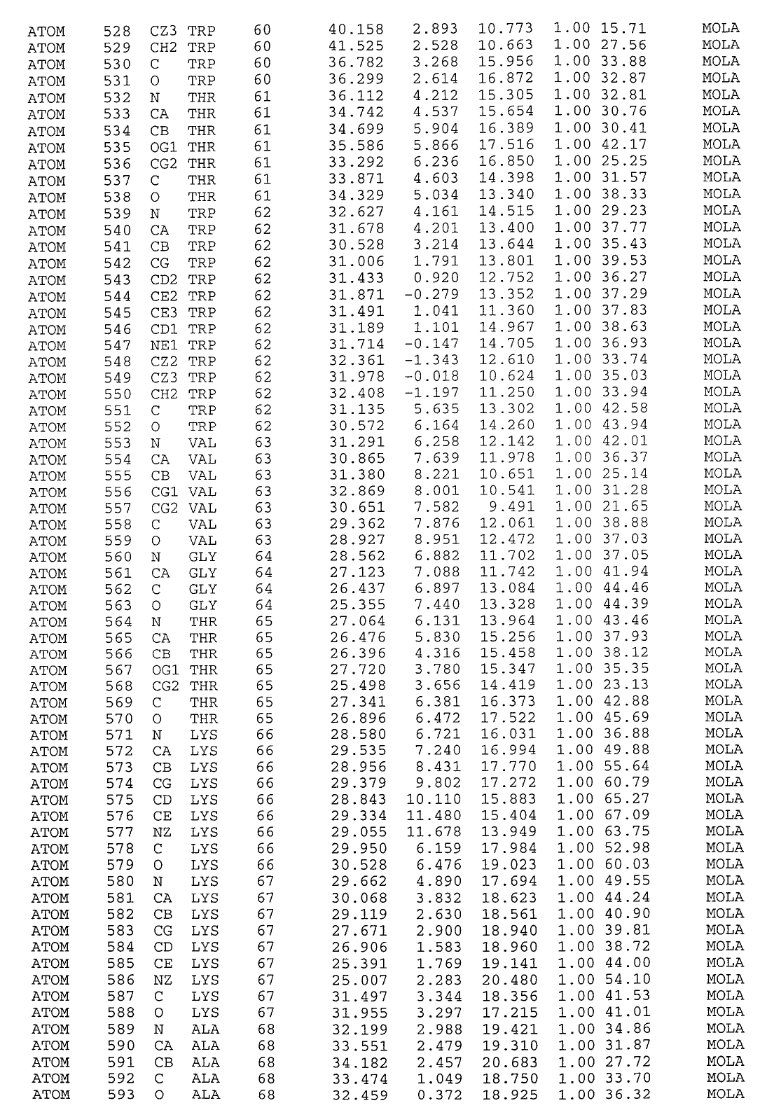

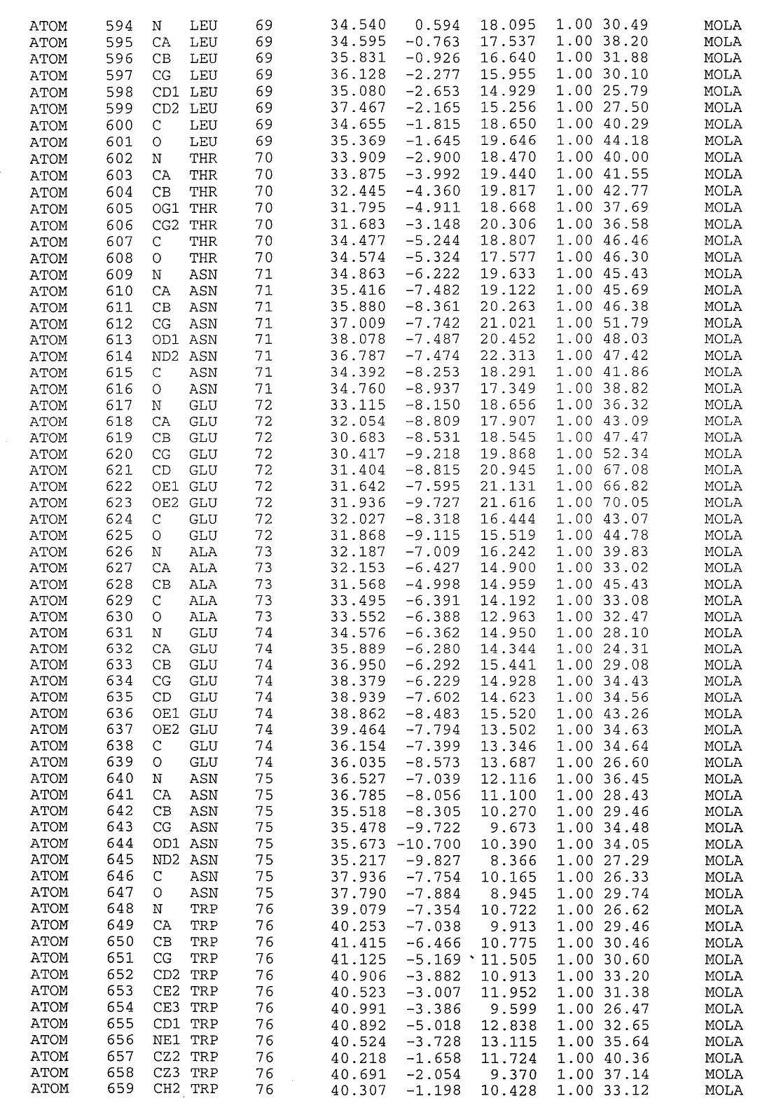

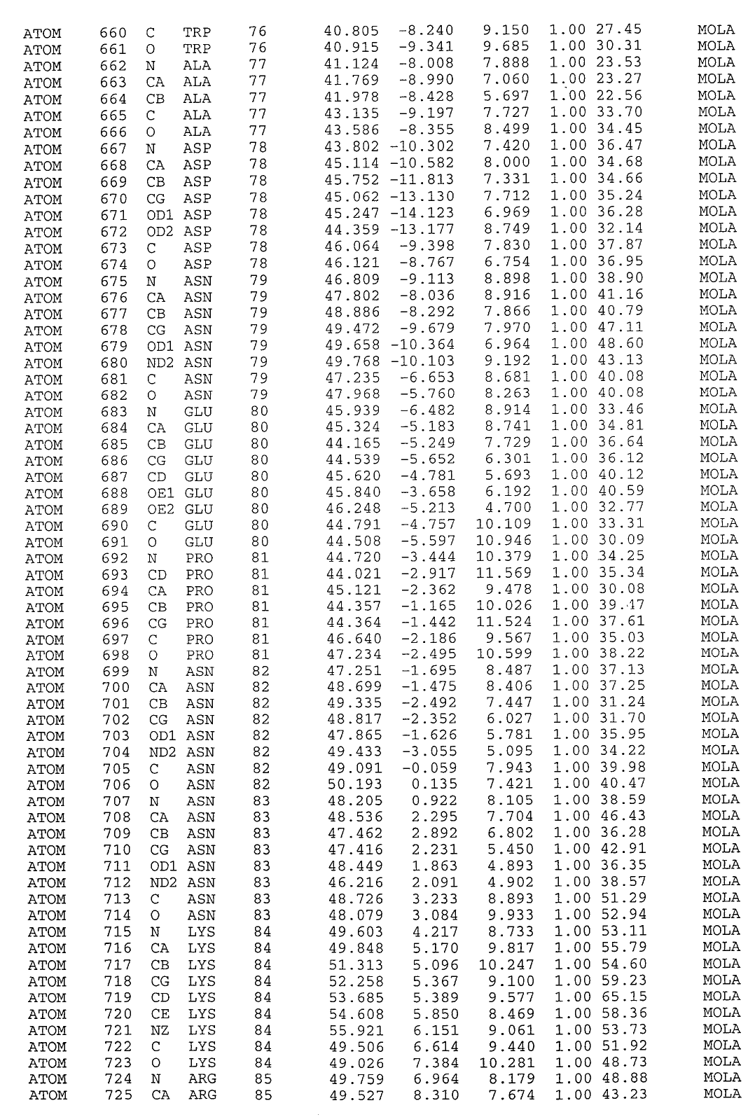

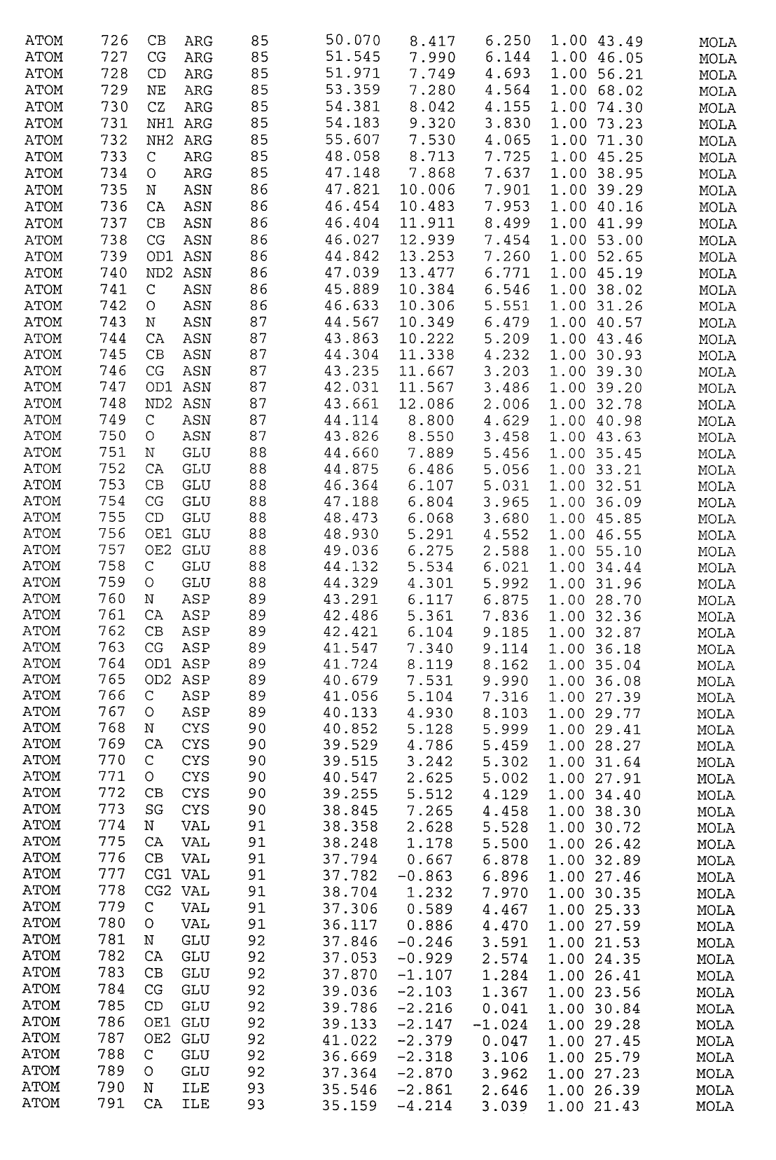

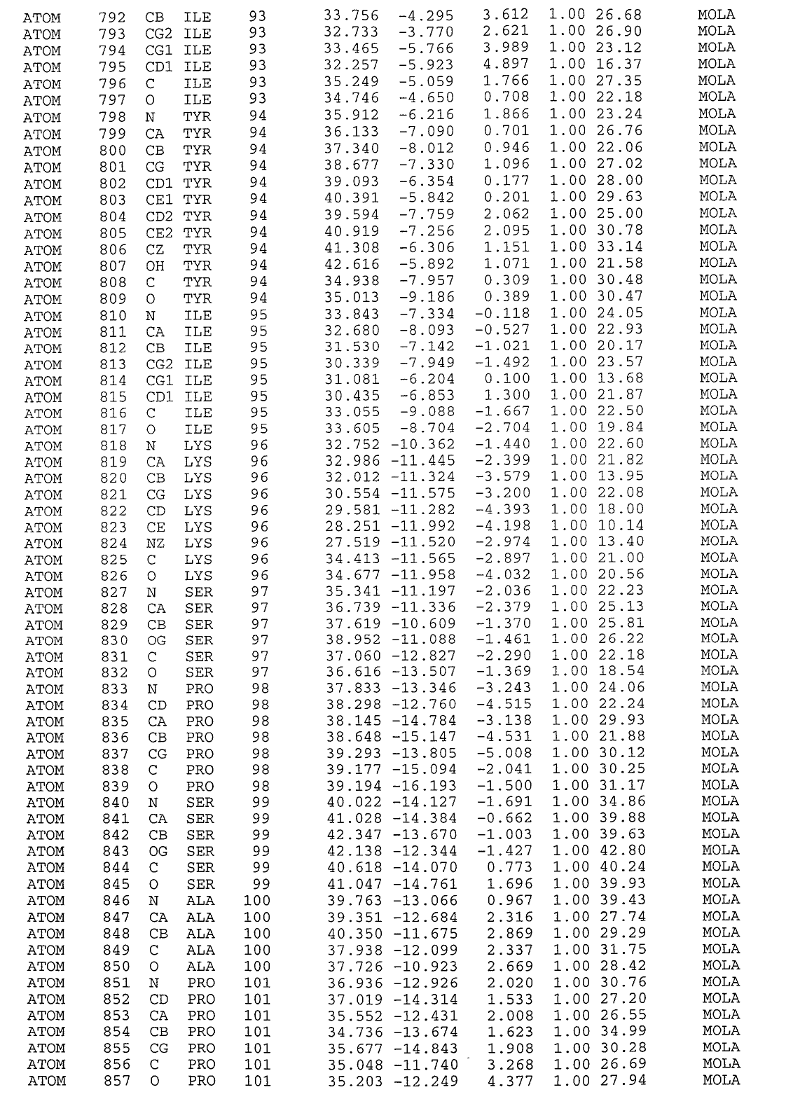

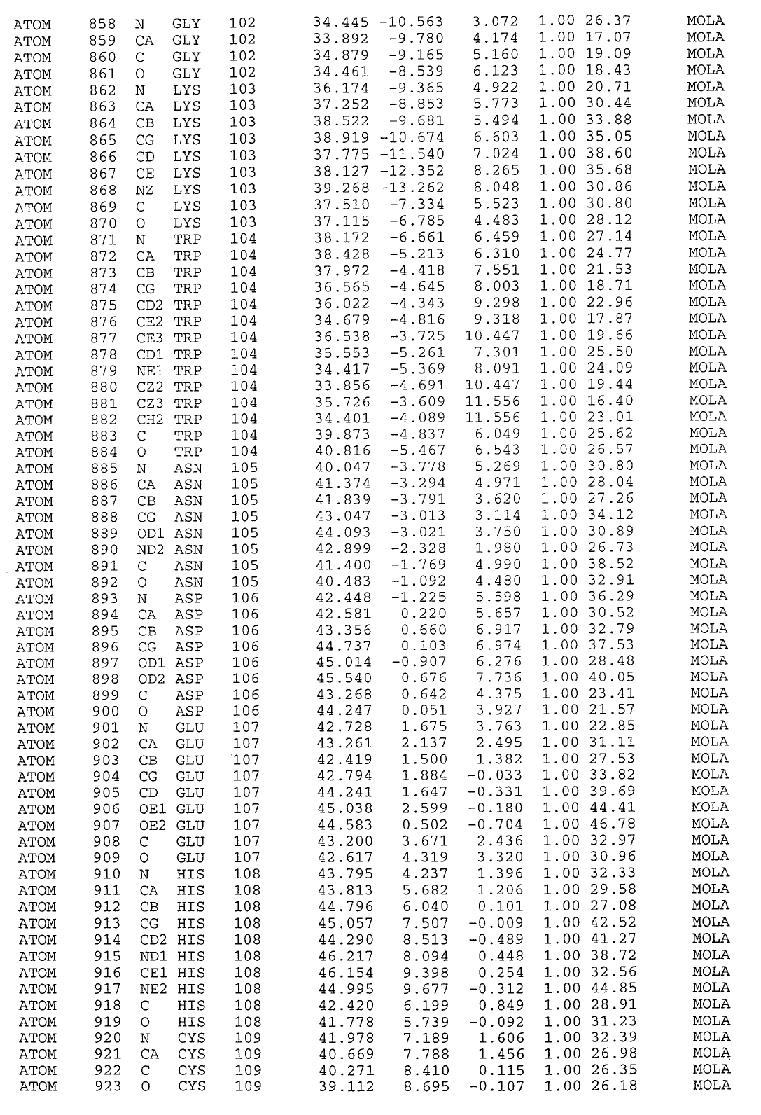

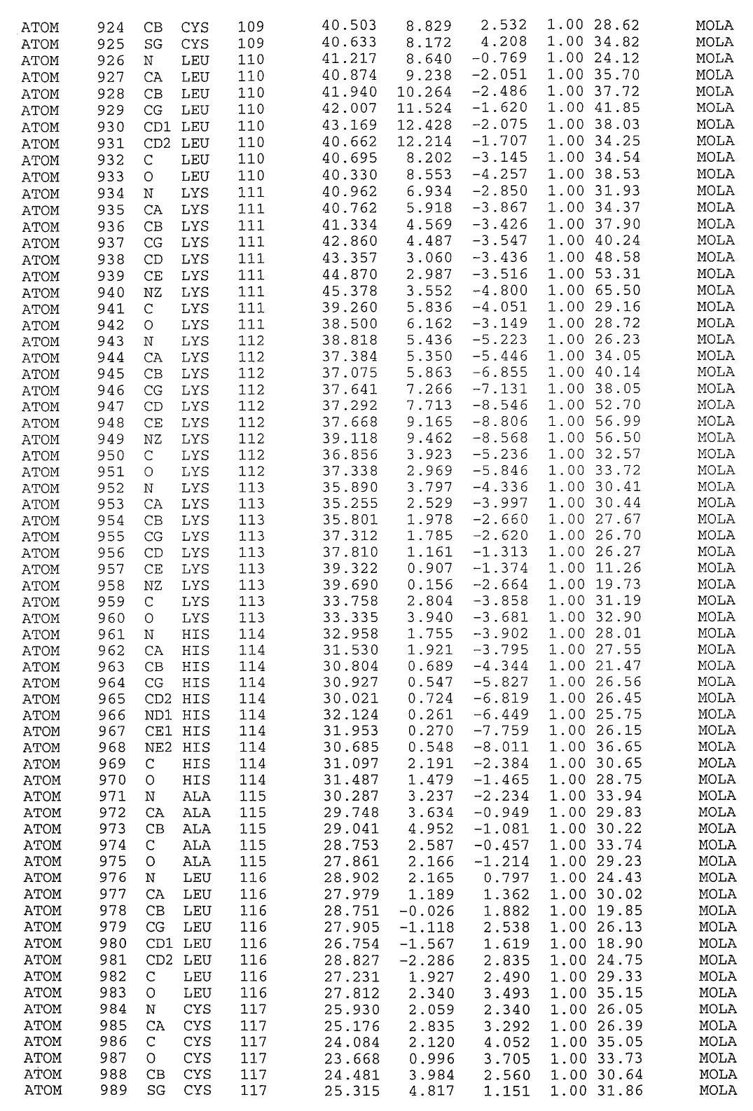

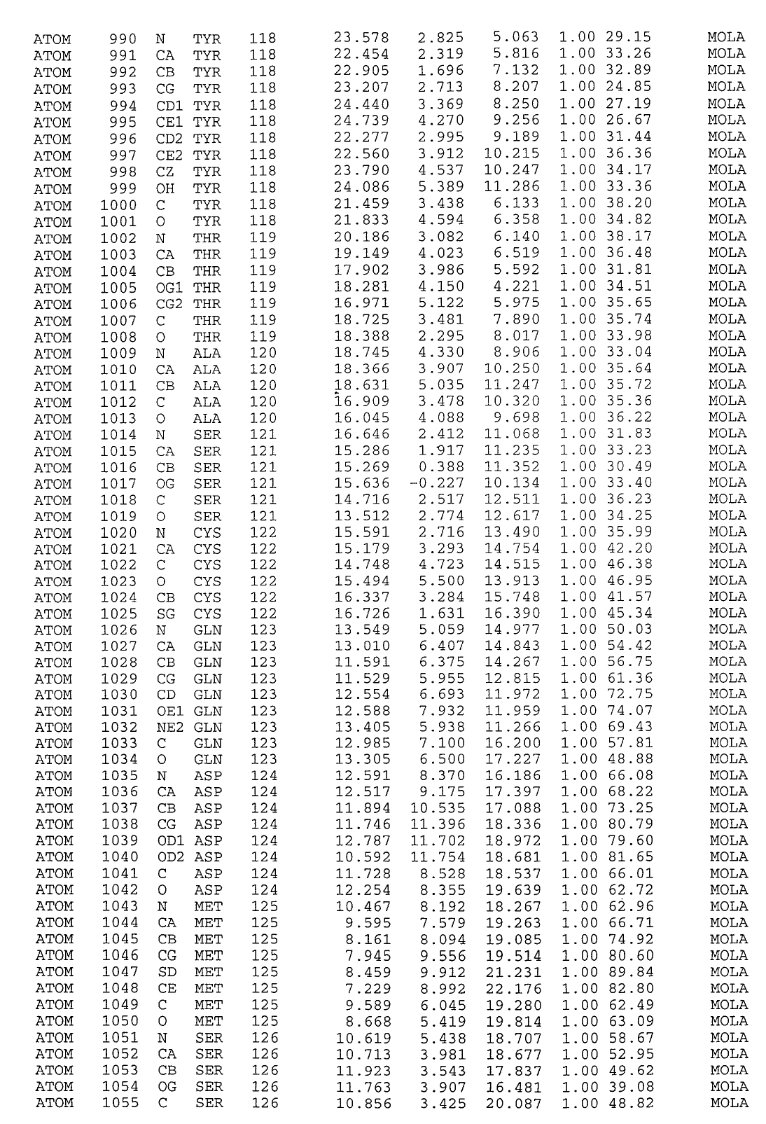

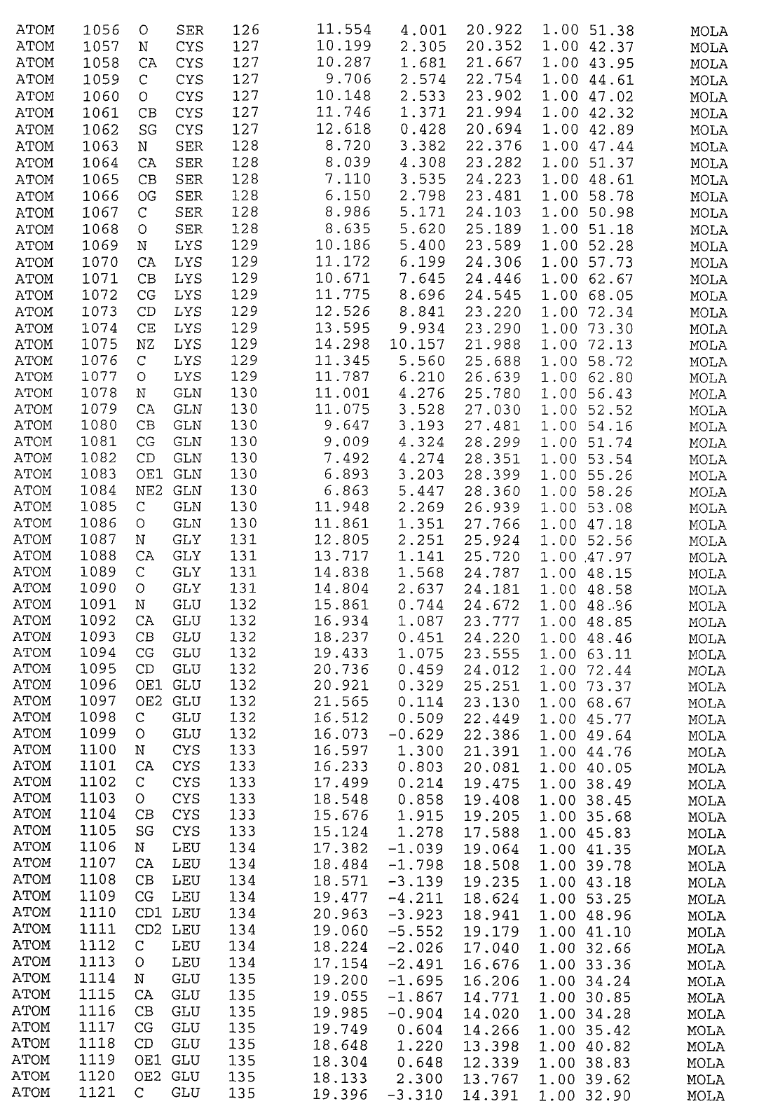

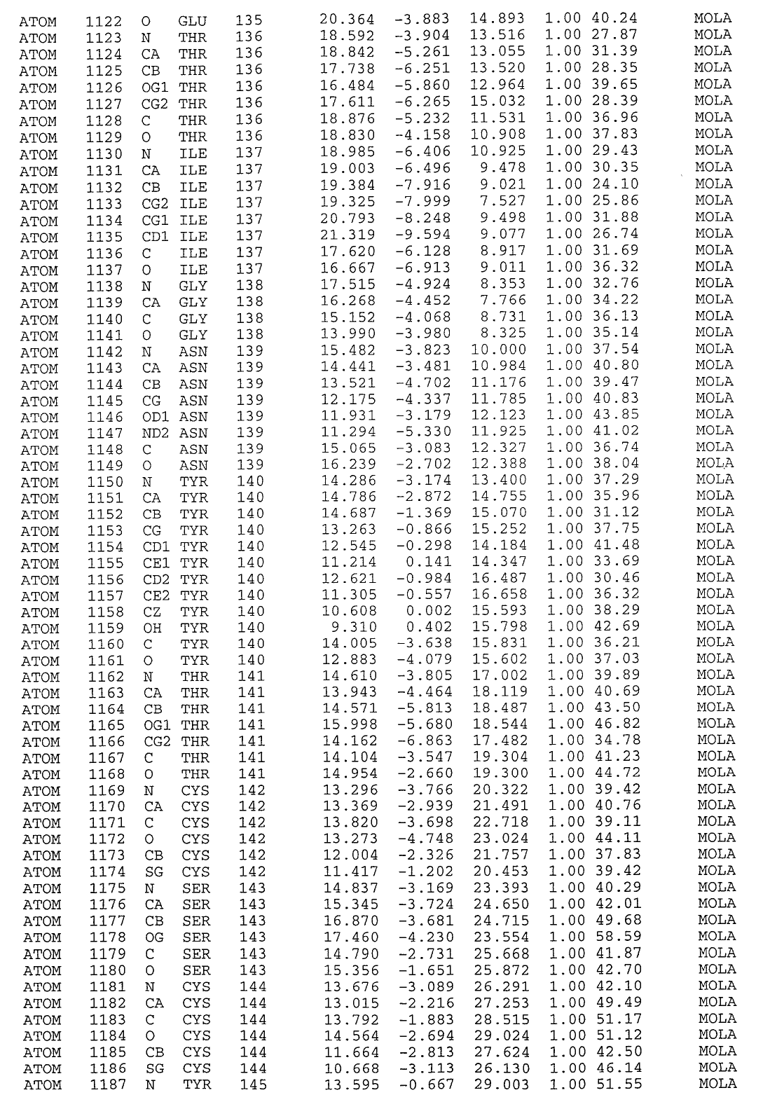

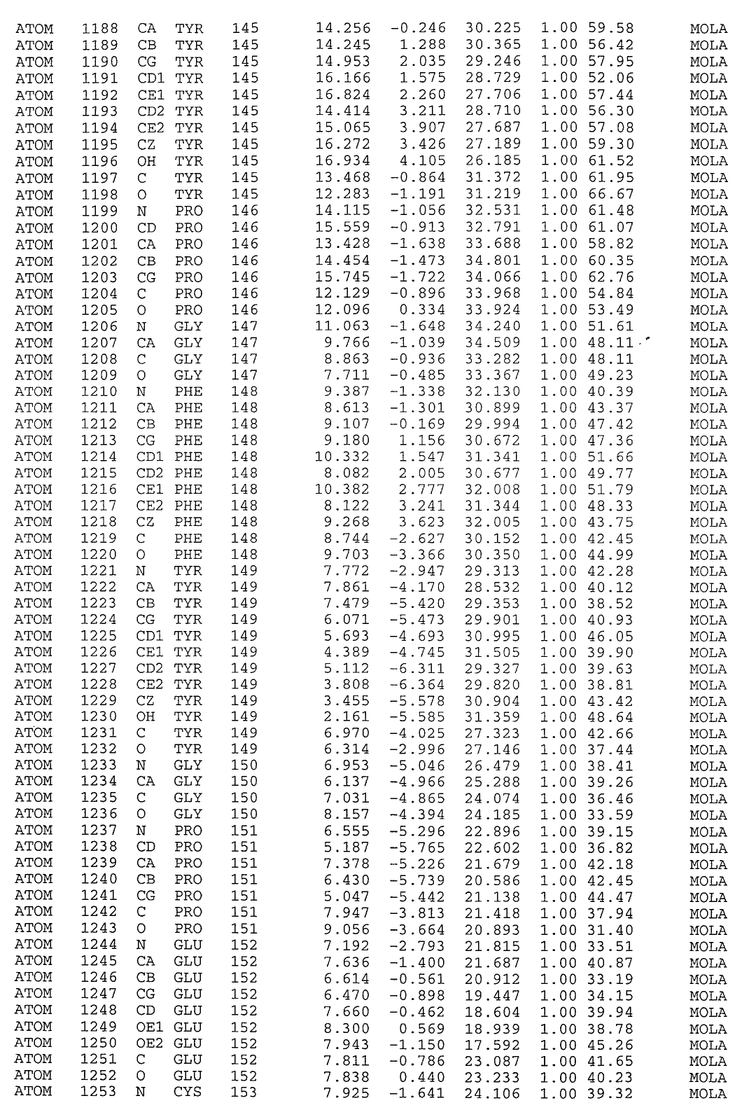

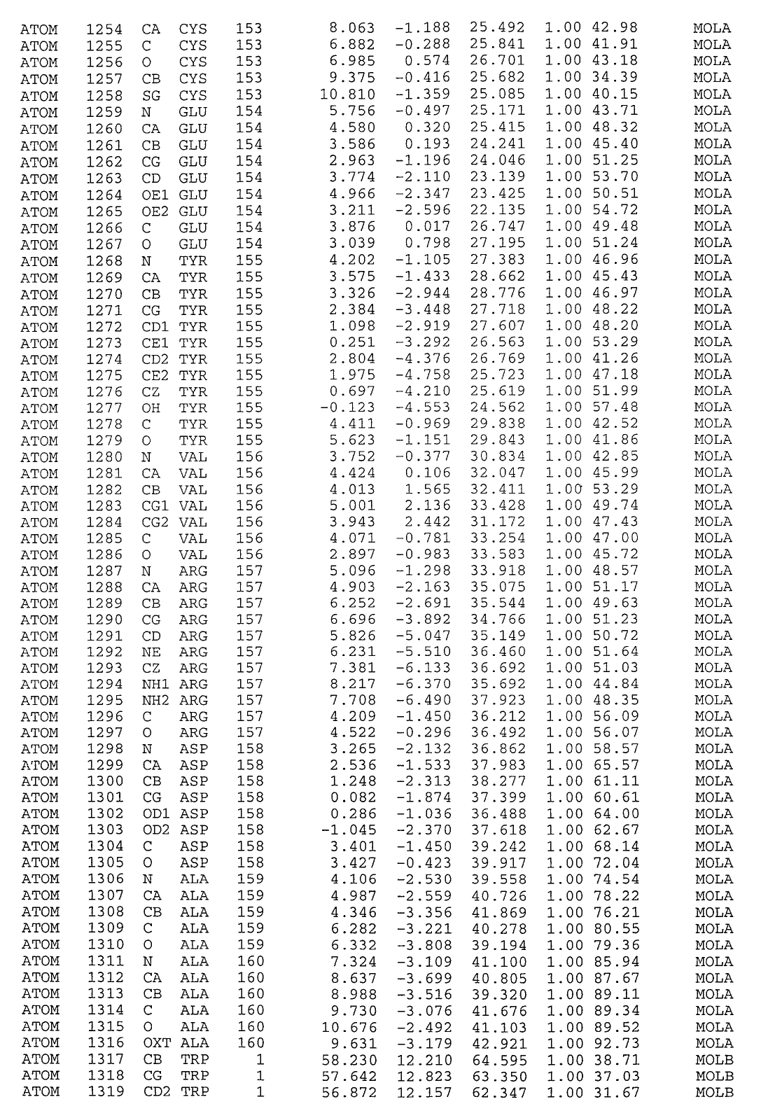

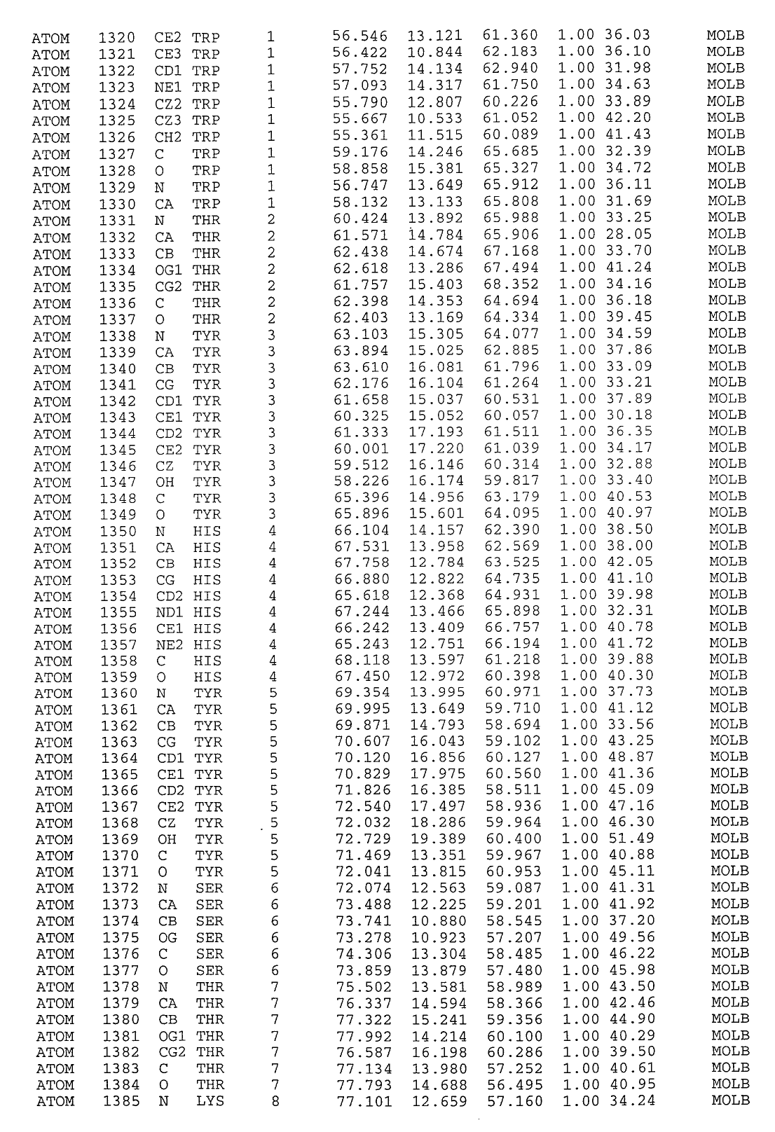

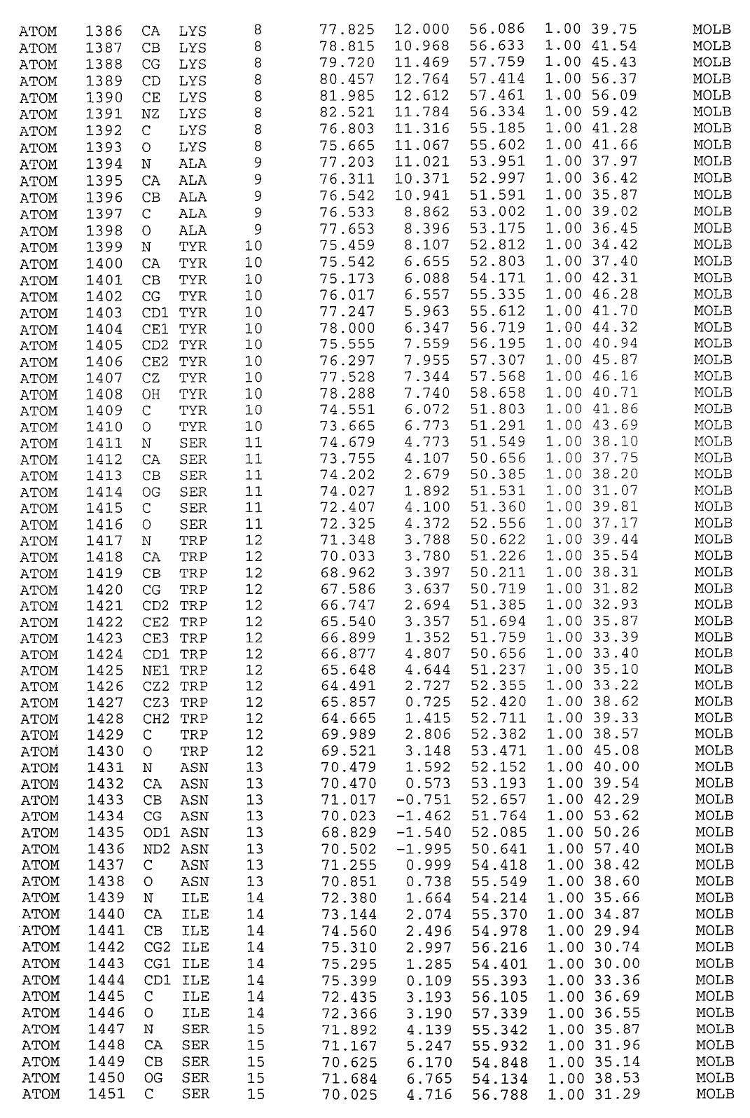

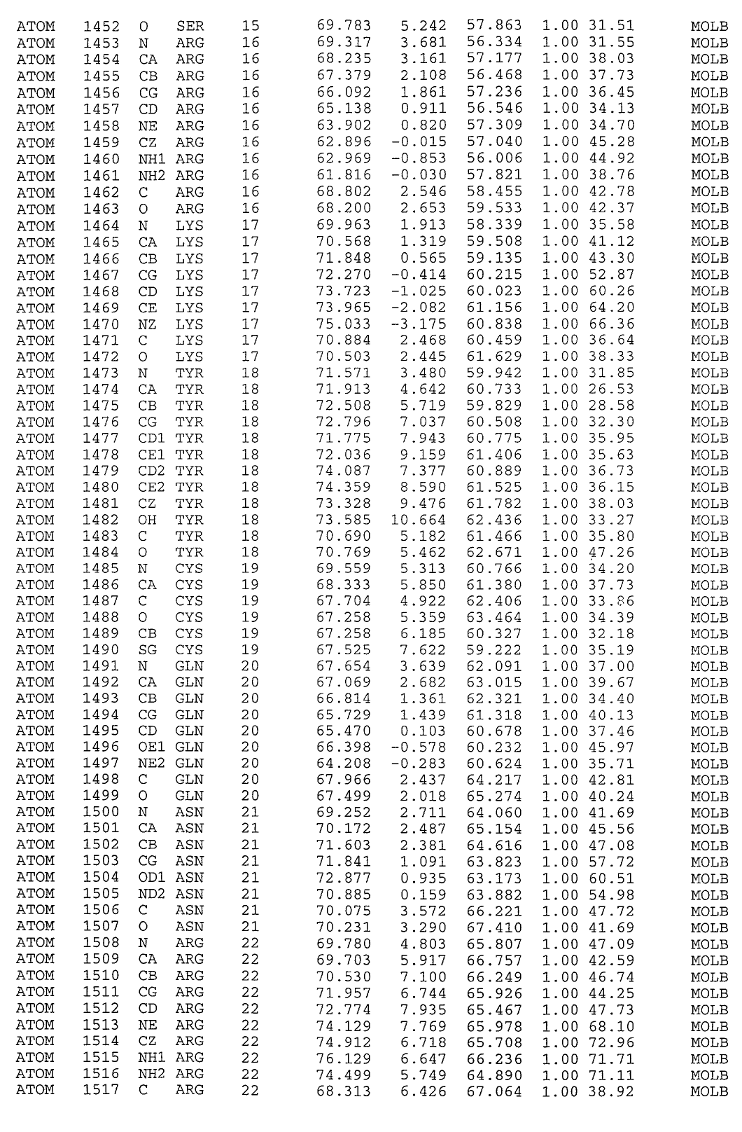

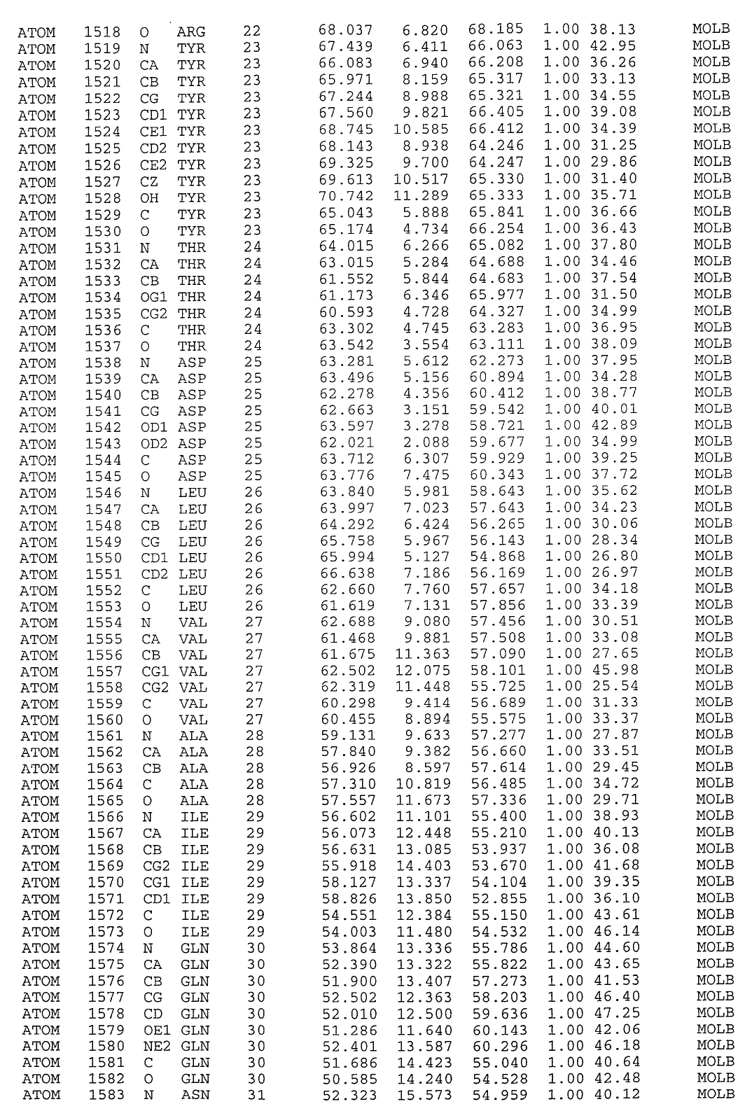

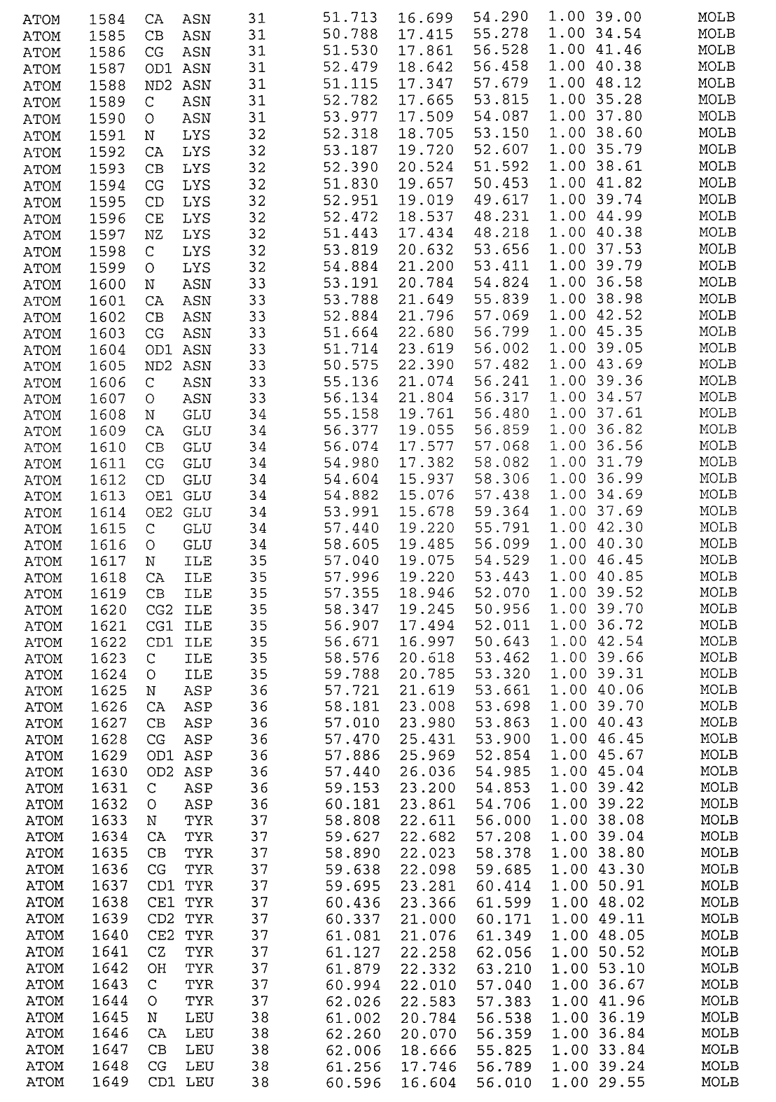

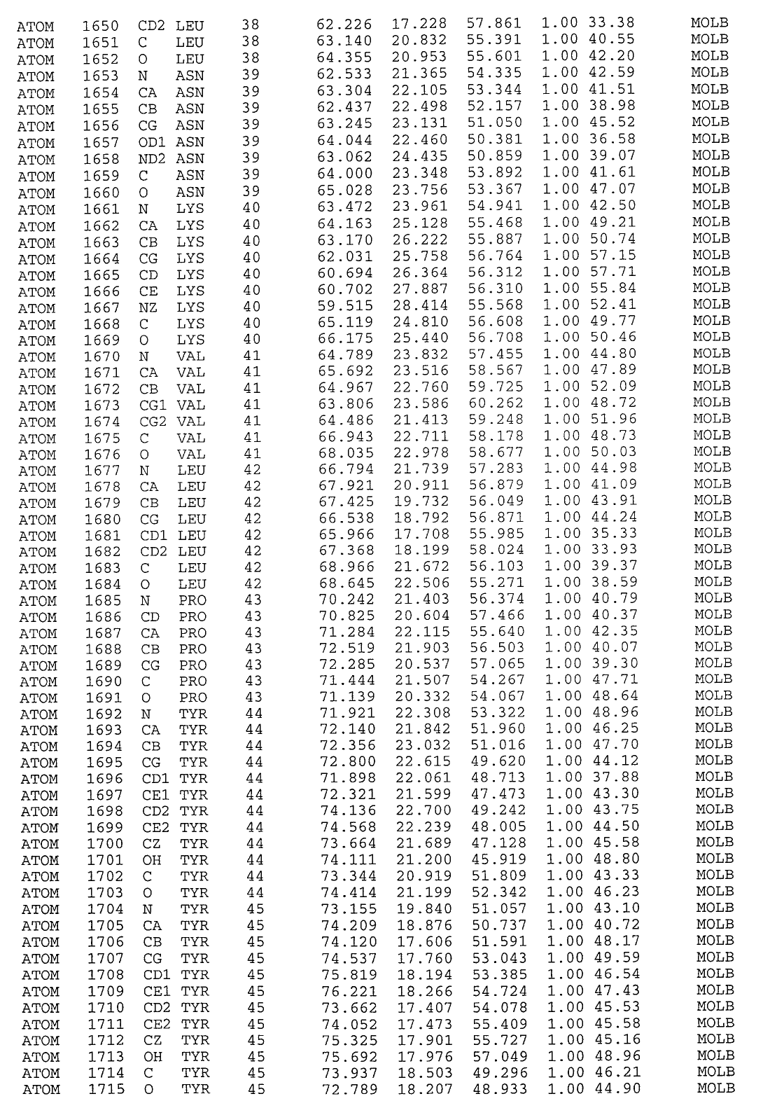

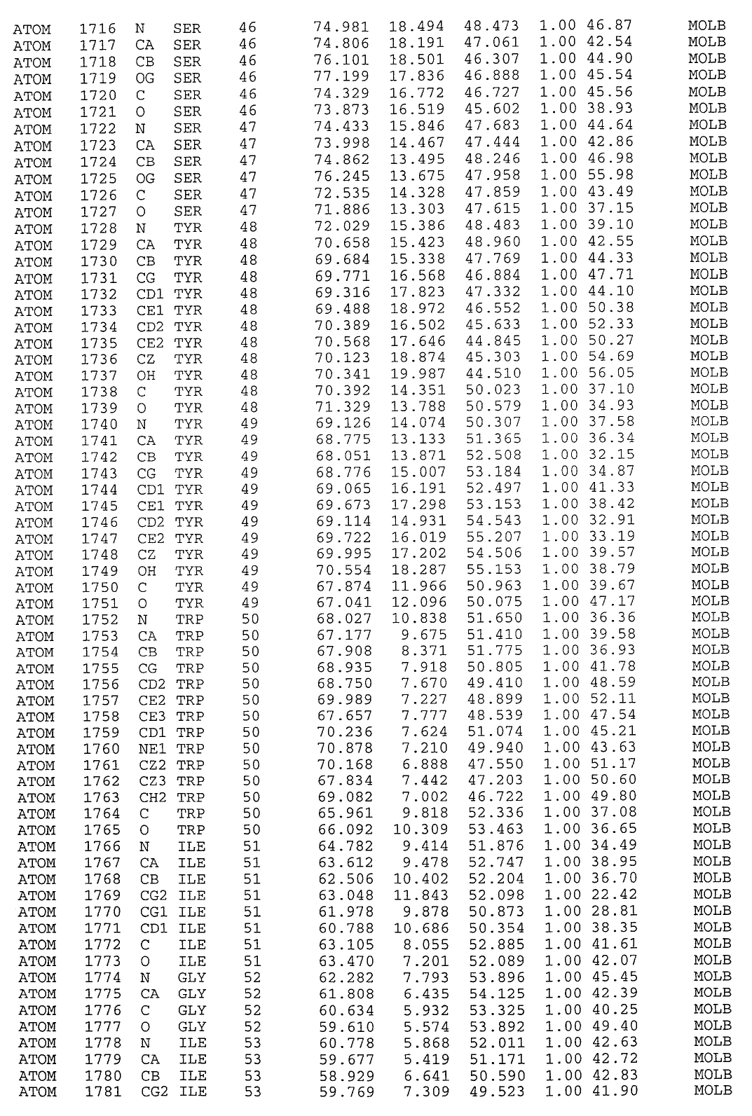

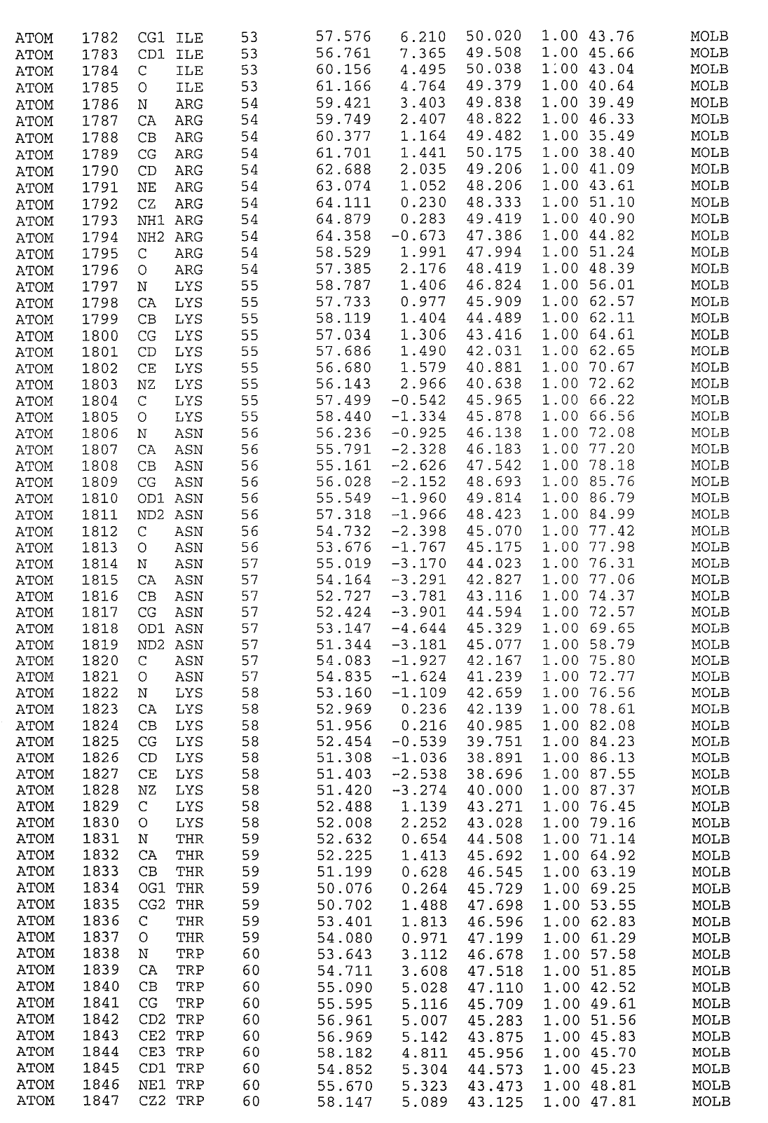

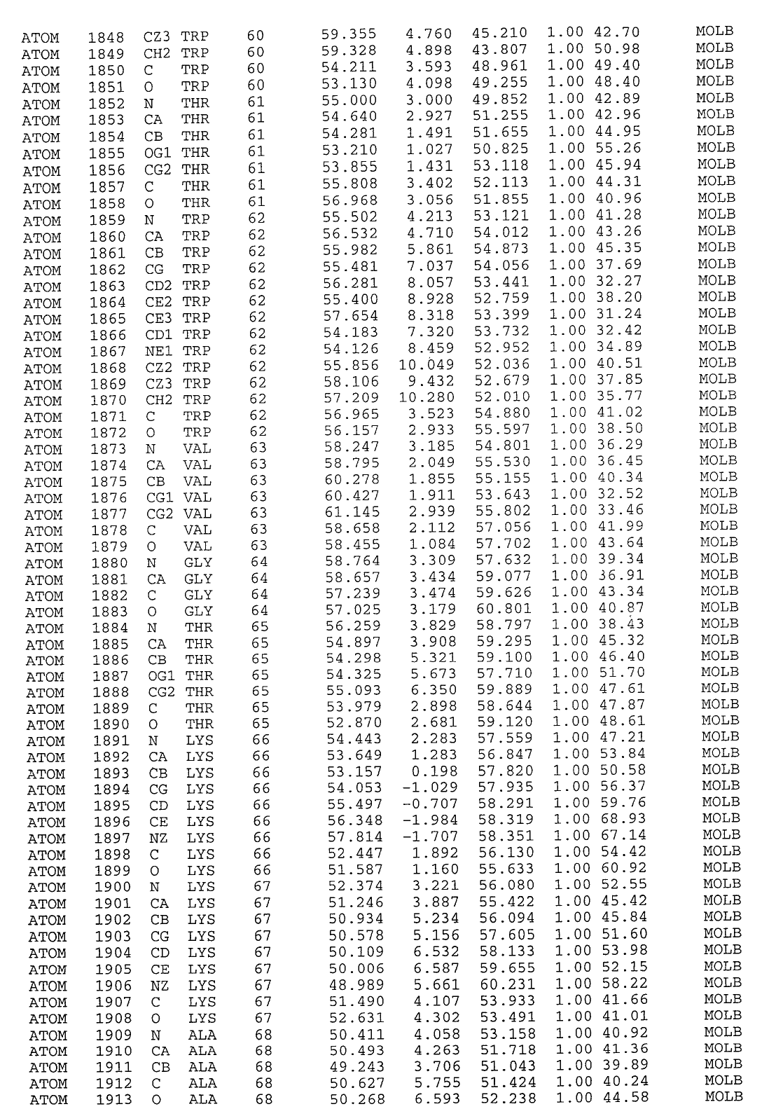

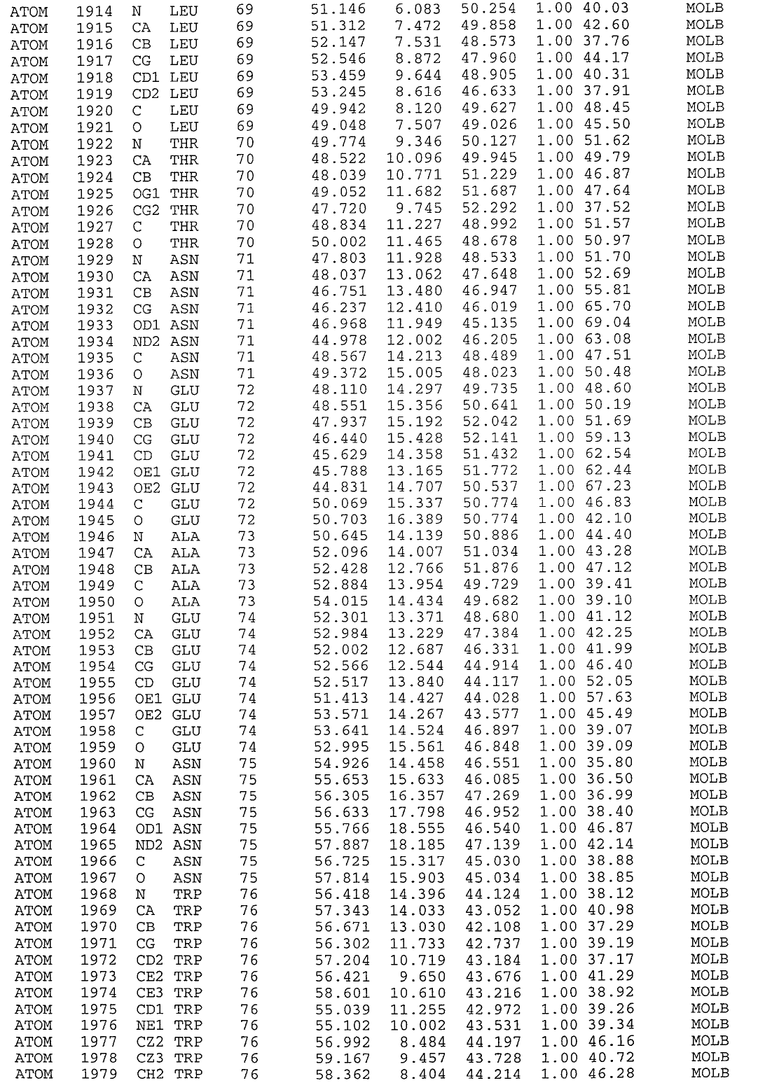

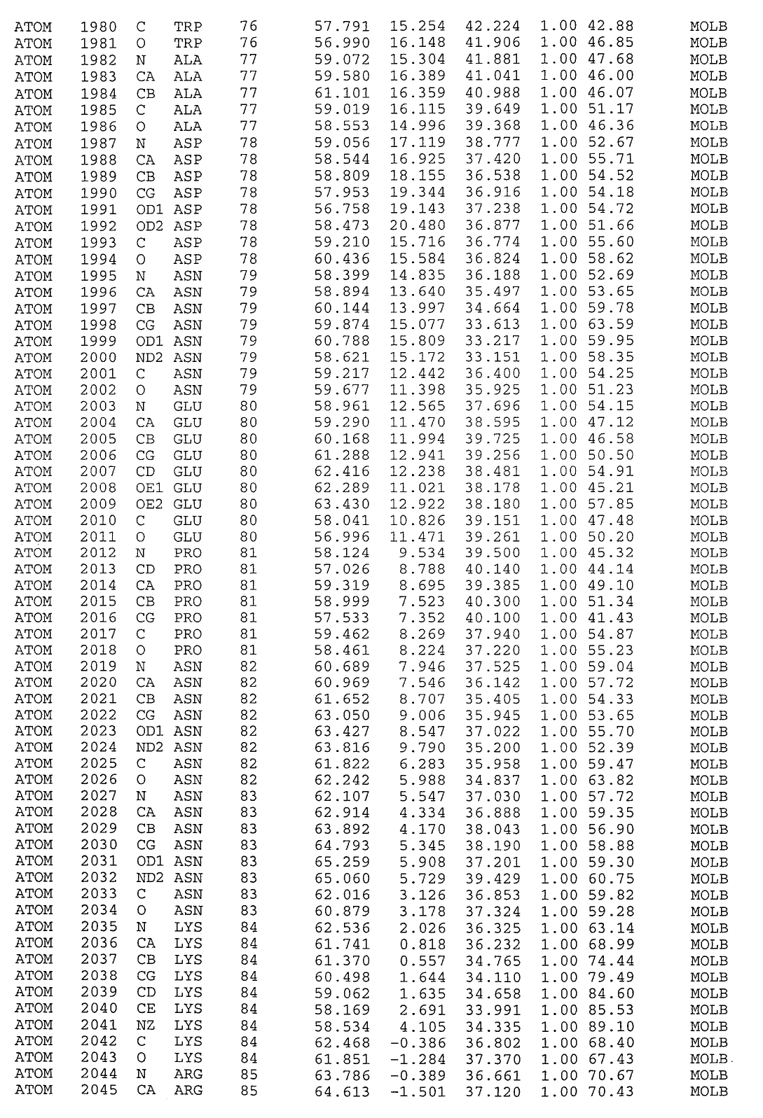

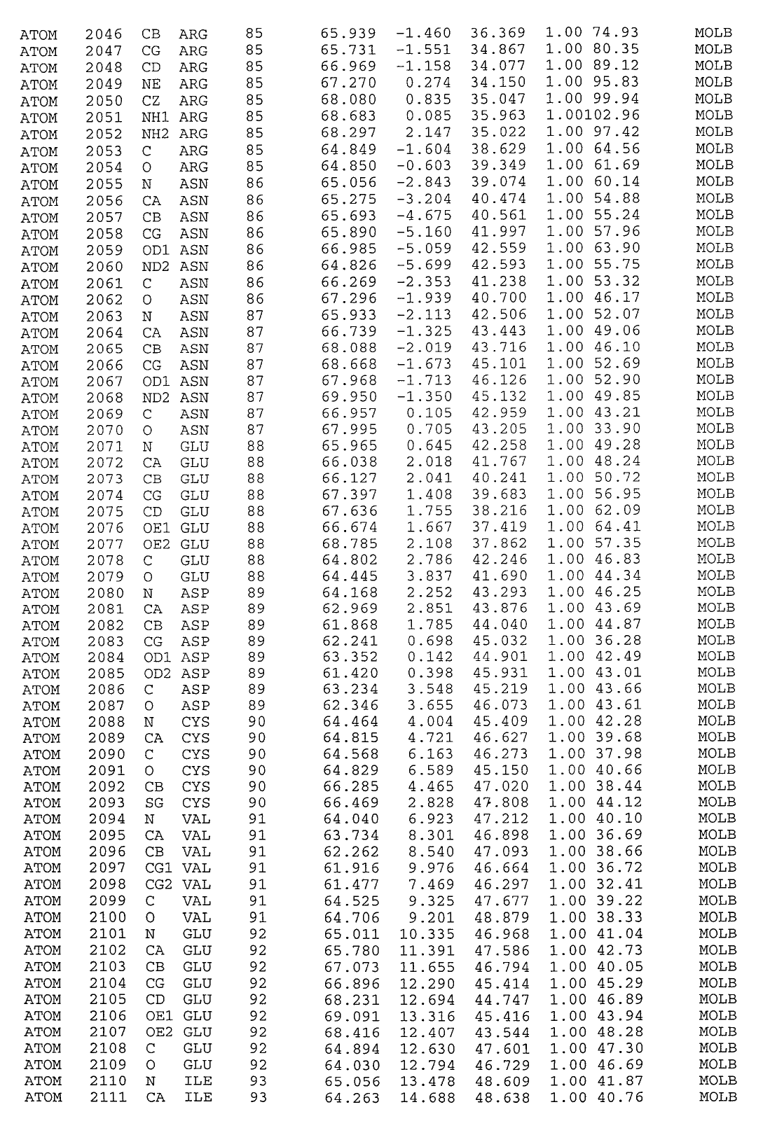

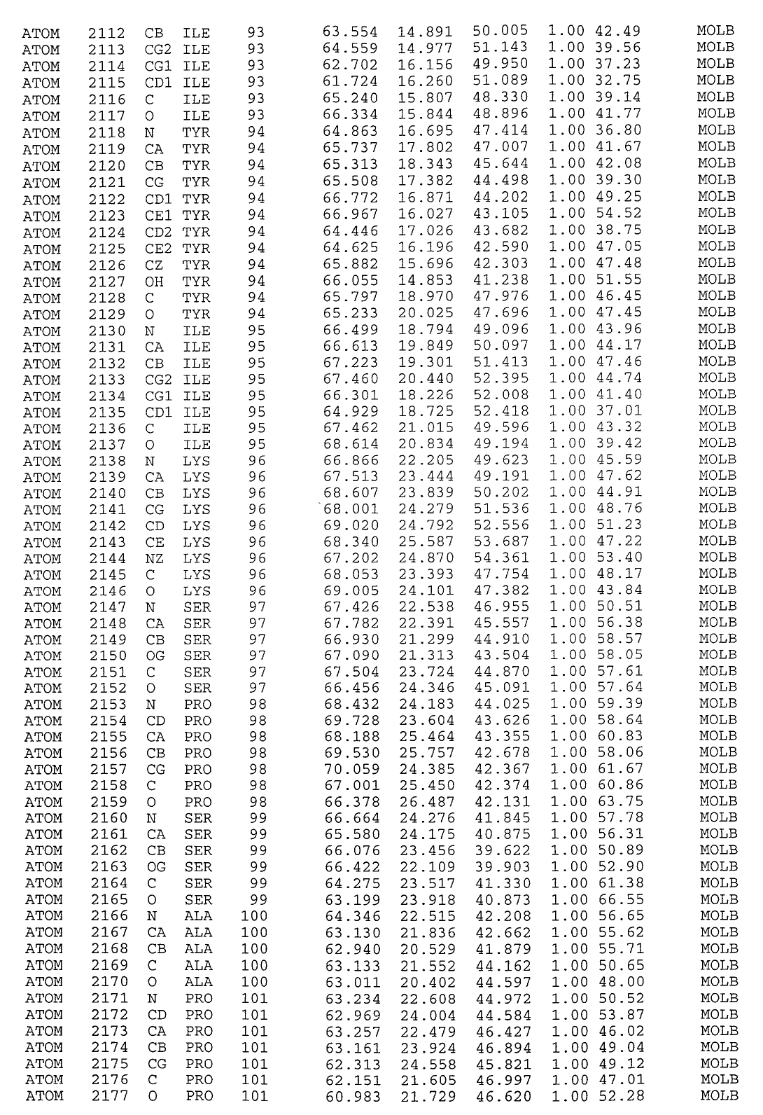

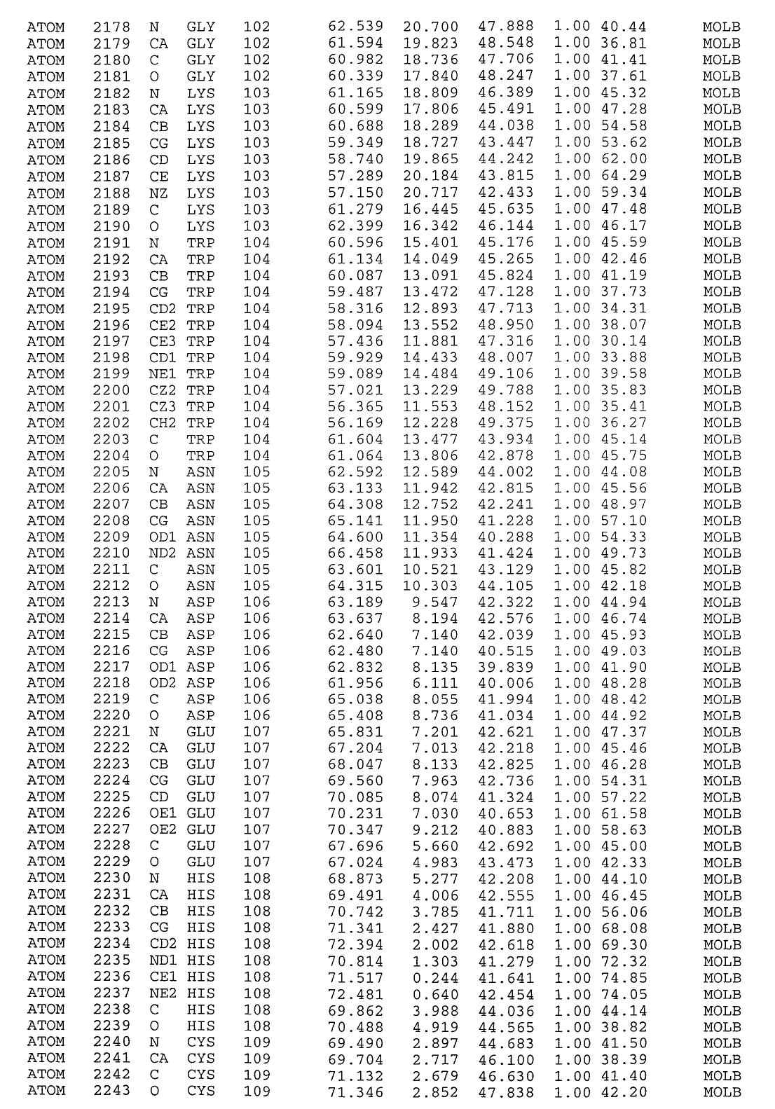

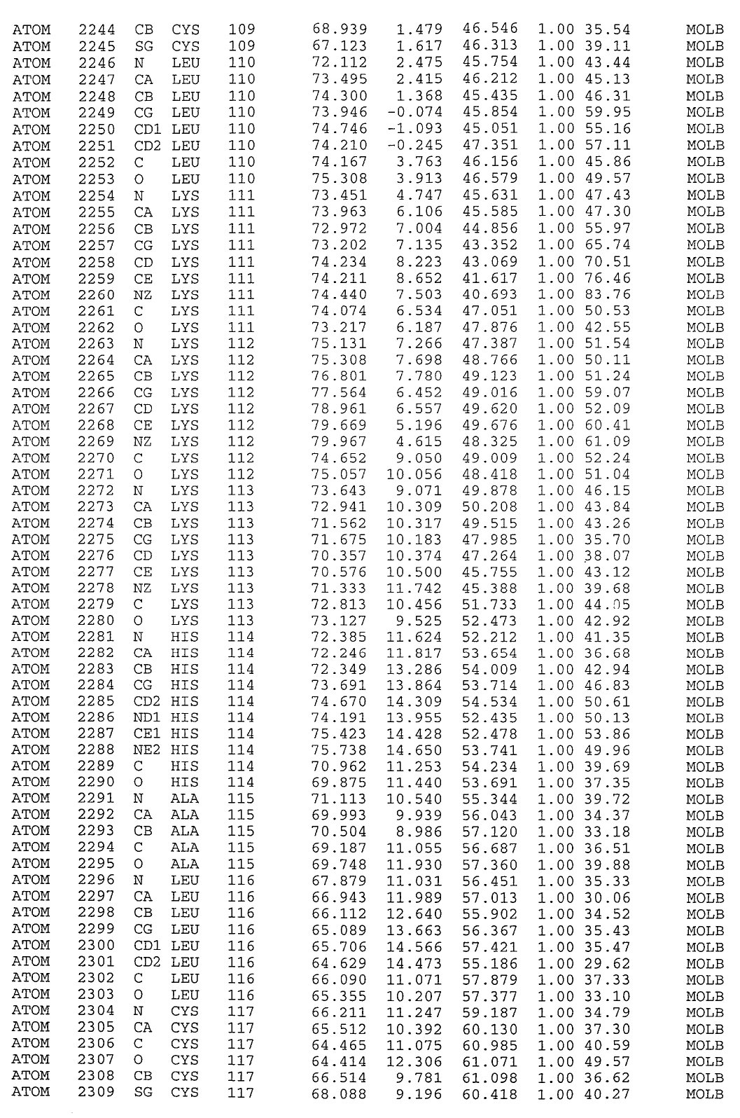

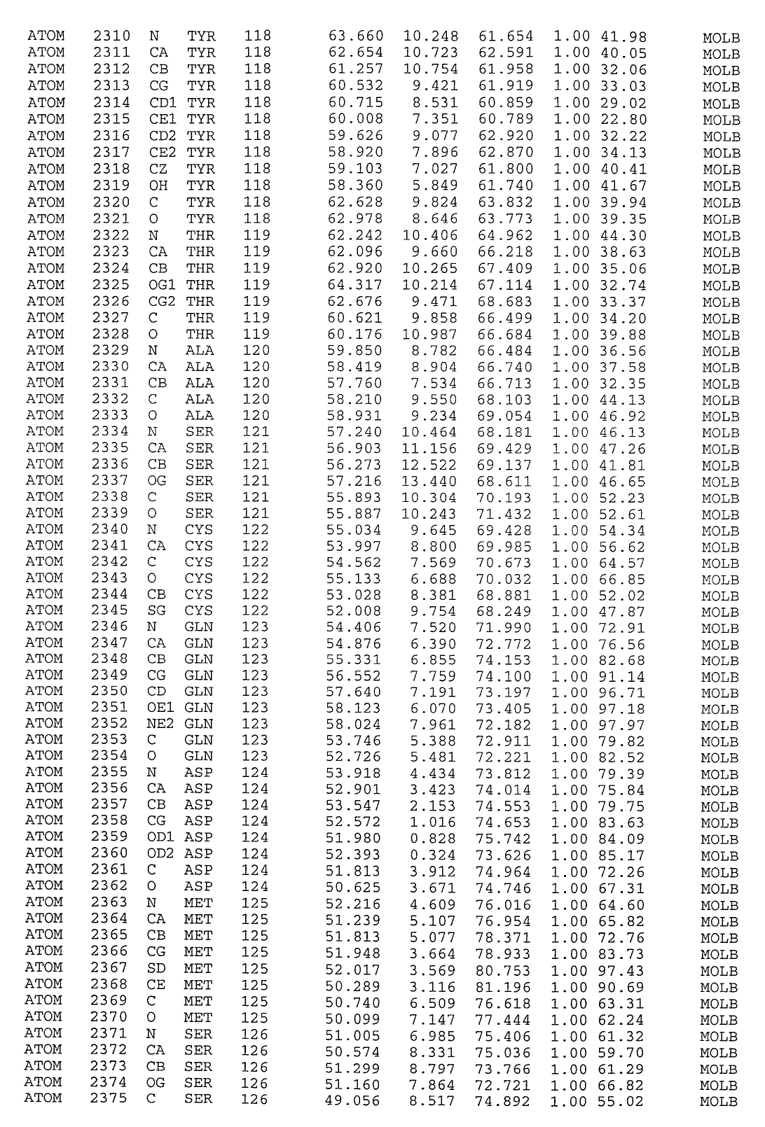

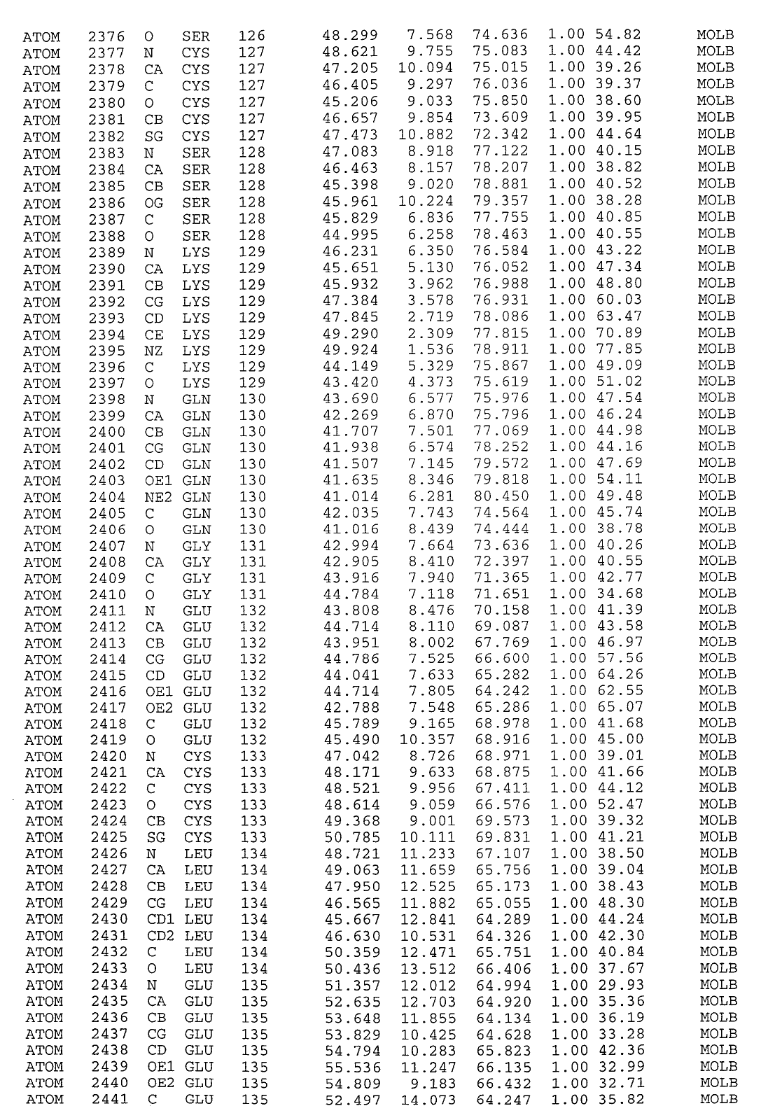

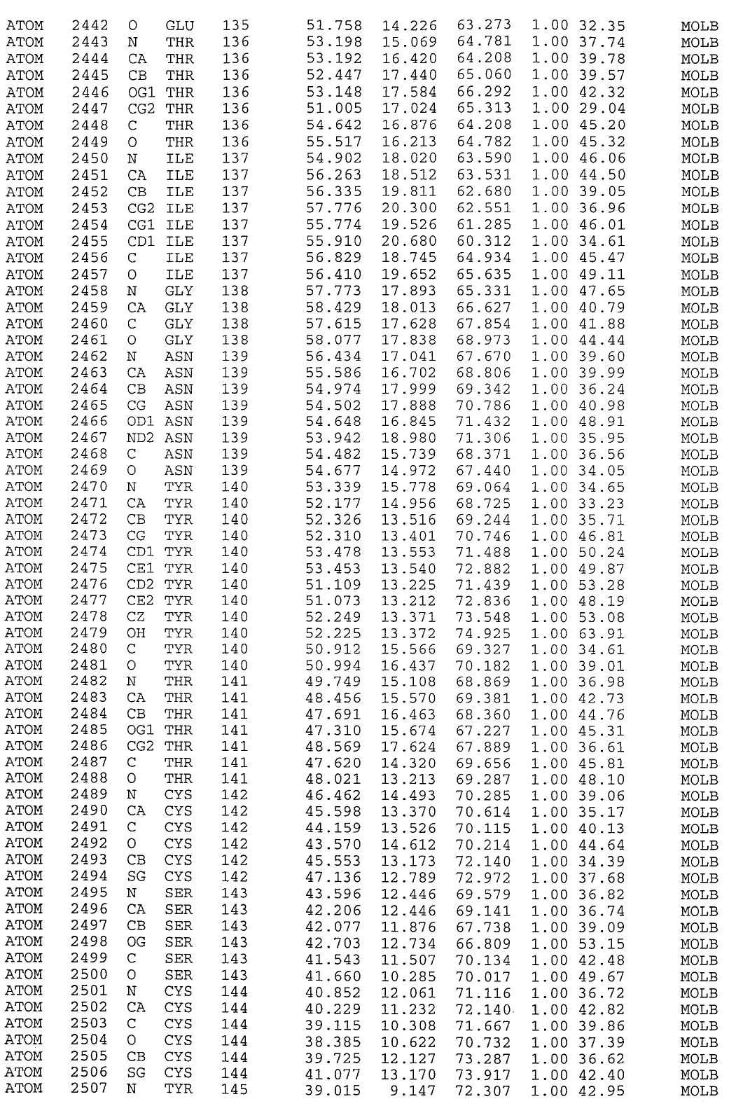

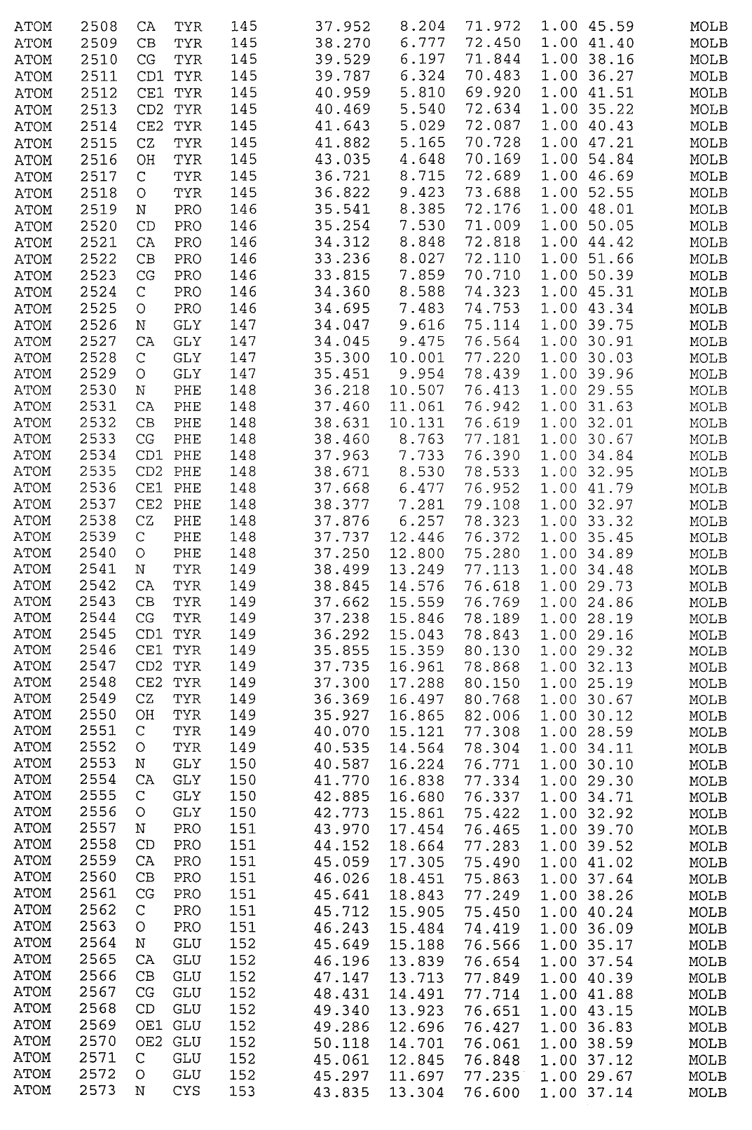

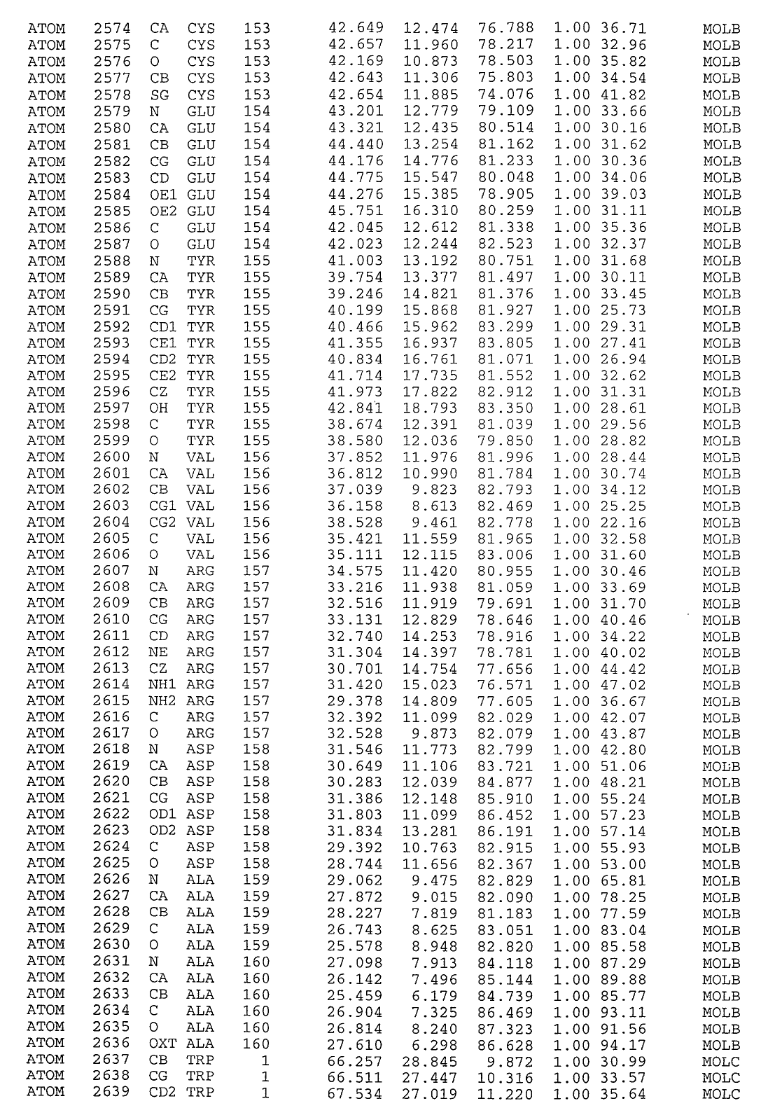

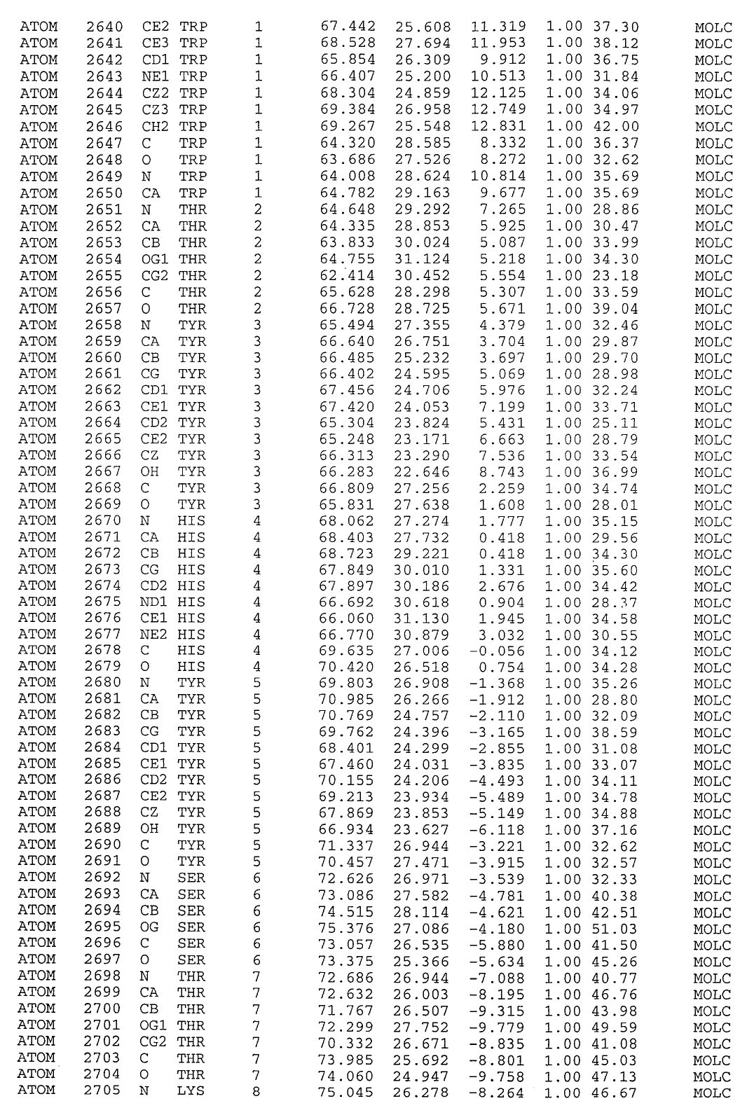

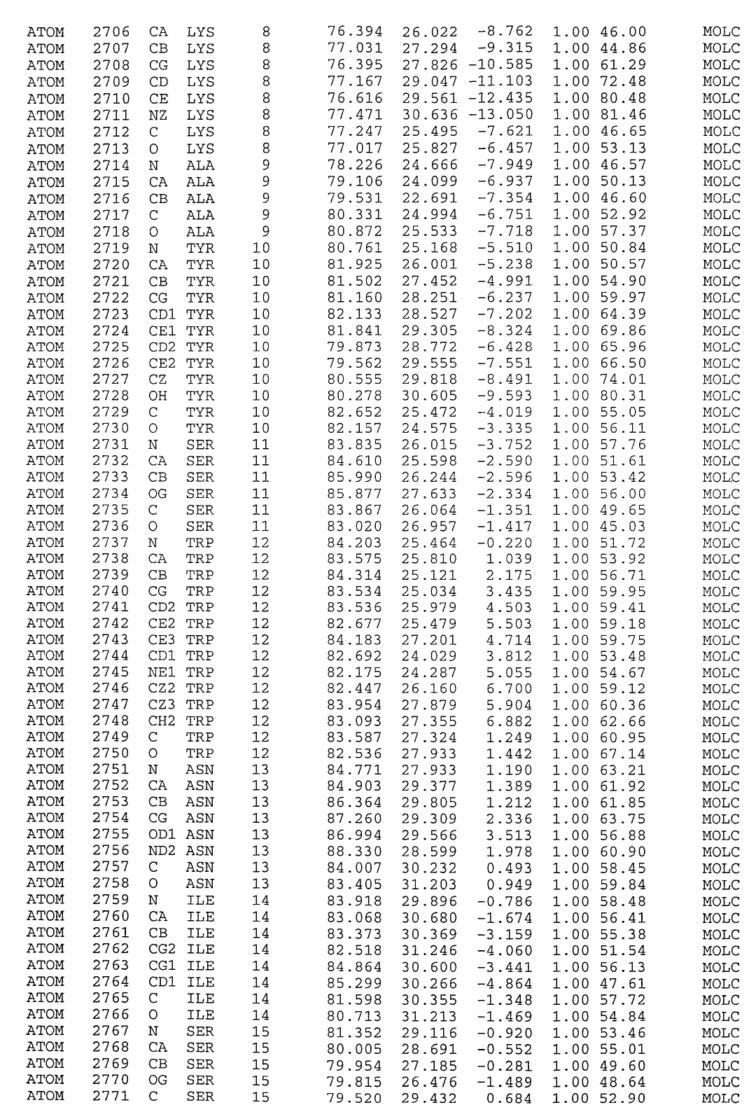

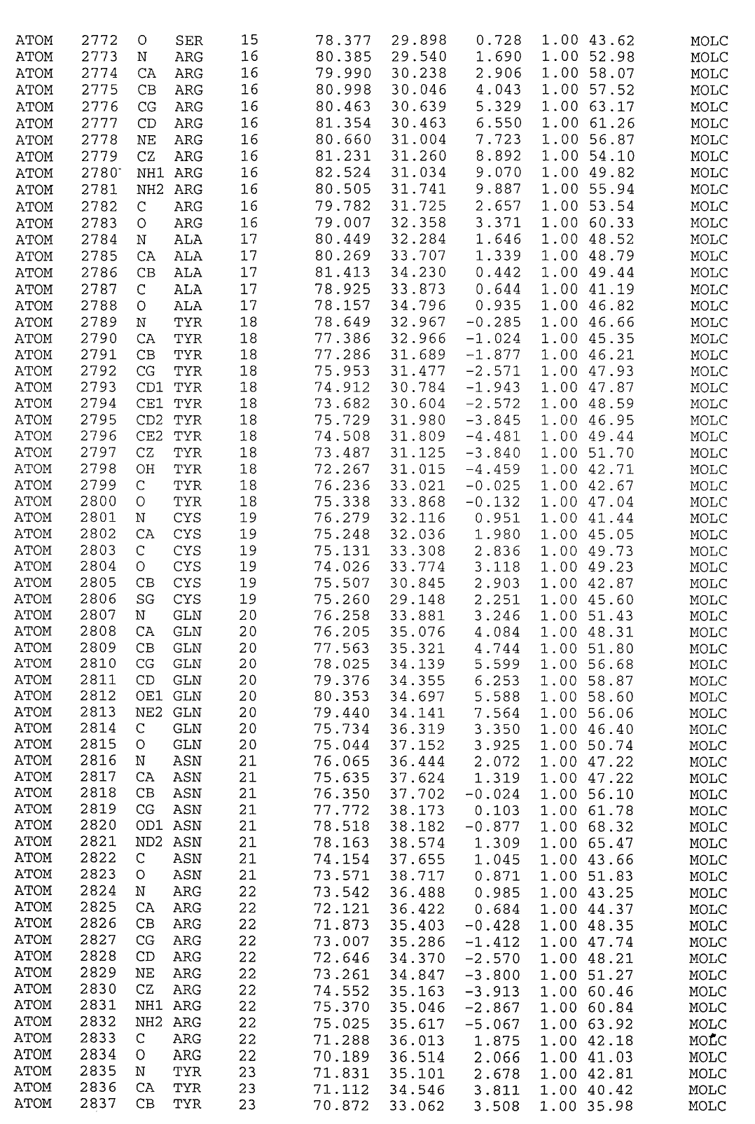

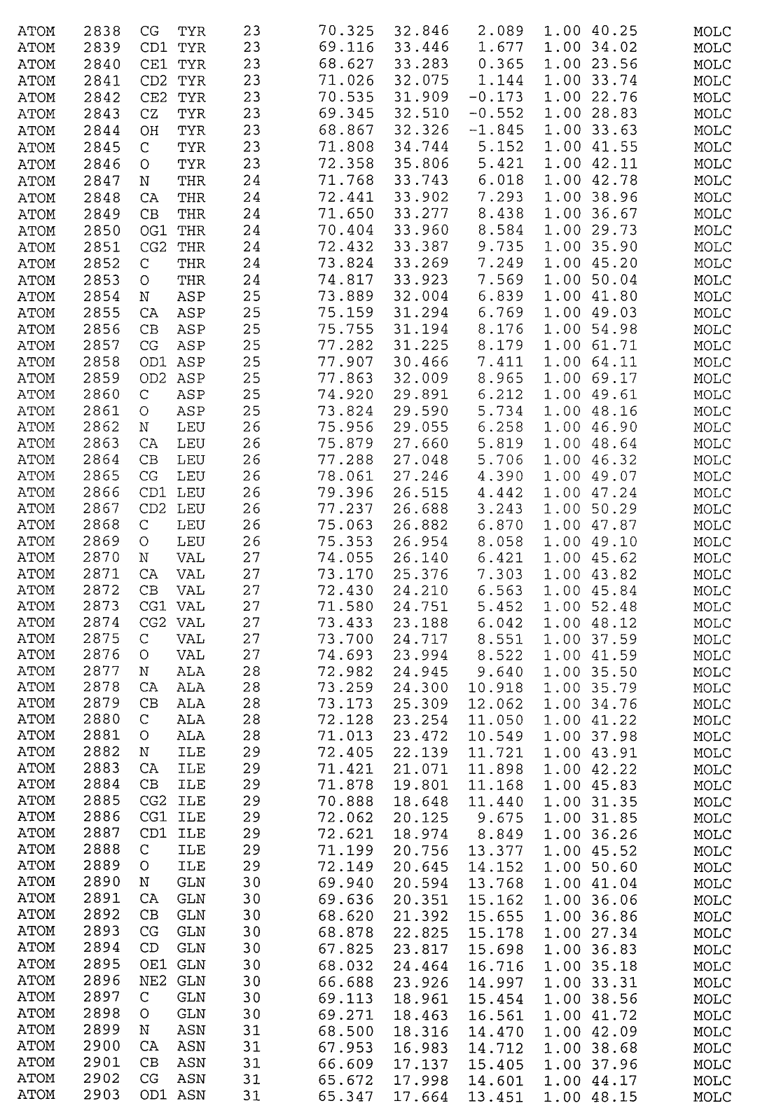

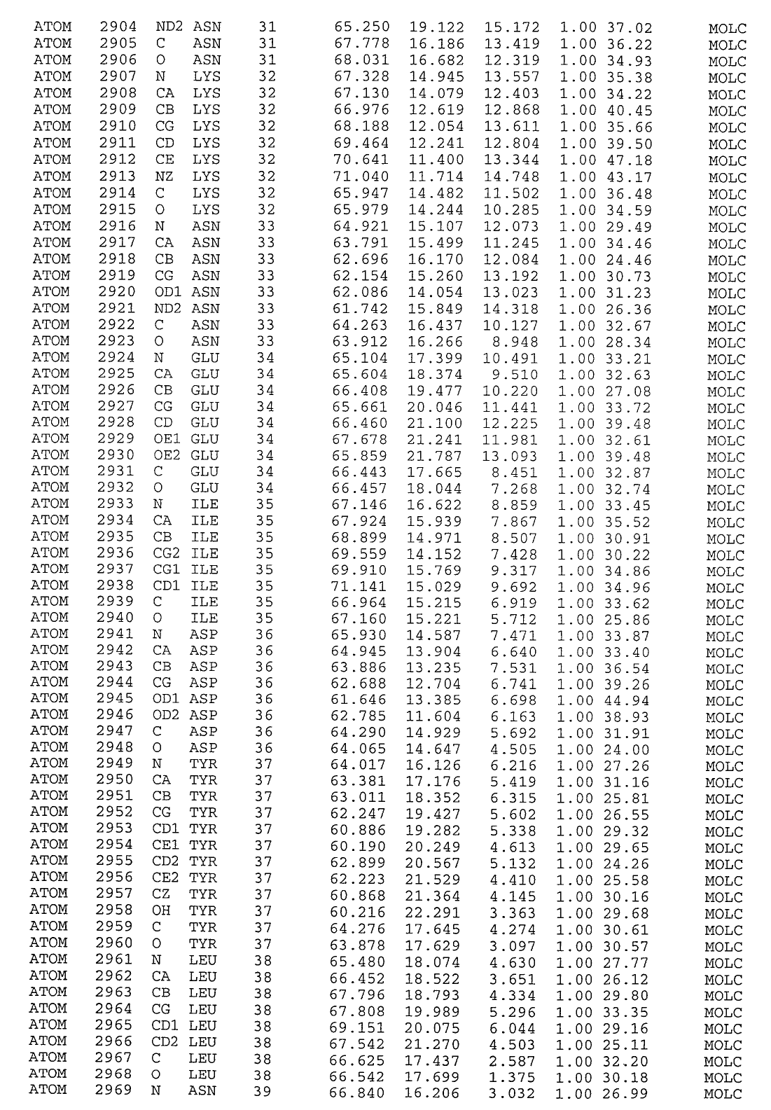

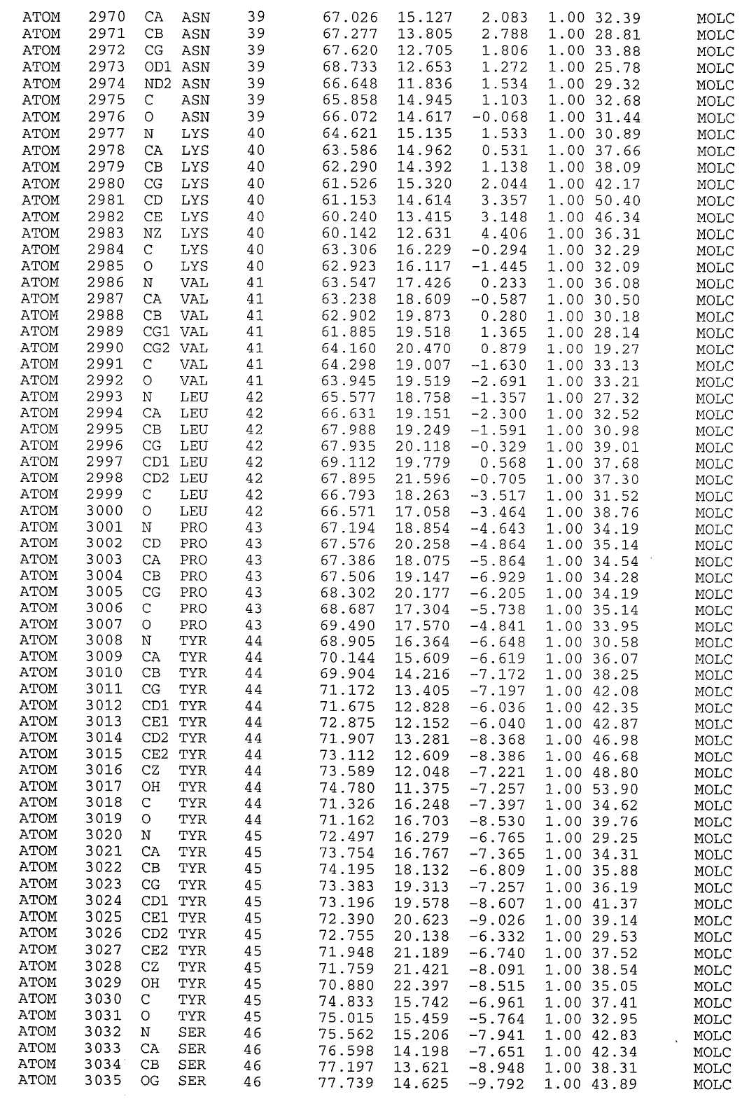

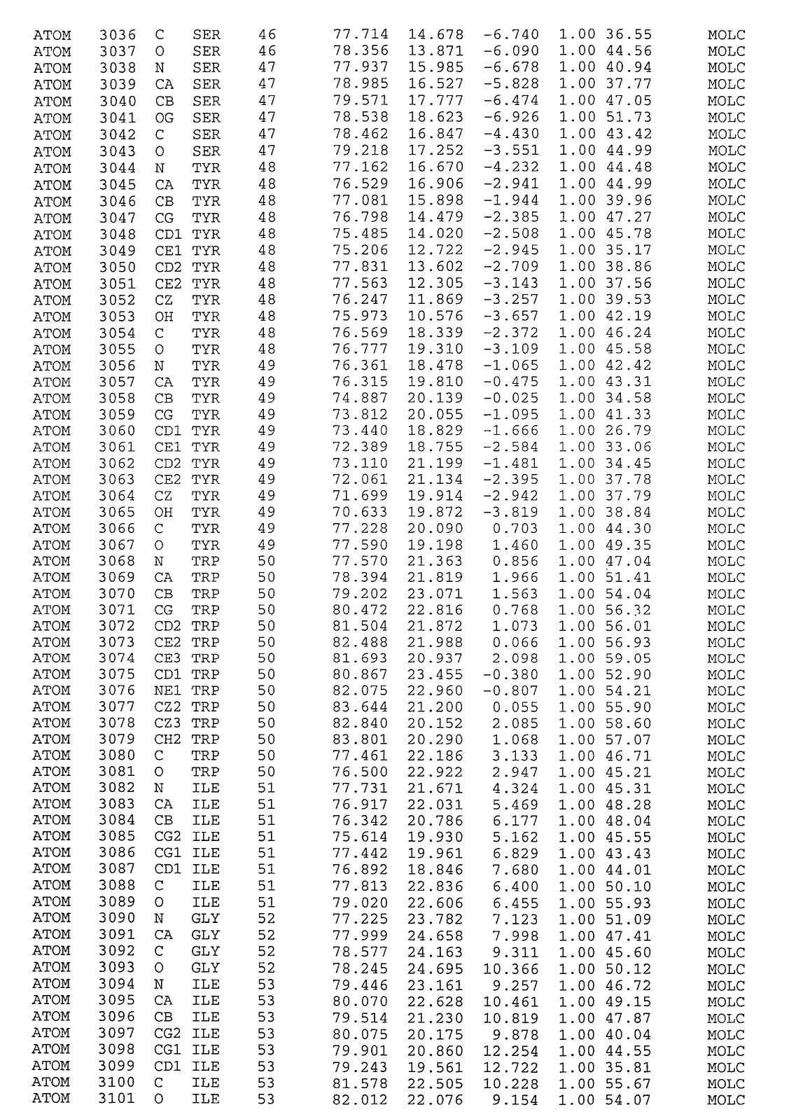

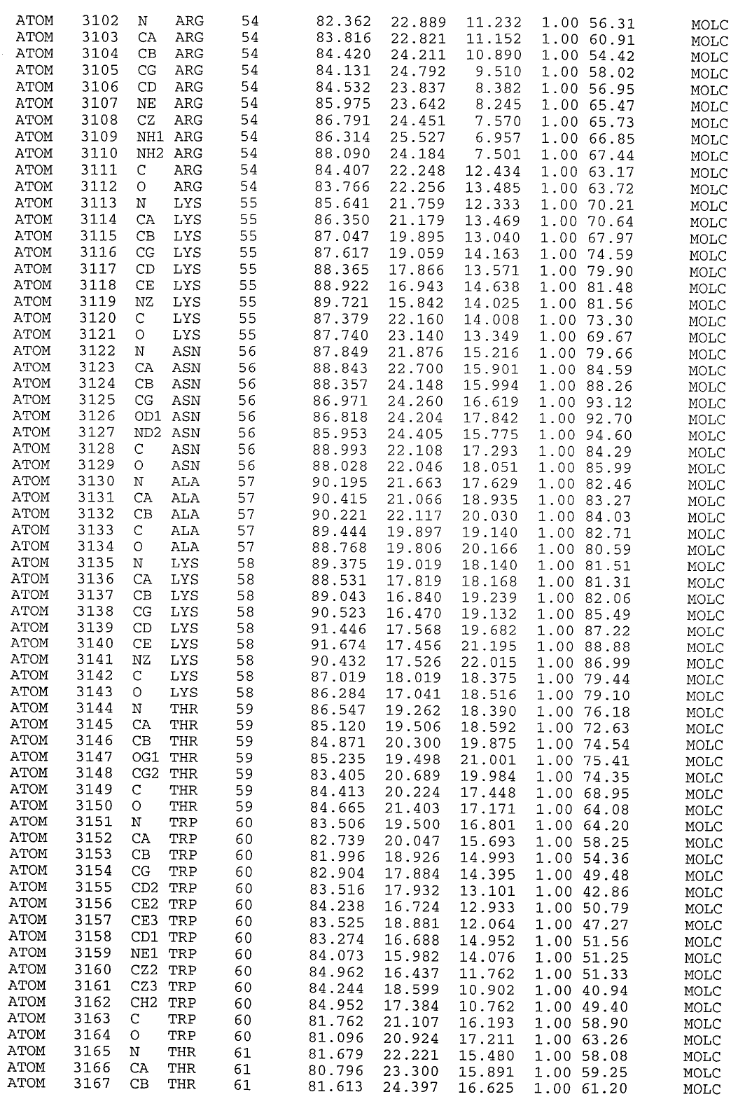

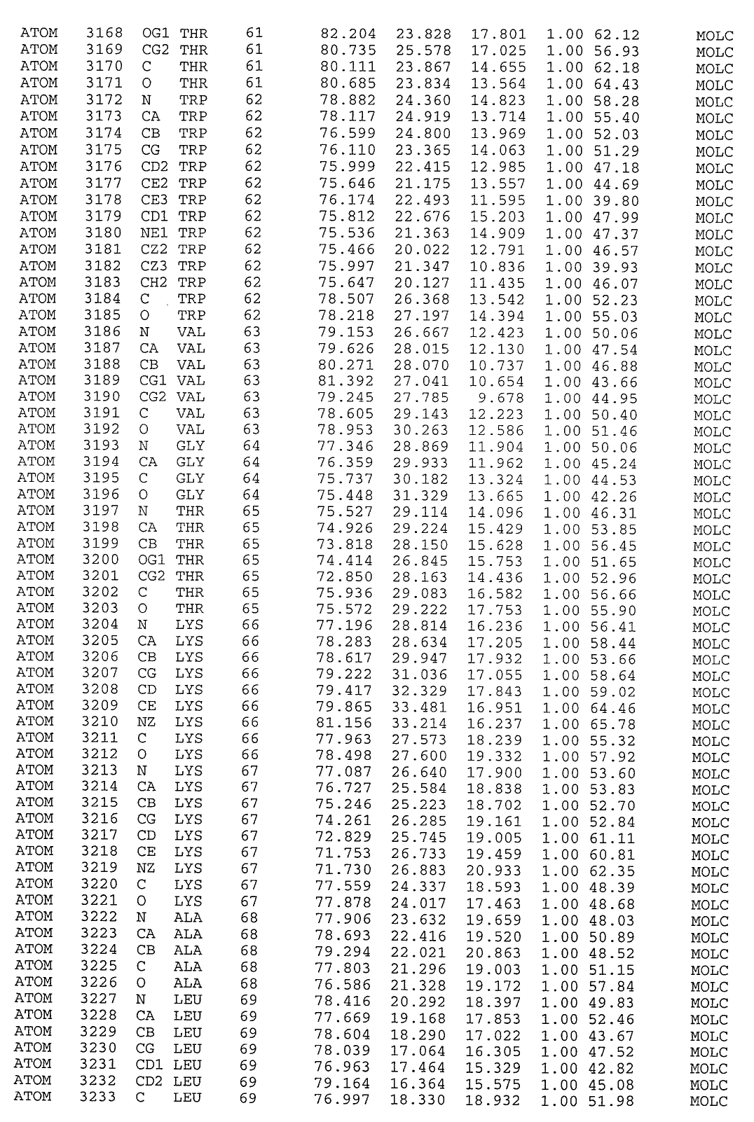

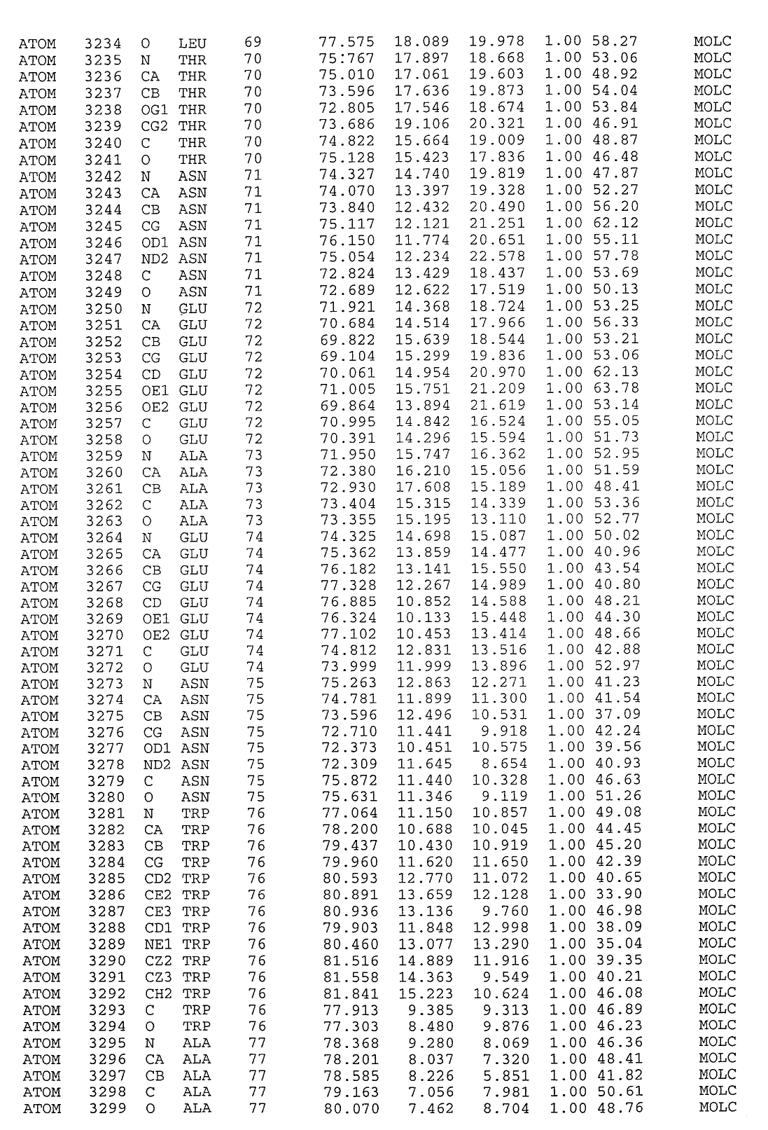

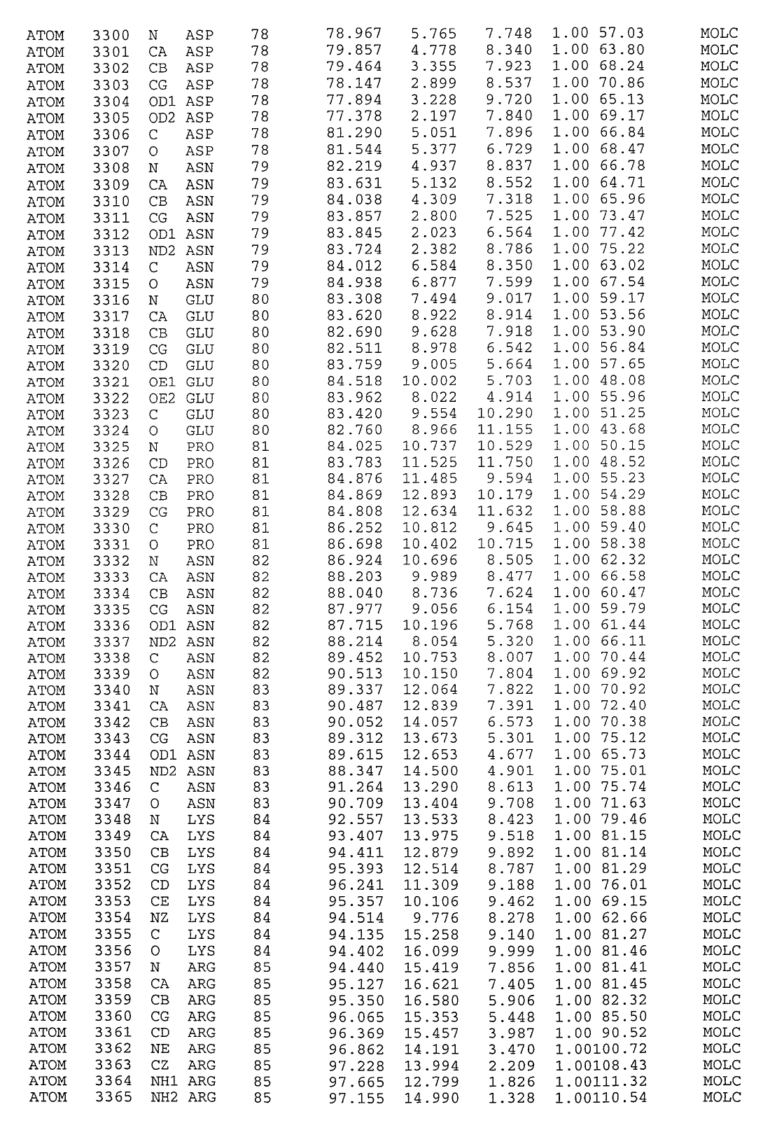

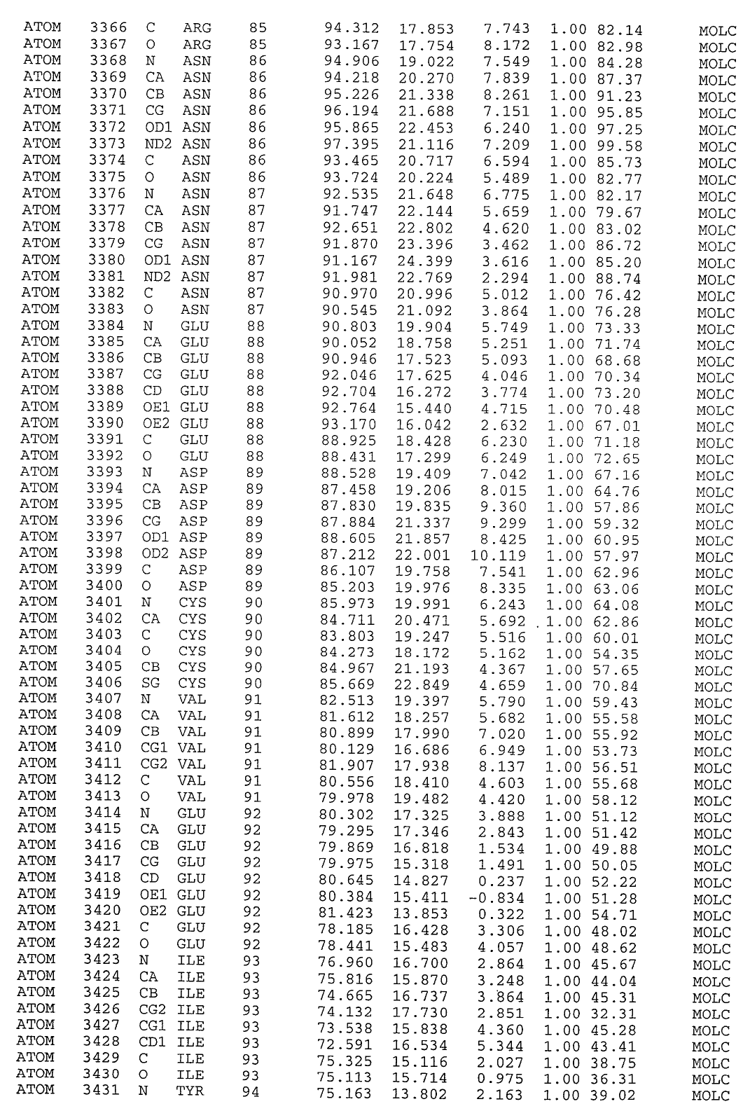

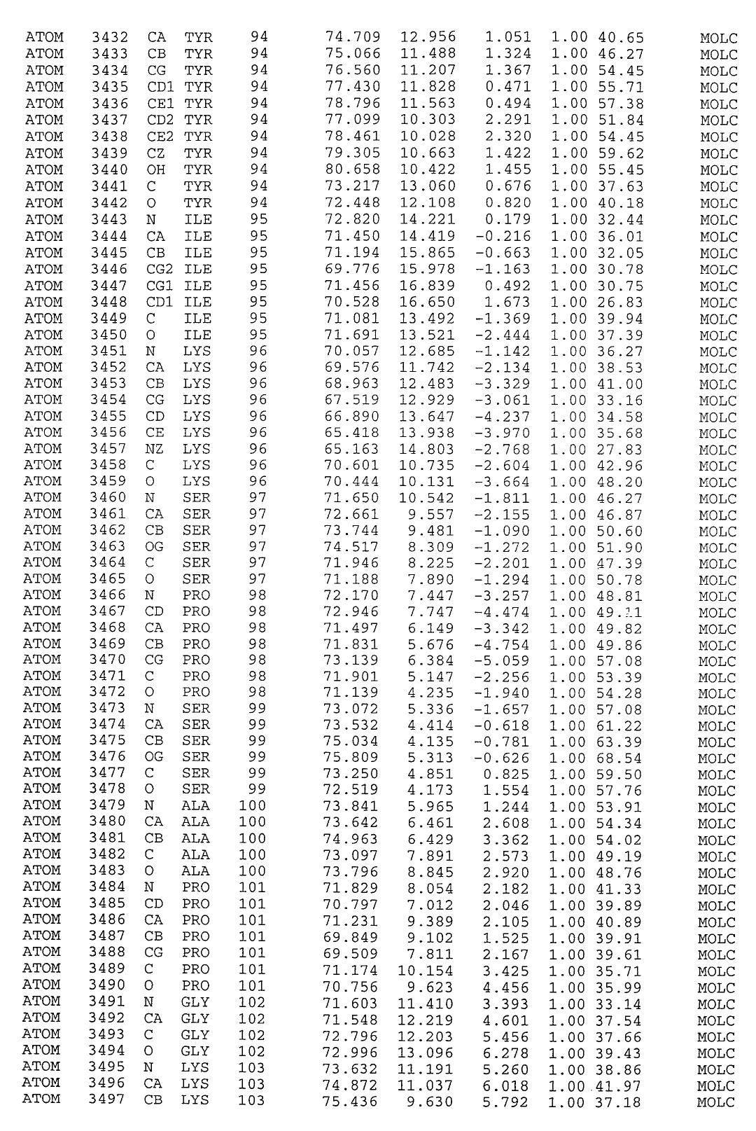

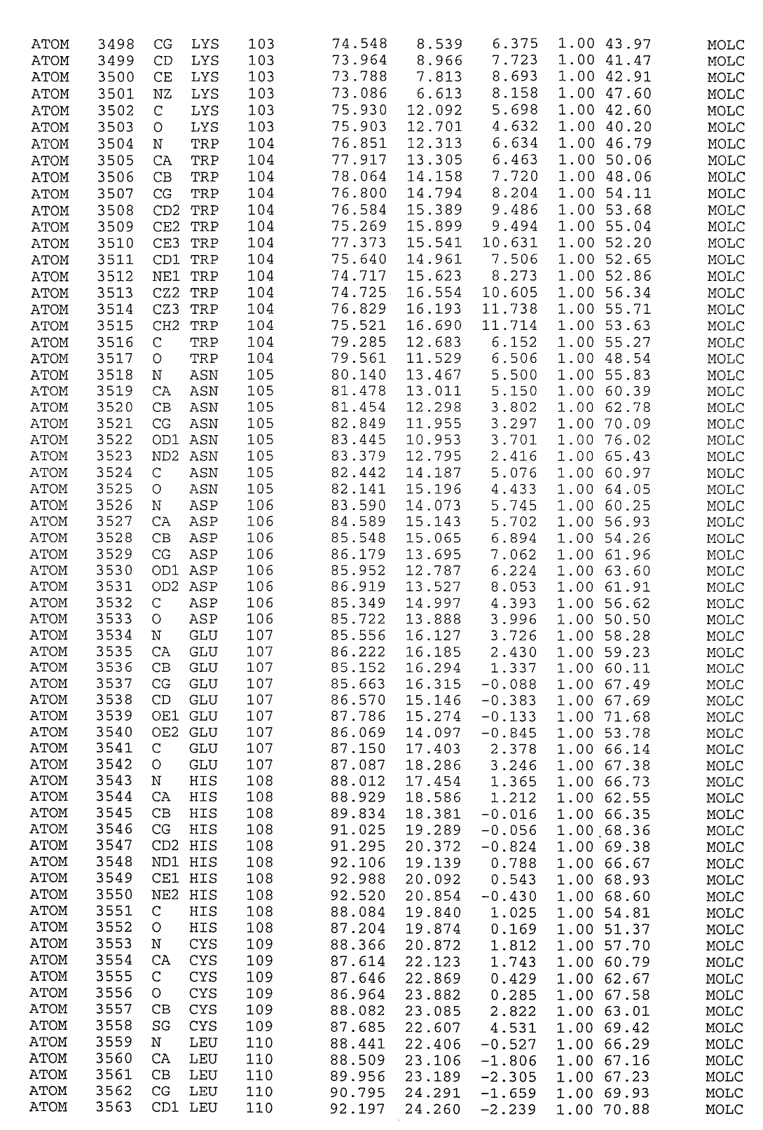

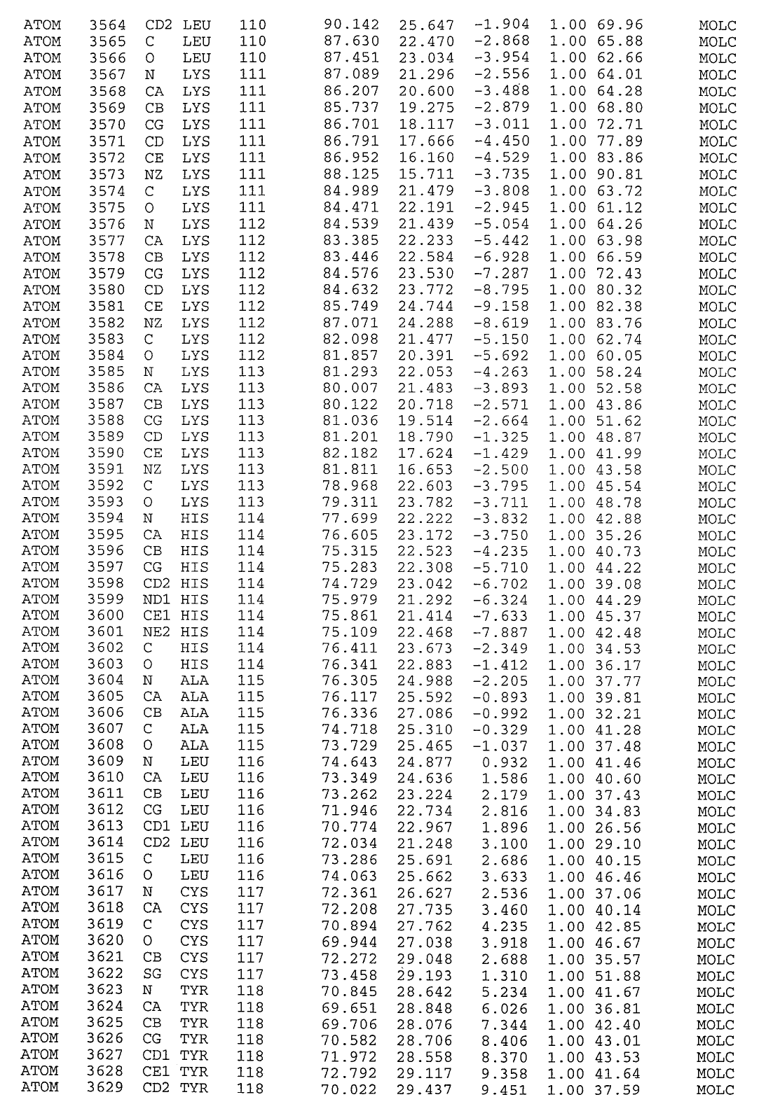

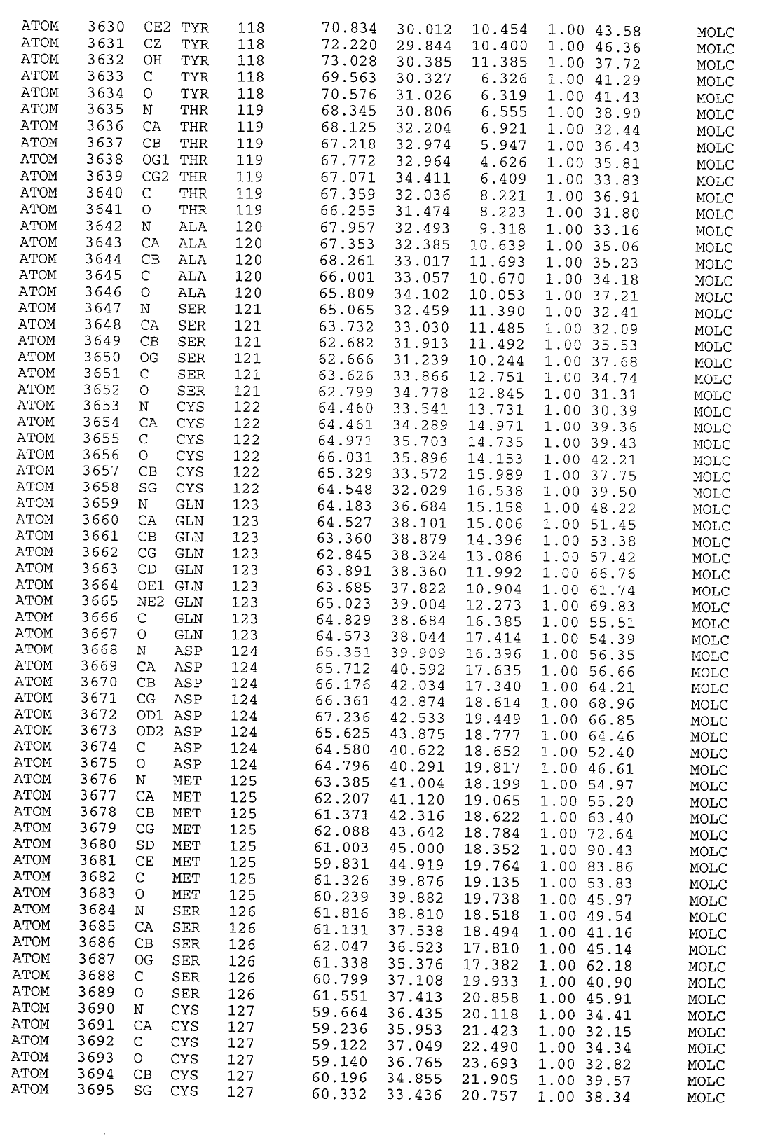

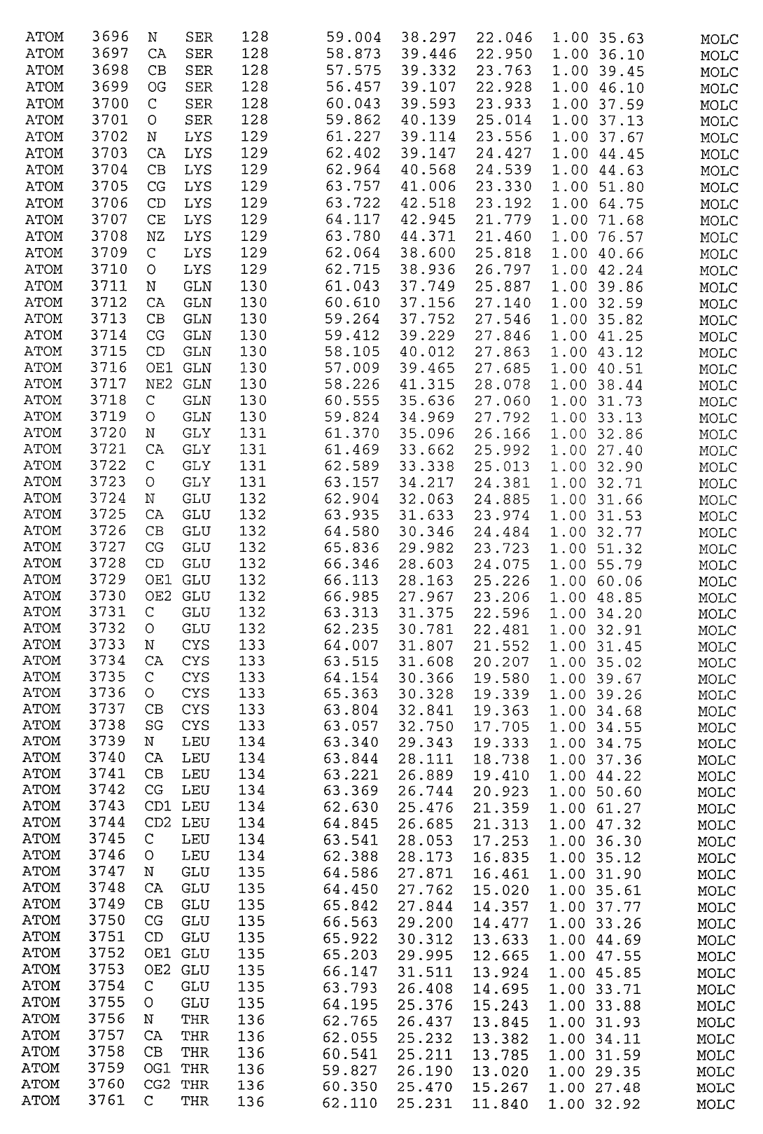

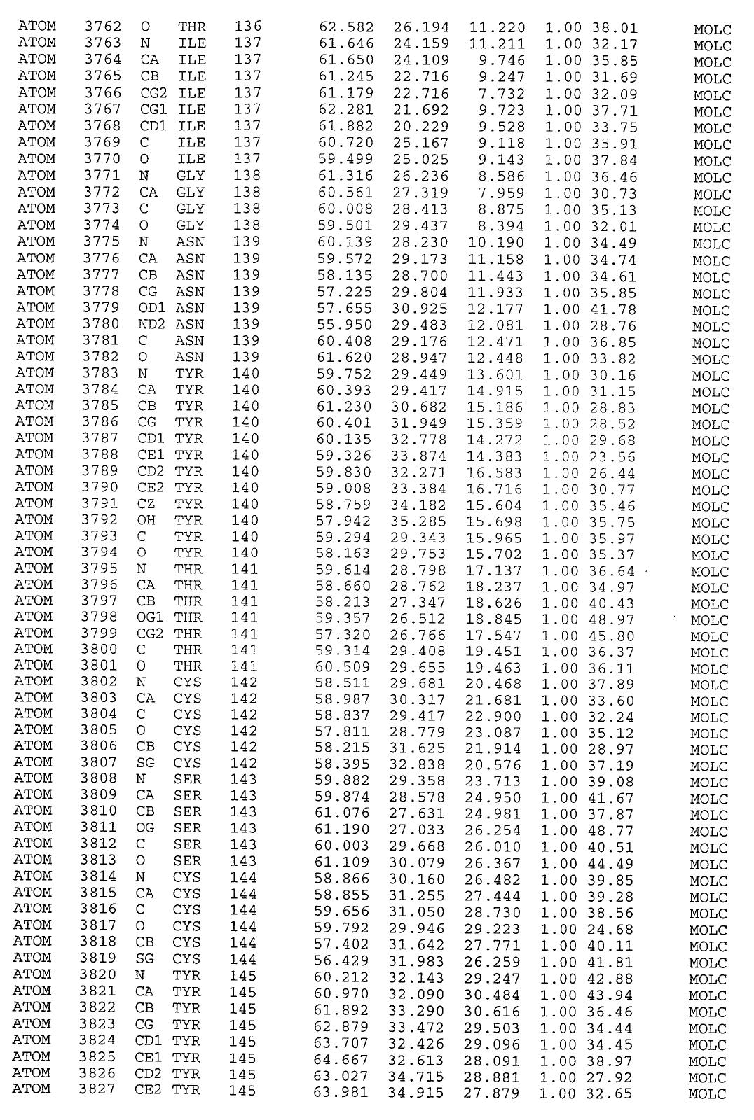

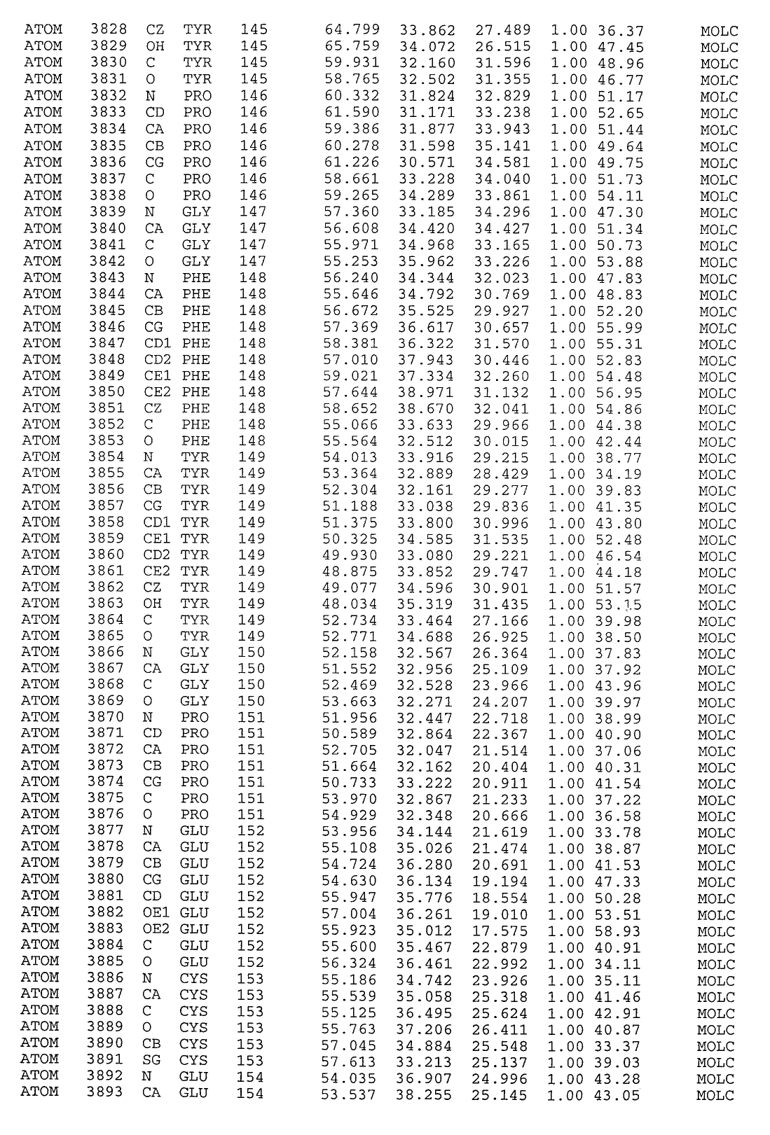

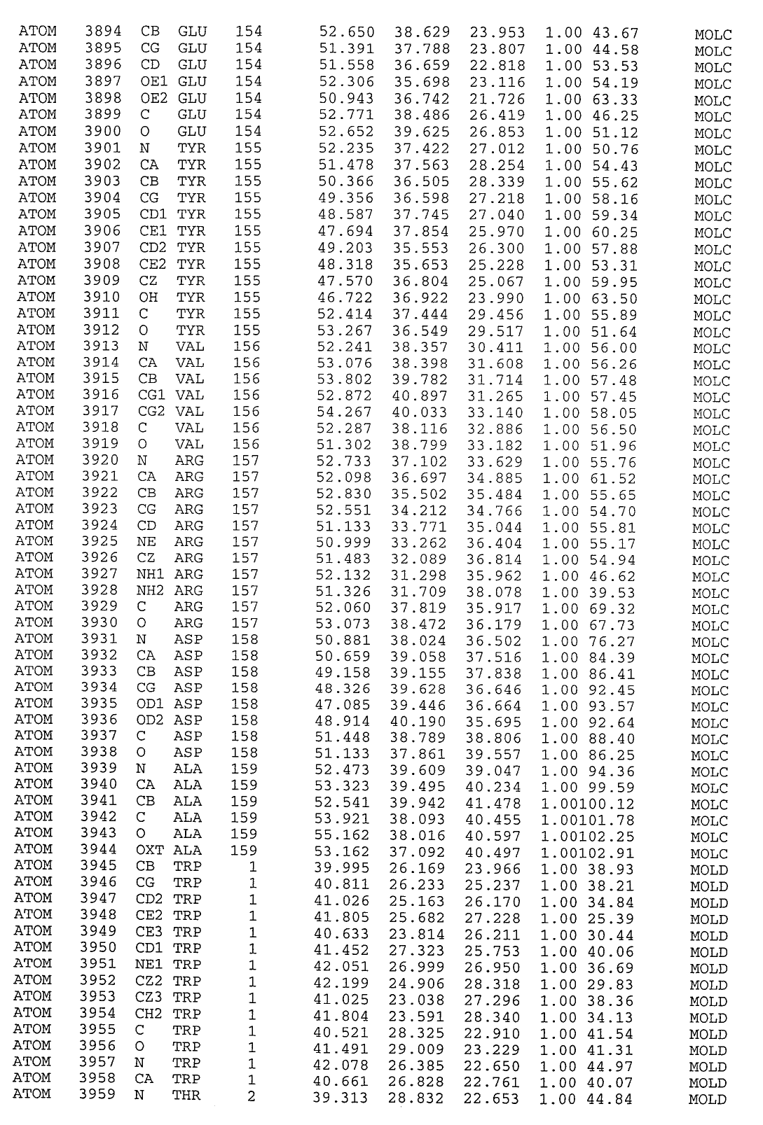

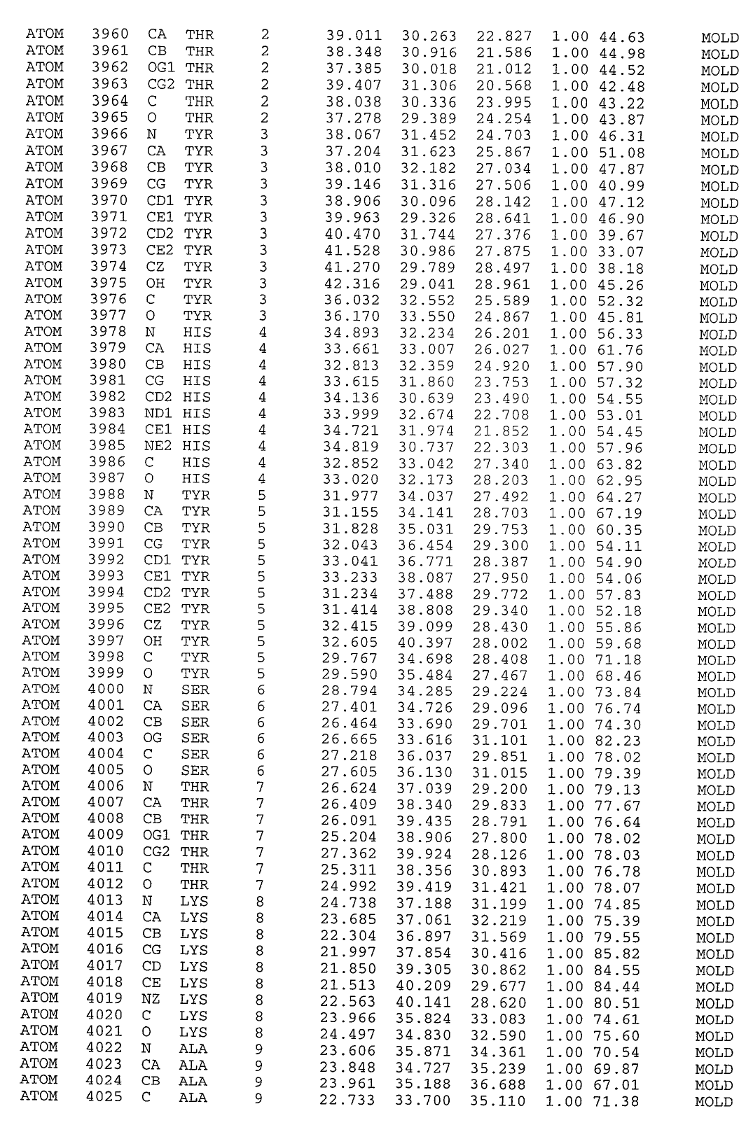

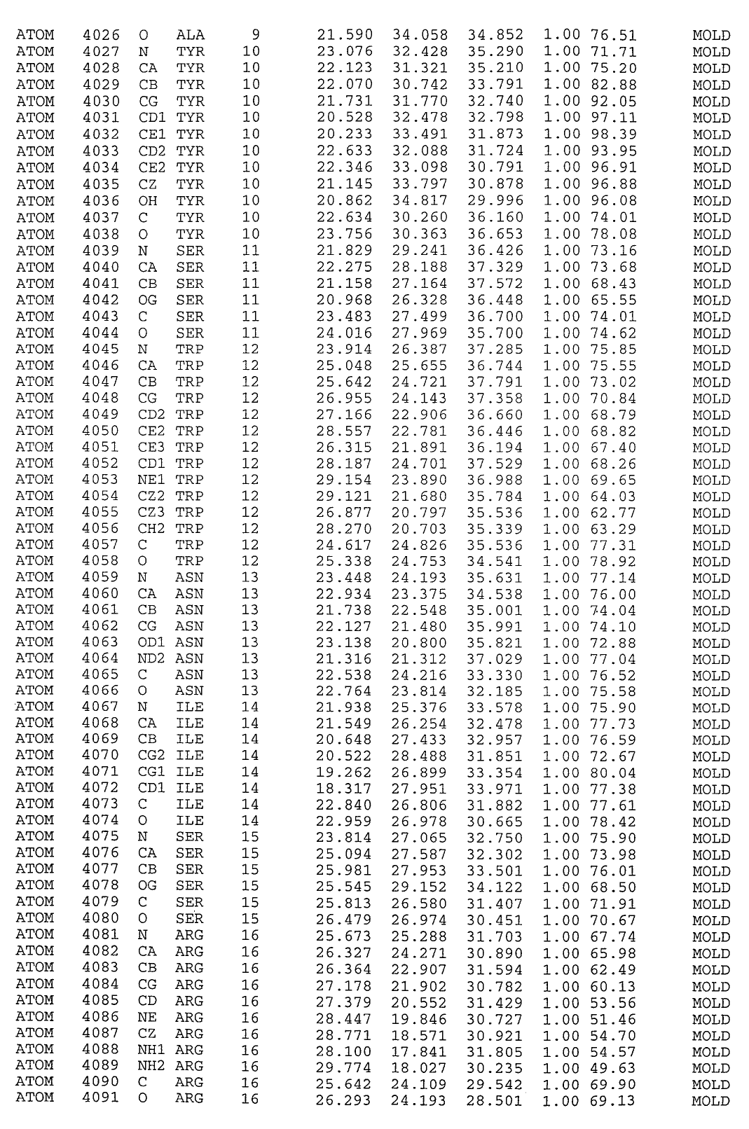

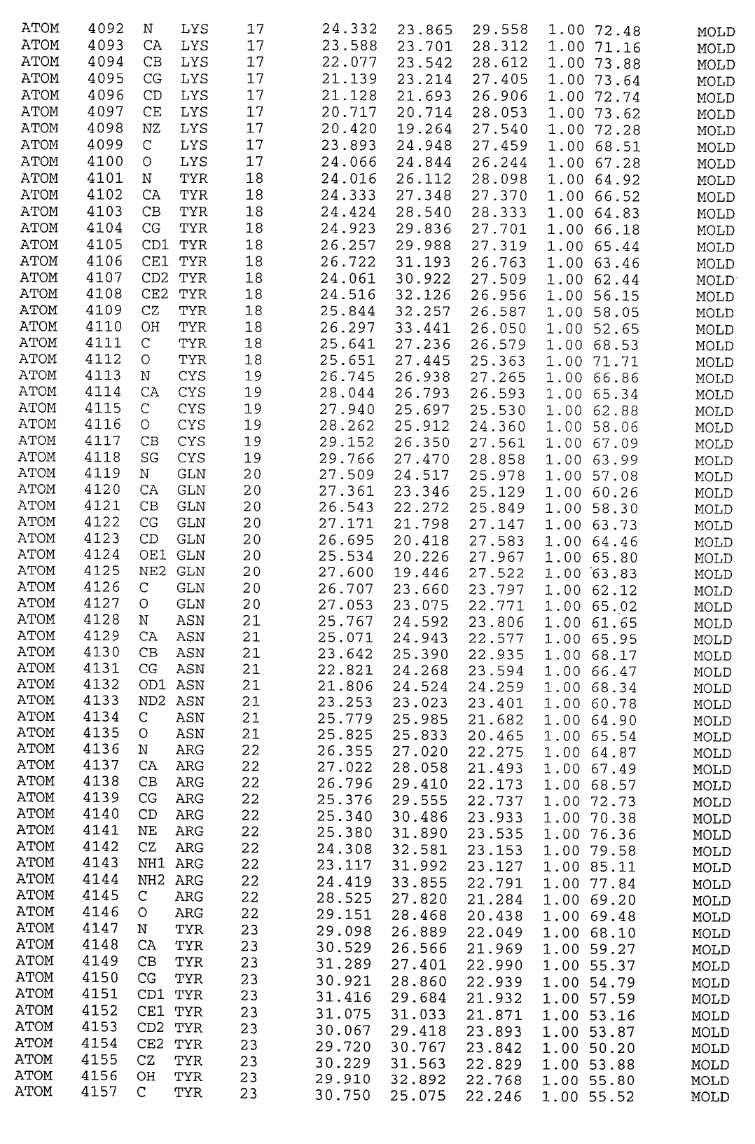

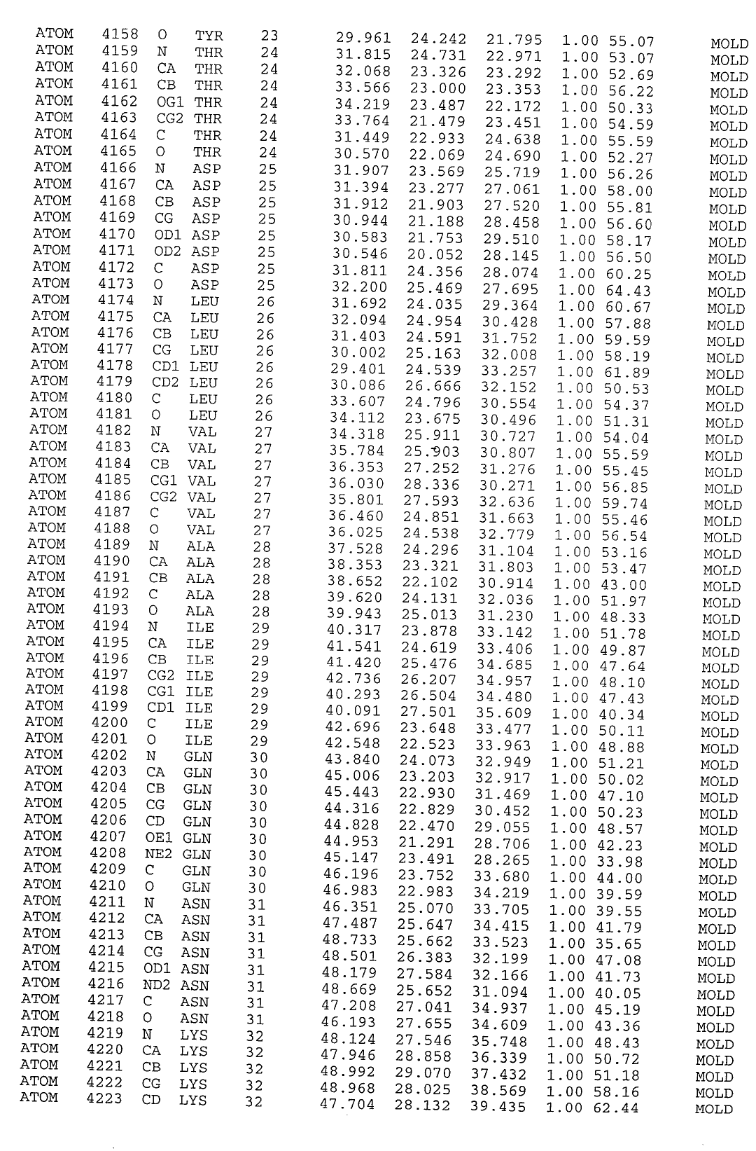

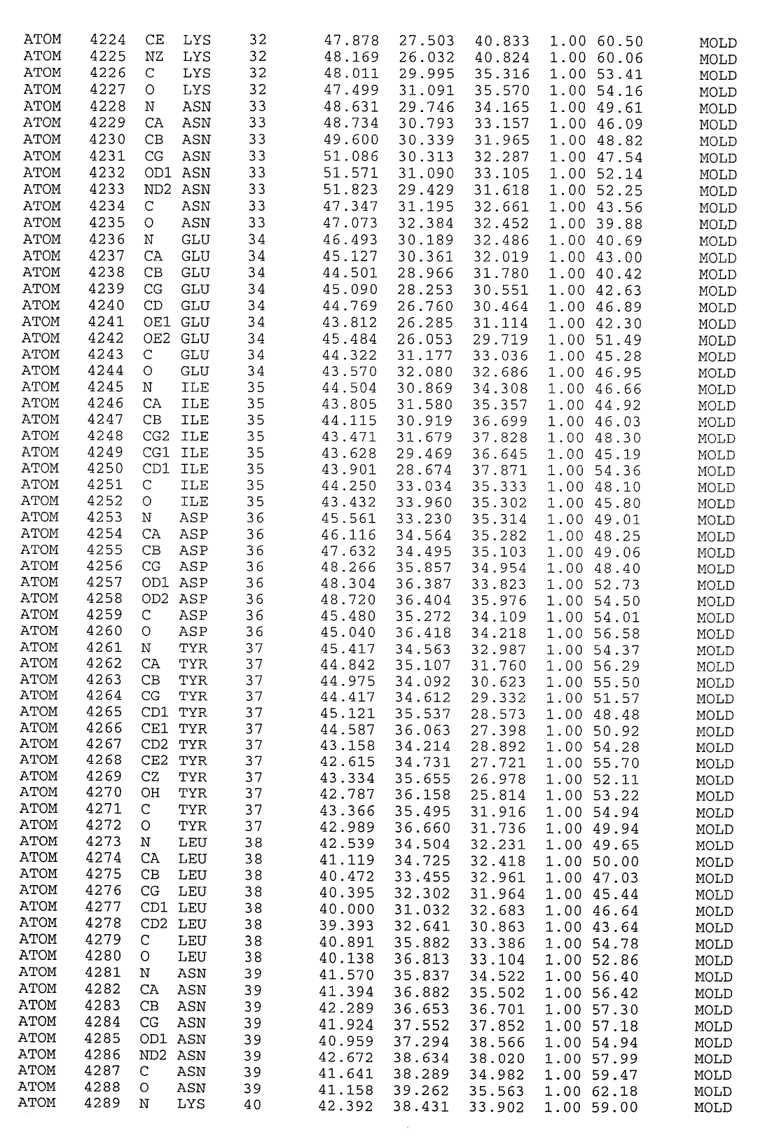

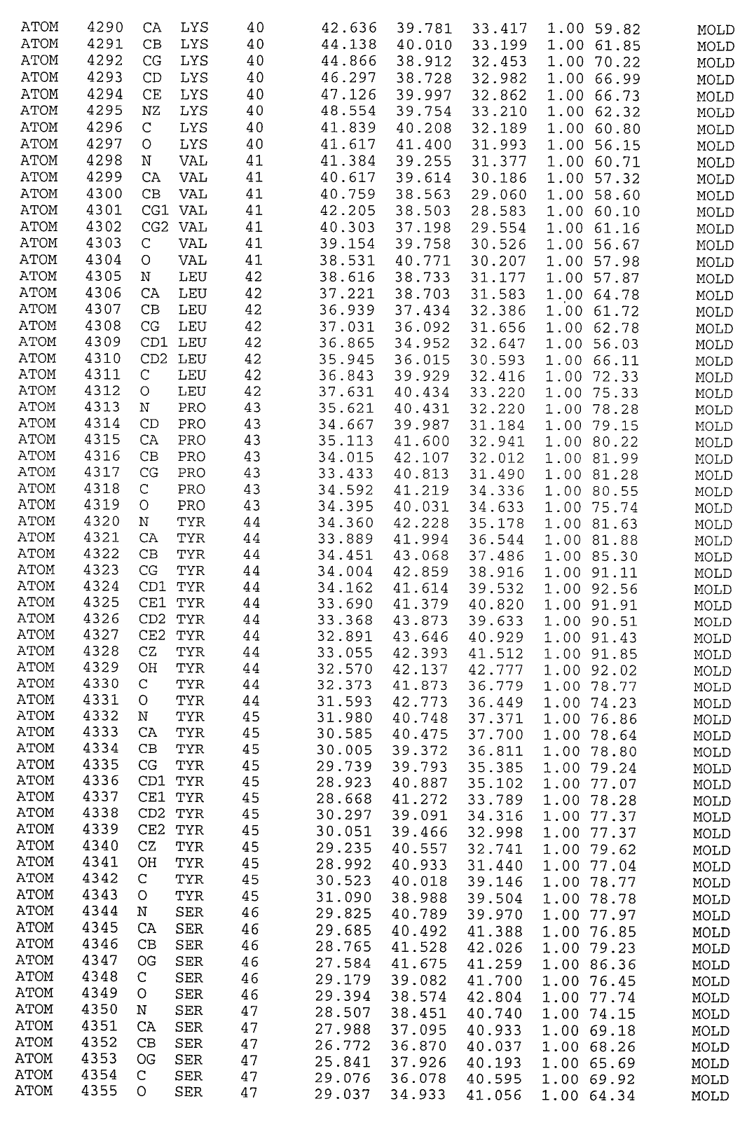

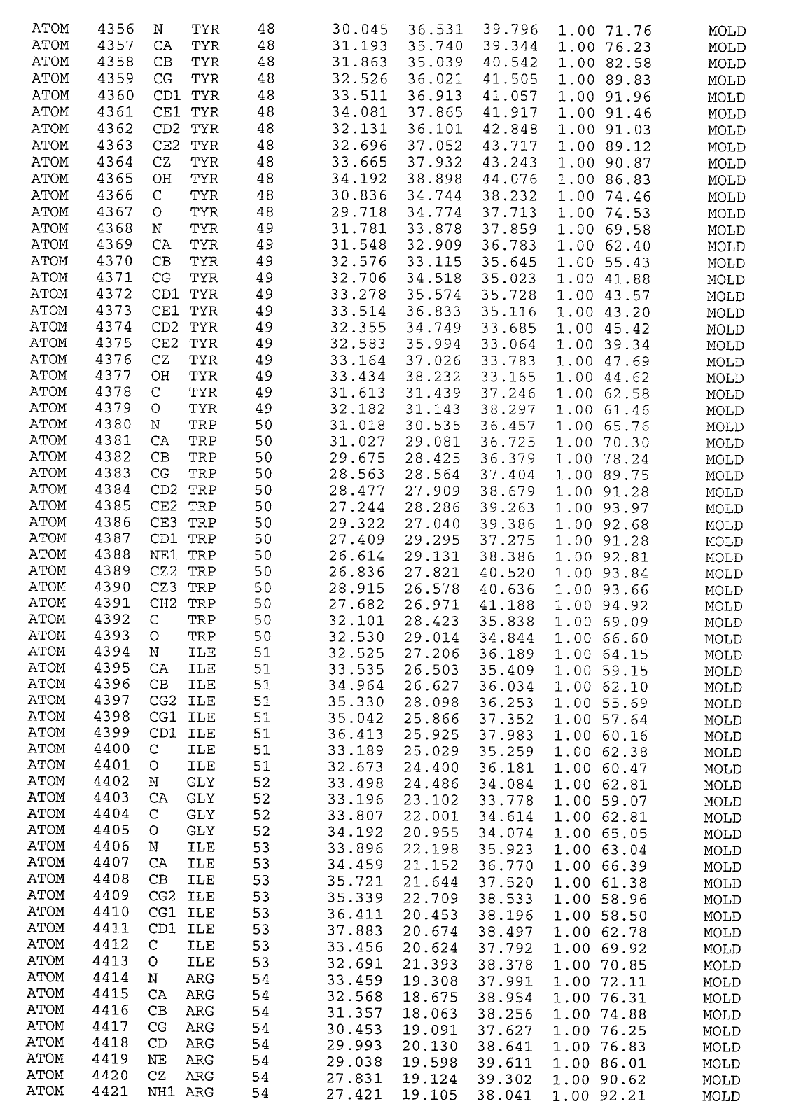

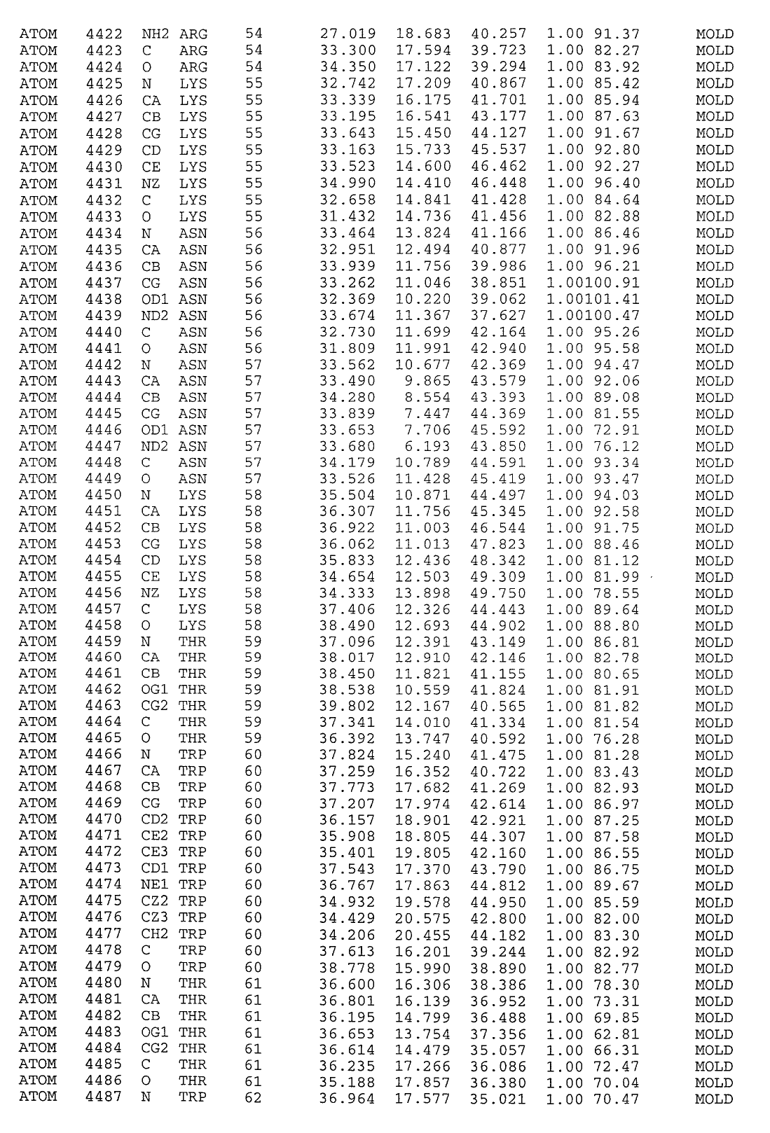

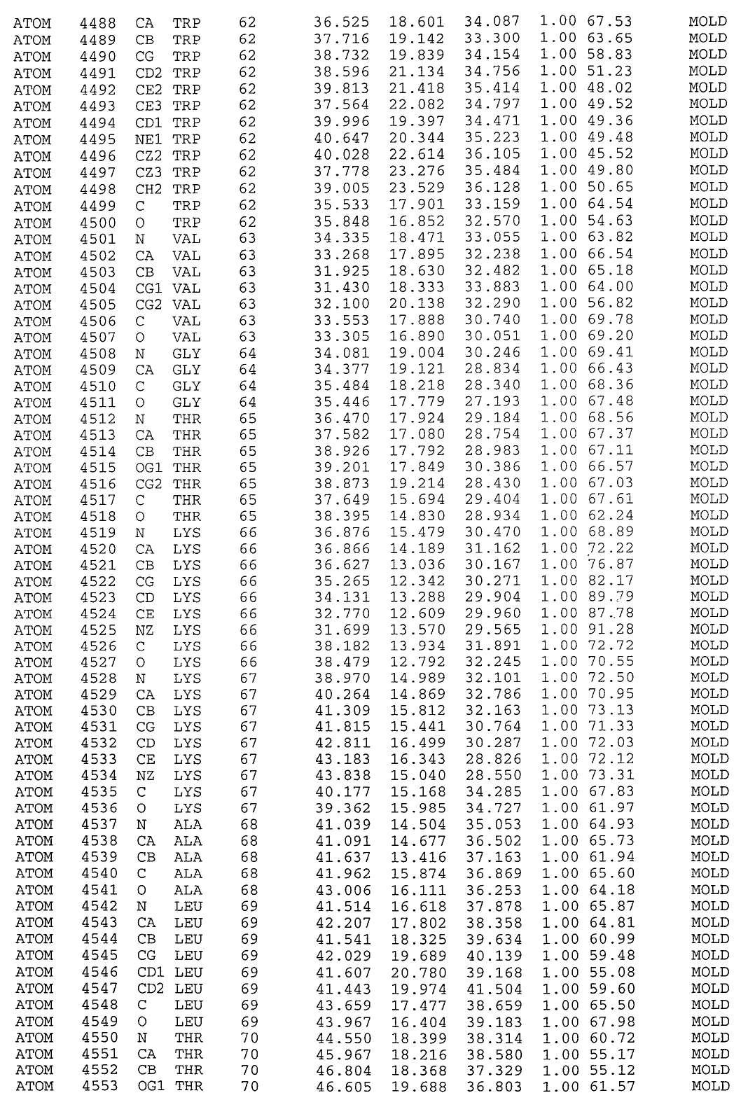

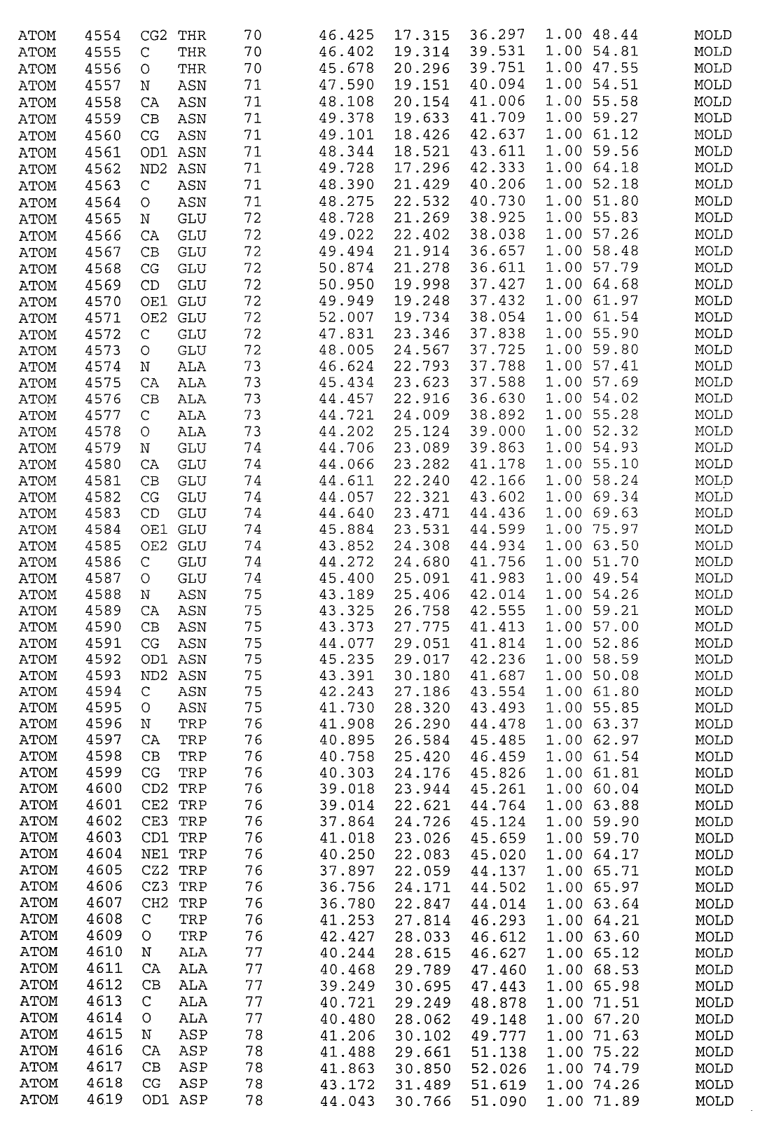

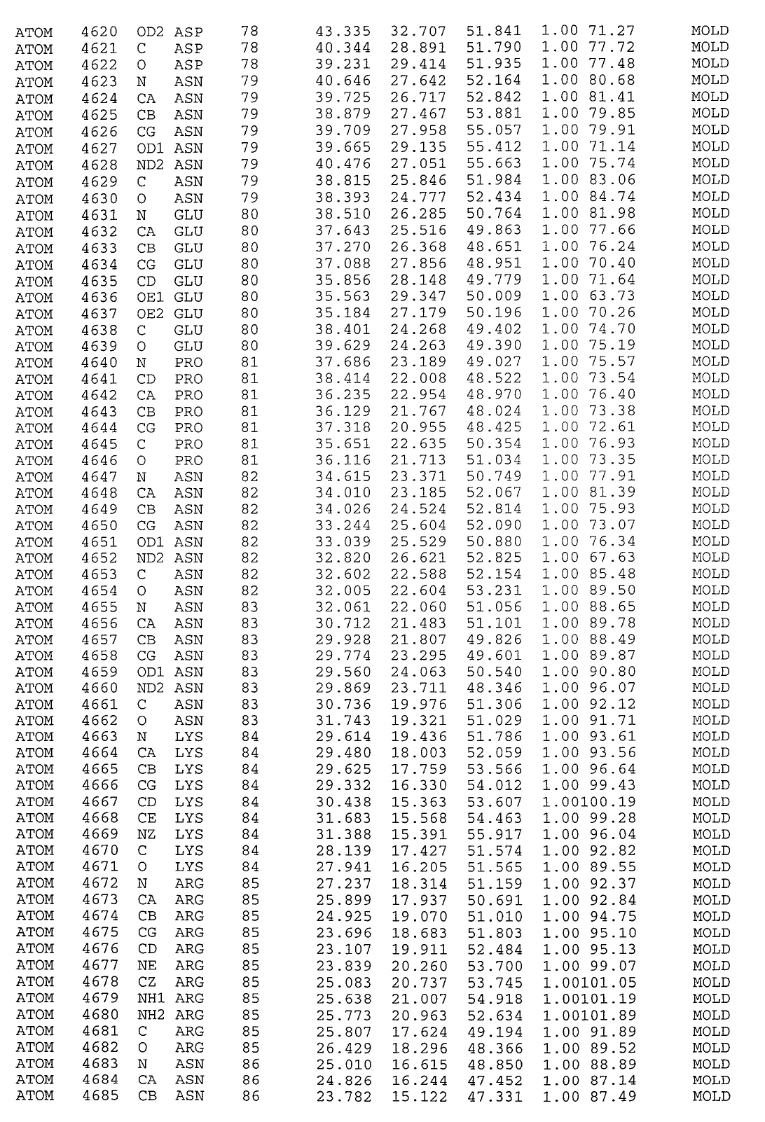

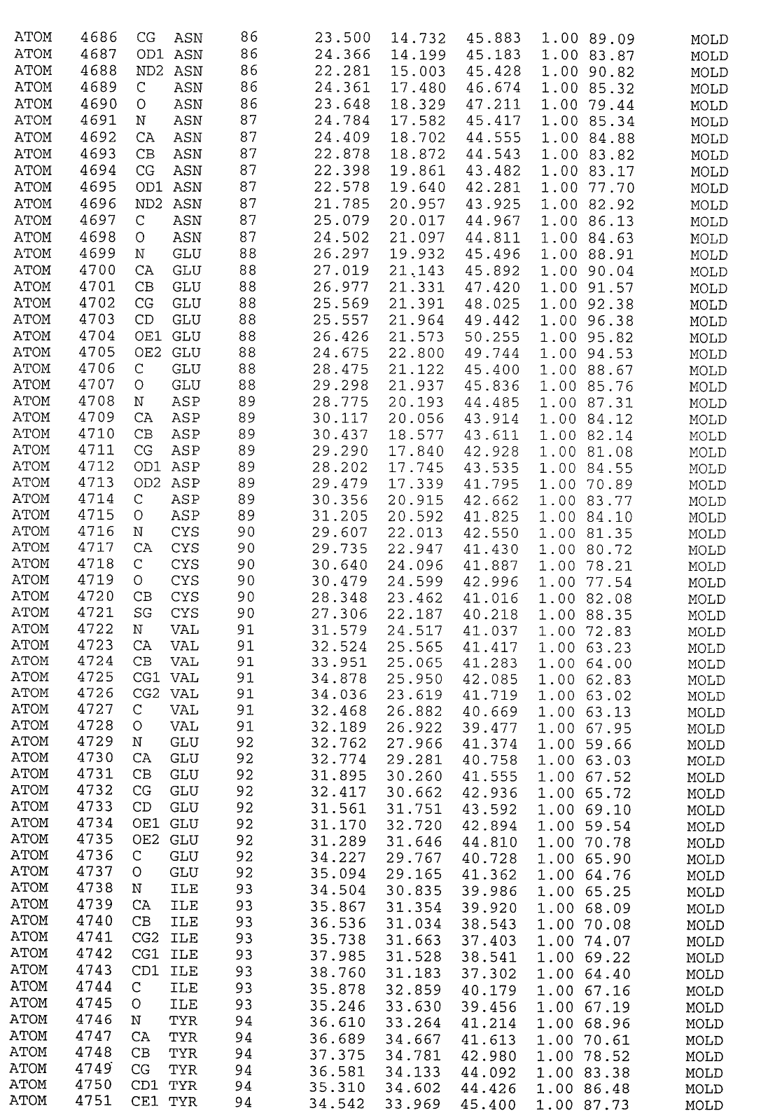

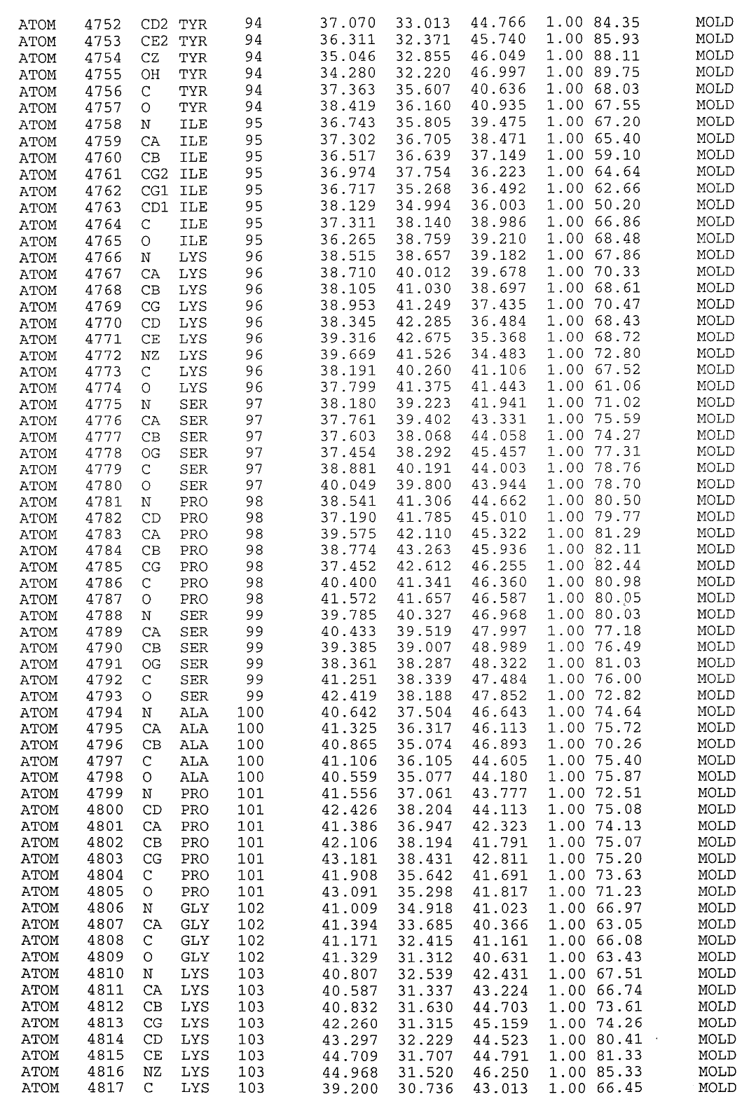

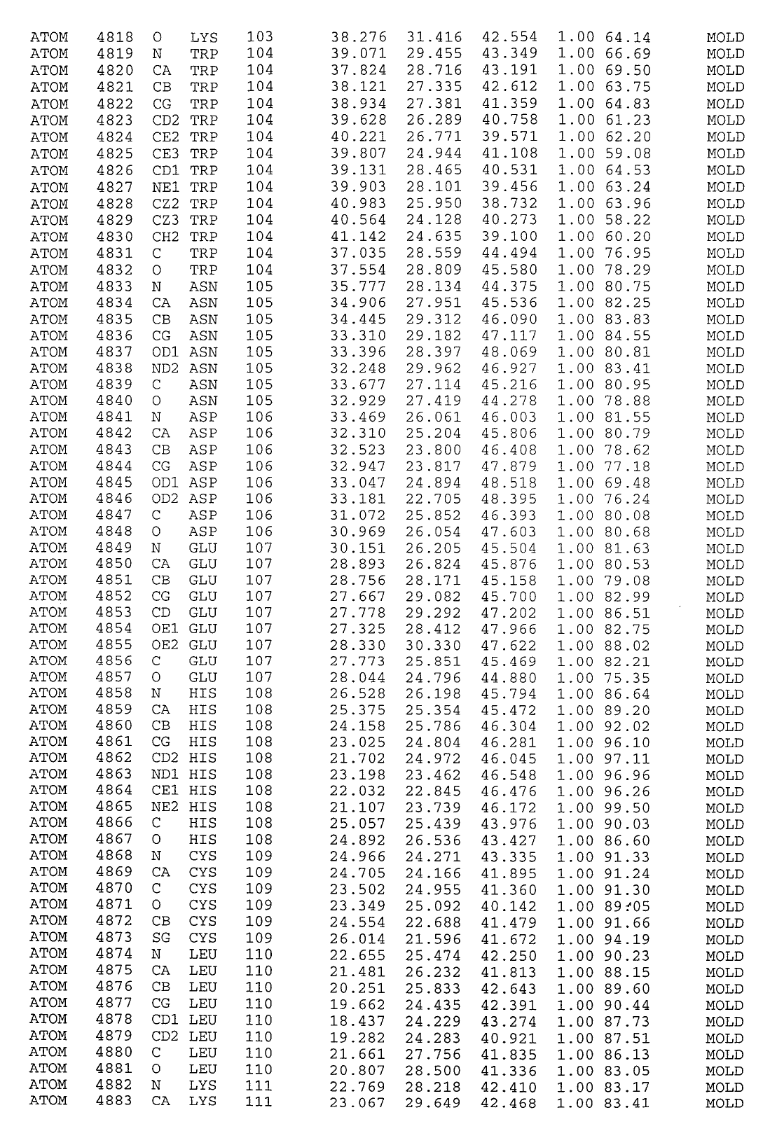

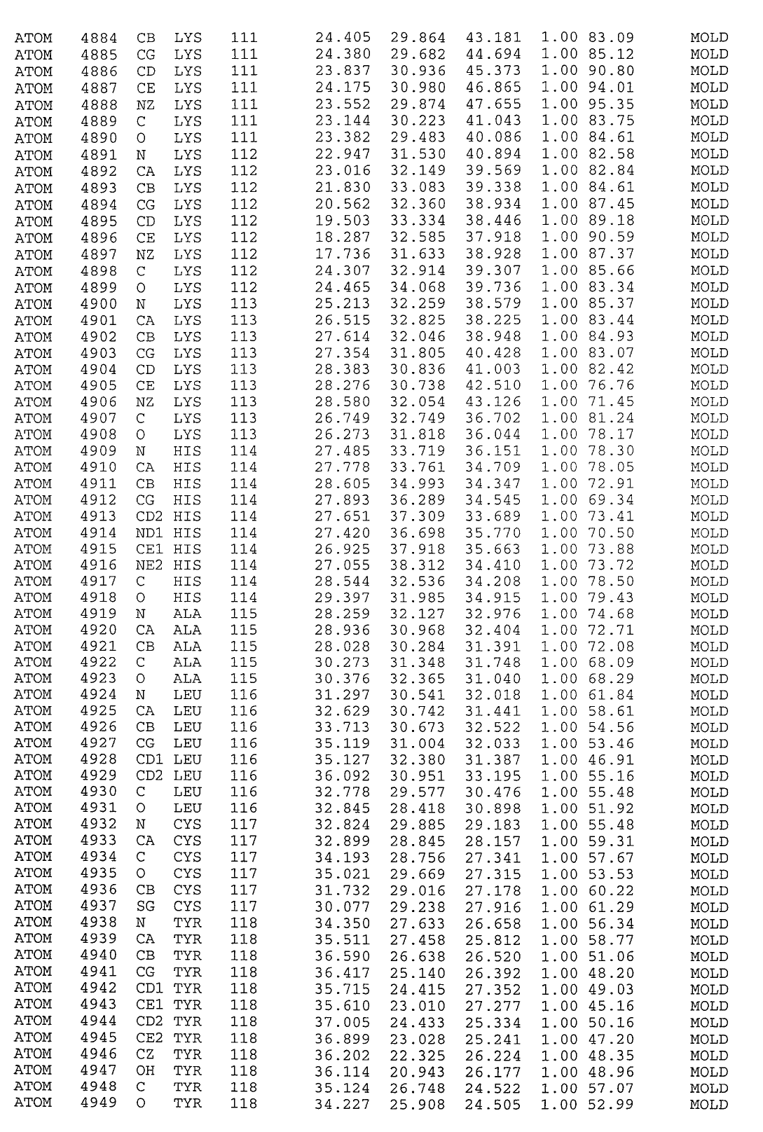

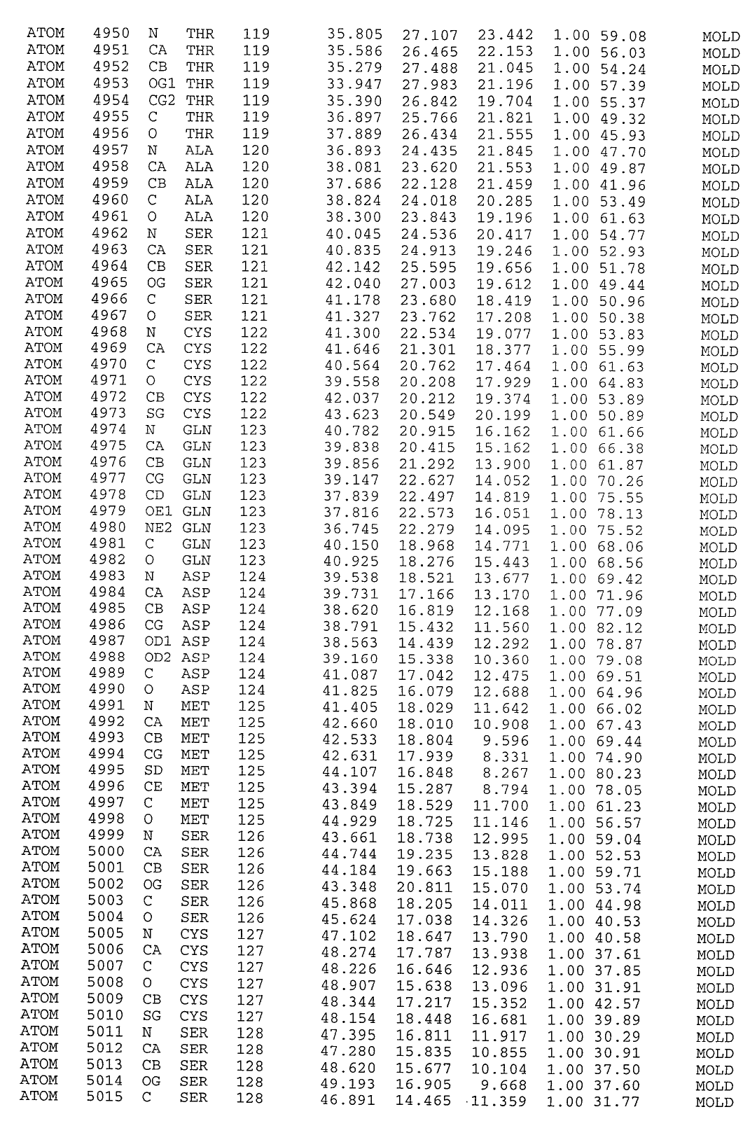

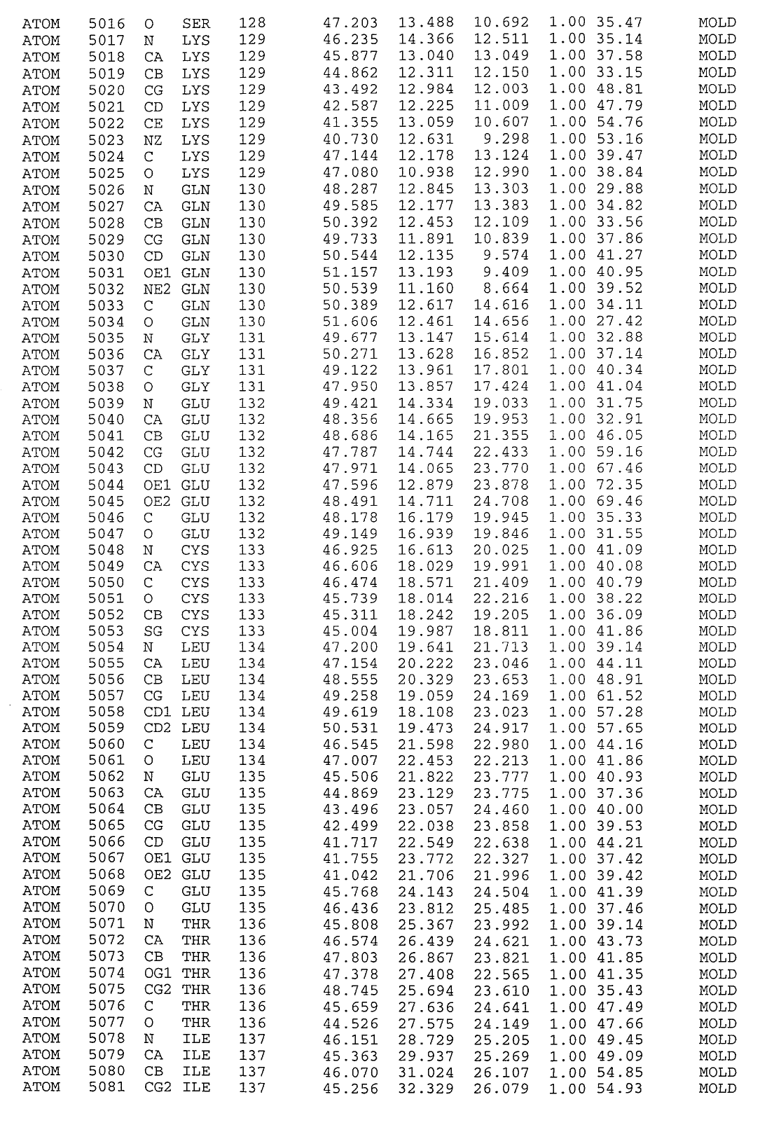

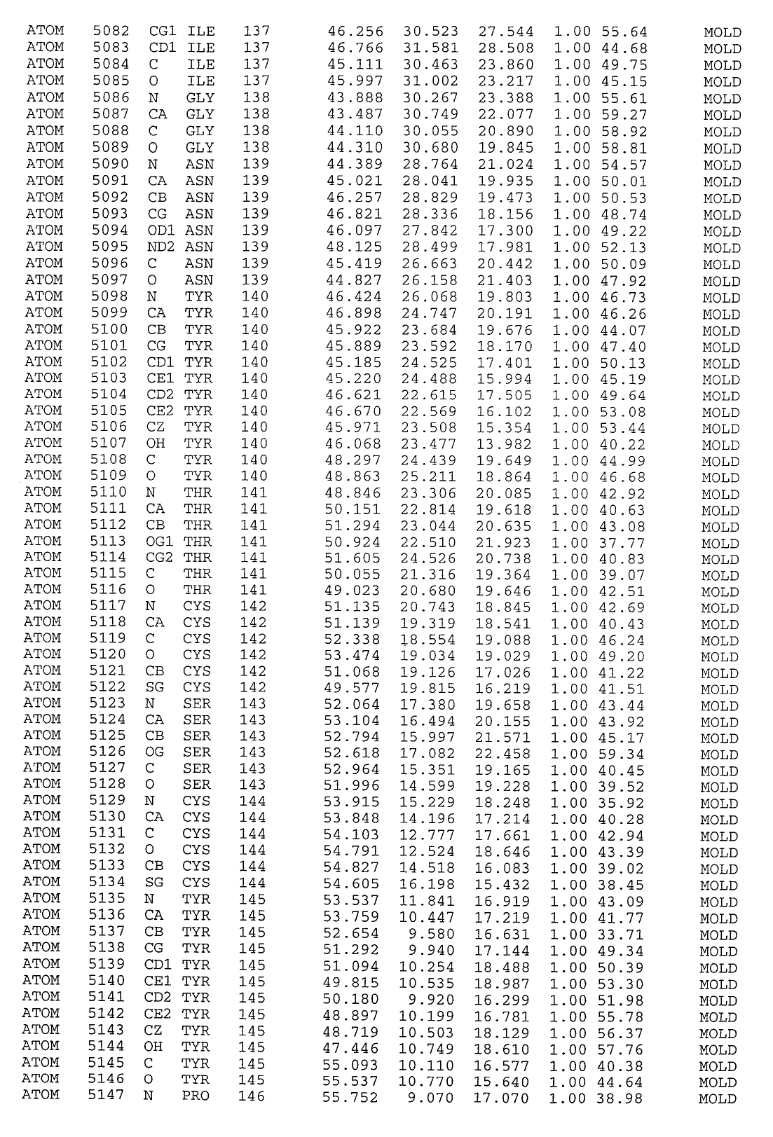

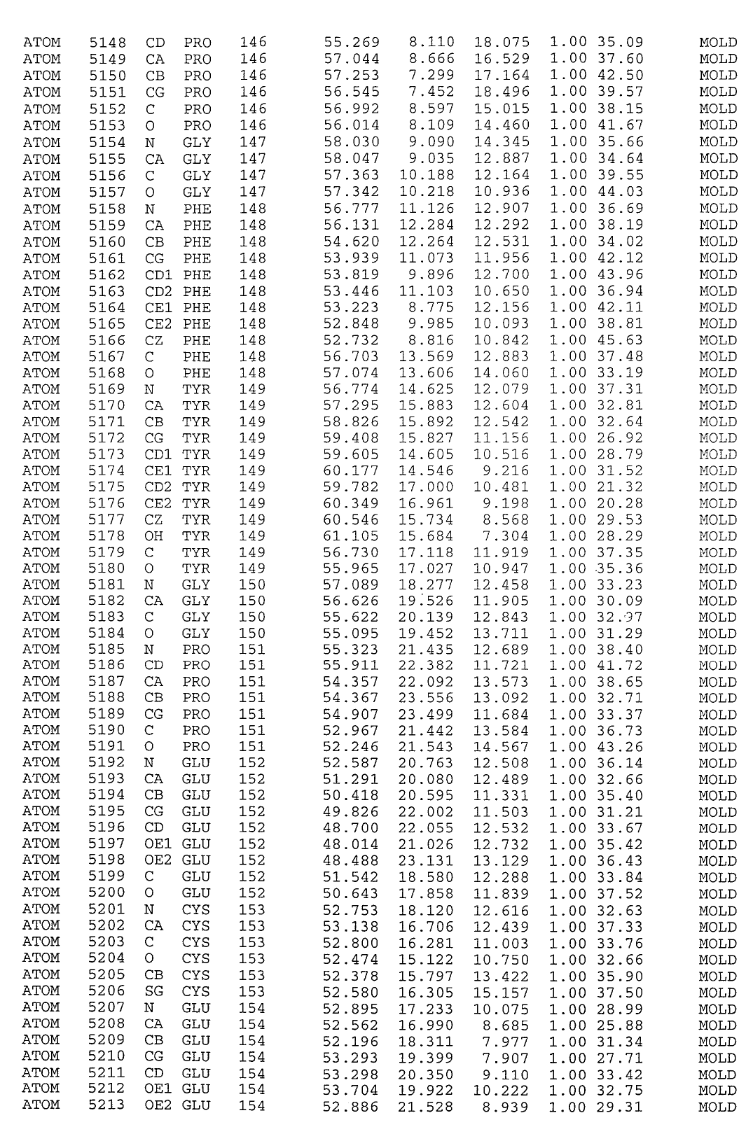

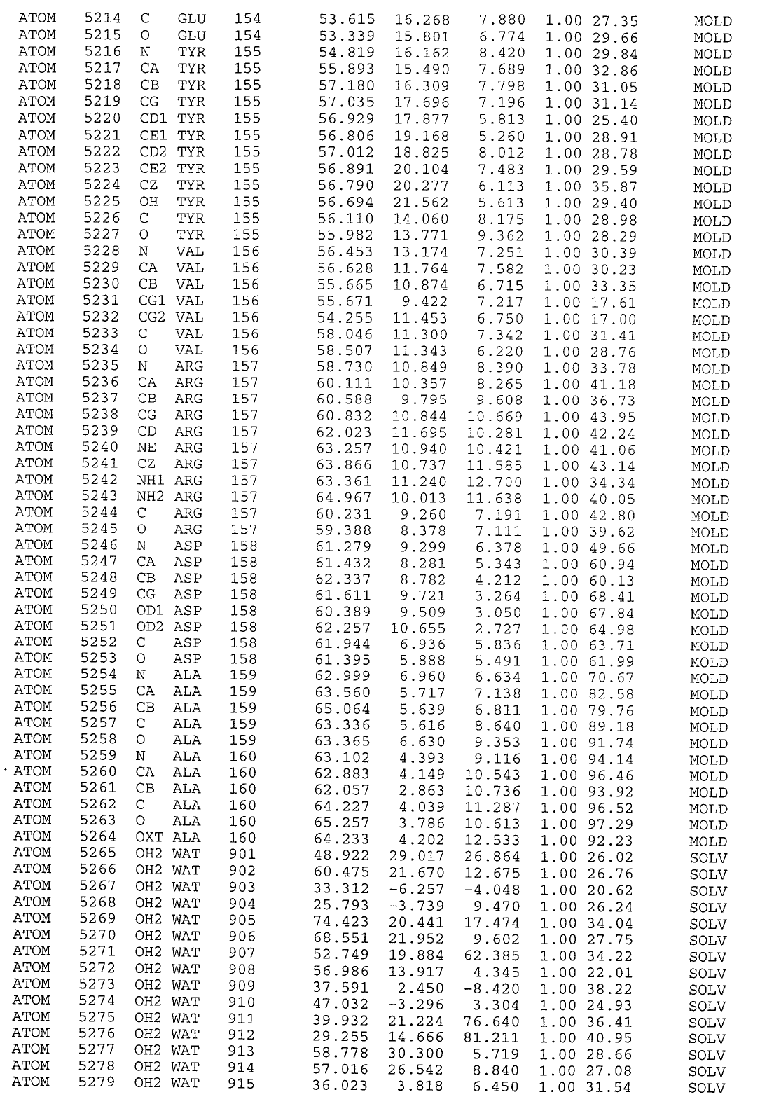

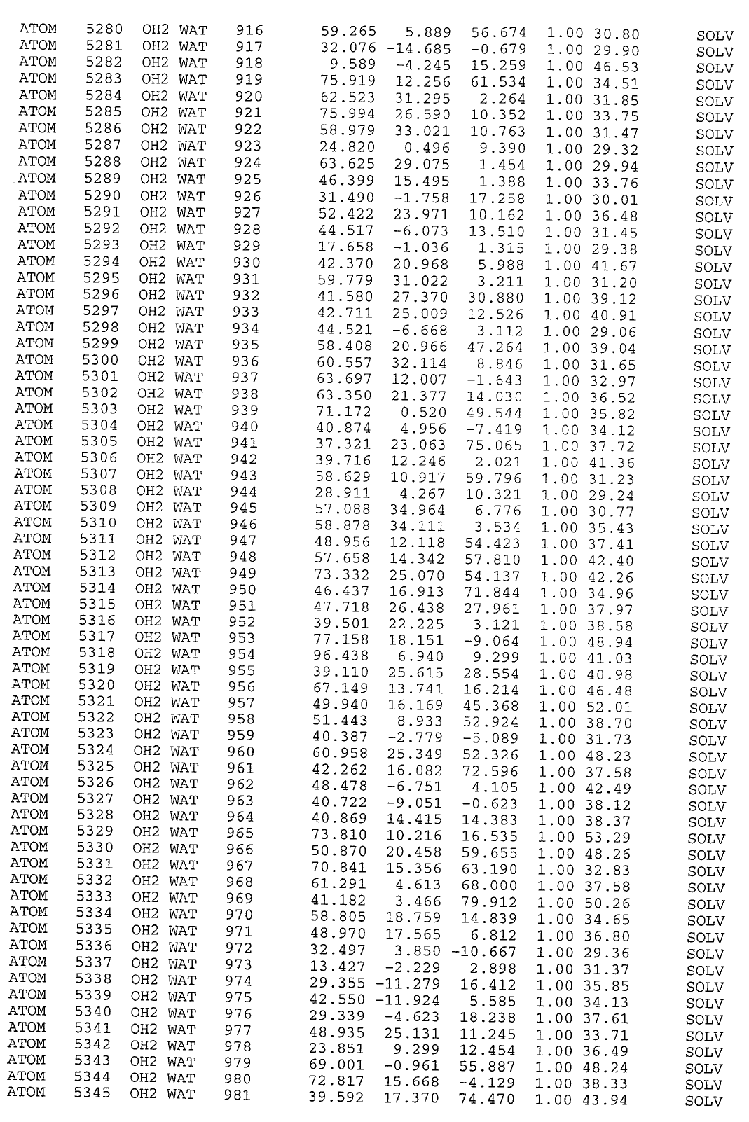

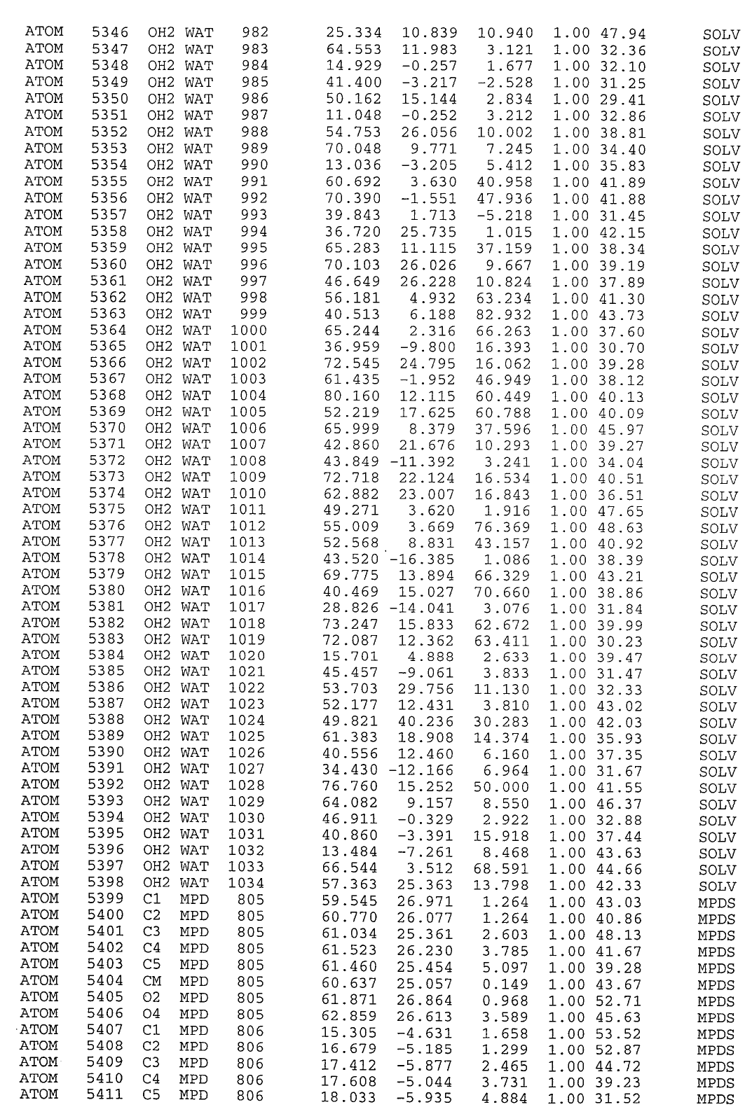

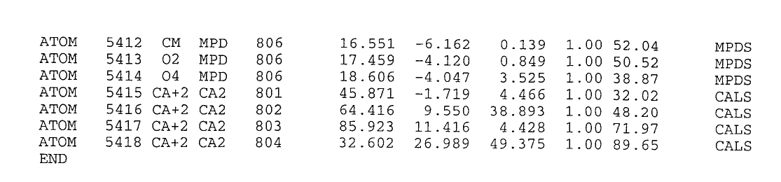

“Structural coordinates” are the Cartesian coordinates corresponding to an atom's spatial relationship to other atoms in a molecule or molecular complex. Structural coordinates may be obtained using x-ray crystallography techniques or NMR techniques, or may be derived using molecular replacement analysis or homology modeling. Various software programs allow for the graphical representation of a set of structural coordinates to obtain a three dimensional representation of a molecule or molecular complex. The structural coordinates of the present invention may be modified from the original sets provided in FIGS. 2, 3, 4 or 5 by mathematical manipulation, such as by inversion or integer additions or subtractions. As such, it is recognized that the structural coordinates of the present invention are relative, and are in no way specifically limited by the actual x, y, z coordinates of FIGS. 2, 3, 4 or 5.

An “agent” shall include a protein, polypeptide, peptide, nucleic acid, including DNA or RNA, molecule, compound or drug.

“Root mean square deviation” is the square root of the arithmetic mean of the squares of the deviations from the mean, and is a way of expressing deviation or variation from the structural coordinates described herein. The present invention includes all embodiments comprising conservative substitutions of the noted amino acid residues resulting in same structural coordinates within the stated root mean square deviation.

It will be obvious to the skilled practitioner that the numbering of the amino acid residues of P-selectin, E-selectin, P-selectin LE, E-selectin LE, PSGL-1 and the PSGL-1 peptide may be different than that set forth herein, and may contain certain conservative amino acid substitutions that yield the same three dimensional structures as those defined by FIGS. 2, 3, 4 or 5 herein. Corresponding amino acids and conservative substitutions in other isoforms or analogues are easily identified by visual inspection of the relevant amino acid sequences or by using commercially available homology software programs (e.g., MODELLAR, MSI, San Diego, Calif.).

“Conservative substitutions” are those amino acid substitutions which are functionally equivalent to the substituted amino acid residue, either by way of having similar polarity, steric arrangement, or by belonging to the same class as the substituted residue (e.g., hydrophobic, acidic or basic), and includes substitutions having an inconsequential effect on the three dimensional structures of P-selectin LE, E-selectin LE, SLe^{X}, the PSGL-1 peptide, the P-selectin LE: SLe^{X} complex, the E-selectin LE: SLe^{X} complex, and the P-selectin LE: PSGL-1 peptide complex, with respect to the use of said structures for the identification and design of agents which interact with P-selectin, E-selectin, P-selectin LE, E-selectin LE, SLe^{X}, PSGL-1, the PSGL-1 peptide, the P-selectin LE: SLe^{X} complex, the E-selectin LE: SLe^{X} complex, the P-selectin LE: PSGL-1 peptide complex, as well as other proteins, peptides, molecules or molecular complexes comprising an SLe^{X} or PSGL-1 binding site, for molecular replacement analyses and/or for homology modeling.

An “active site” refers to a region of a molecule or molecular complex that, as a result of its shape and charge potential, favorably interacts or associates with another agent (including, without limitation, a protein, polypeptide, peptide, nucleic acid, including DNA or RNA, molecule, compound or drug) via various covalent and/or non-covalent binding forces. As such, an active site of the present invention may include, for example, the actual site of SLe^{X} or PSGL-1 binding with P-selectin LE or E-selectin LE, as well as accessory binding sites adjacent or proximal to the actual site of SLe^{X} or PSGL-1 binding that nonetheless may affect P-selectin LE or E-selectin LE activity upon interaction or association with a particular agent, either by direct interference with the actual site of SLe^{X} or PSGL-1 binding or by indirectly affecting the steric conformation or charge potential of the P-selectin LE or E-selectin LE and thereby preventing or reducing binding of SLe^{X} or PSGL-1 to P-selectin LE or E-selectin LE at the actual site of SLe^{X} or PSGL-1 binding. As used herein, an “active site” also includes analog residues of P-selectin LE and E-selectin LE which exhibit observable NMR perturbations in the presence of a binding ligand, such as SLe^{X} or PSGL-1. While such residues exhibiting observable NMR perturbations may not necessarily be in direct contact with or immediately proximate to ligand binding residues, they may be critical P-selectin LE and E-selectin LE residues for rational drug design protocols.

The present invention first provides a crystallized P-selectin LE. In a particular embodiment, P-selectin LE comprises the amino acid residues set forth in FIG. 6A (underlined), including conservative substitutions. The crystal of the present invention effectively diffracts X-rays for the determination of the structural coordinates of P-selectin LE, and is characterized as being in plate form with space group P2₁, and having unit cell parameters of a=81.0 Å, b=60.8 Å, c=91.4 Å, and beta=103.6°. Further, a crystallographic asymmetric unit of the crystallized P-selectin LE contains four molecules of P-selectin LE.

The present invention also provides a crystallized complex comprising P-selectin LE and SLe^{X}. In a particular embodiment, the amino acid sequence of P-selectin LE is set forth in FIG. 6A (underlined), and includes conservative substitutions. The crystal complex of the present invention effectively diffracts X-rays for the determination of the structural coordinates of the complex of P-selectin LE and SLe^{X}, and is characterized as being in plate form with space group P2₁, and having unit cell parameters of a=81.1 Å, b=60.5 Å, c=91.4 Å, and beta=103.3°. Further, the crystallized complex of the present invention consists of one molecule of the P-selectin LE: SLe^{X} complex in the asymmetric crystal unit.

The present invention further provides a crystallized complex comprising E-selectin LE and SLe^{X}. In a particular embodiment, the amino acid sequence of E-selectin LE is set forth in FIG. 6B (underlined), and includes conservative substitutions. The crystal complex of the present invention effectively diffracts X-rays for the determination of the structural coordinates of the complex of E-selectin LE and SLe^{X}, and is characterized as being in rod form with space group P2₁2₁2₁, and having unit cell parameters of a=34.5 Å, b=72.4 Å, and c=77.6 Å. Further, the crystallized complex of the present invention consists of one molecule of the E-selectin LE: SLe^{X} complex in the asymmetric crystal unit.

The present invention still further provides a crystallized complex comprising P-selectin LE and a PSGL-1 peptide. The crystal complex of the present invention effectively diffracts X-rays for the determination of the structural coordinates of the complex of P-selectin LE and PSGL-1 peptide, and is characterized as being in bipyramidal form with space group I222 and having unit cell parameters of a=63.4 Å, b=96.8 Å, and c=187.3 Å. Further, the crystallized complex of the present invention consists of one molecule of the PE-selectin LE:PSGL-1 peptide complex in the asymmetric crystal unit.

Once a crystal or crystal complex of the present invention is grown, X-ray diffraction data can be collected by a variety of means in order to obtain the atomic coordinates of the crystallized molecule or molecular complex. With the aid of specifically designed computer software, such crystallographic data can be used to generate a three dimensional structure of the molecule or molecular complex. Various methods used to generate and refine the three dimensional structure of a crystallized molecule or molecular structure are well known to those skilled in the art, and include, without limitation, multiwavelength anomalous dispersion (MAD), multiple isomorphous replacement, reciprocal space solvent flattening, molecular replacement, and single isomorphous replacement with anomalous scattering (SIRAS).

Accordingly, the present invention also provides the three dimensional structure of P-selectin LE as derived by x-ray diffraction data of the P-selectin LE crystal. Specifically, the three dimensional structure of P-selectin LE is defined by the structural coordinates shown in FIG. 2, ± a root mean square deviation from the backbone atoms of the amino acids of not more than 1.5 Å, preferably not more than 1.0 Å, and most preferably not more than 0.5 Å. The structural coordinates of the three dimensional structure of P-selectin LE are useful for a number of applications, including, but not limited to, the visualization, identification and characterization of various active sites of P-selectin LE, including the SLe^{X} binding site. The active site structures may then be used to design agents which interact with P-selectin LE, as well as P-selectin LE complexed with SLe^{X}, PSGL-1, or related molecules.

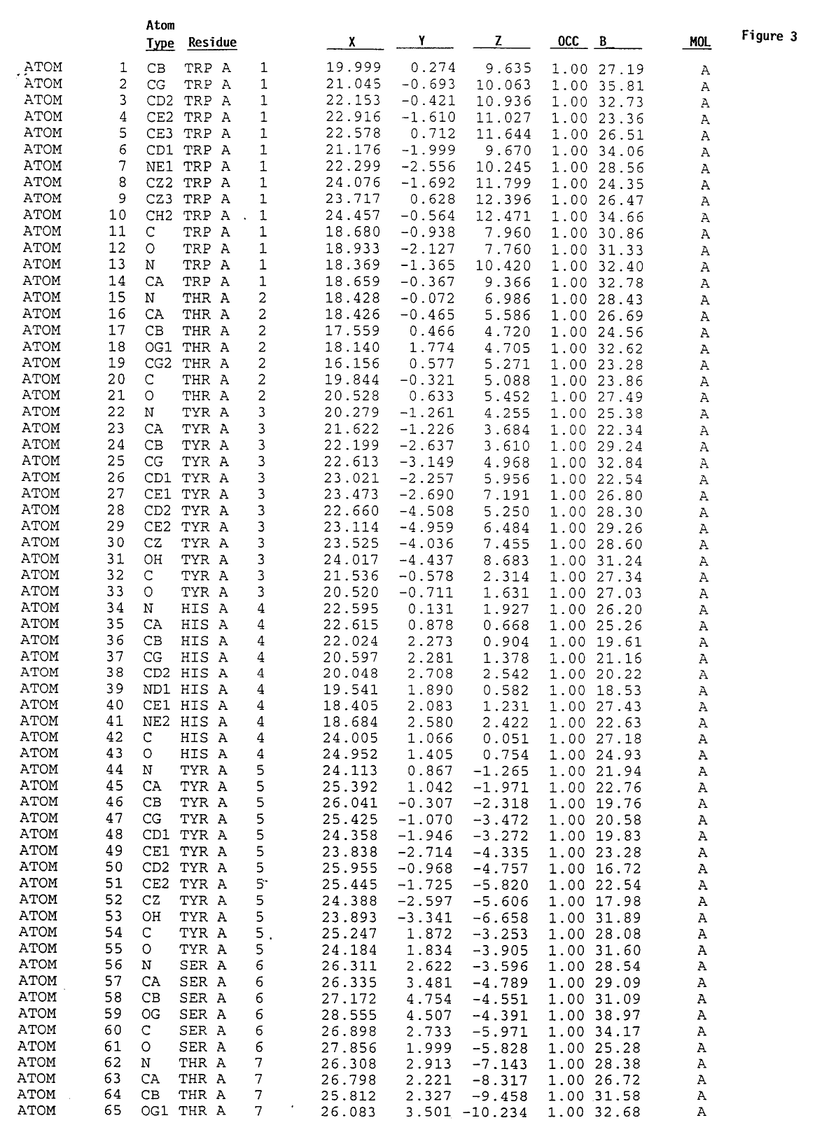

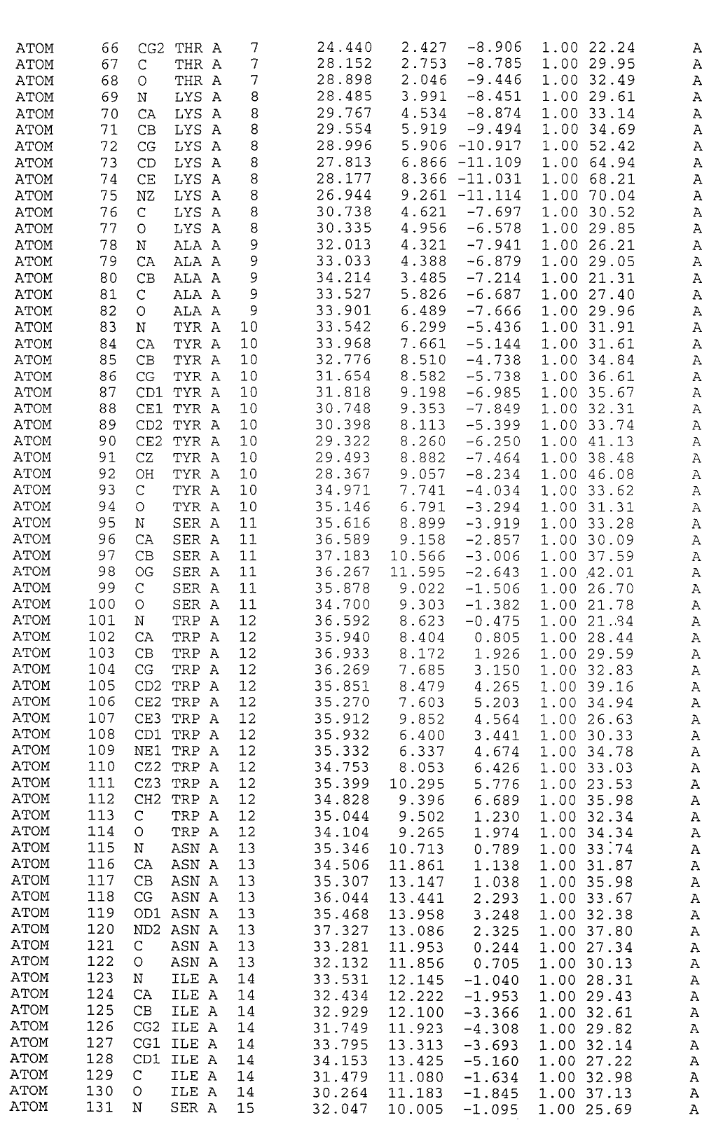

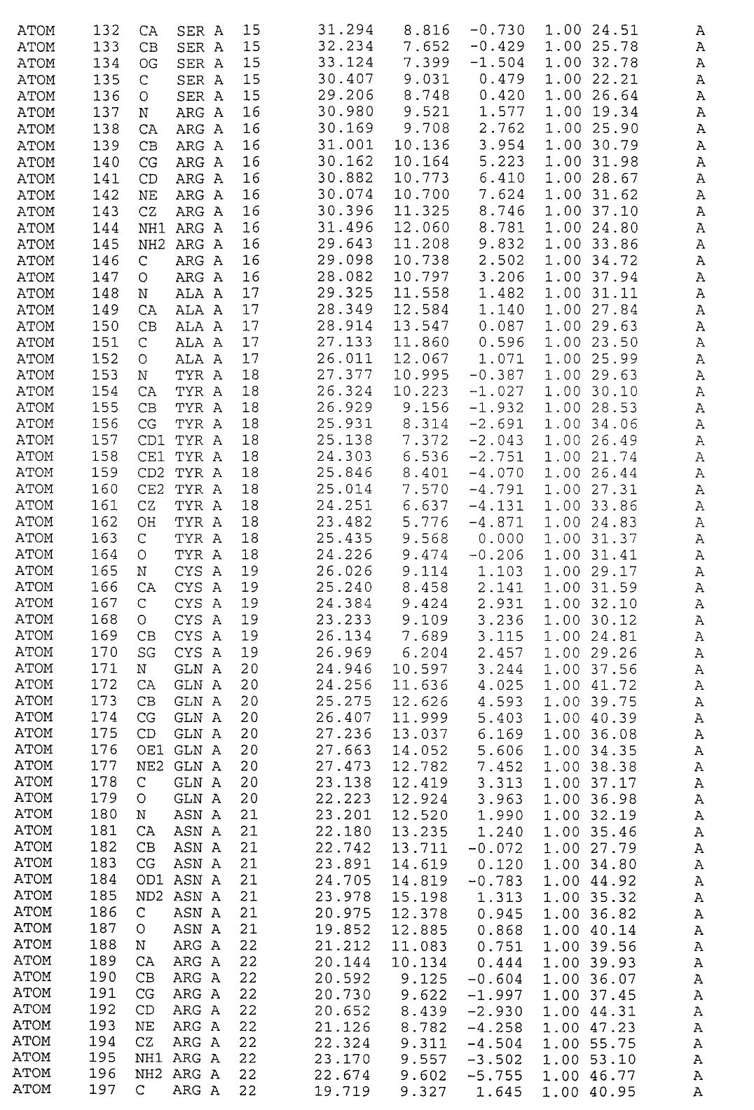

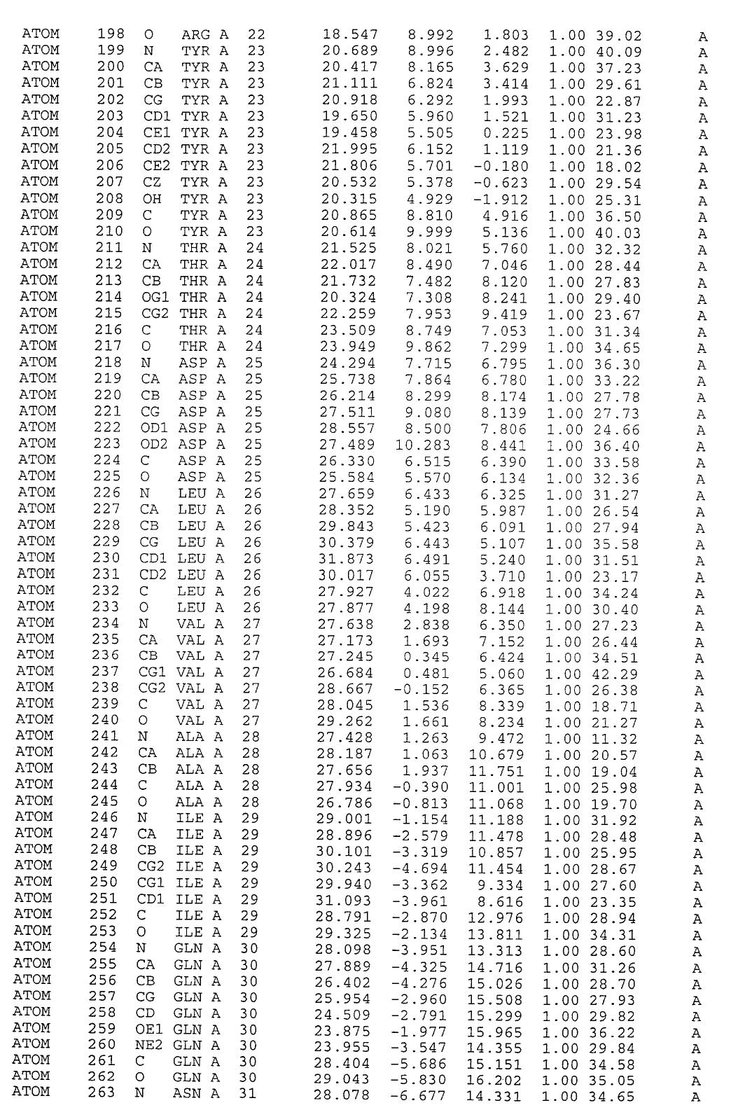

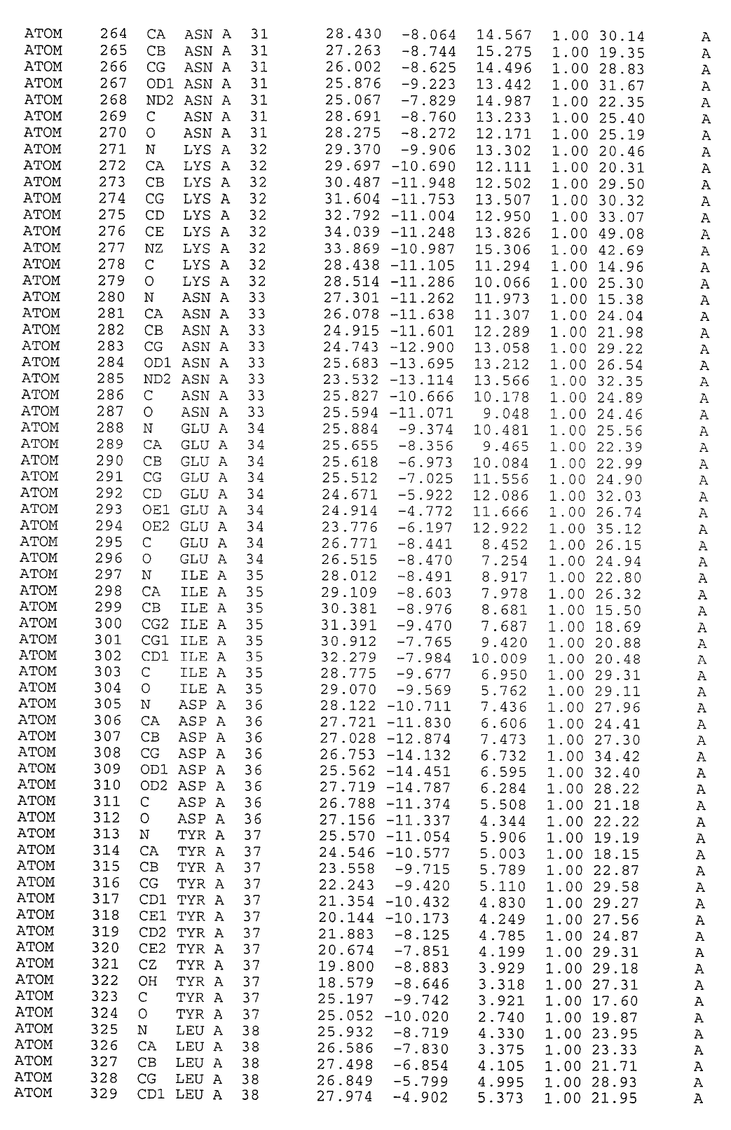

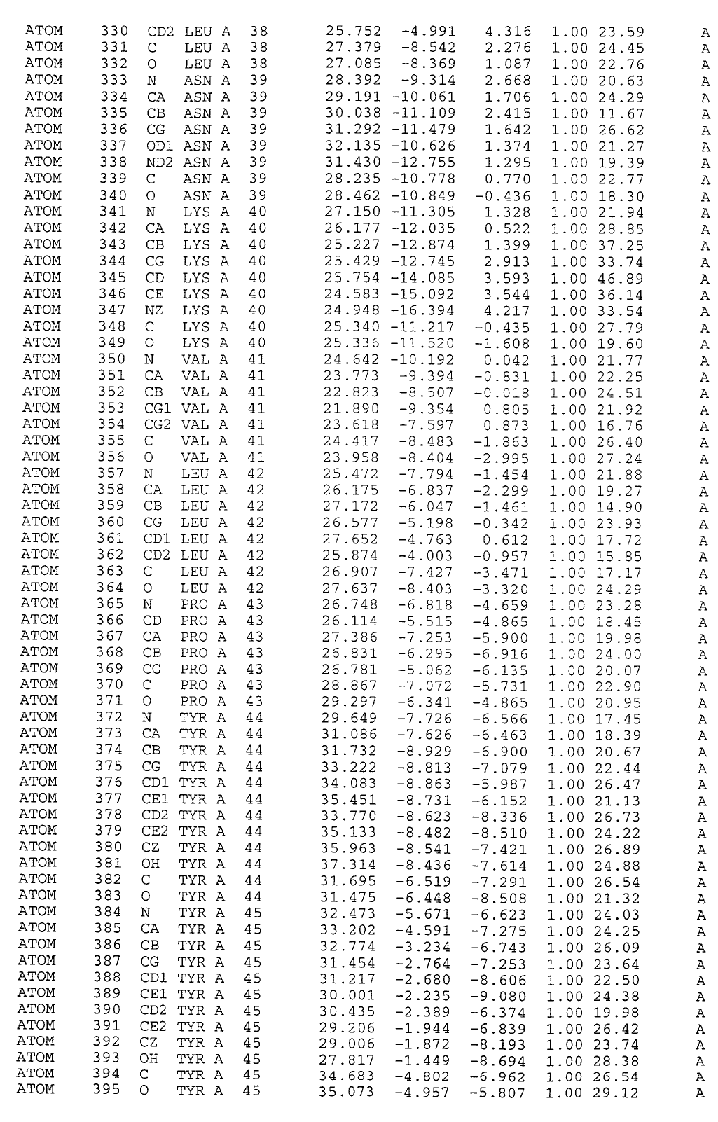

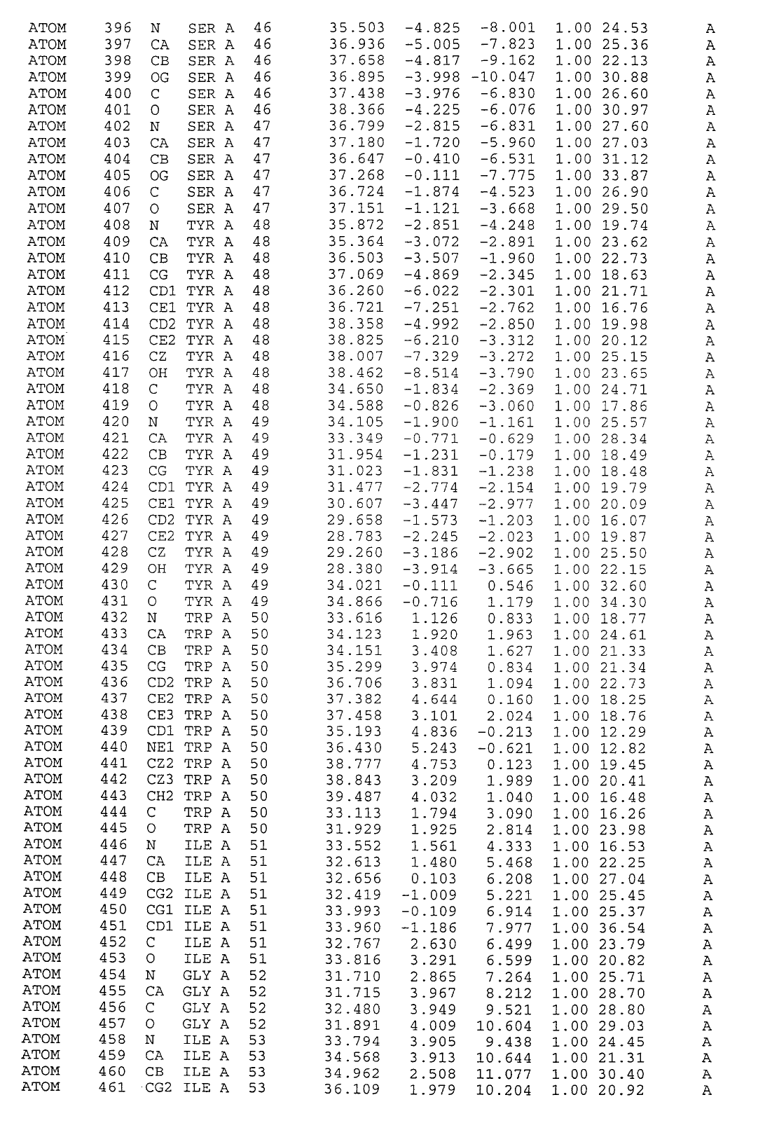

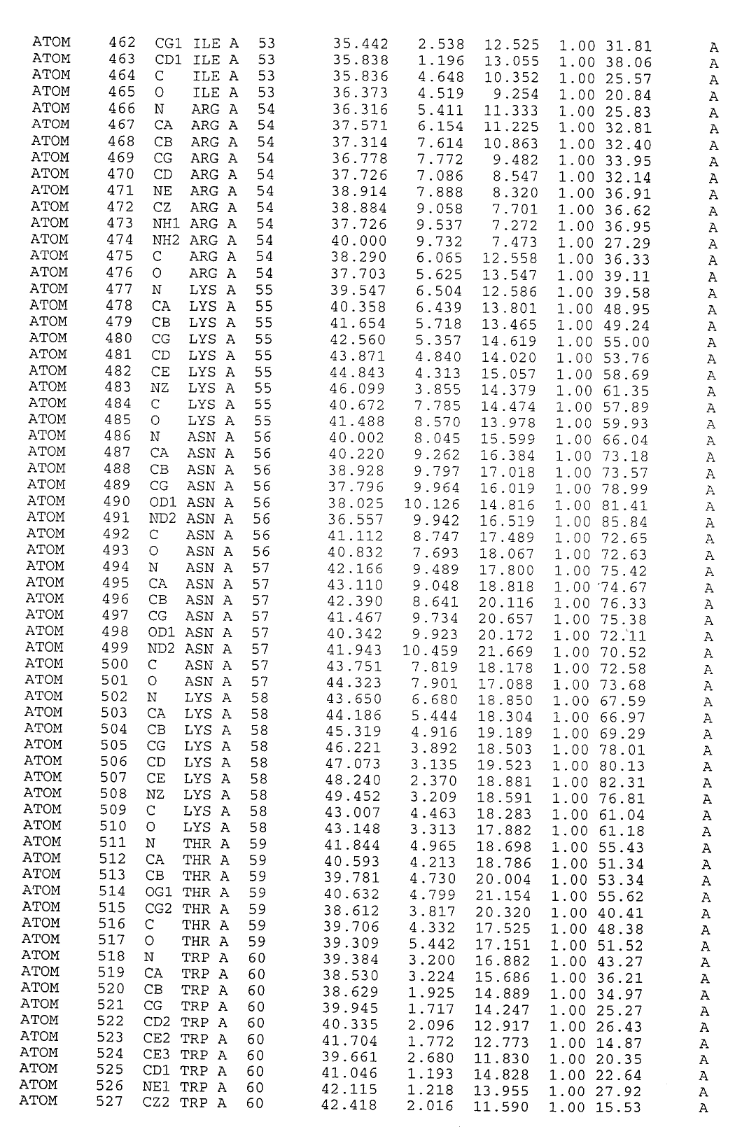

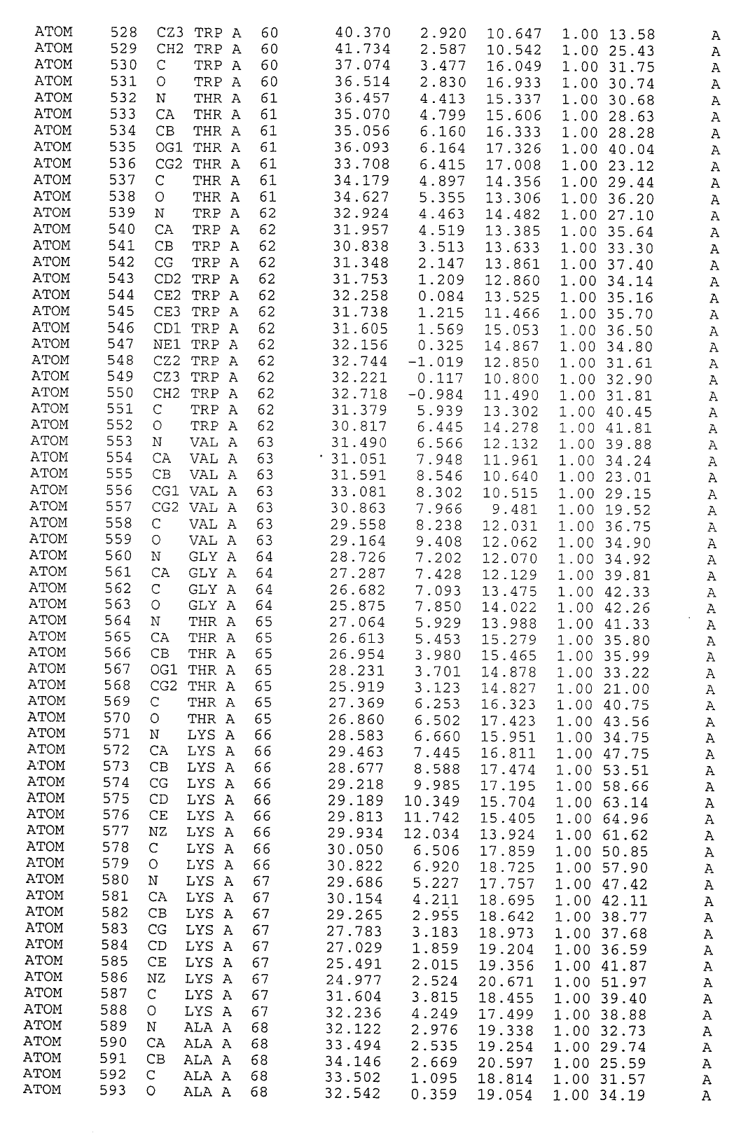

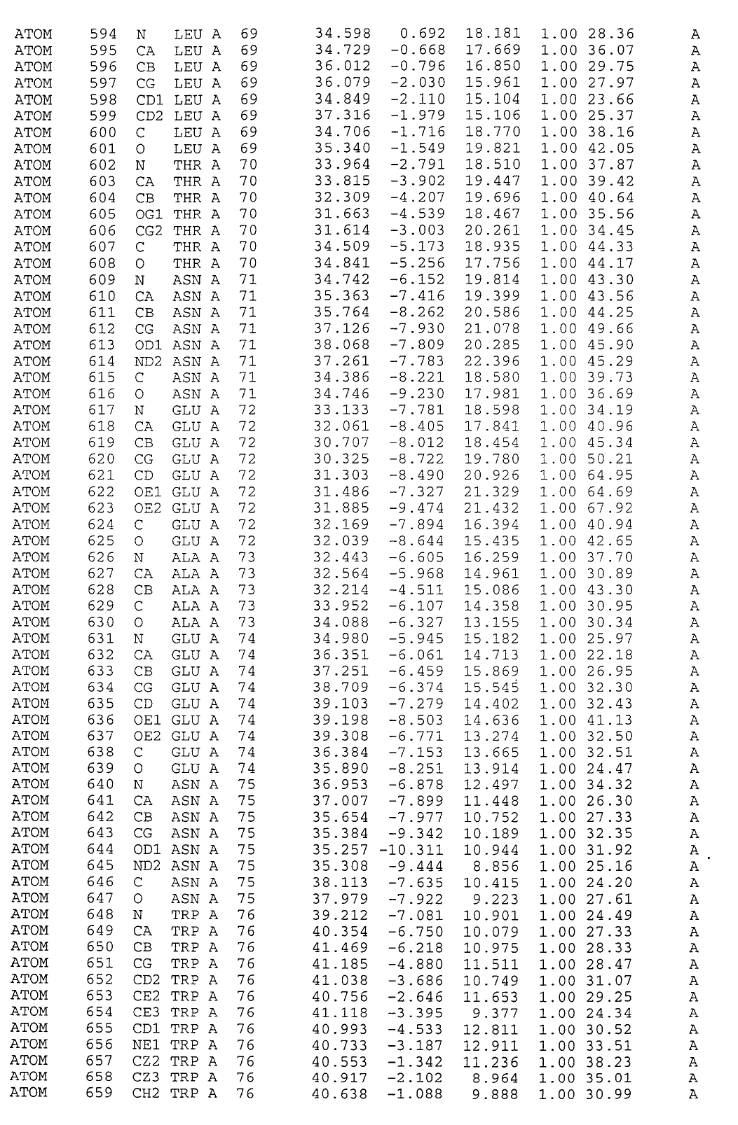

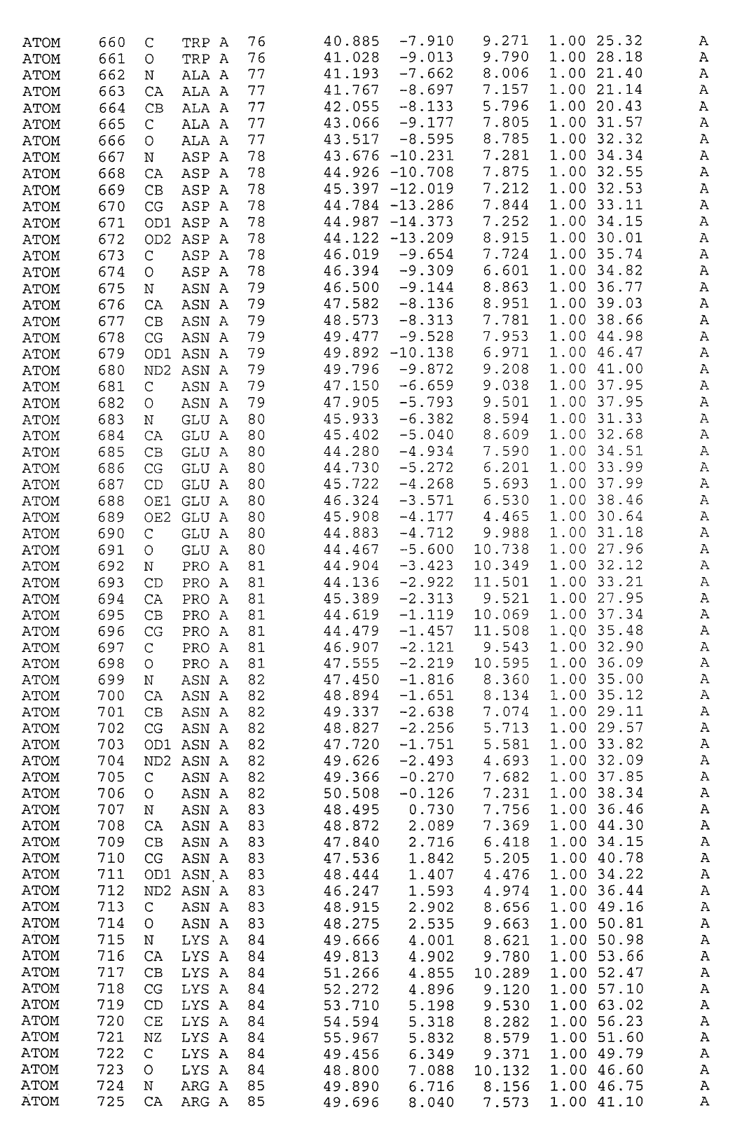

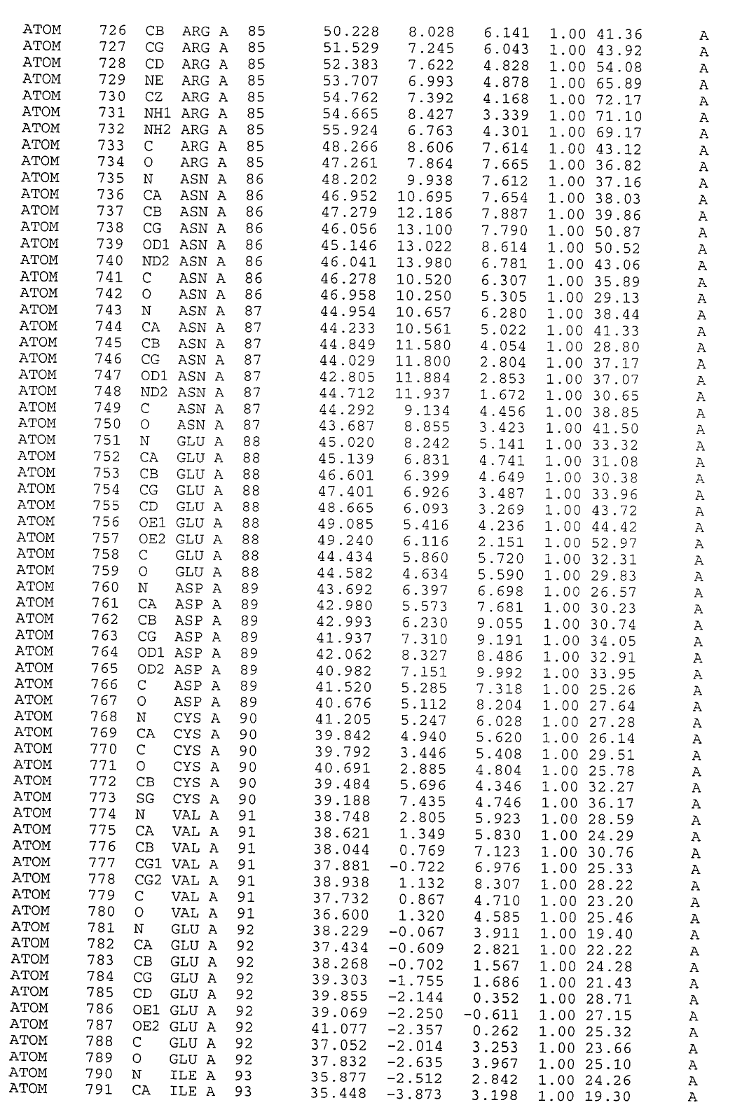

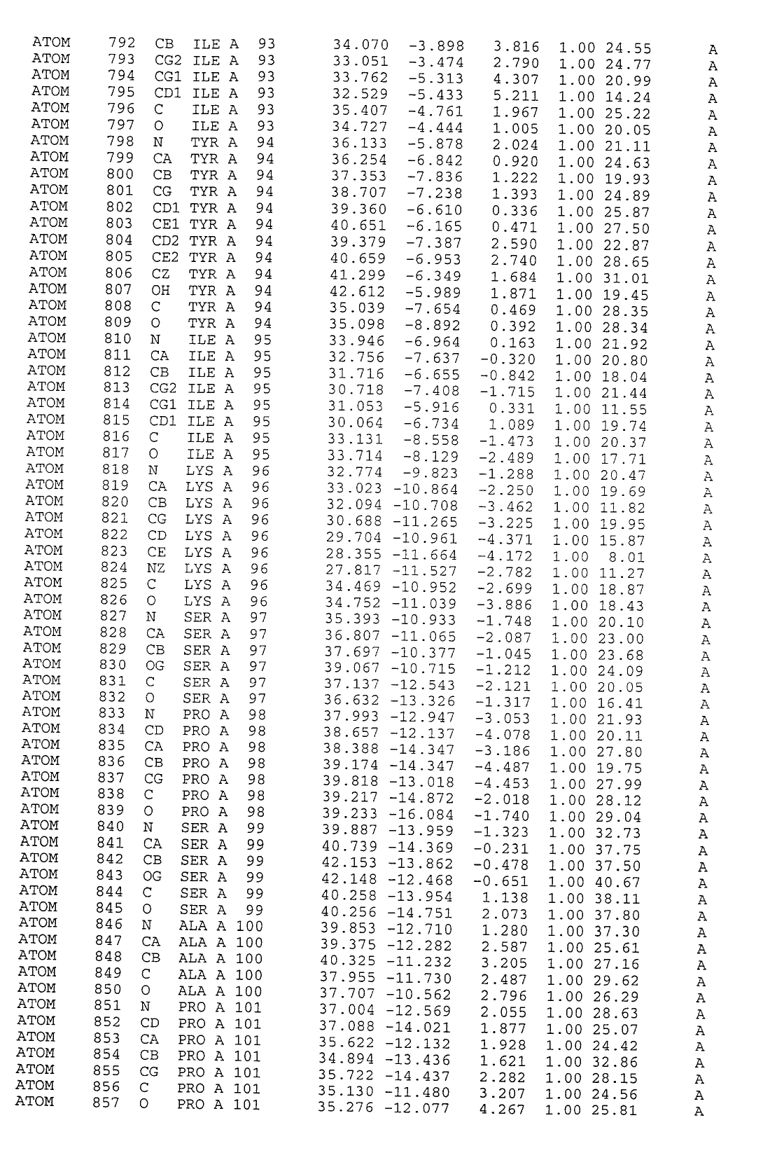

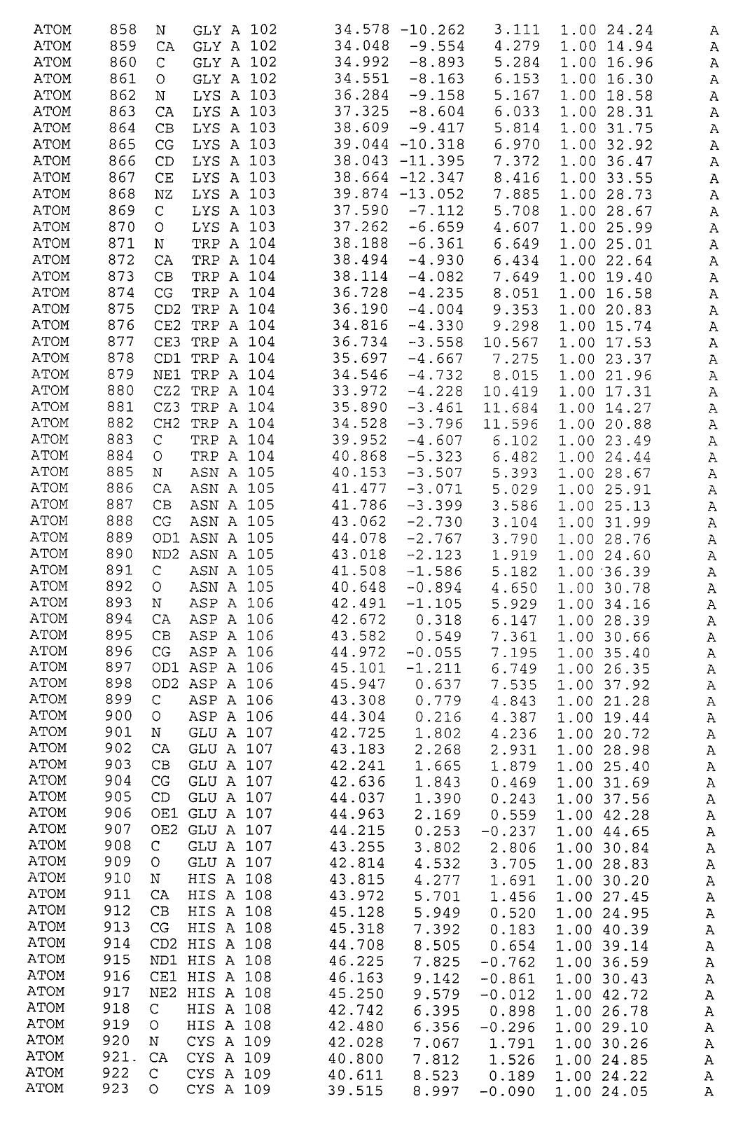

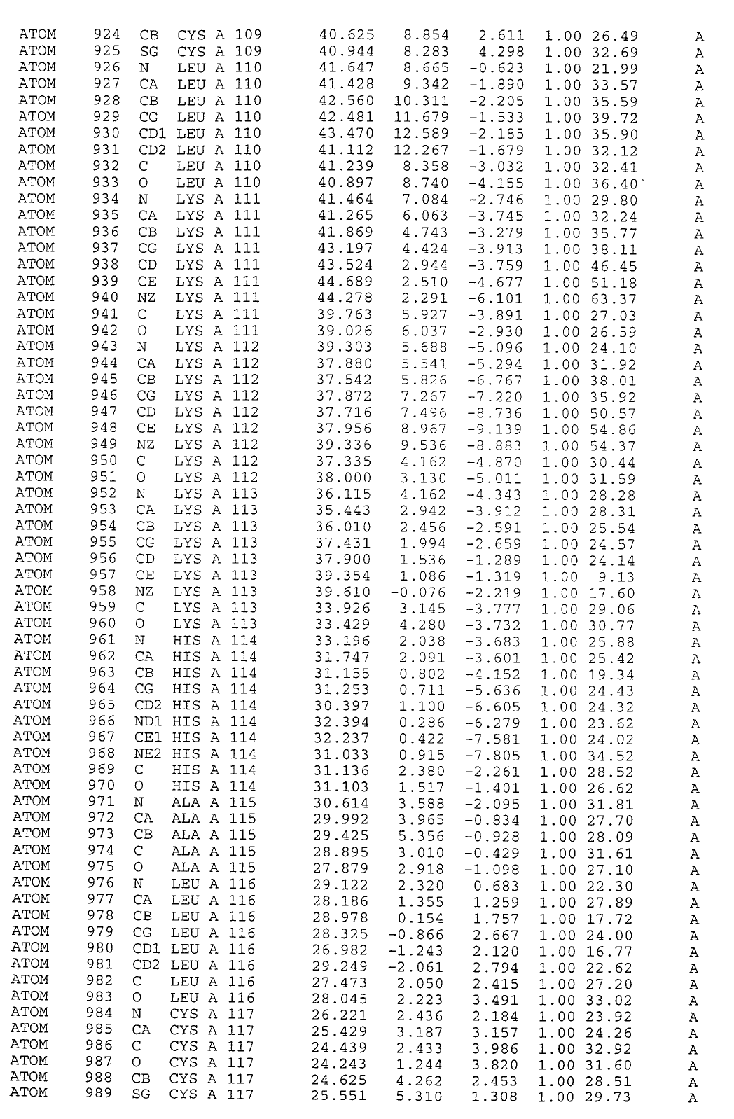

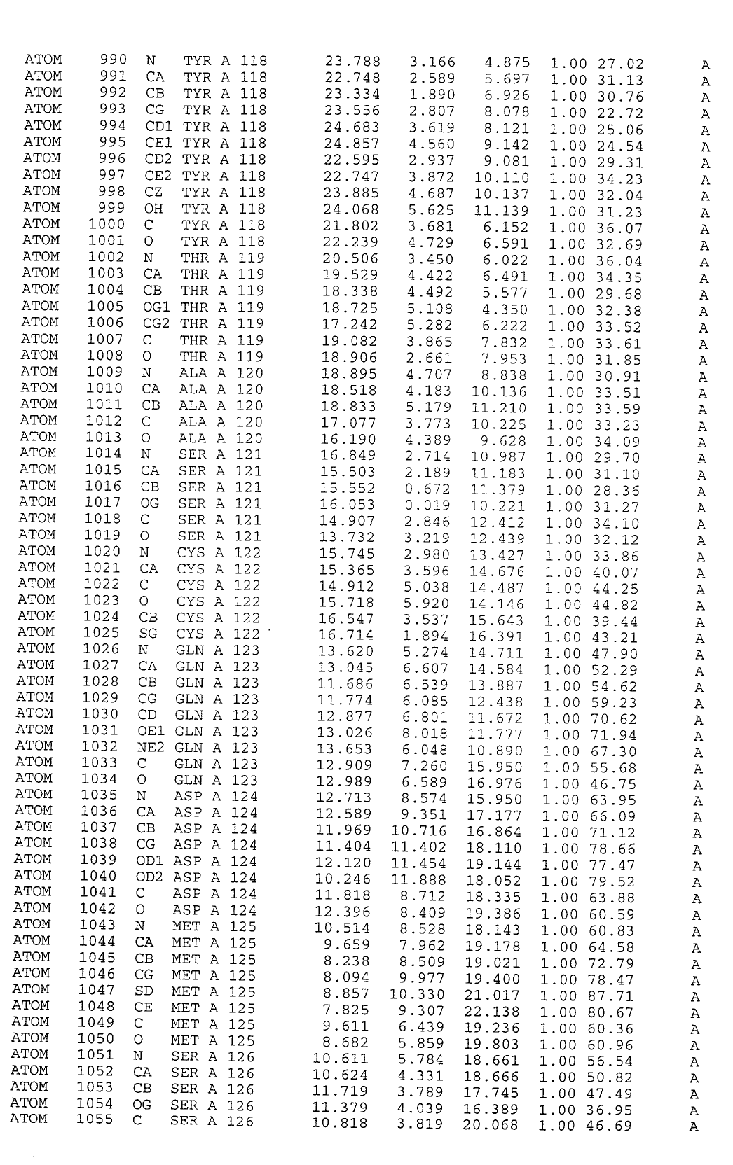

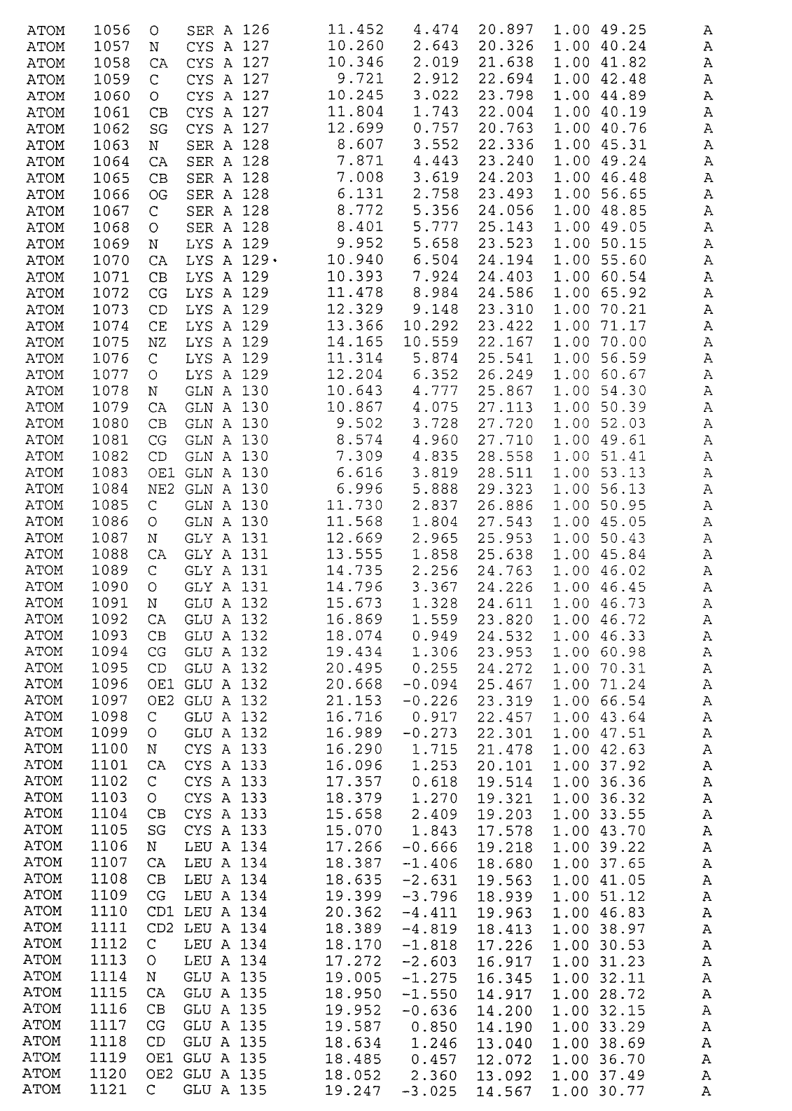

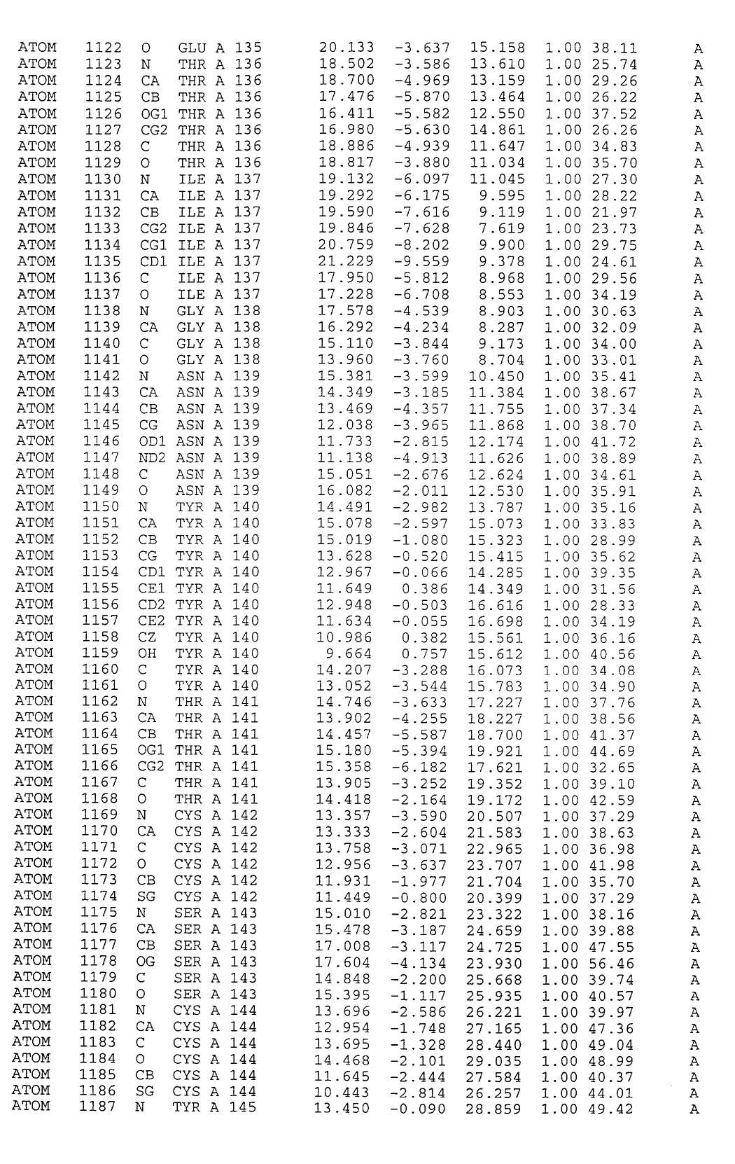

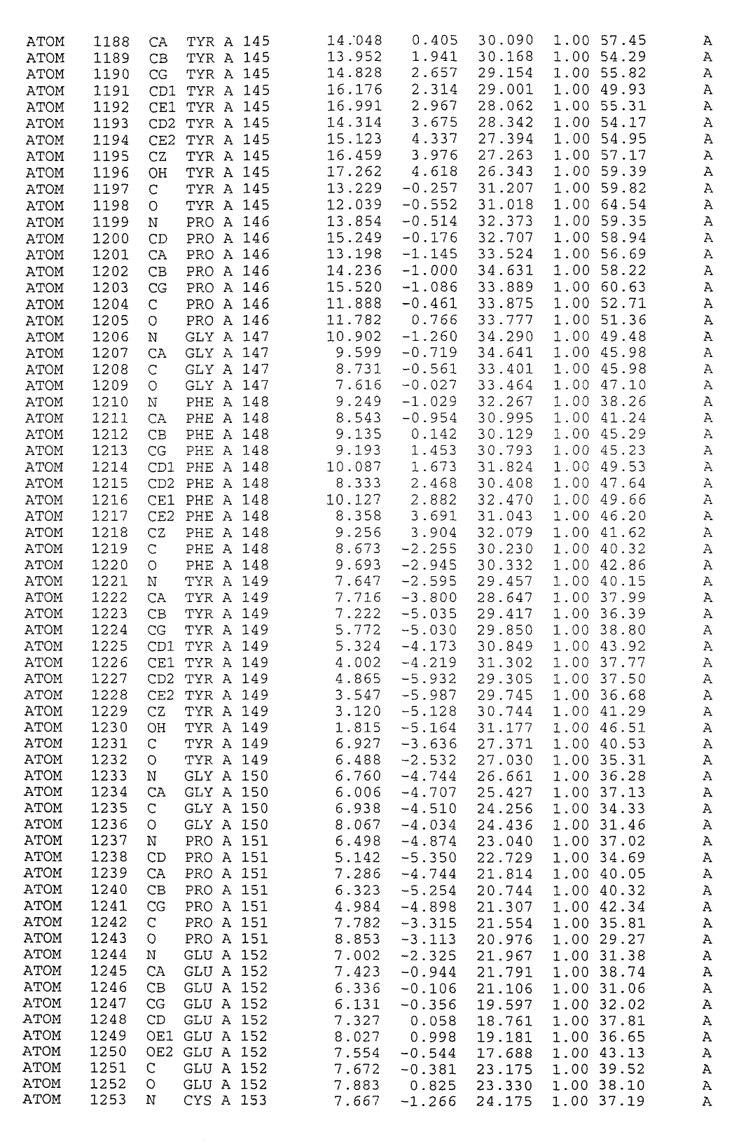

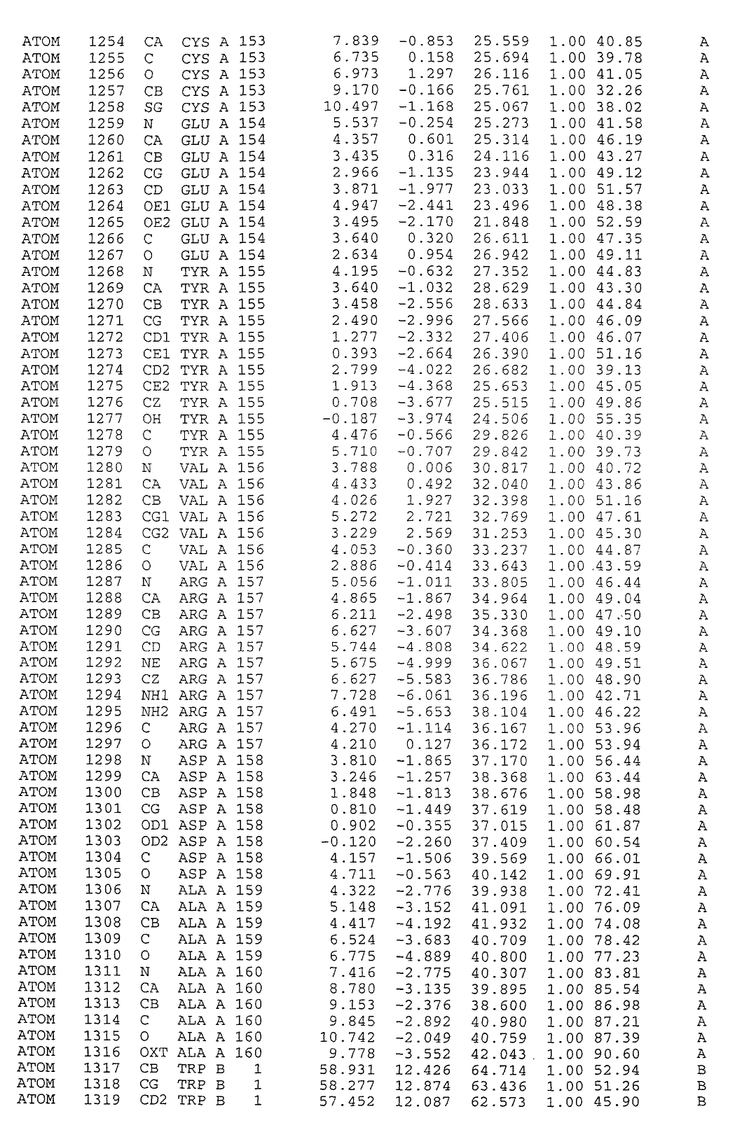

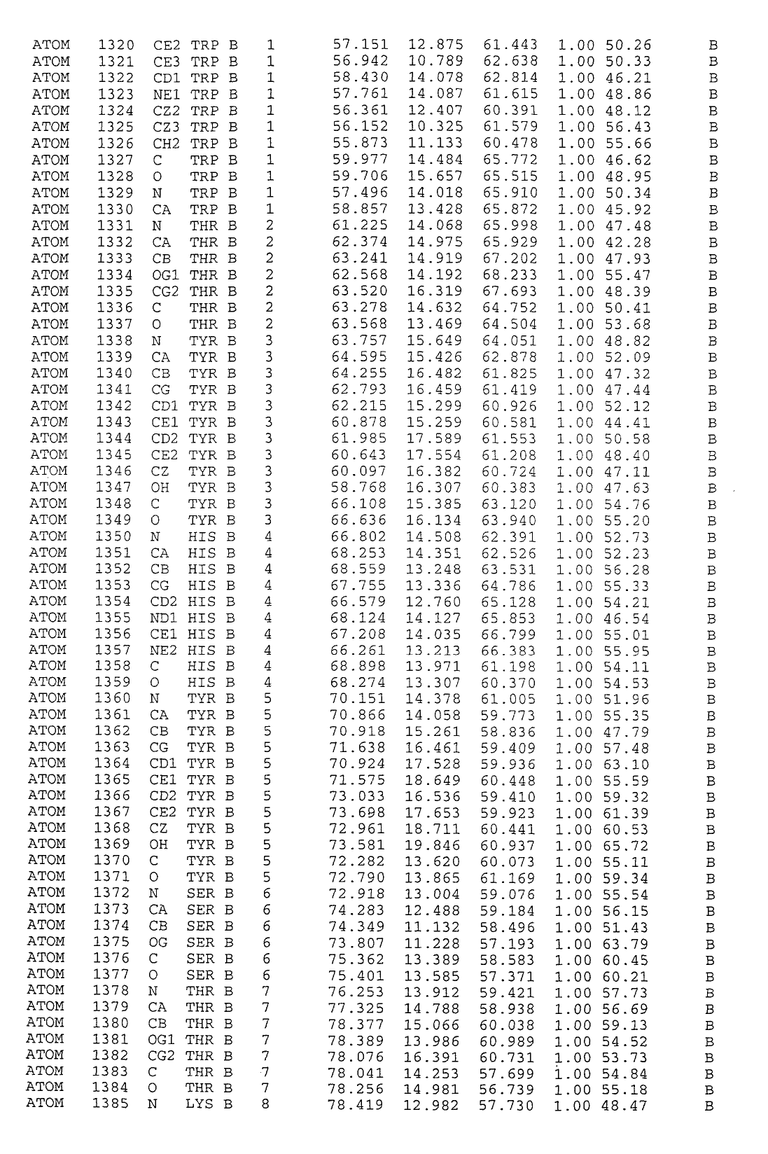

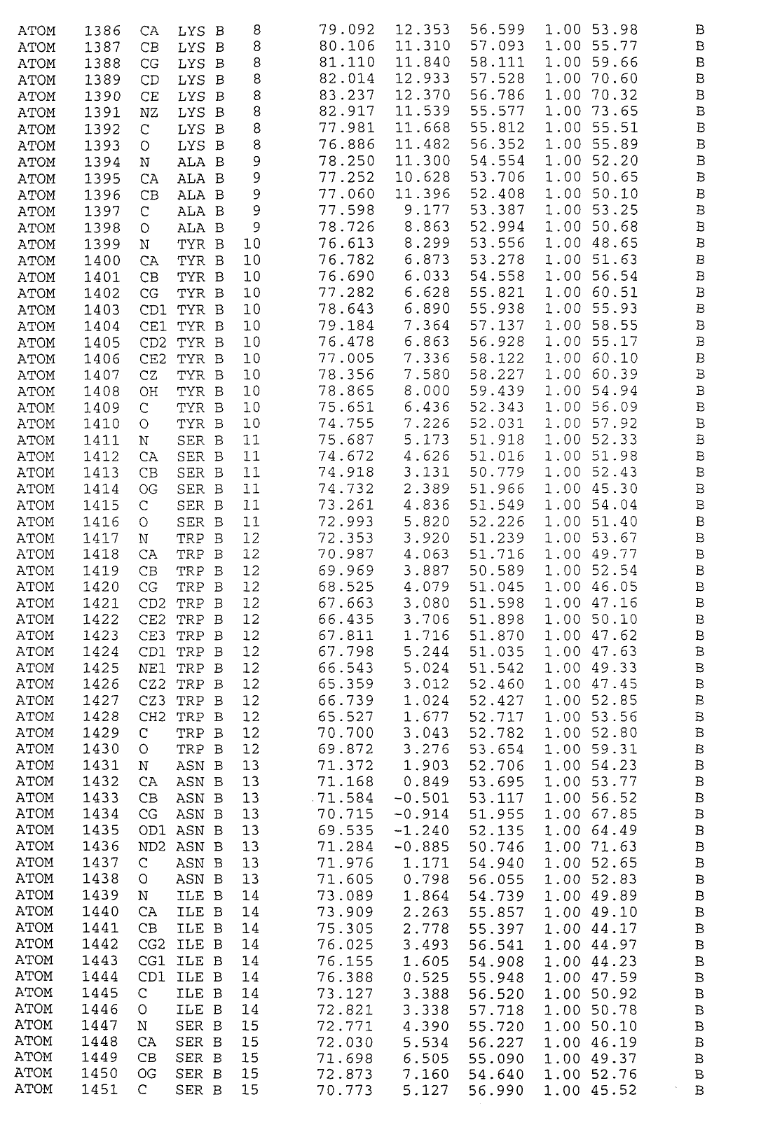

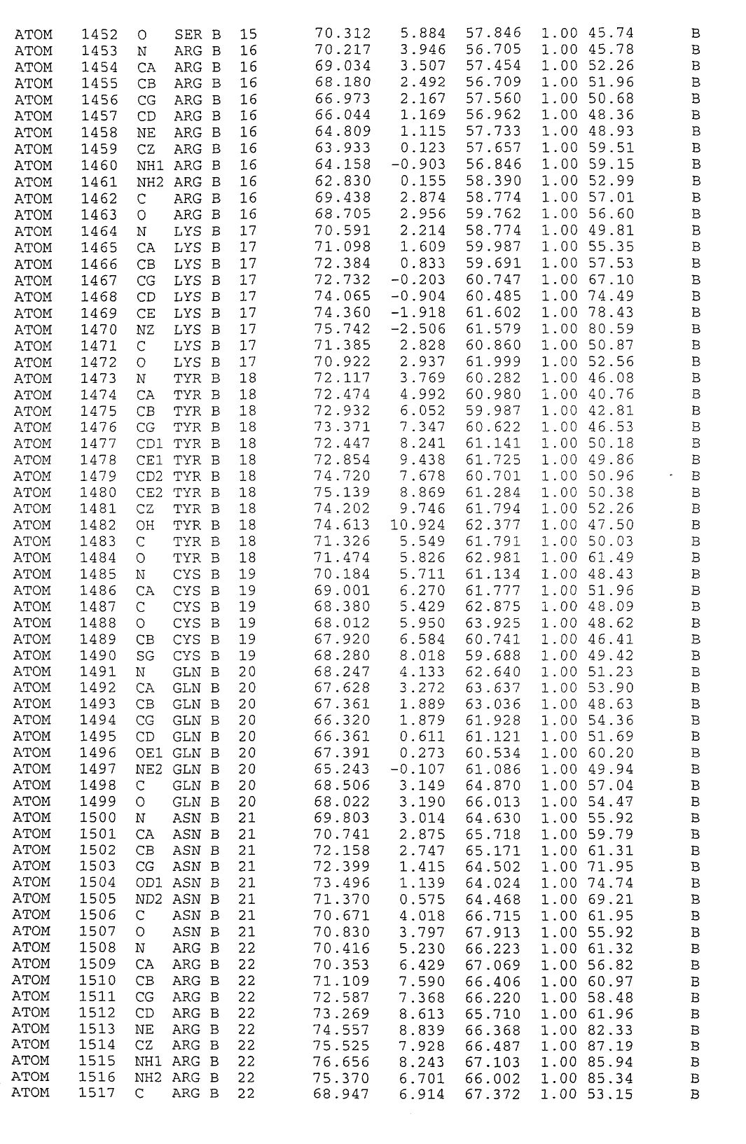

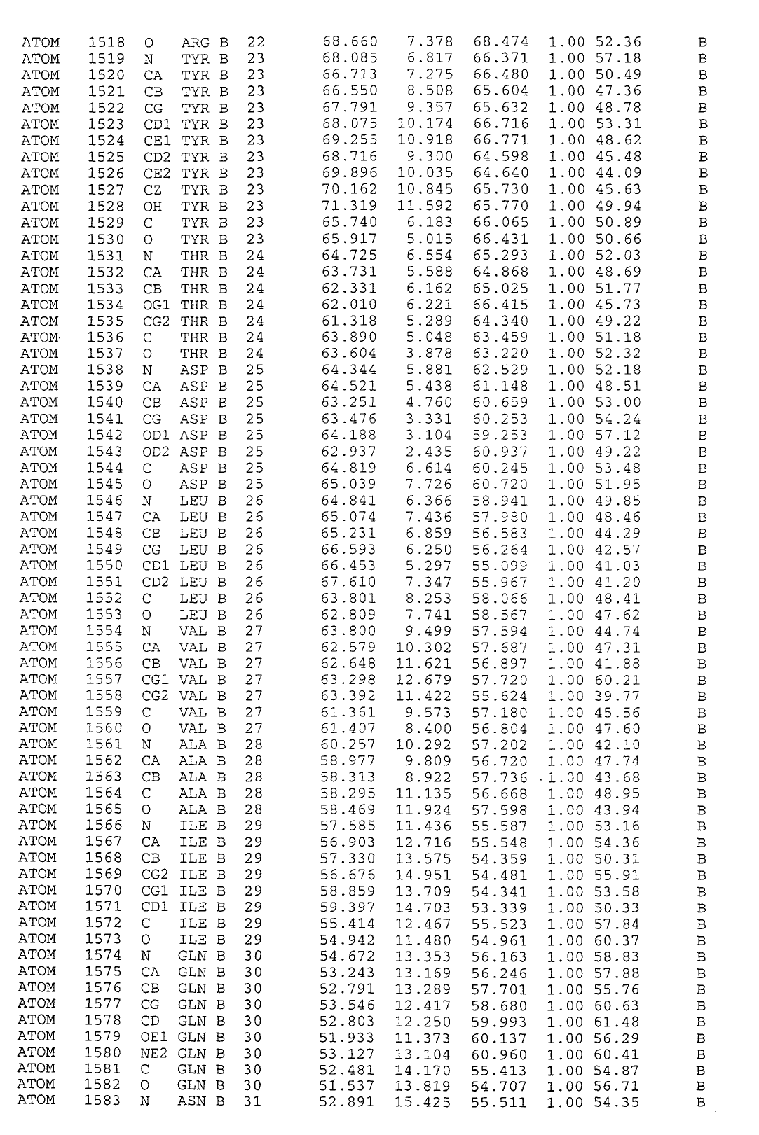

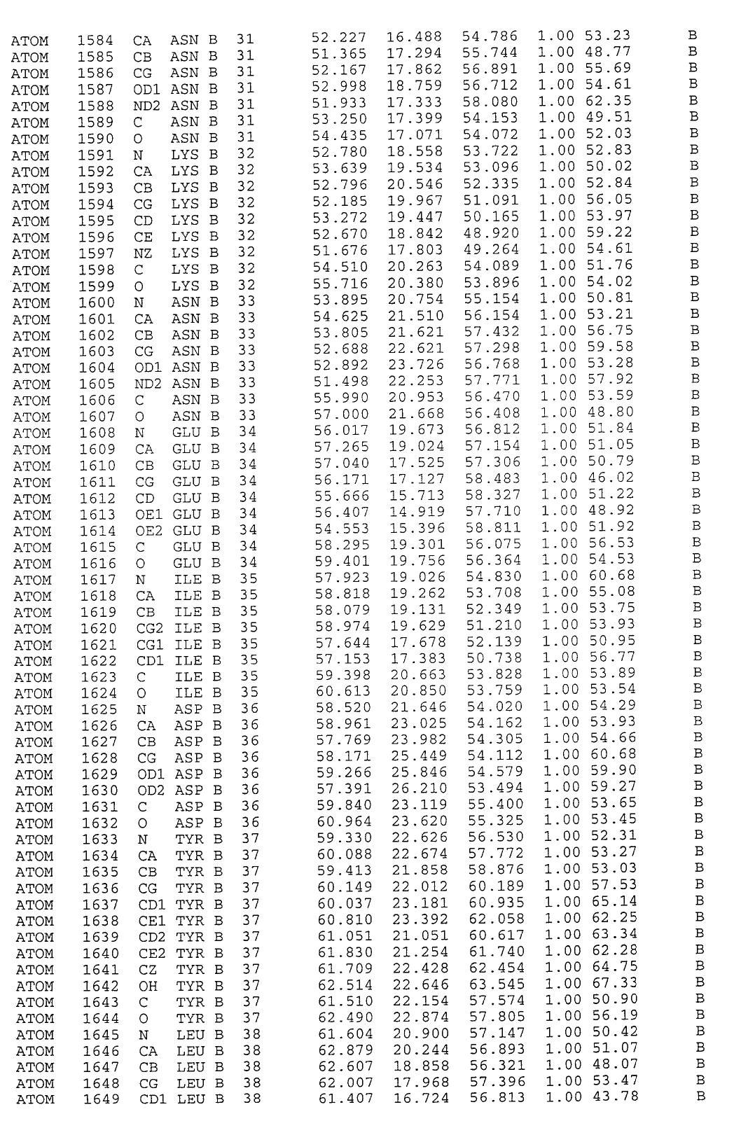

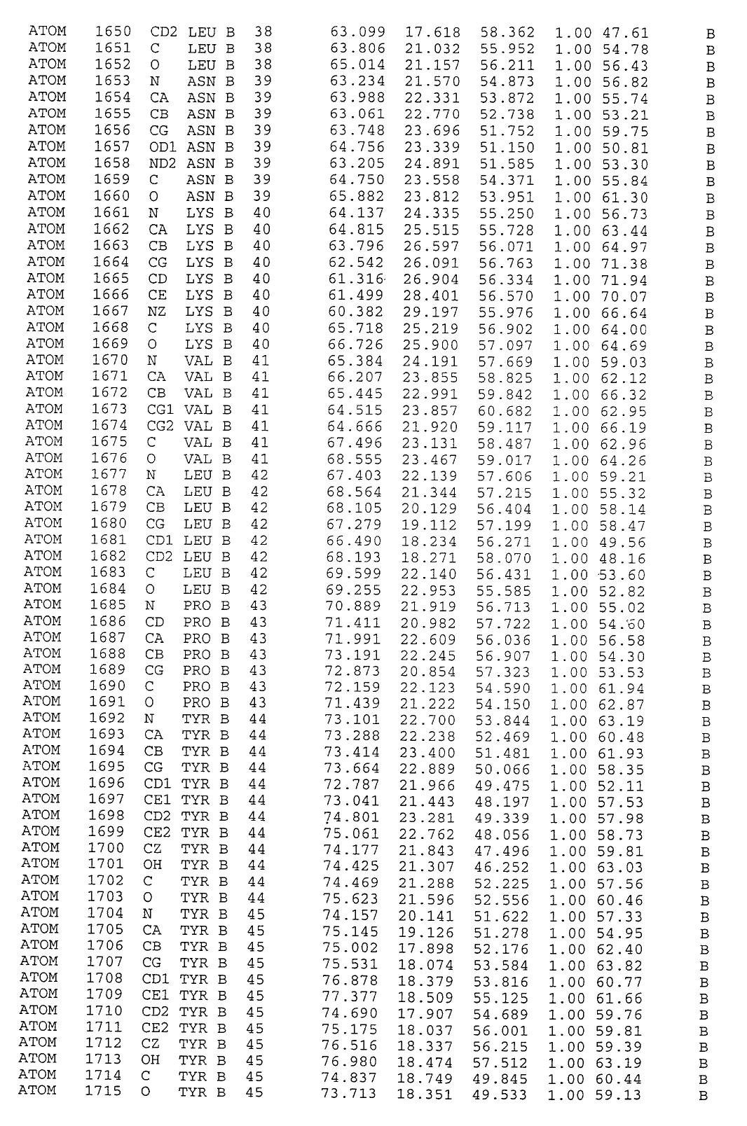

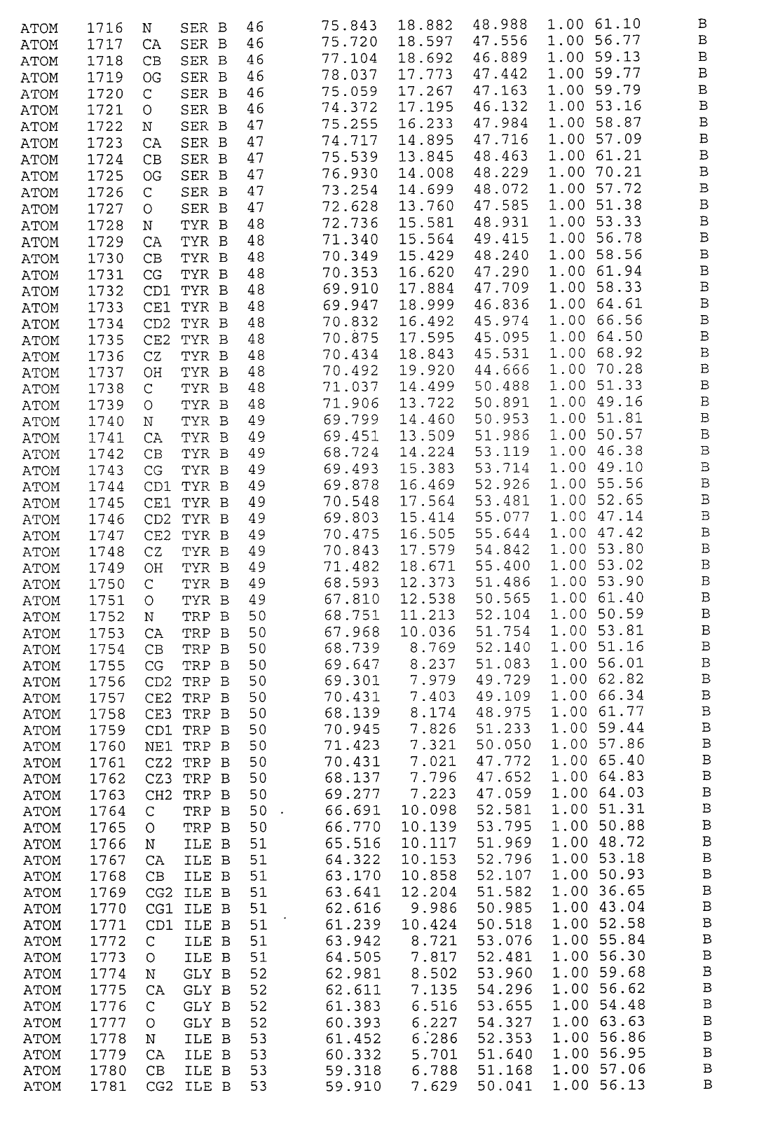

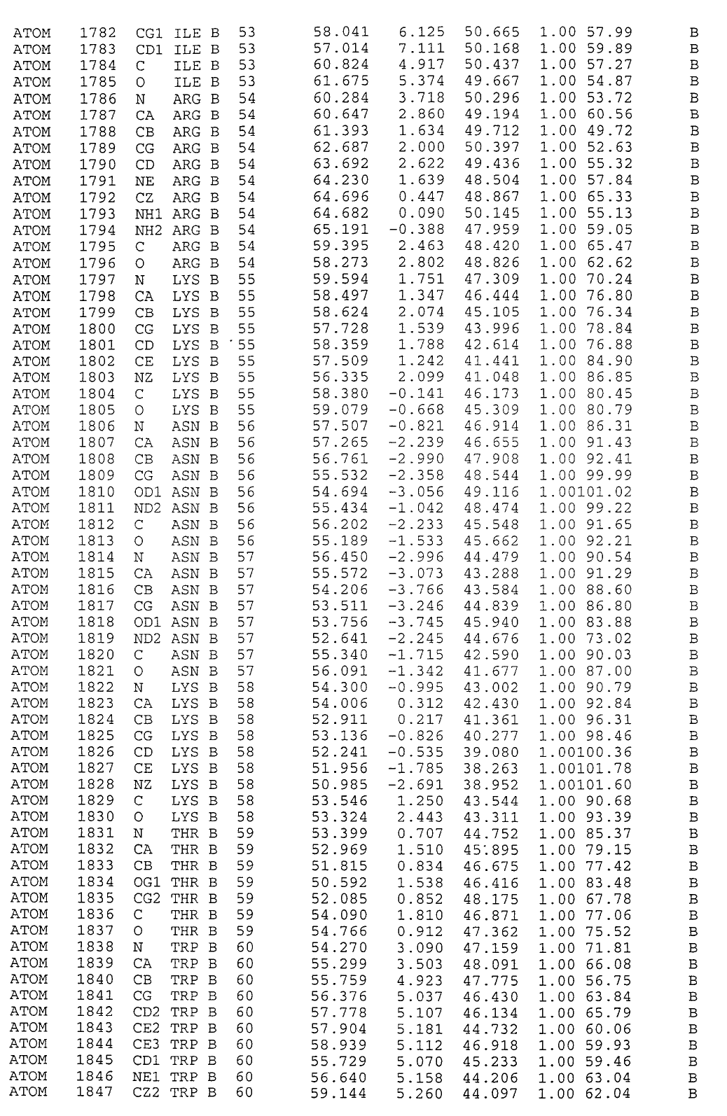

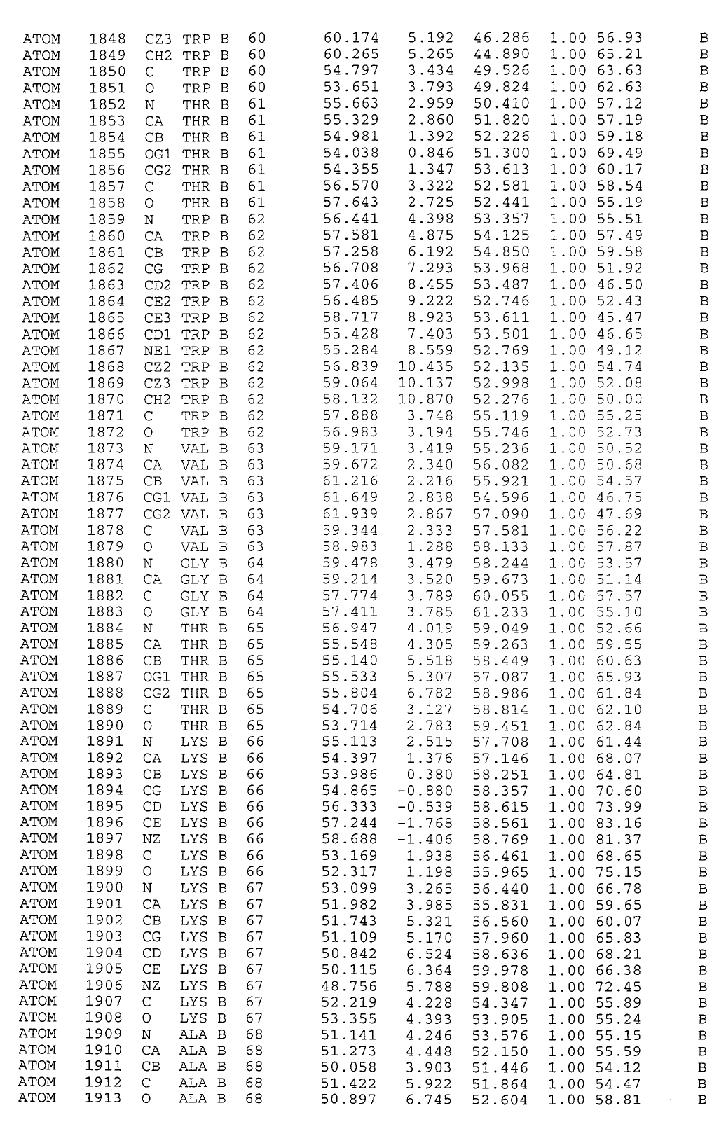

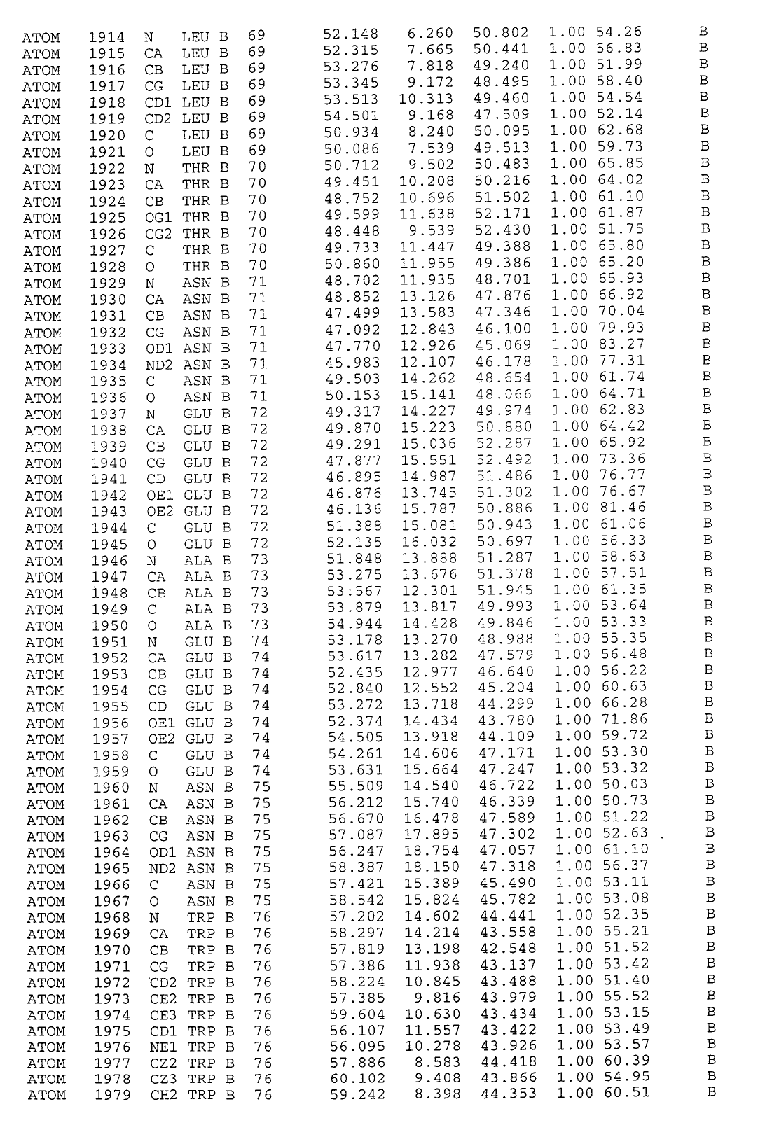

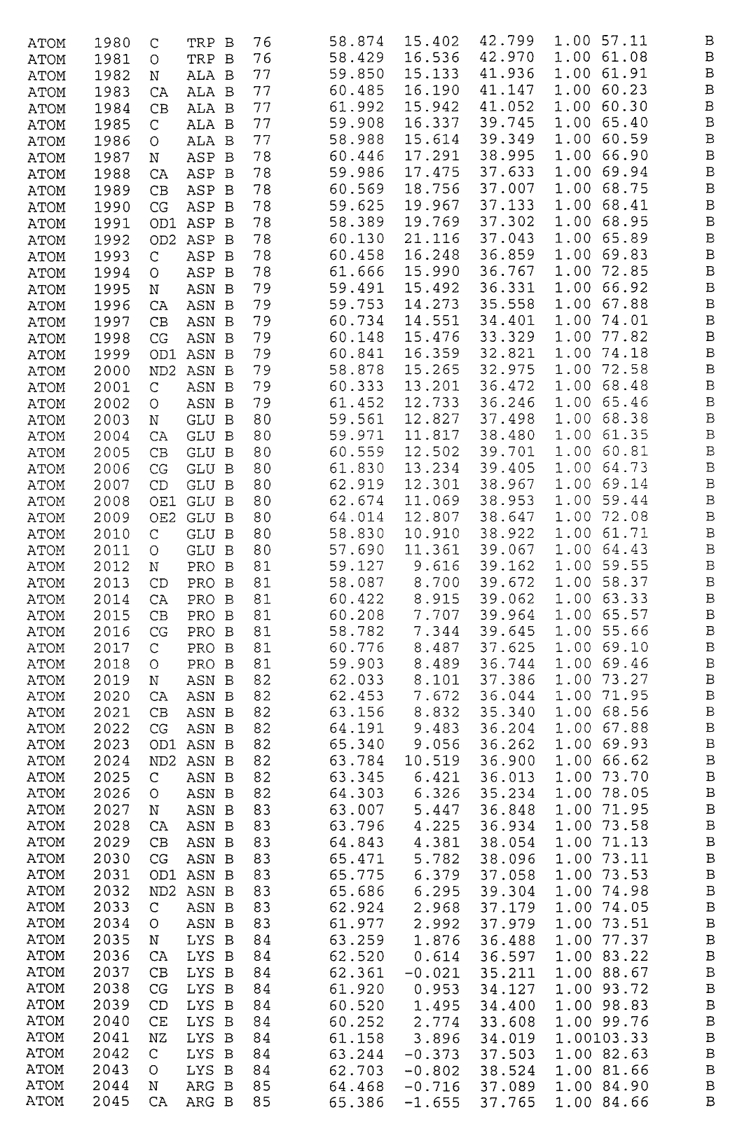

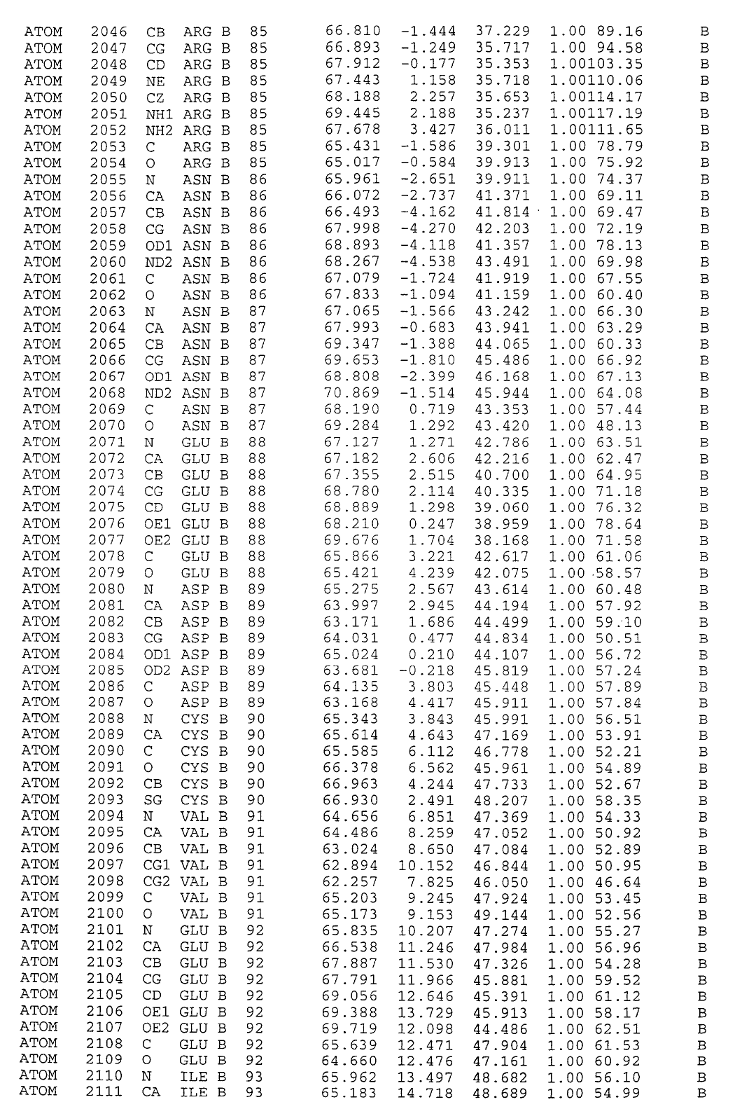

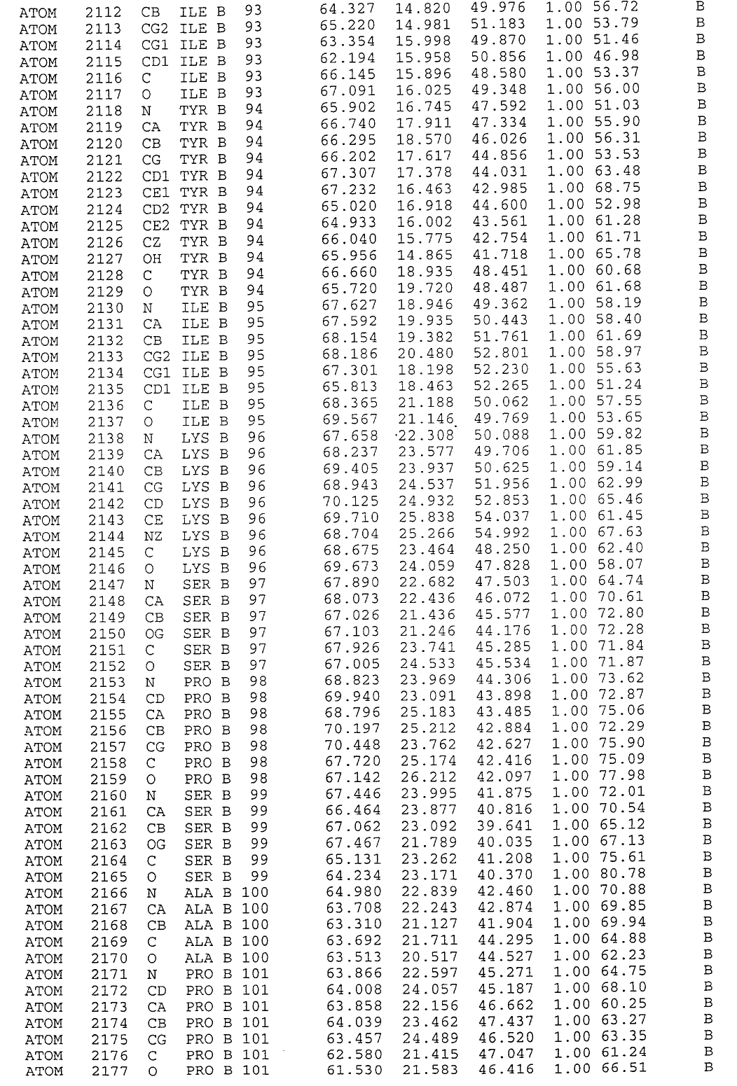

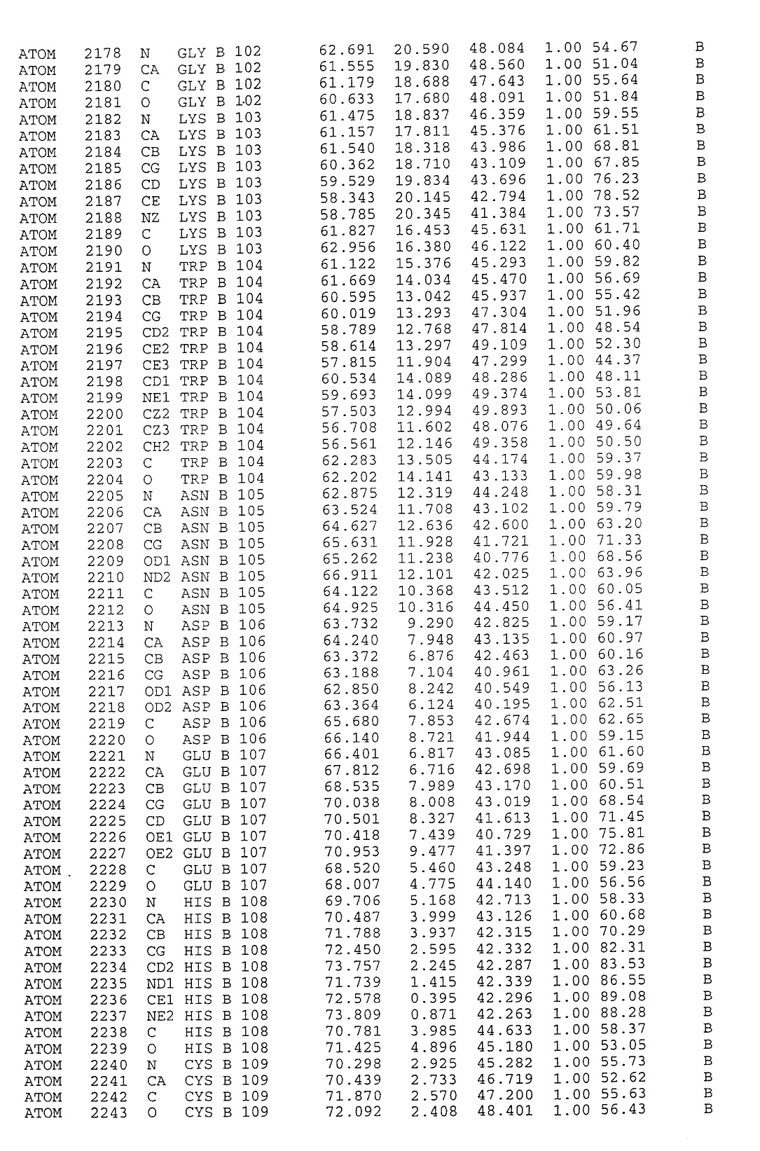

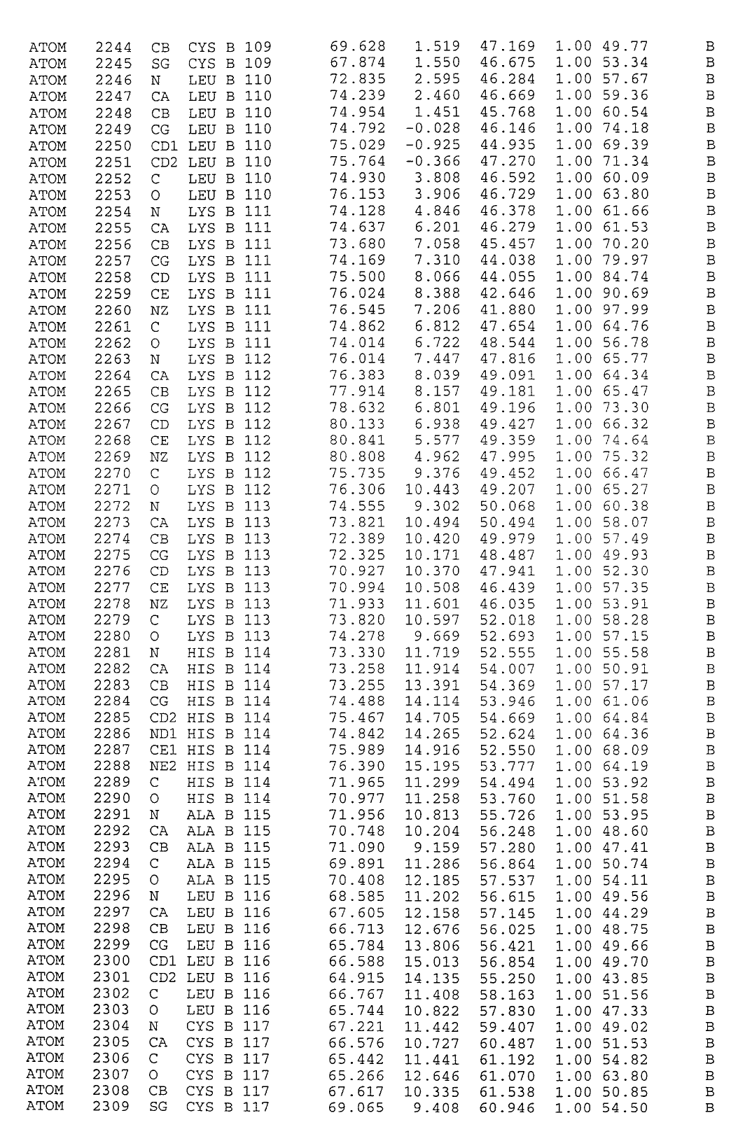

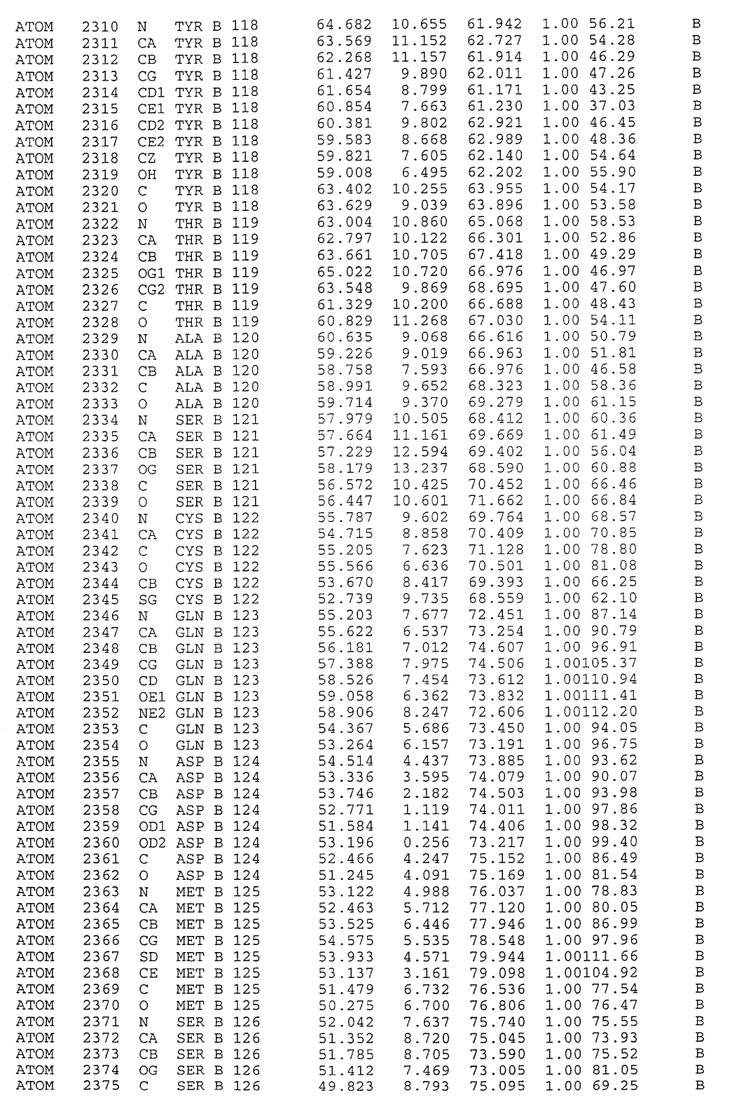

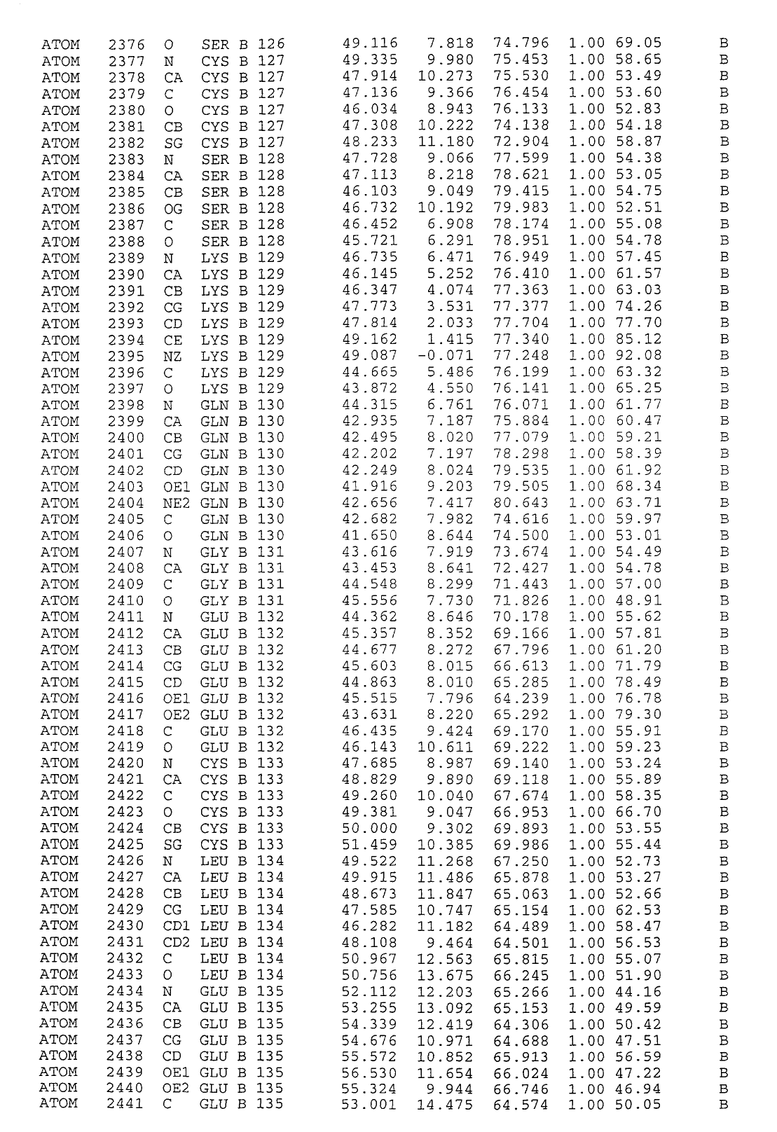

In addition, the present invention provides the three dimensional structures of P-selectin LE and SLe^{X} as derived by x-ray diffraction data of the P-selectin LE:SLe^{X} crystal. Specifically, the three dimensional structures of P-selectin LE and SLe^{X} are defined by the structural coordinates shown in FIG. 3, ± a root mean square deviation from the backbone atoms of the amino acids of not more than 1.5 Å, preferably not more than 1.0 Å, and most preferably not more than 0.5 Å. The structural coordinates of P-selectin LE and SLe^{X} are useful for a number of applications, including, but not limited to, the visualization, identification and characterization of various active sites of P-selectin LE, SLe^{X}and the P-selectin LE:SLe^{X} complex, including the SLe^{X} binding site. The active site structures may then be used to design agents with interact with P-selectin LE, SLe^{X}, as well as P-selectin LE complexed with SLe^{X}, PSGL-1, or related molecules.

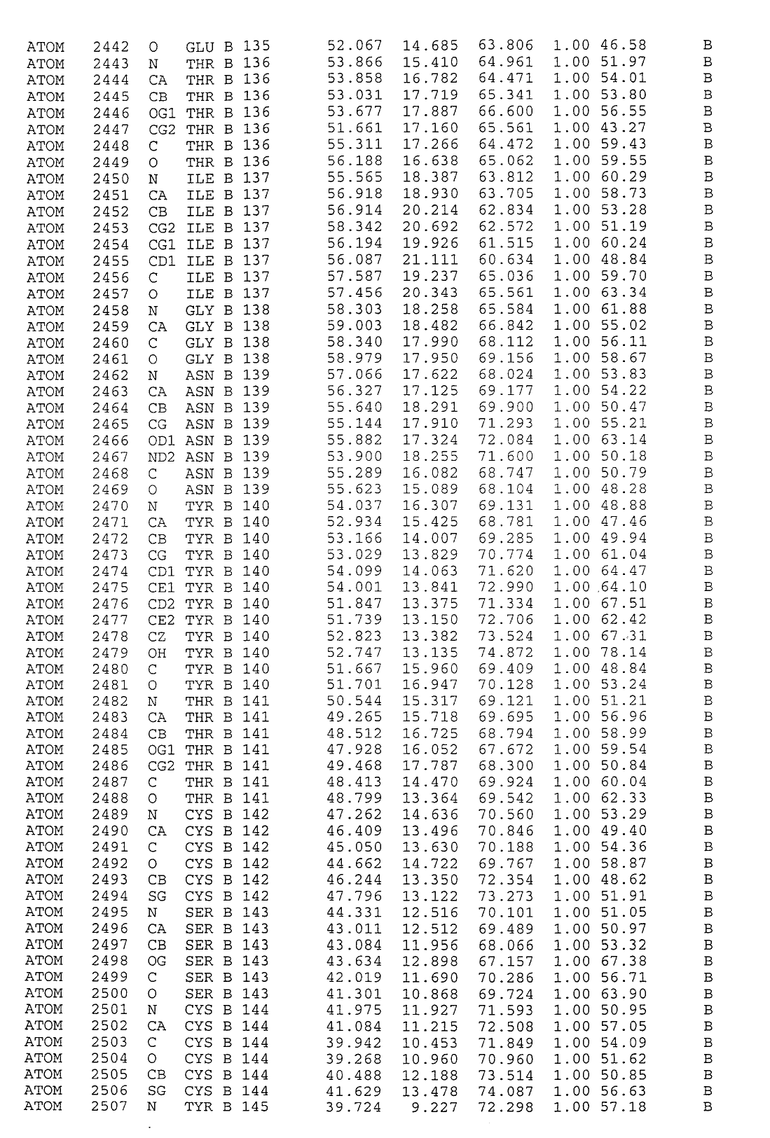

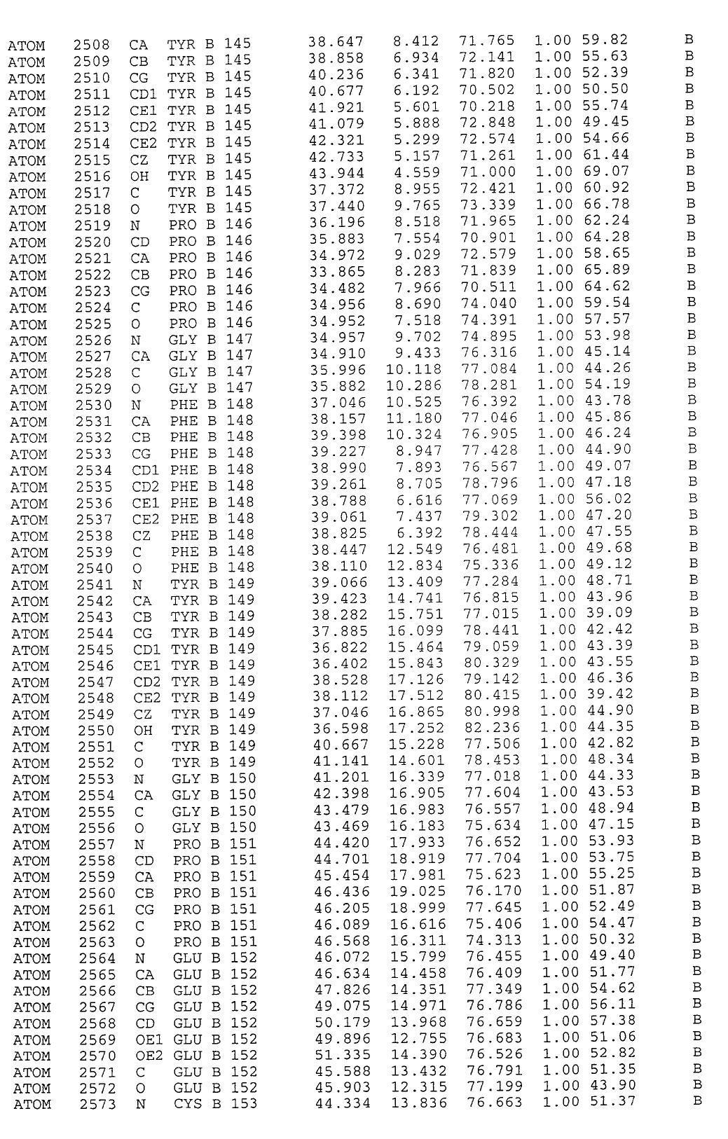

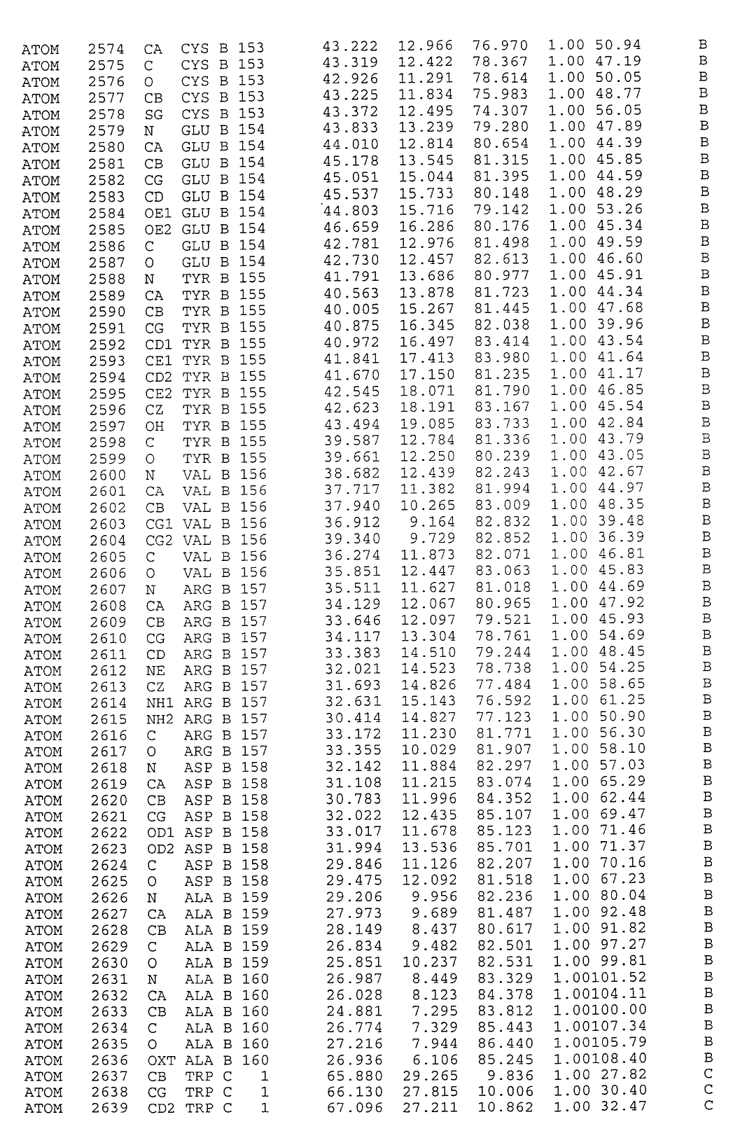

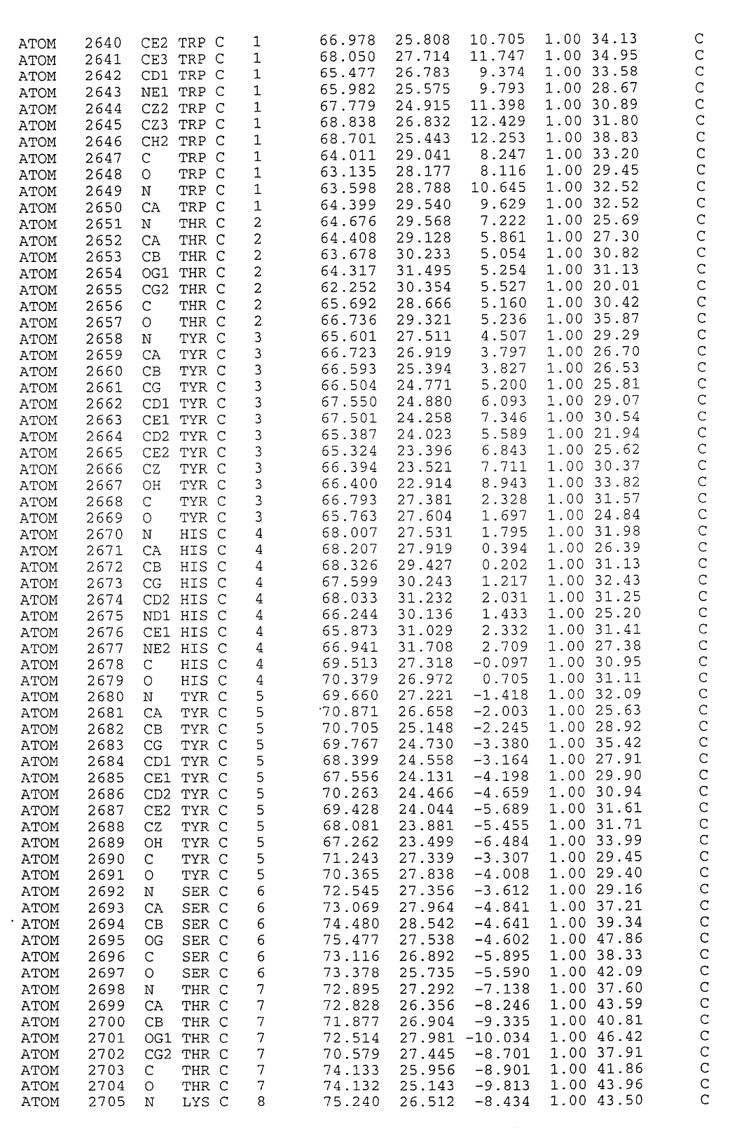

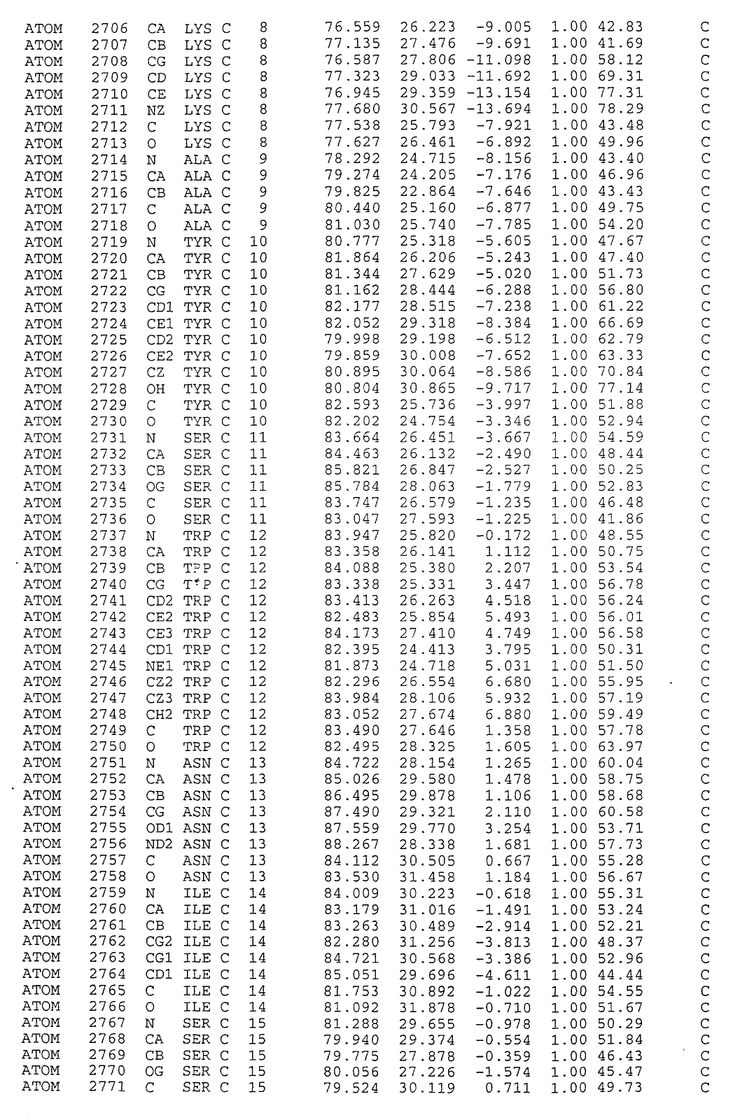

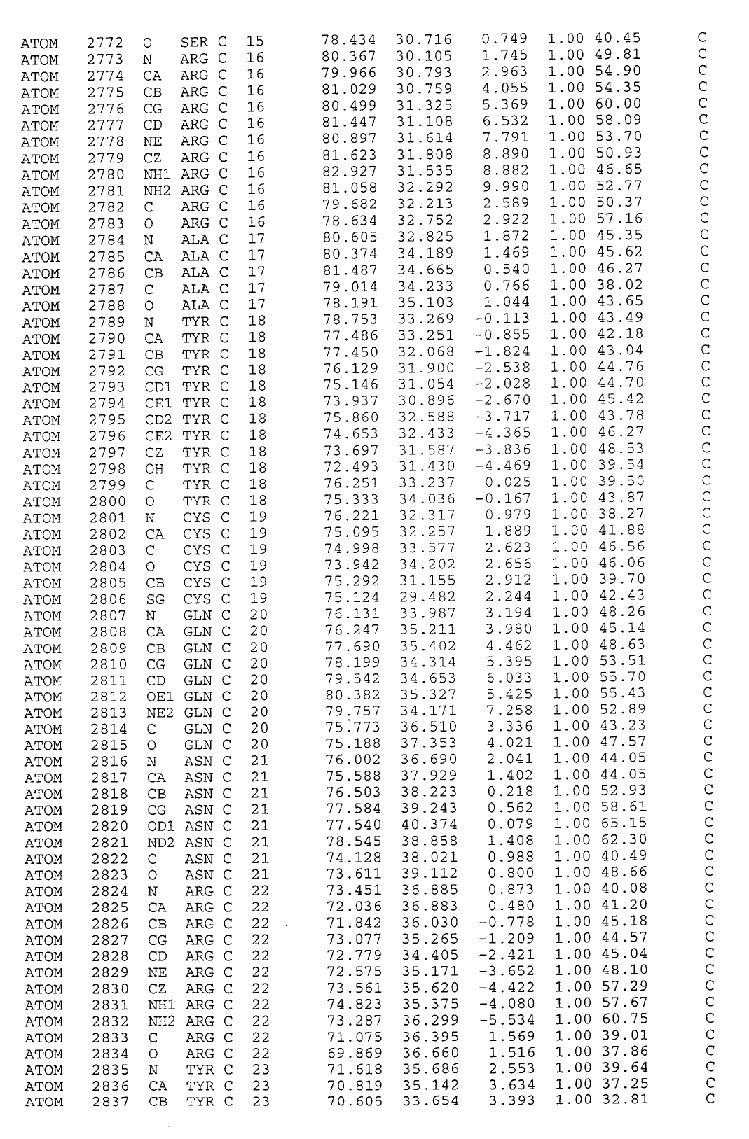

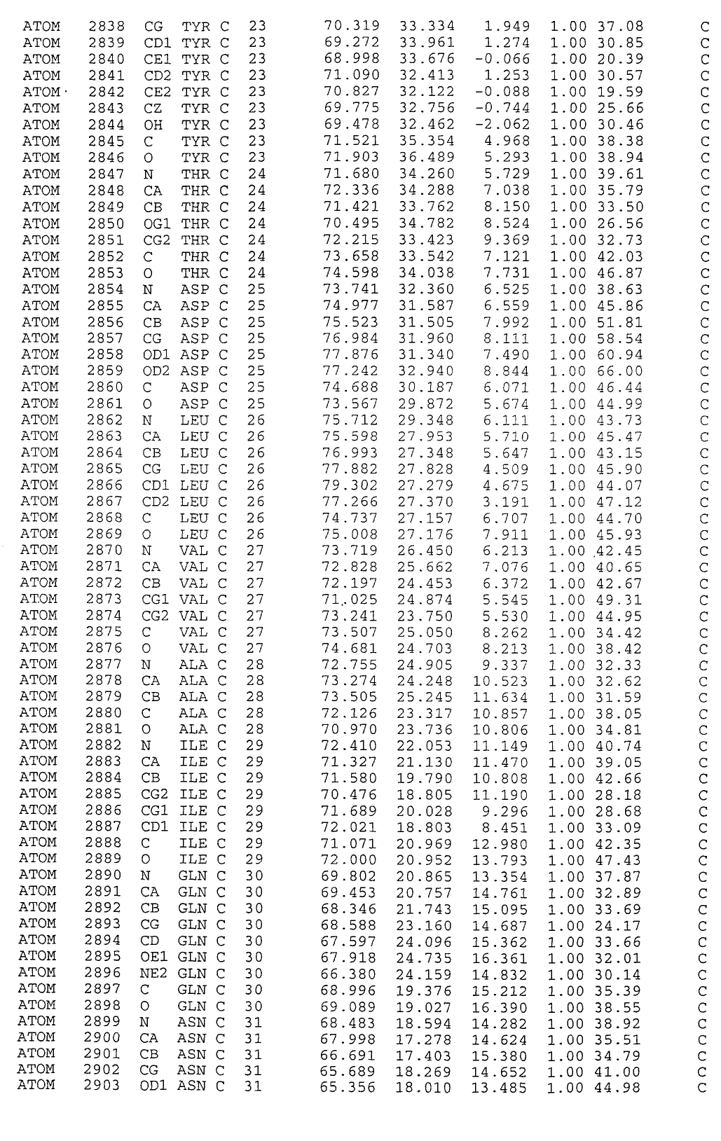

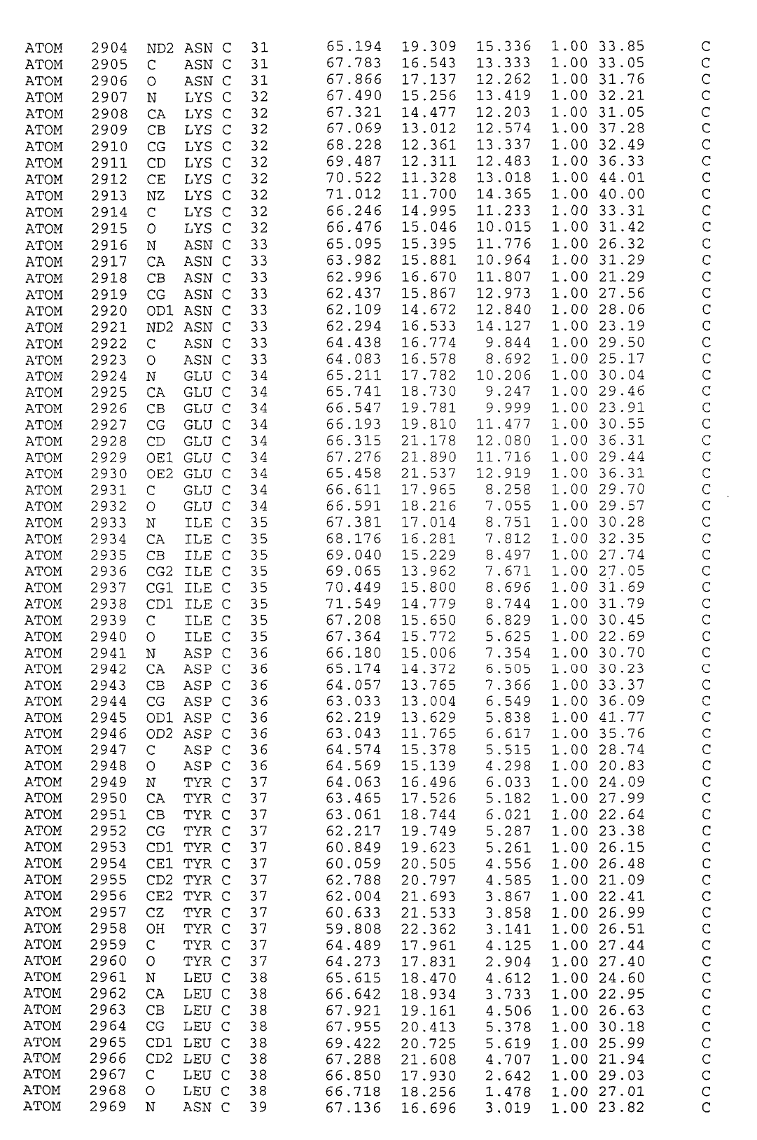

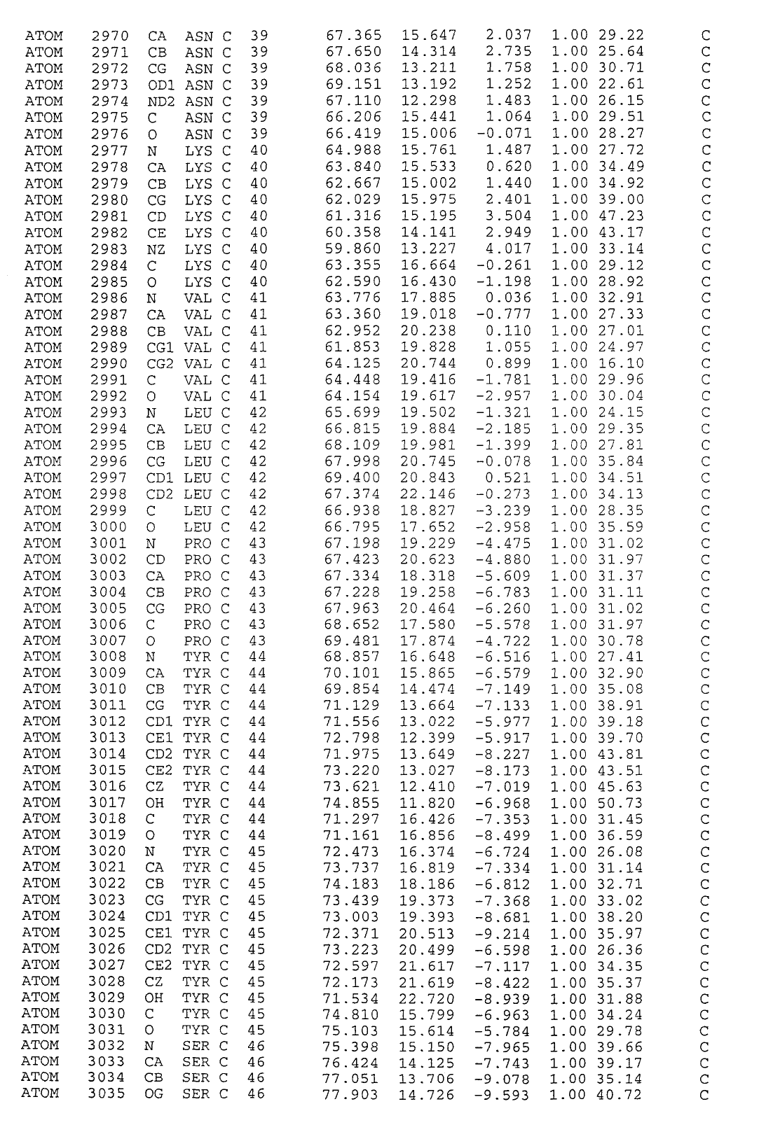

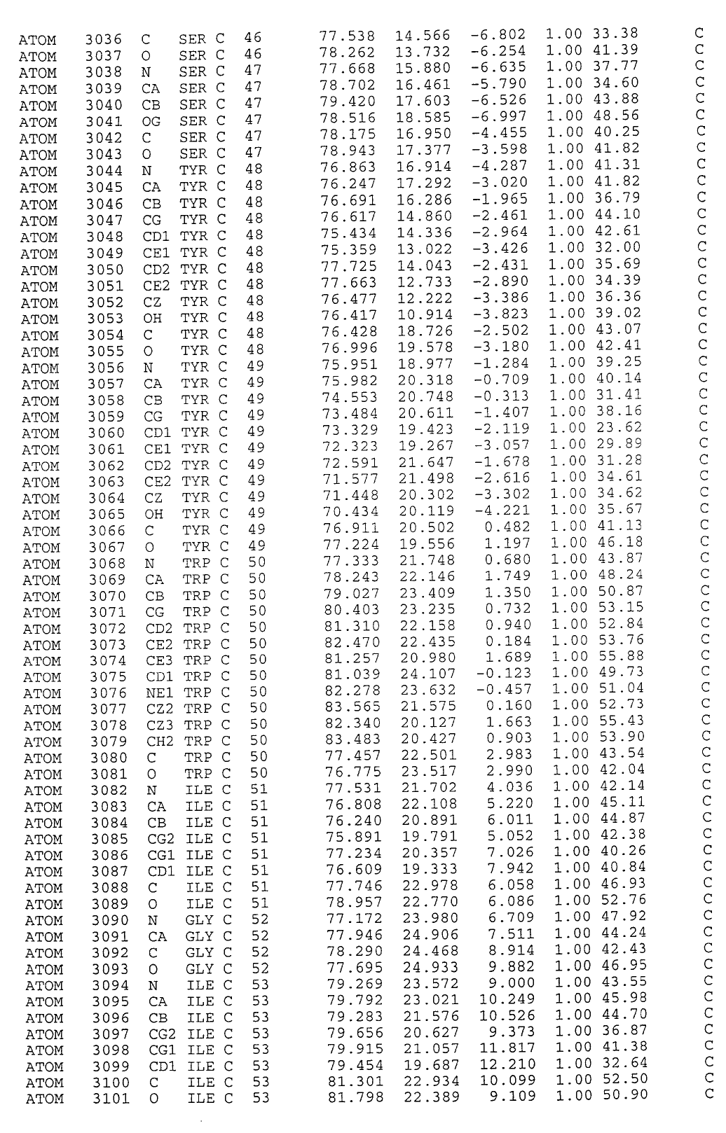

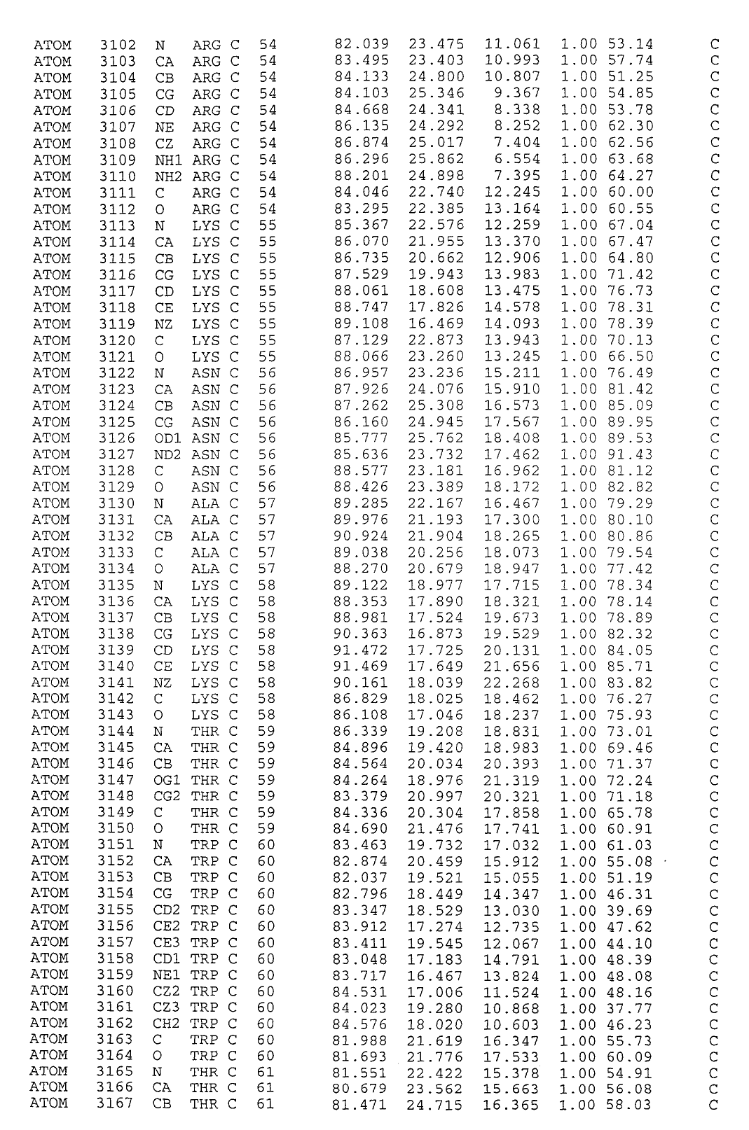

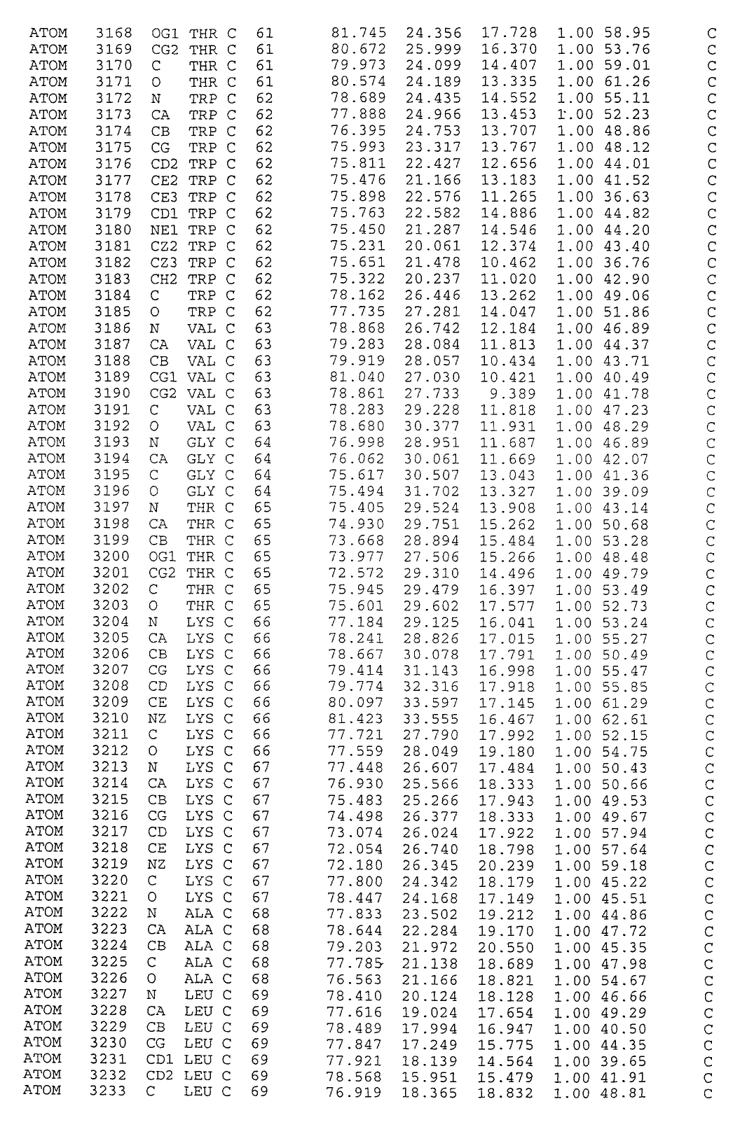

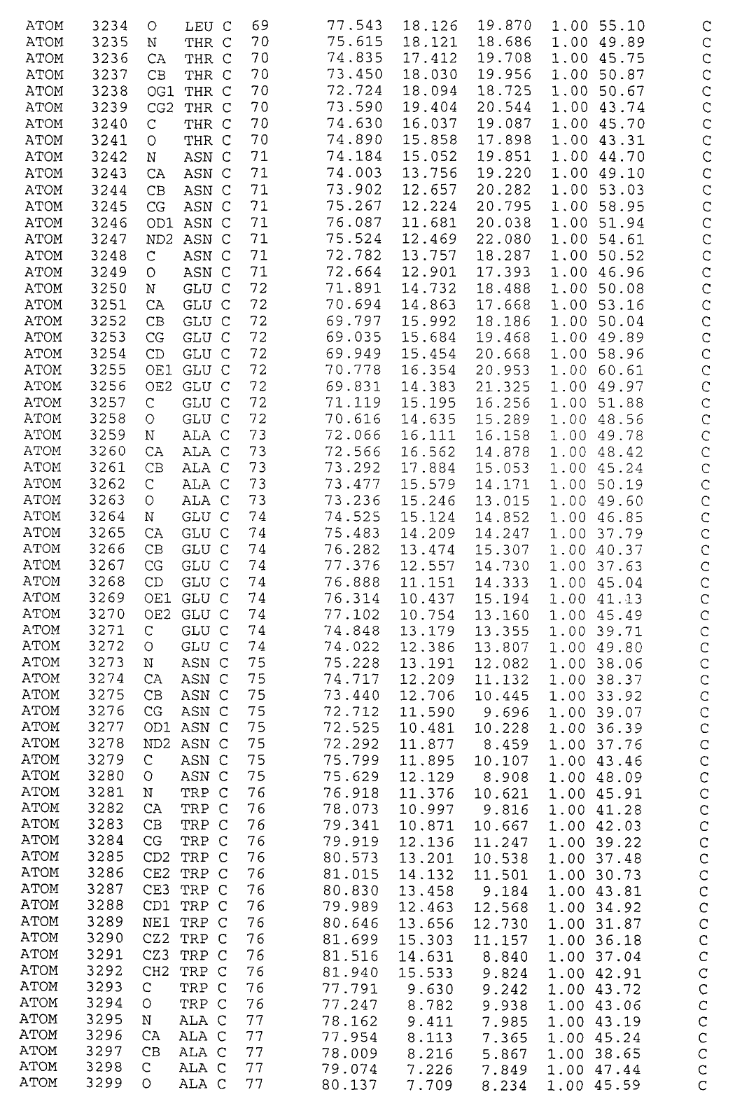

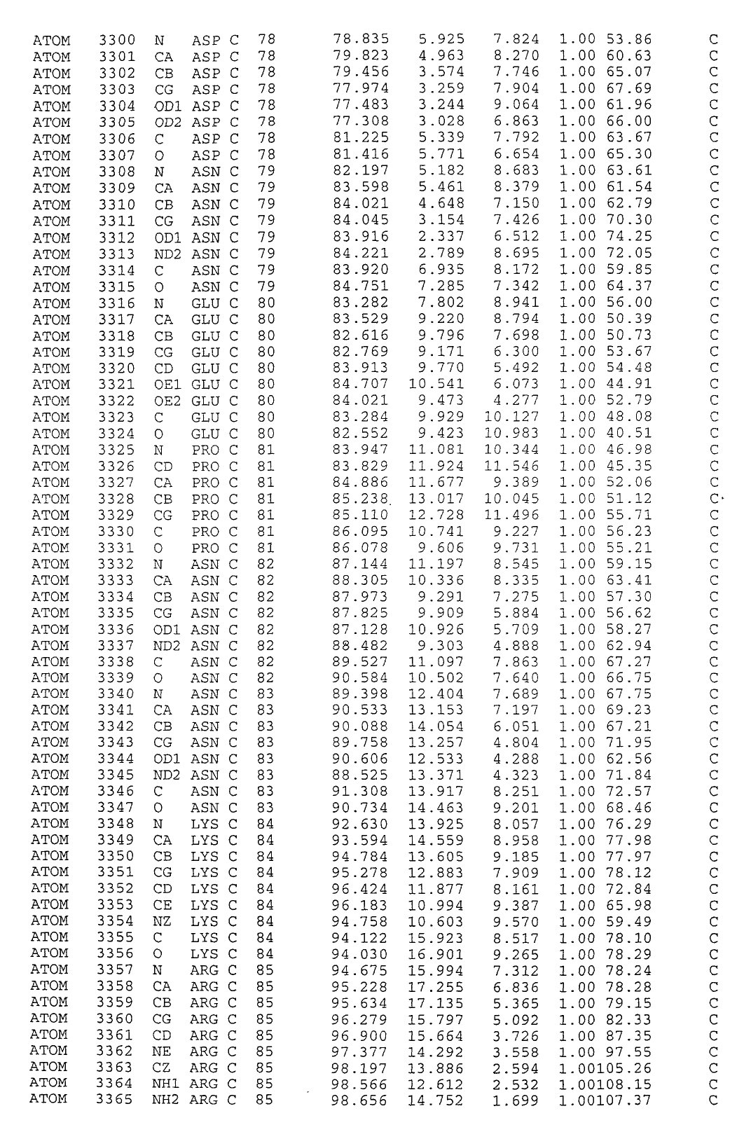

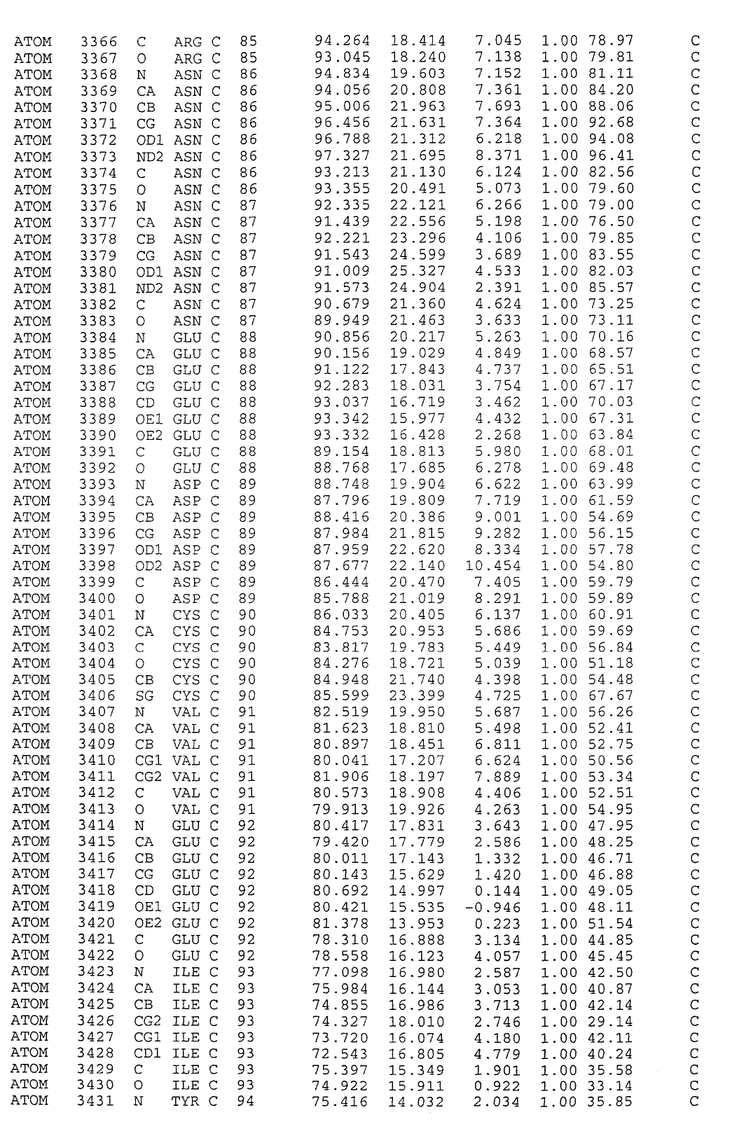

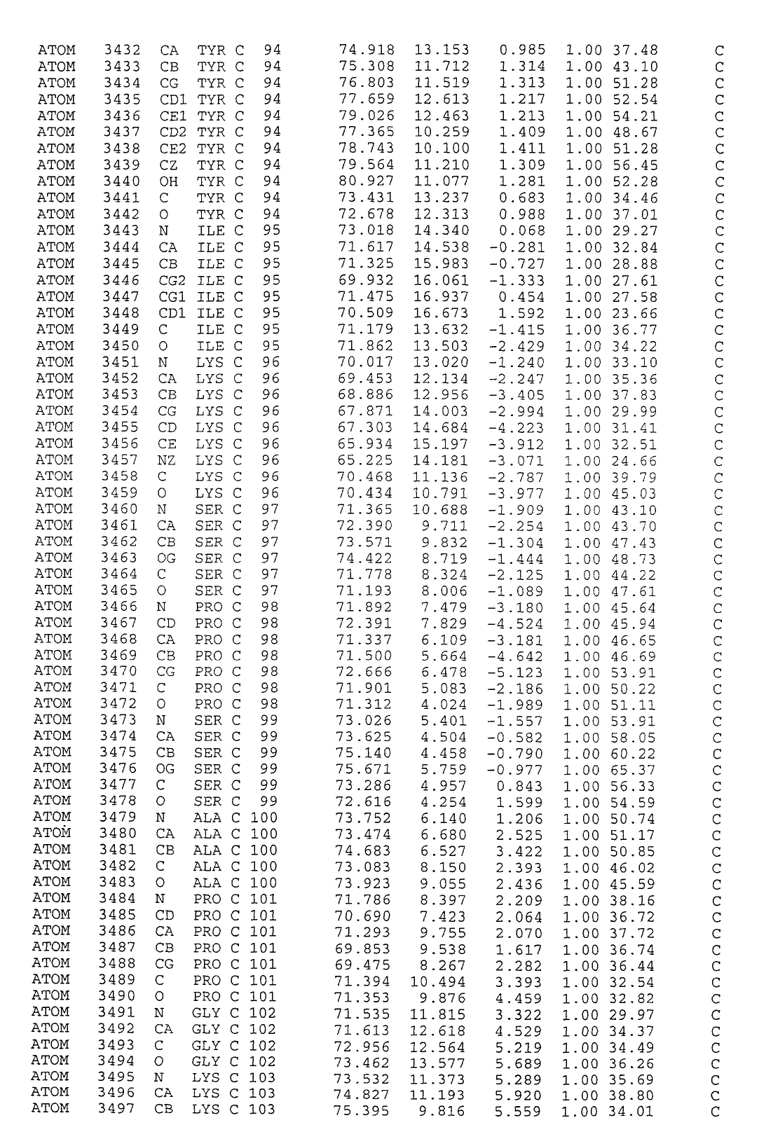

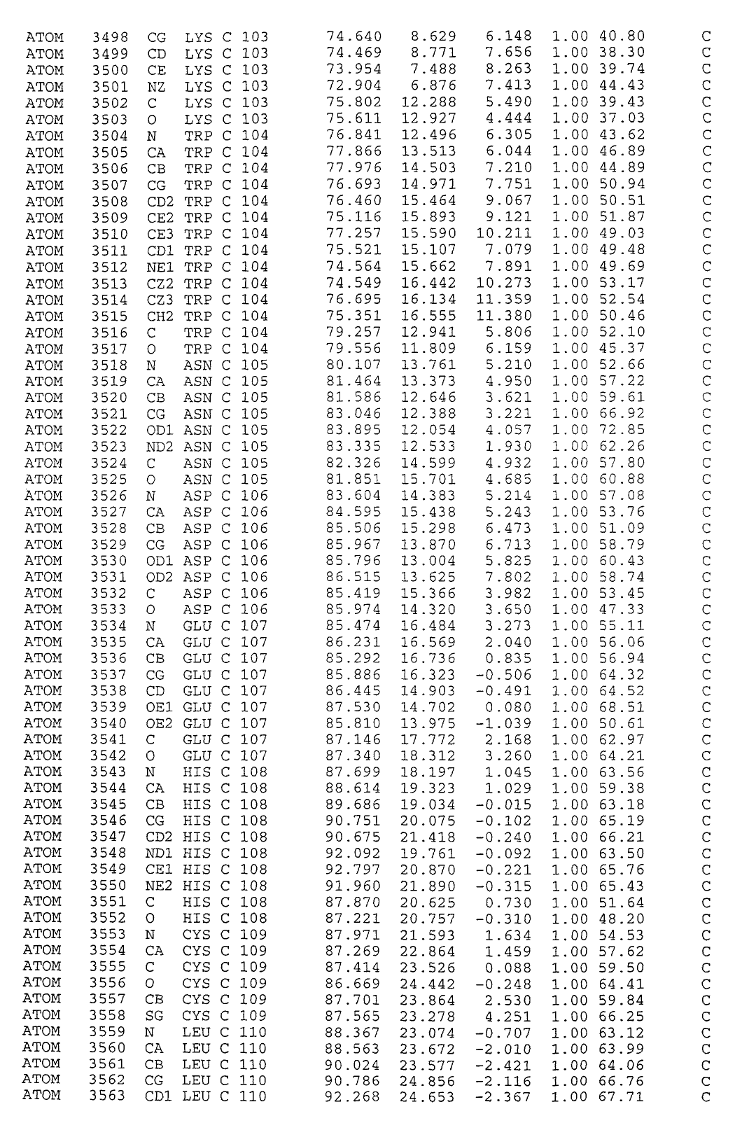

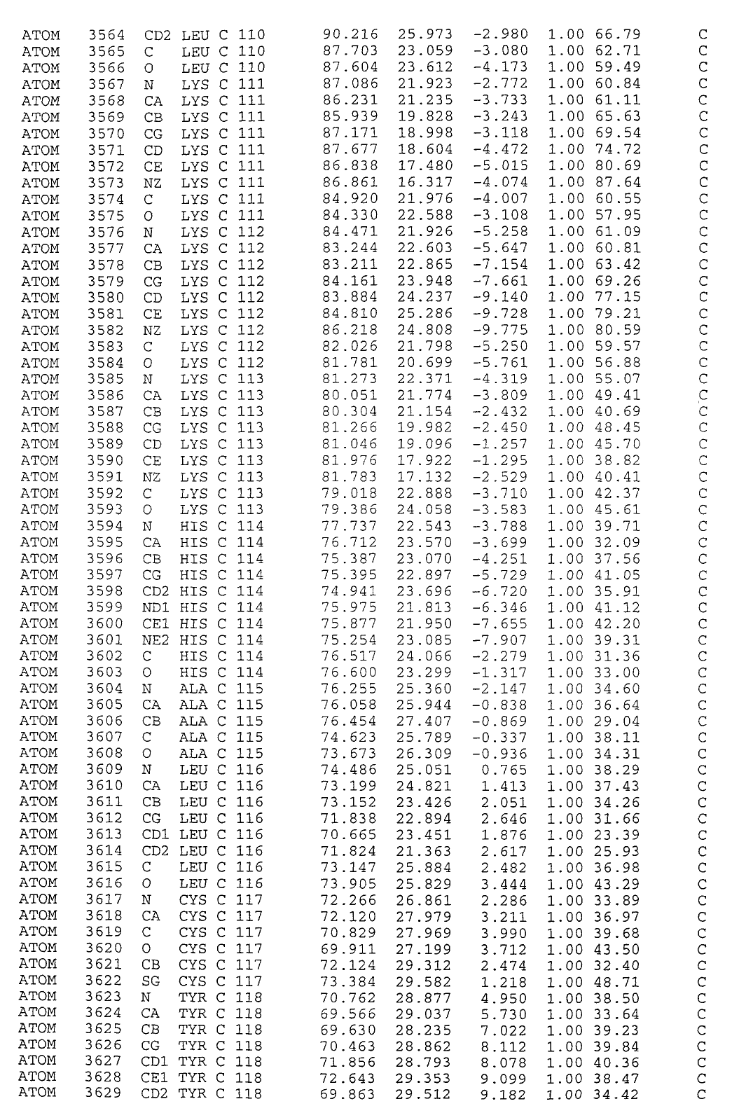

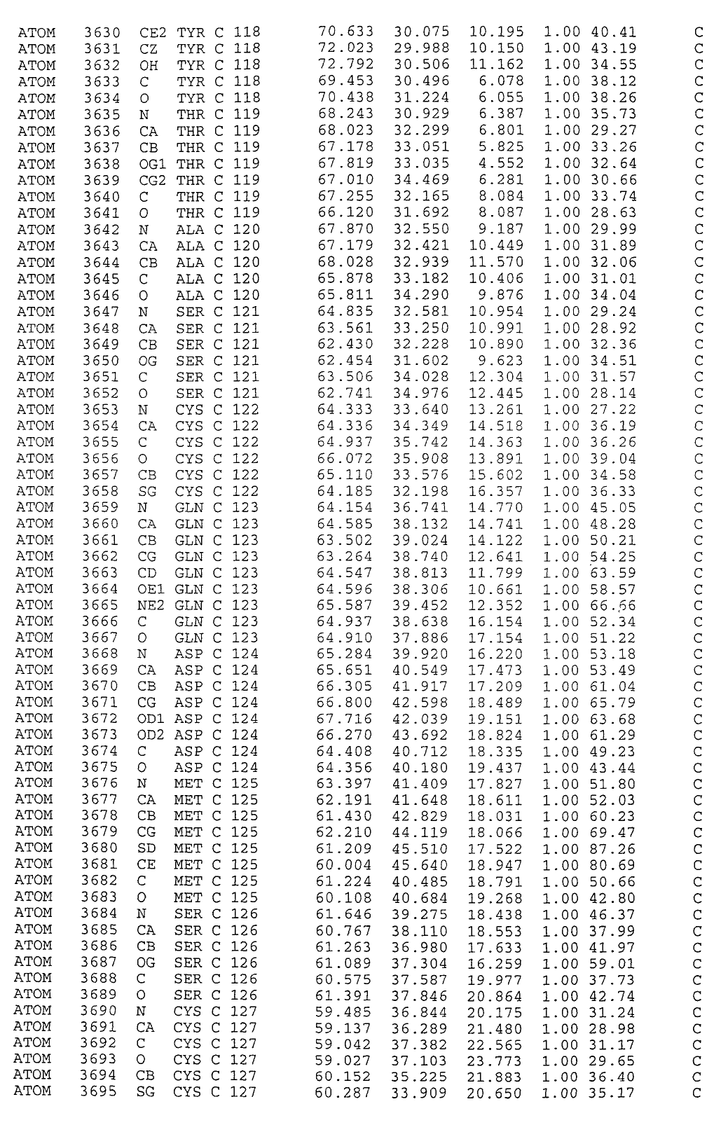

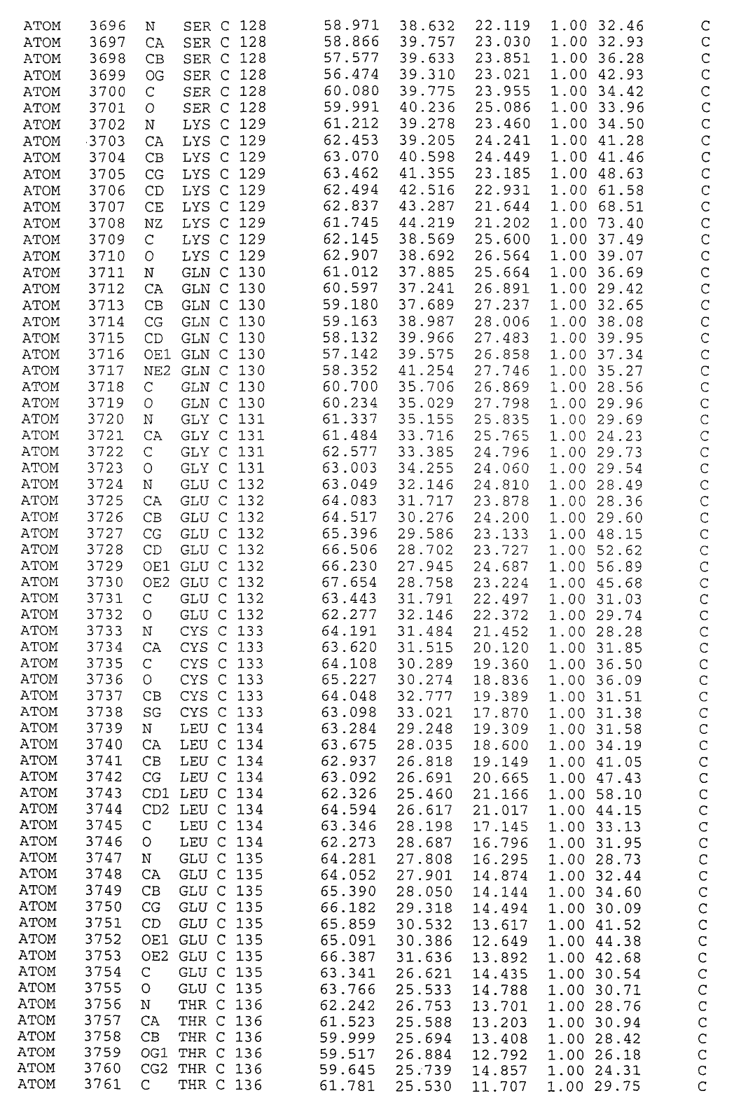

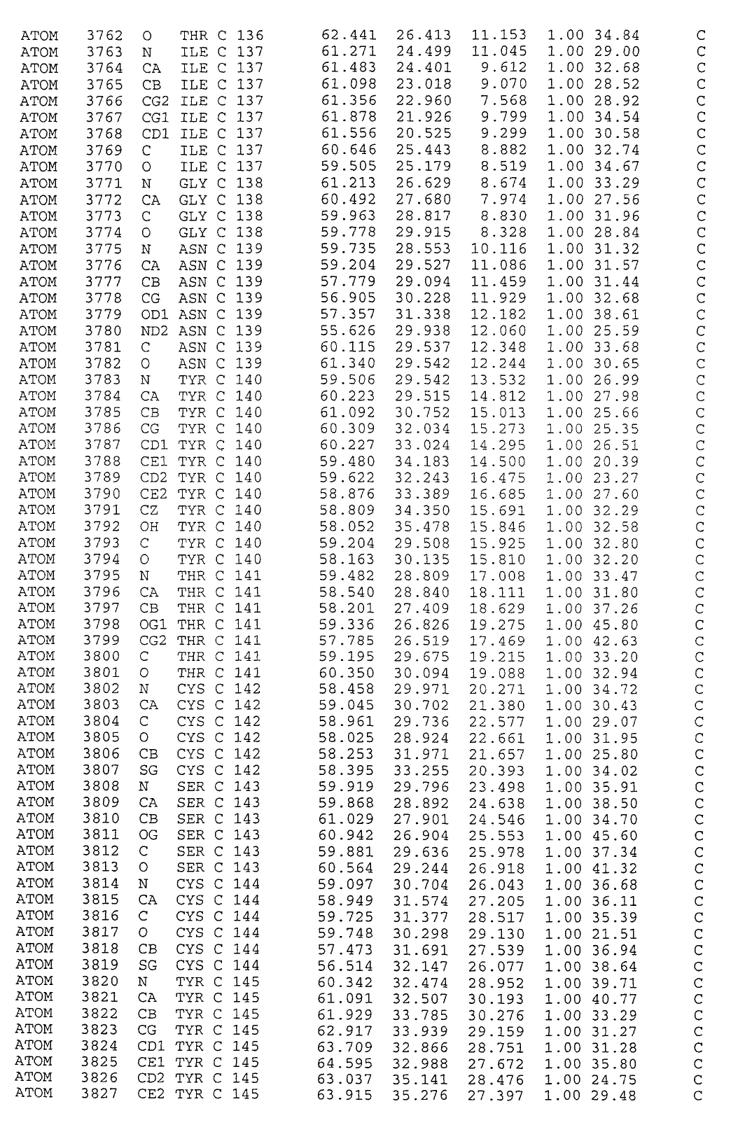

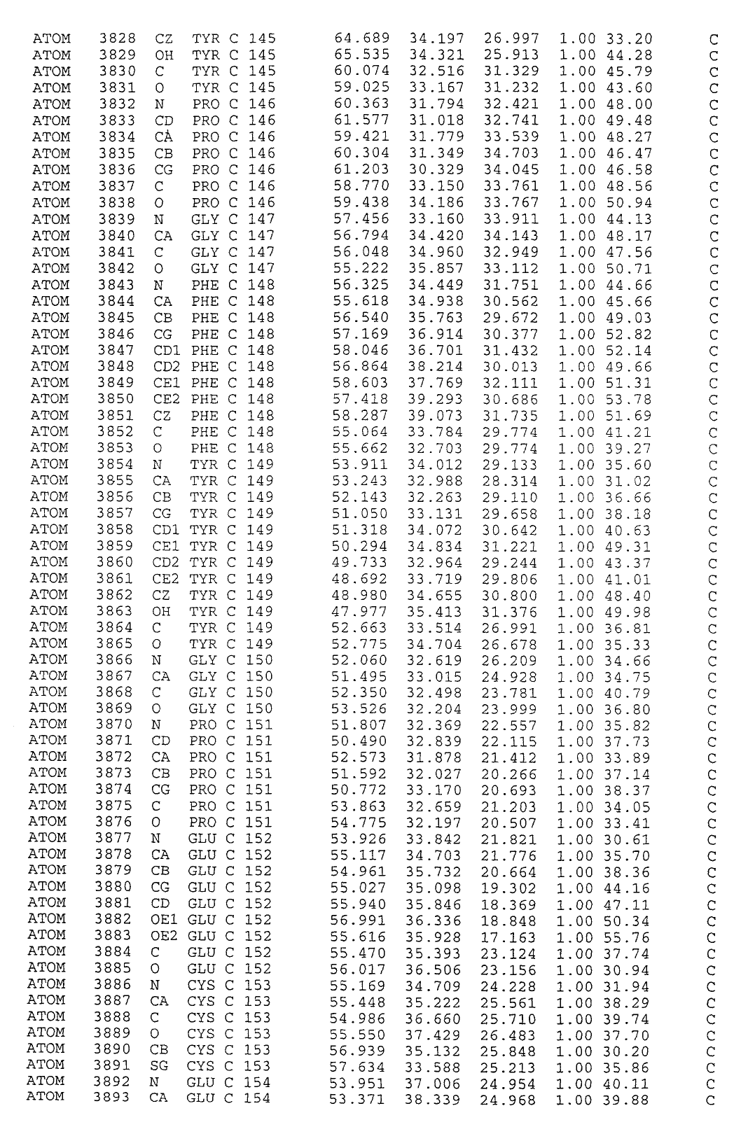

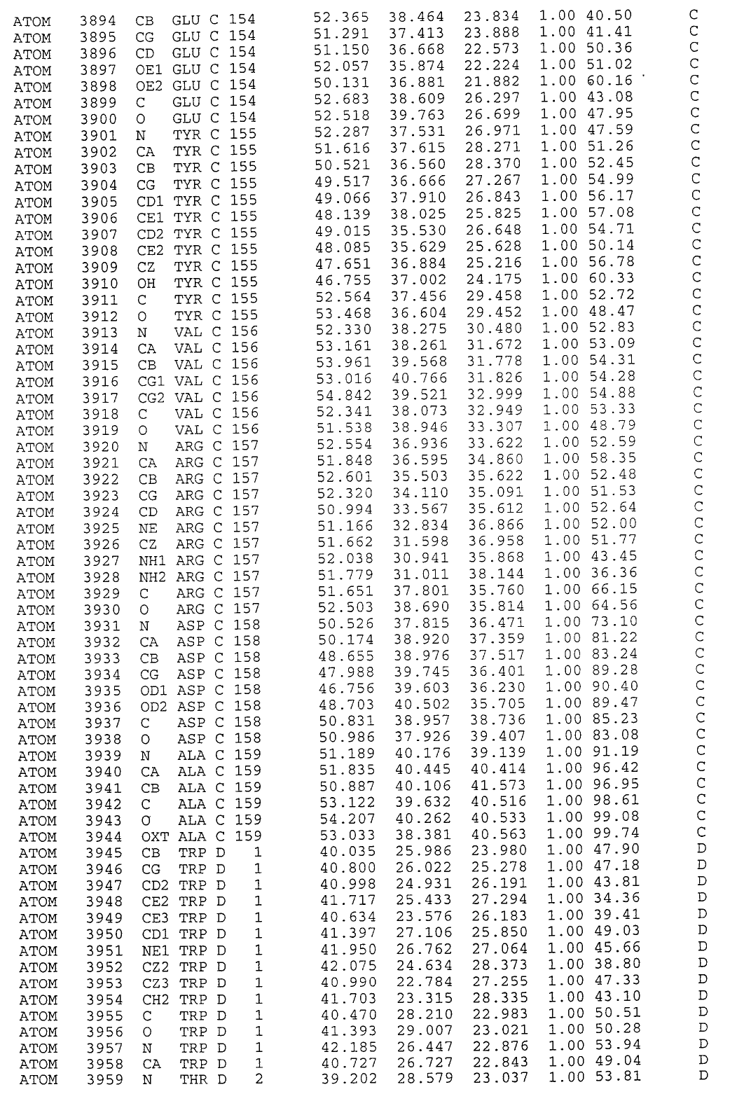

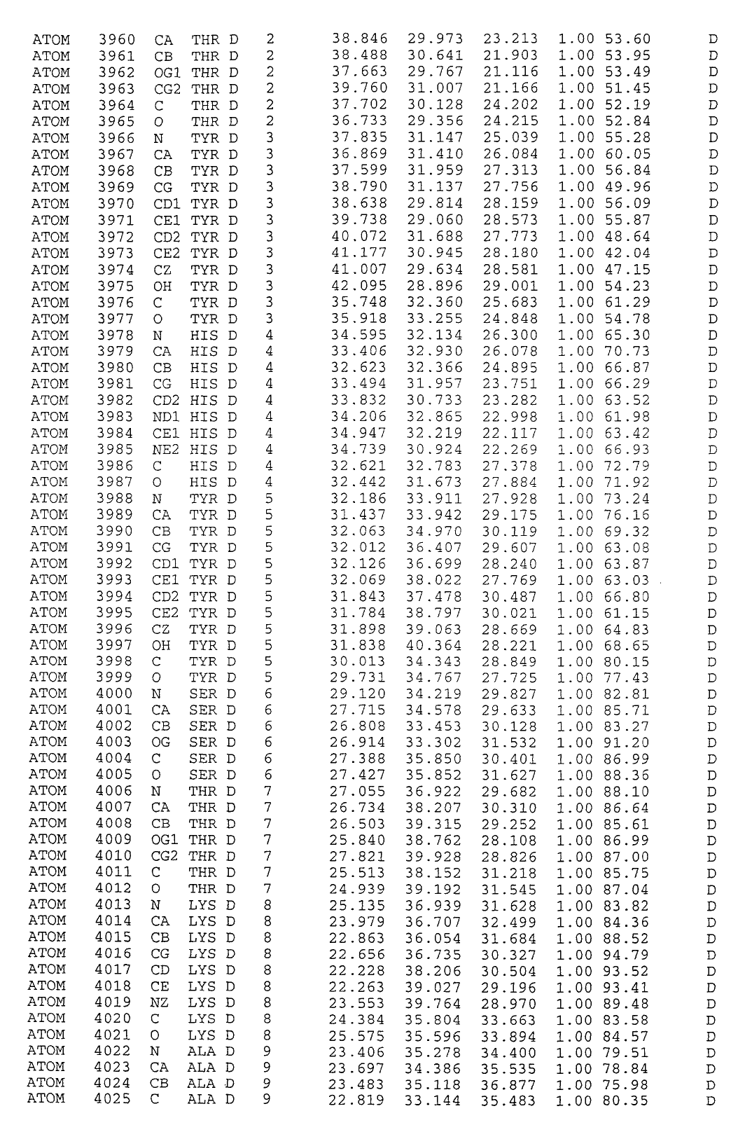

The present invention further provides the three dimensional structures of E-selectin LE and SLe^{X} as derived by x-ray diffraction data of the E-selectin LE: SLe^{X} crystal. Specifically, the three dimensional structures of E-selectin LE and SLe^{X} are defined by the structural coordinates shown in FIG. 4, ± a root mean square deviation from the backbone atoms of the amino acids of not more than 1.5 Å, preferably not more than 1.0 Å, and most preferably not more than 0.5 Å. The structural coordinates of E-selectin LE and SLe^{X} are useful for a number of applications, including, but not limited to, the visualization, identification and characterization of various active sites of E-selectin LE, SLe^{X}and the E-selectin LE:SLe^{X} complex, including the SLe^{X} binding site. The active site structures may then be used to design agents which interact with E-selectin LE, SLe^{X}, as well as E-selectin LE complexed with SLe^{X}, PSGL-1, or related molecules.

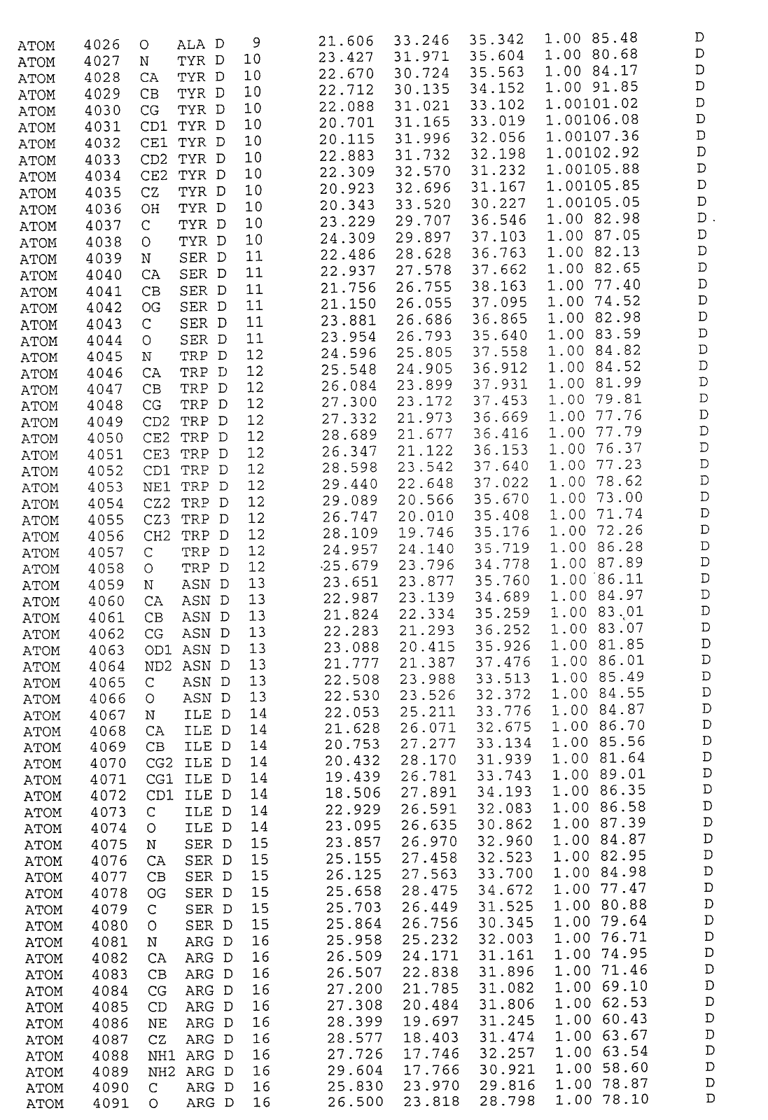

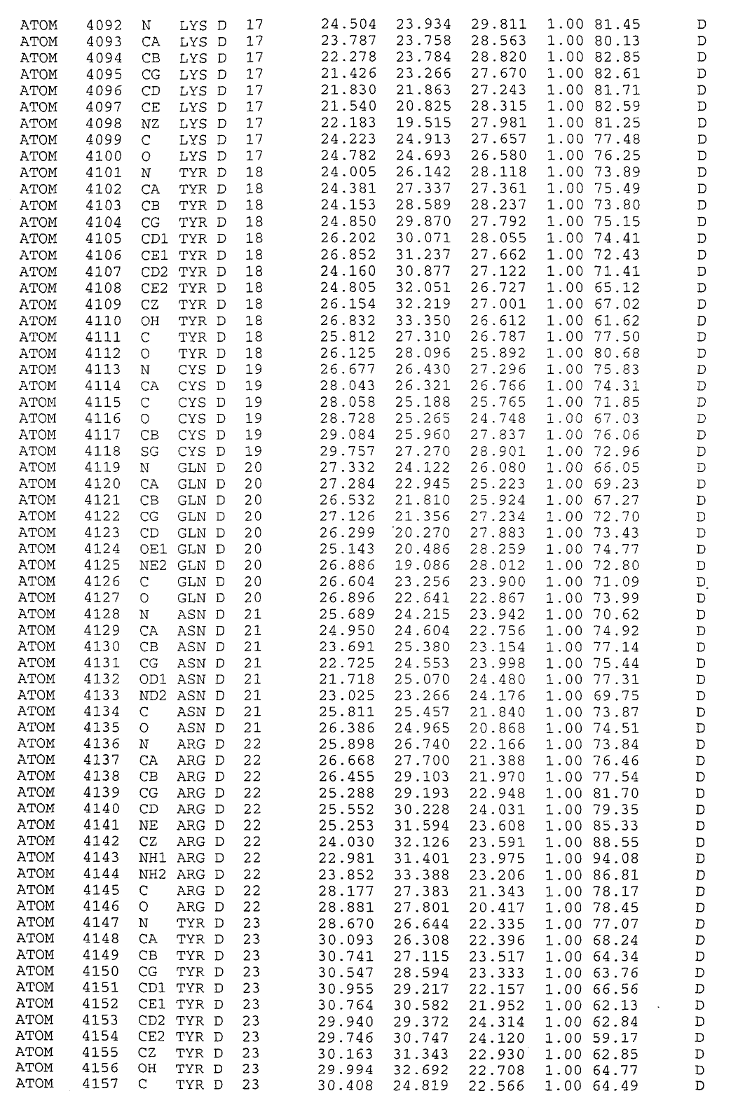

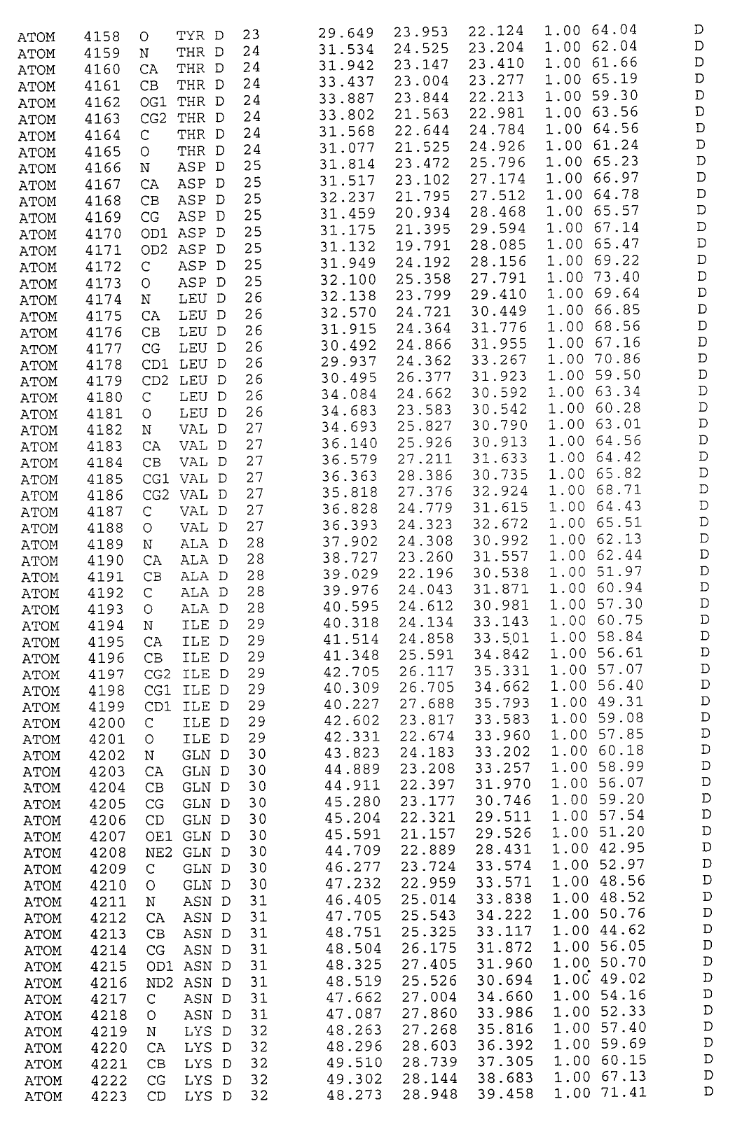

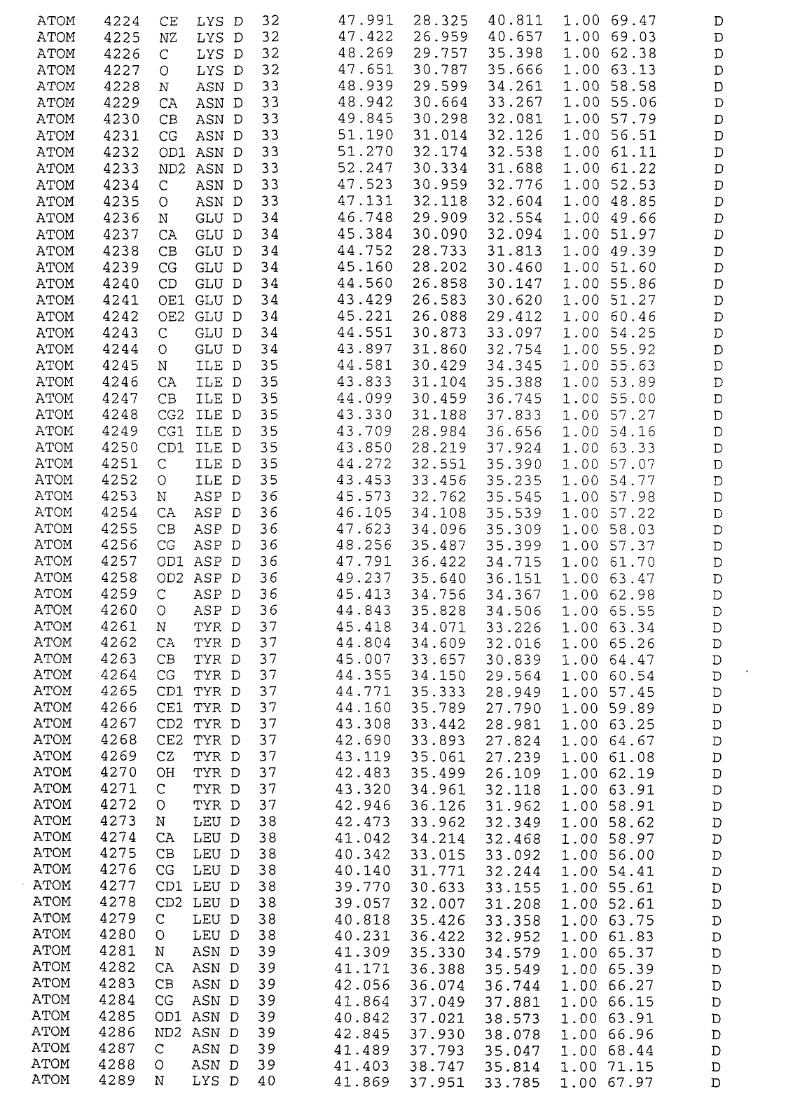

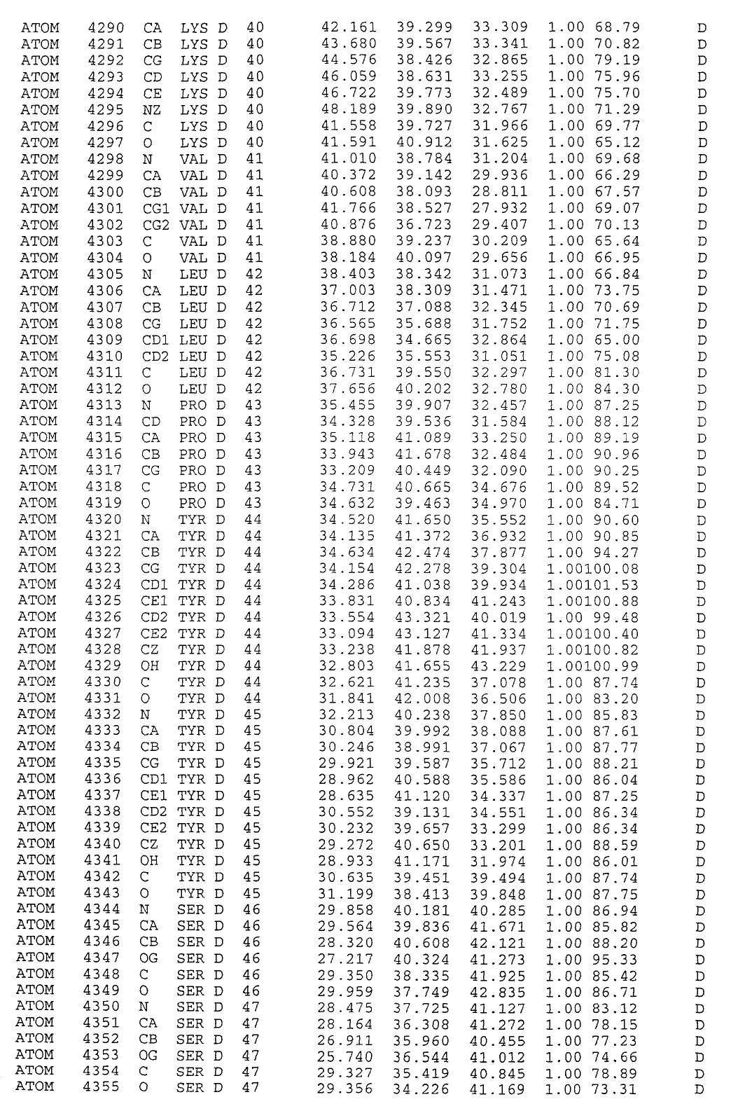

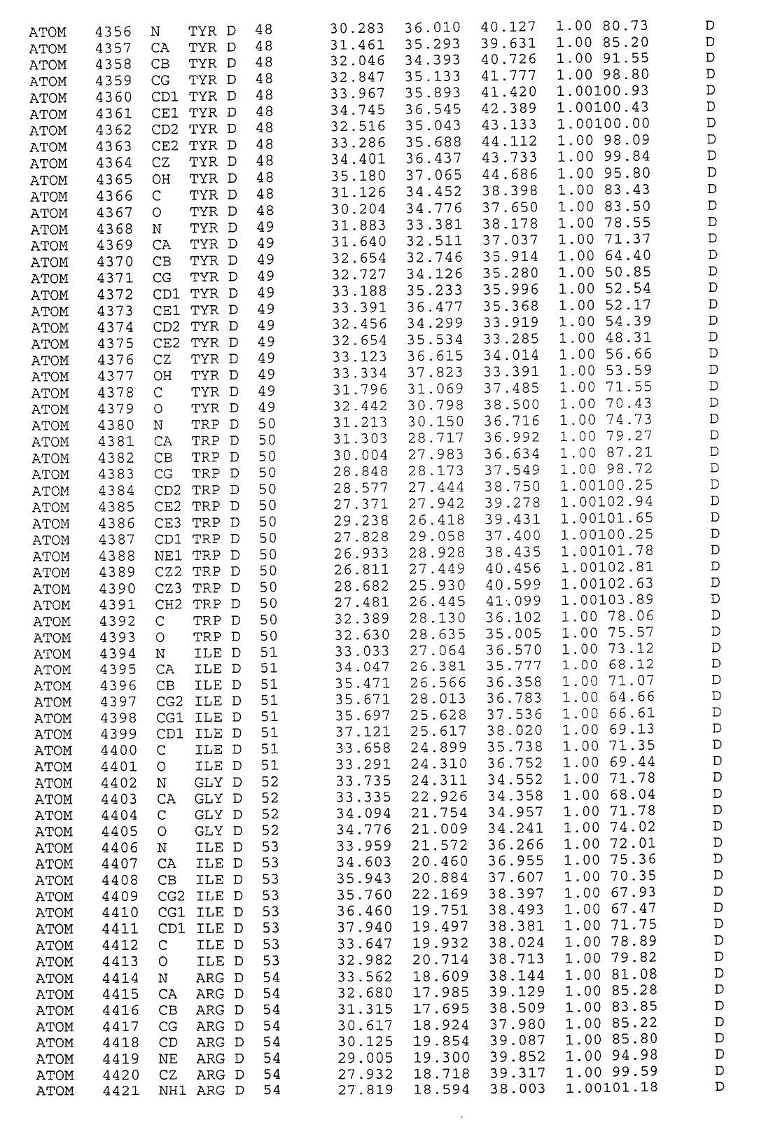

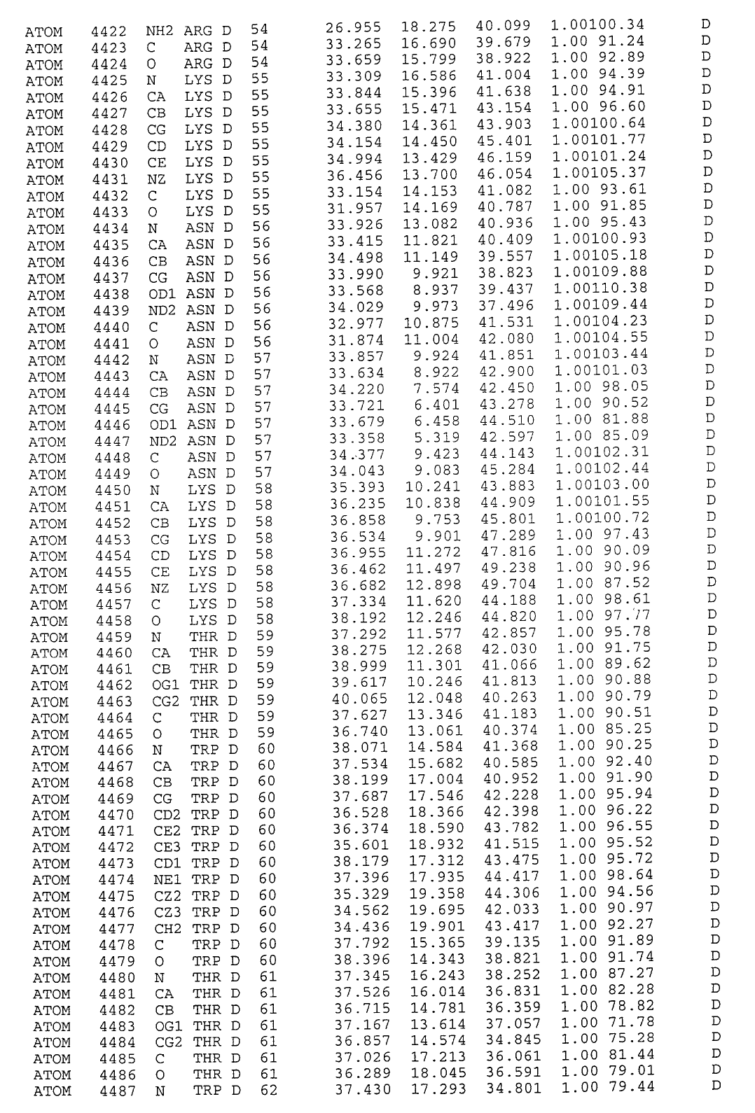

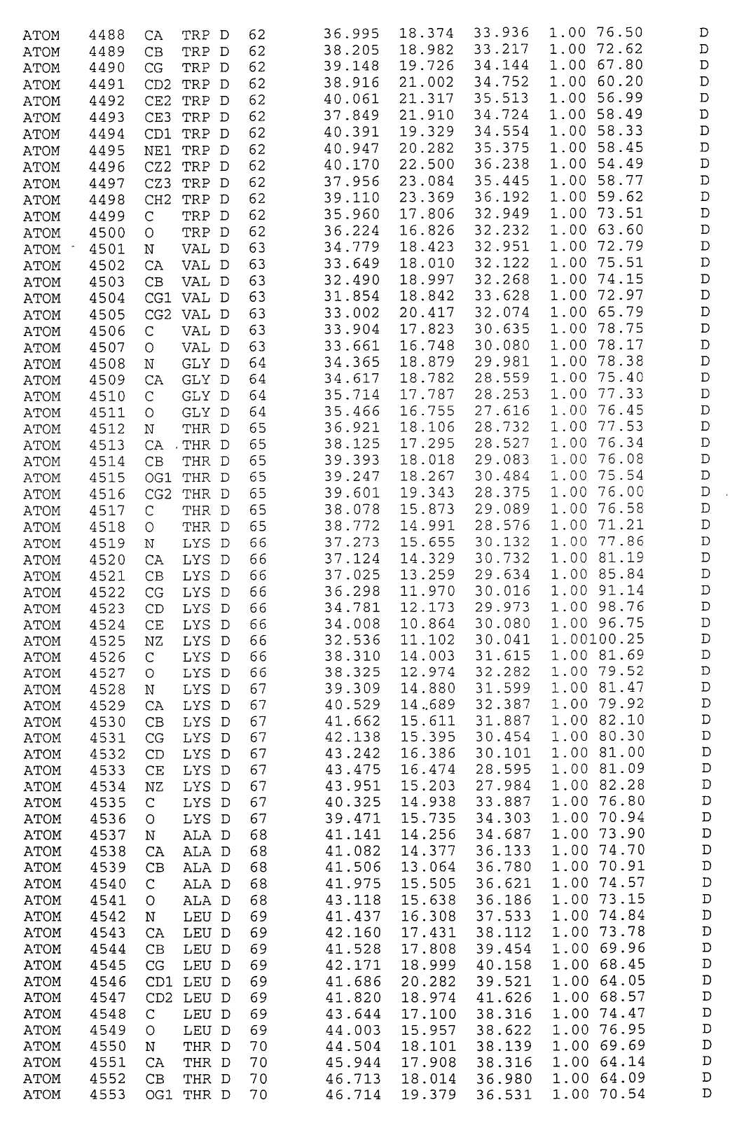

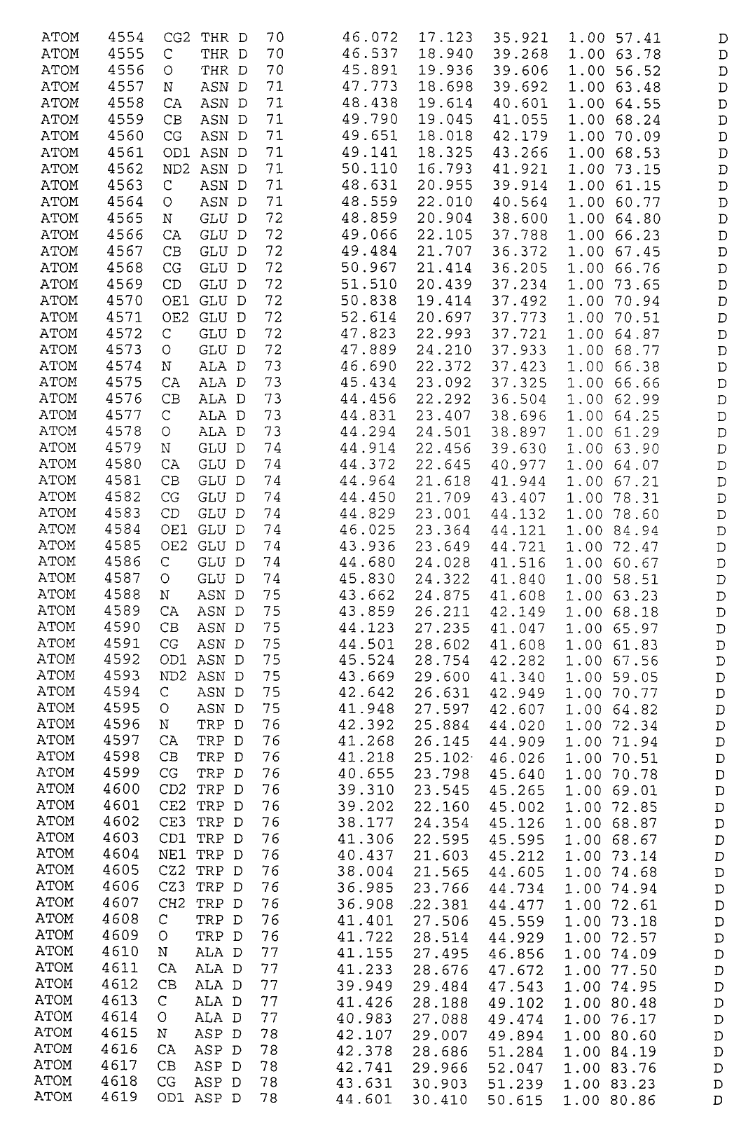

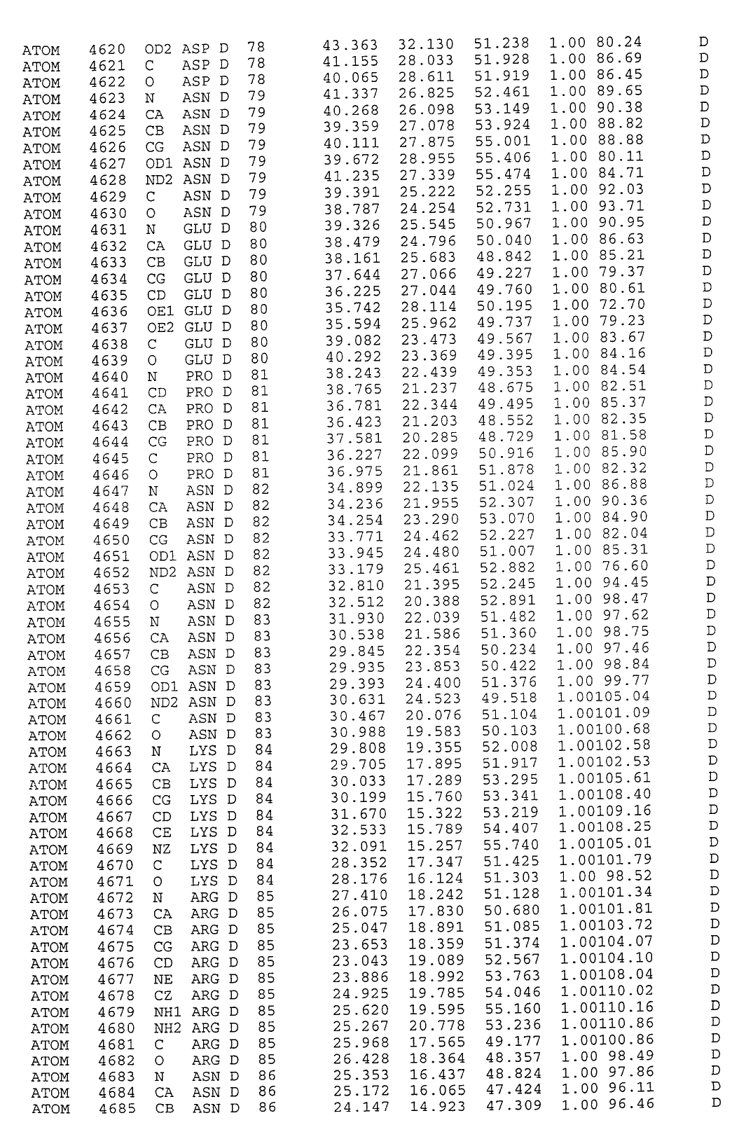

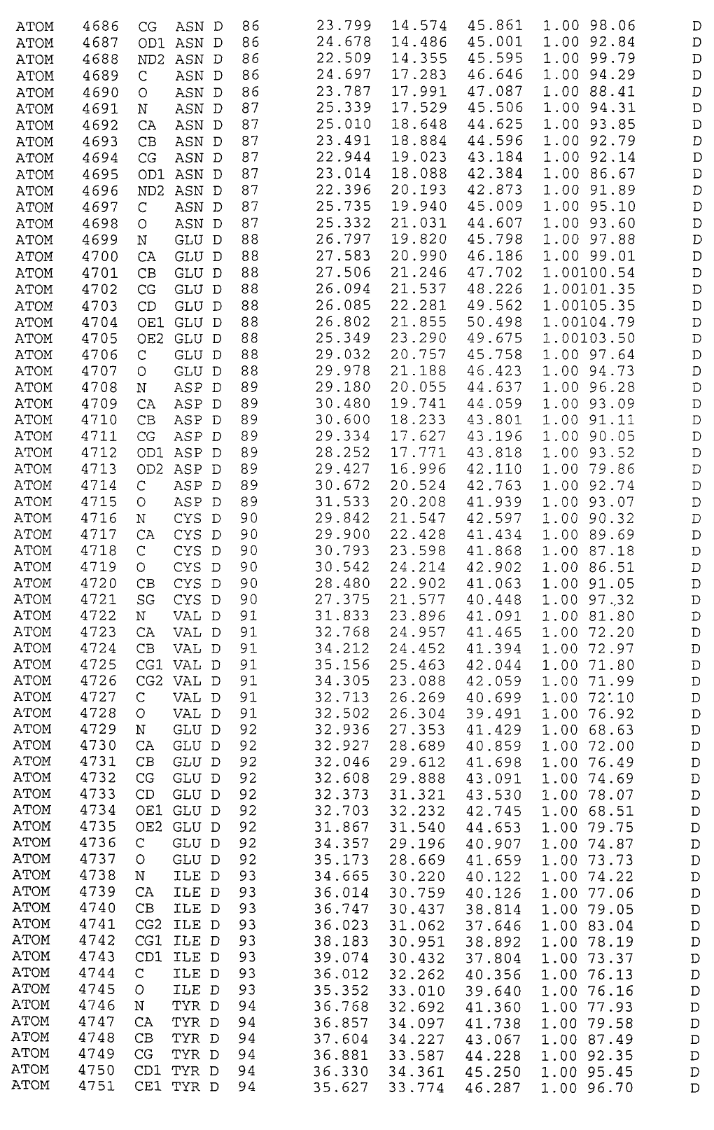

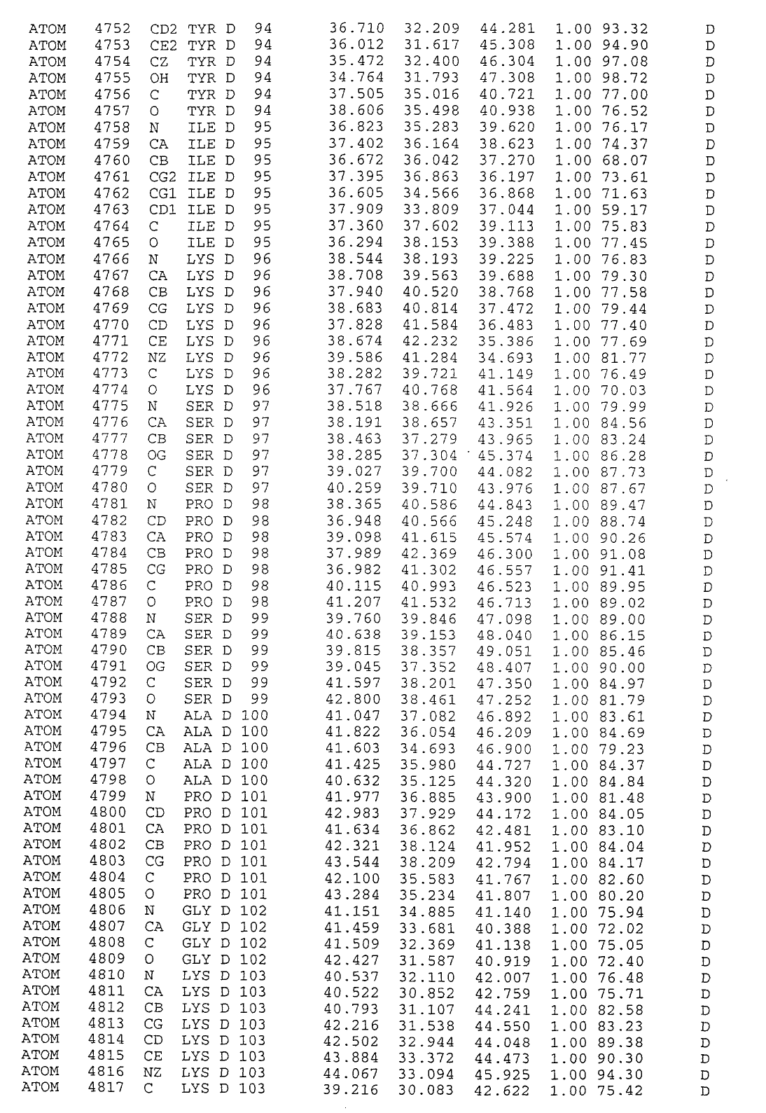

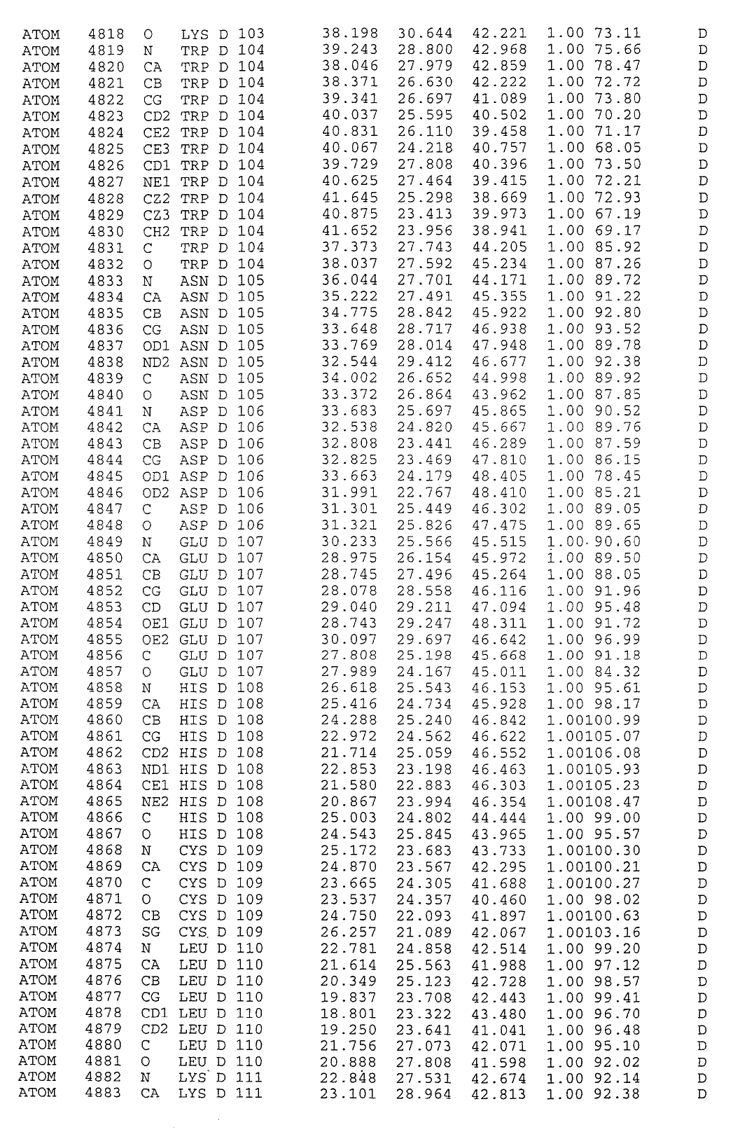

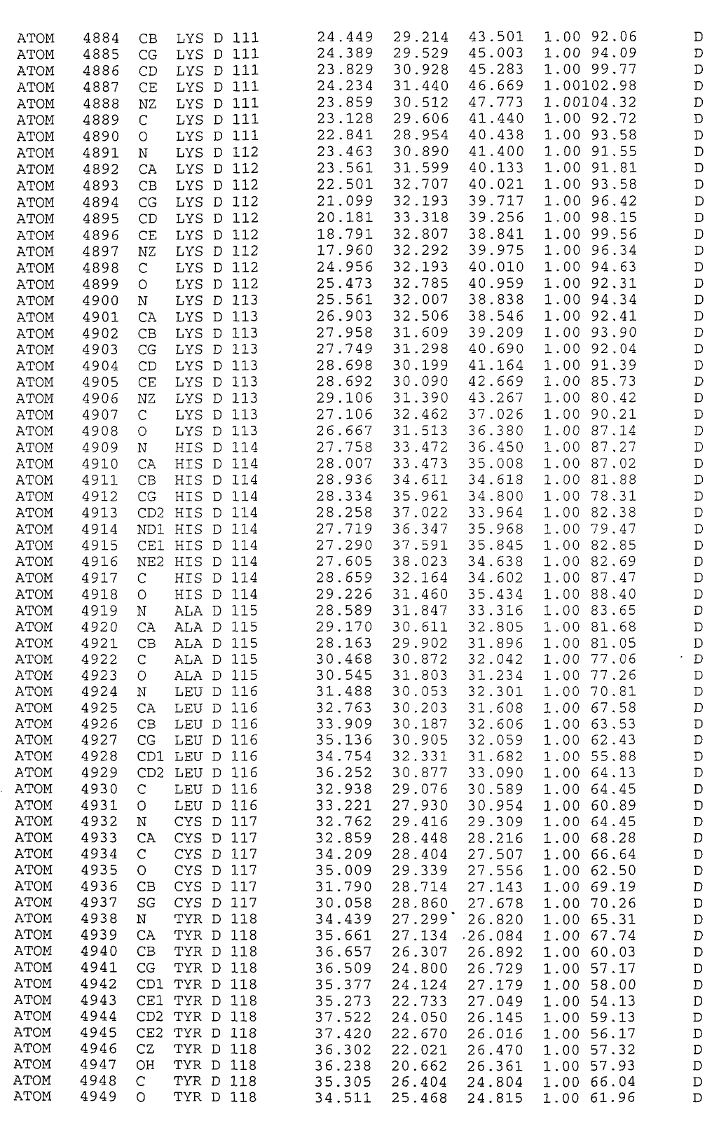

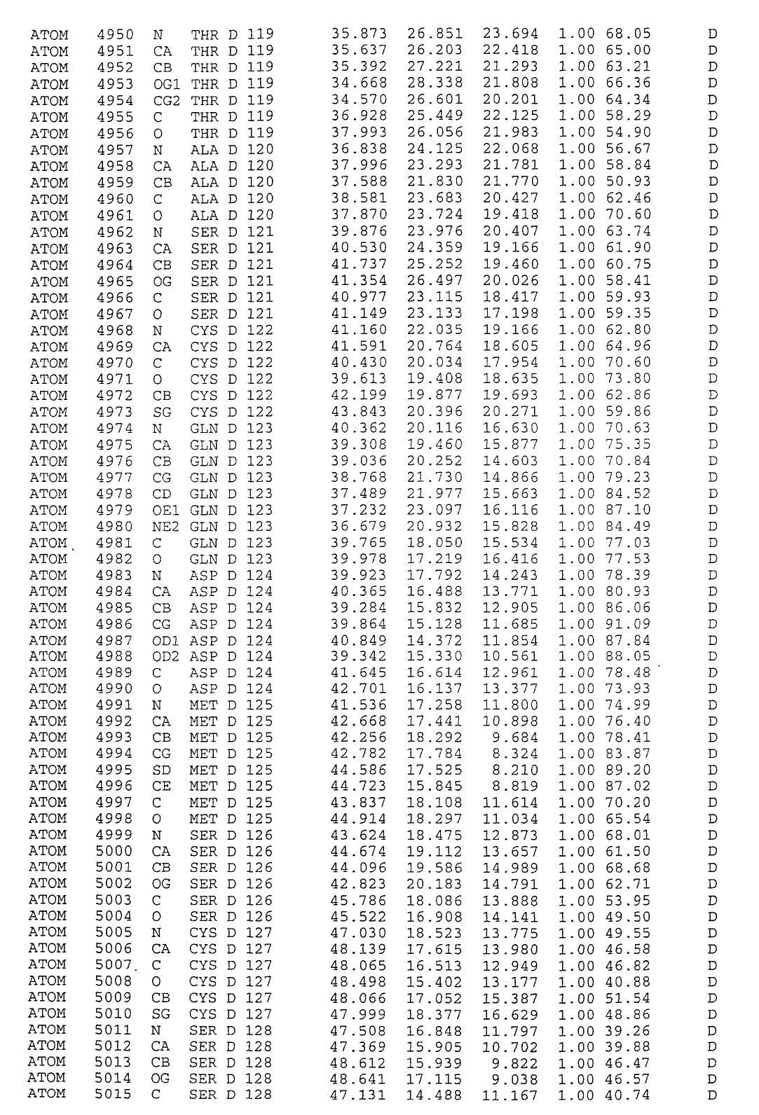

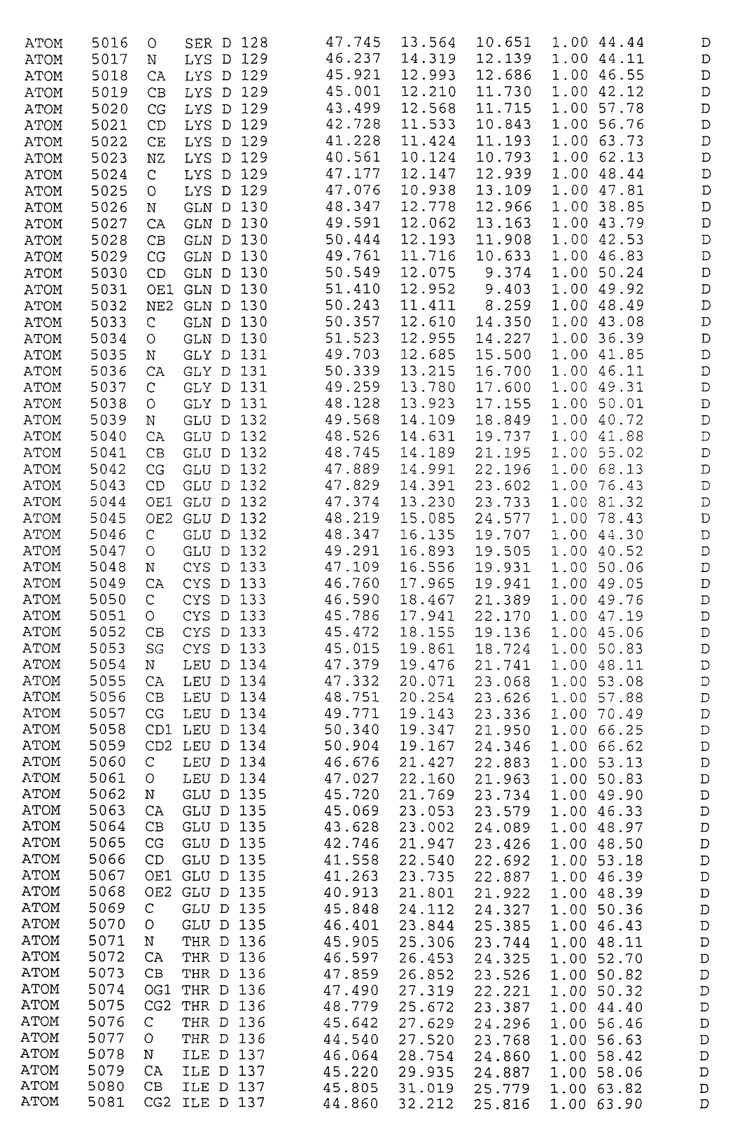

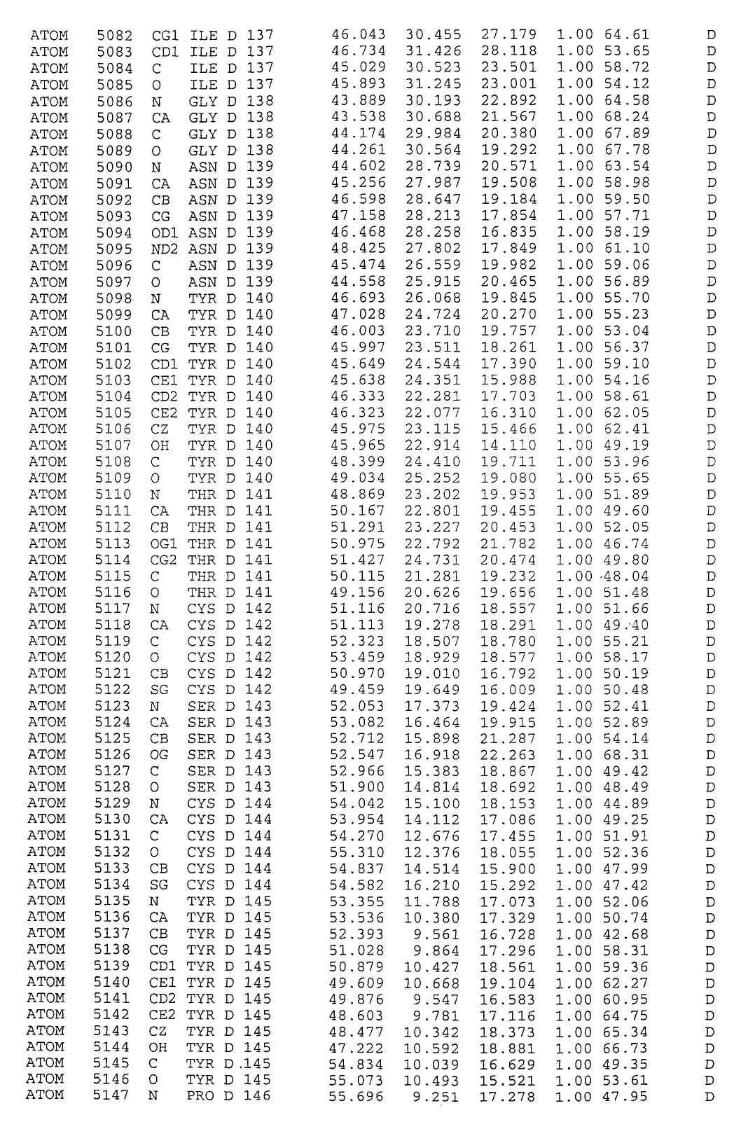

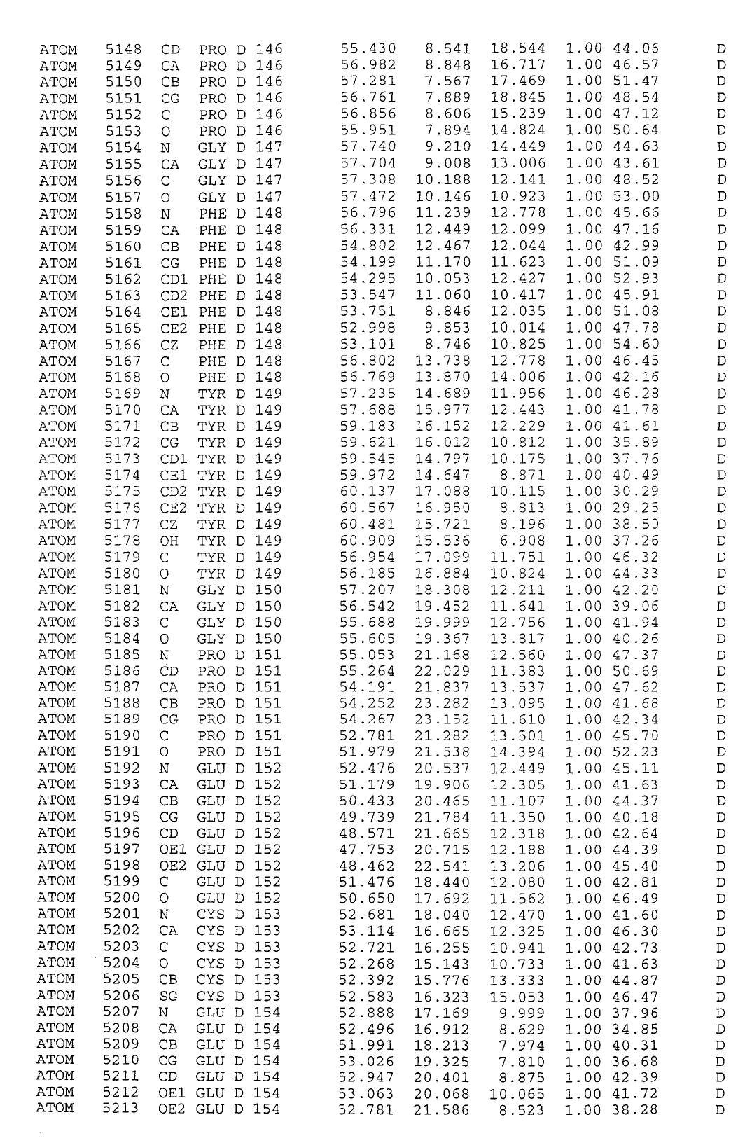

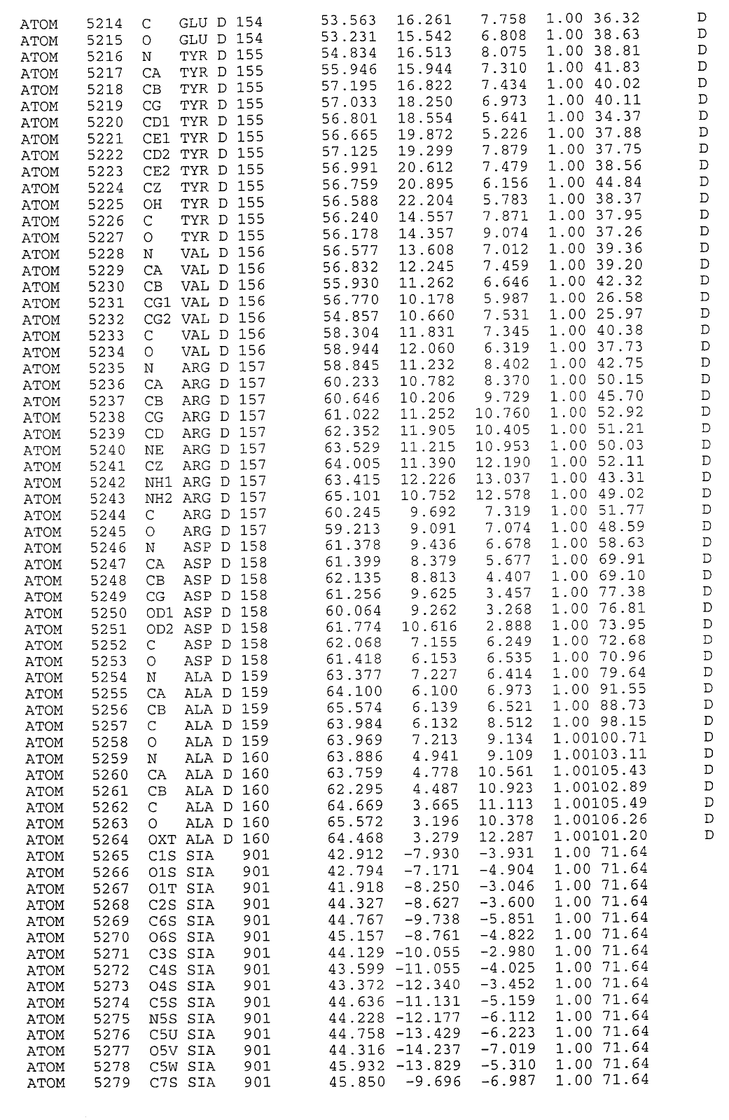

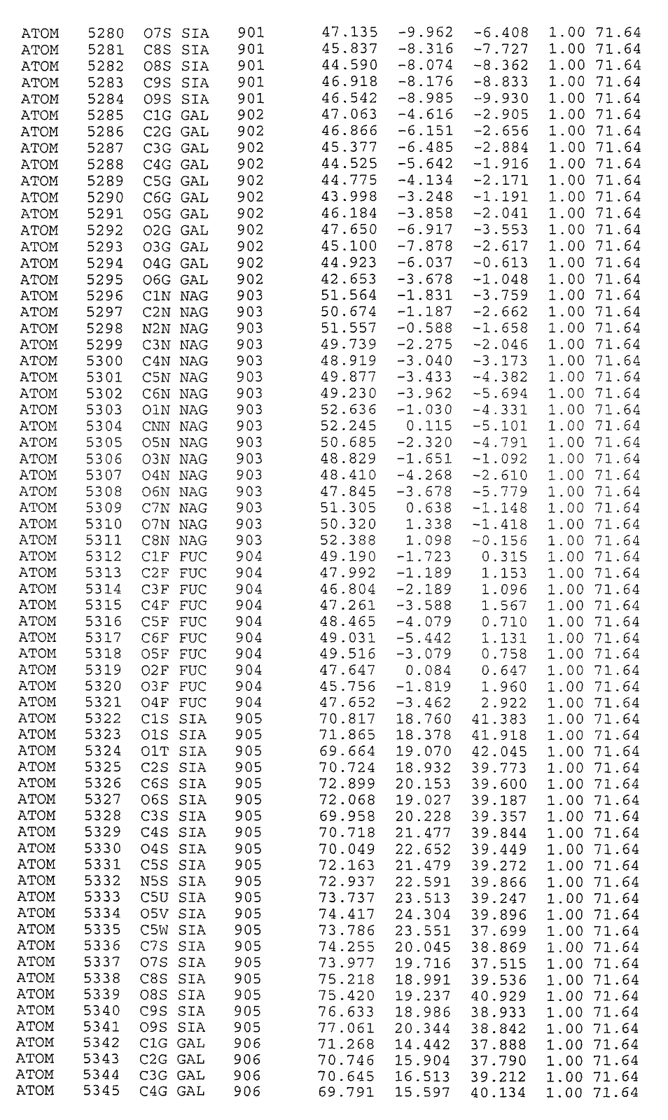

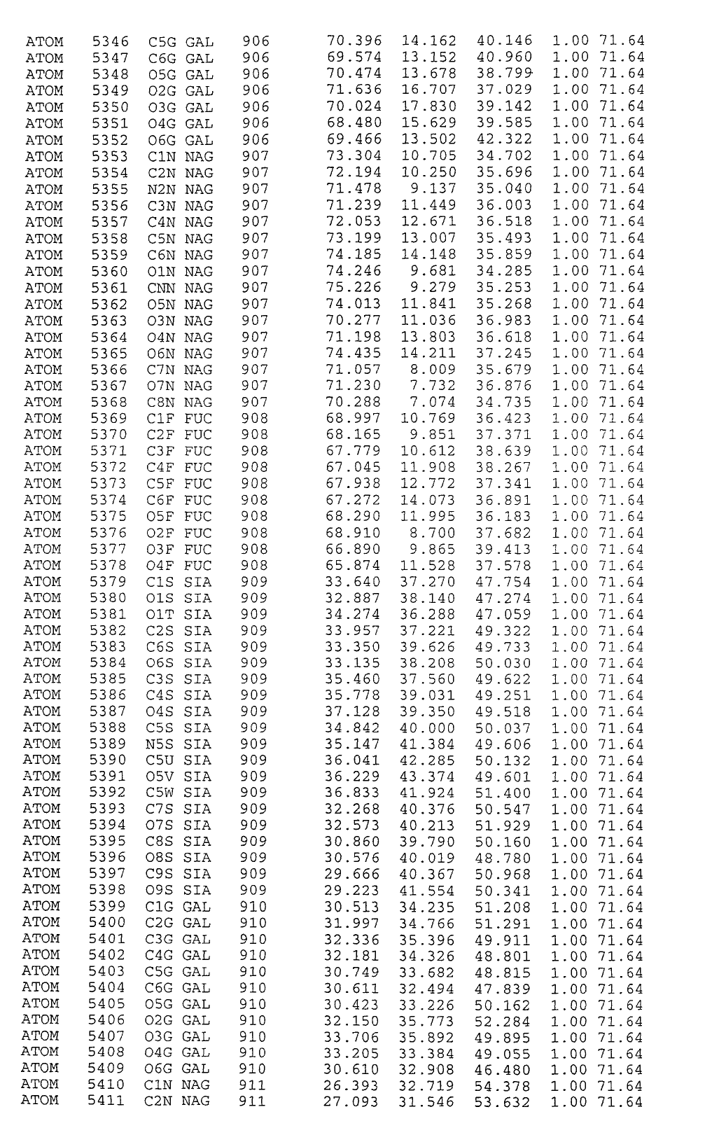

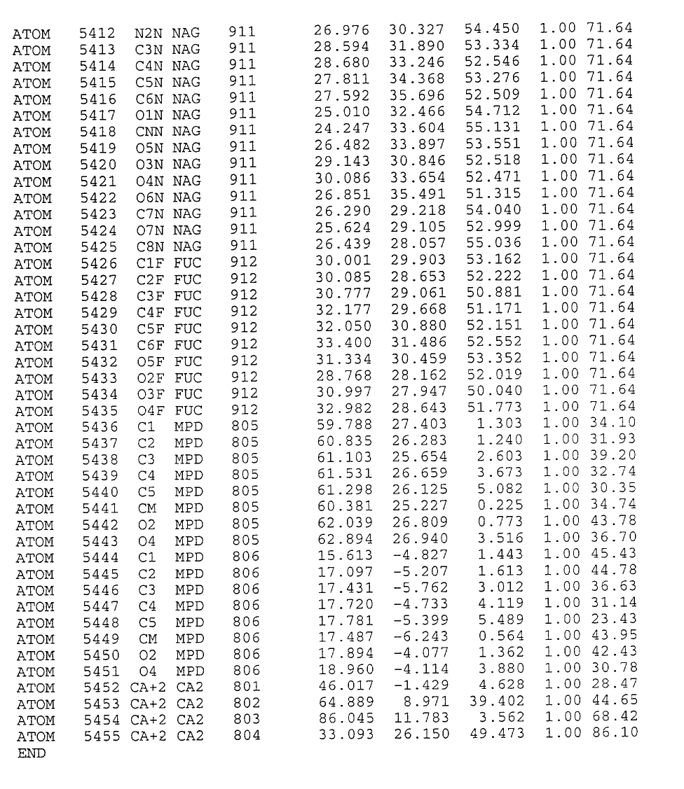

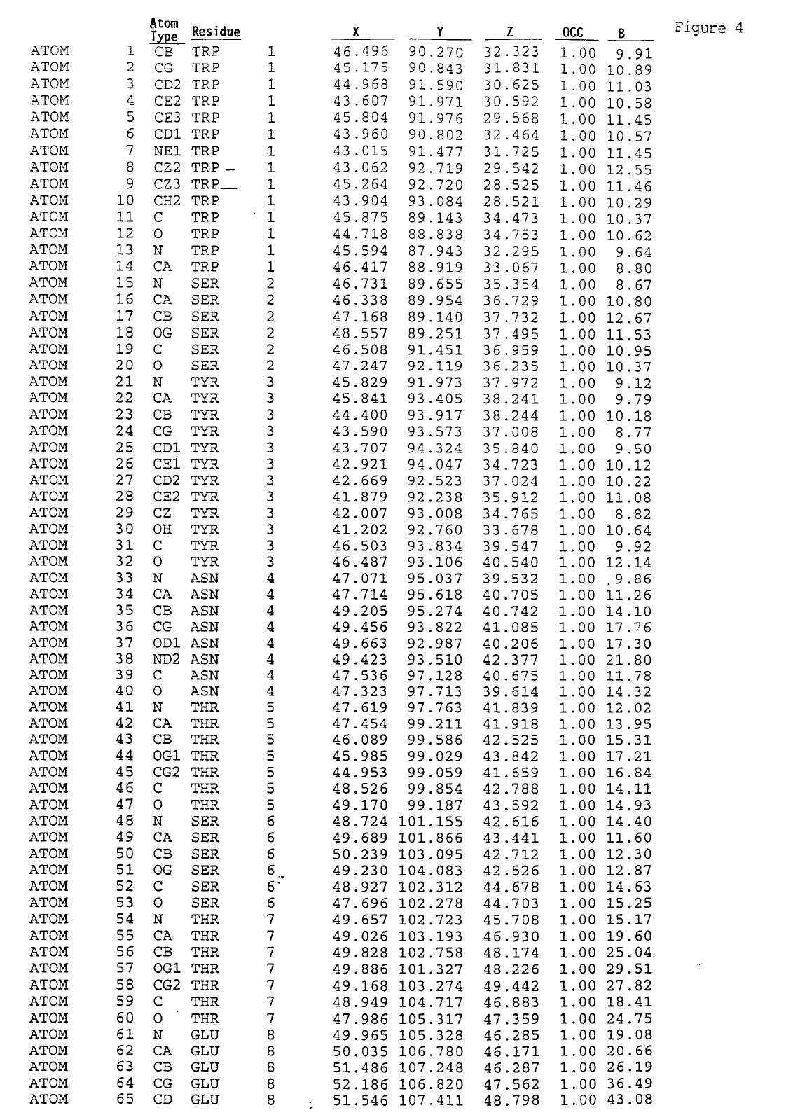

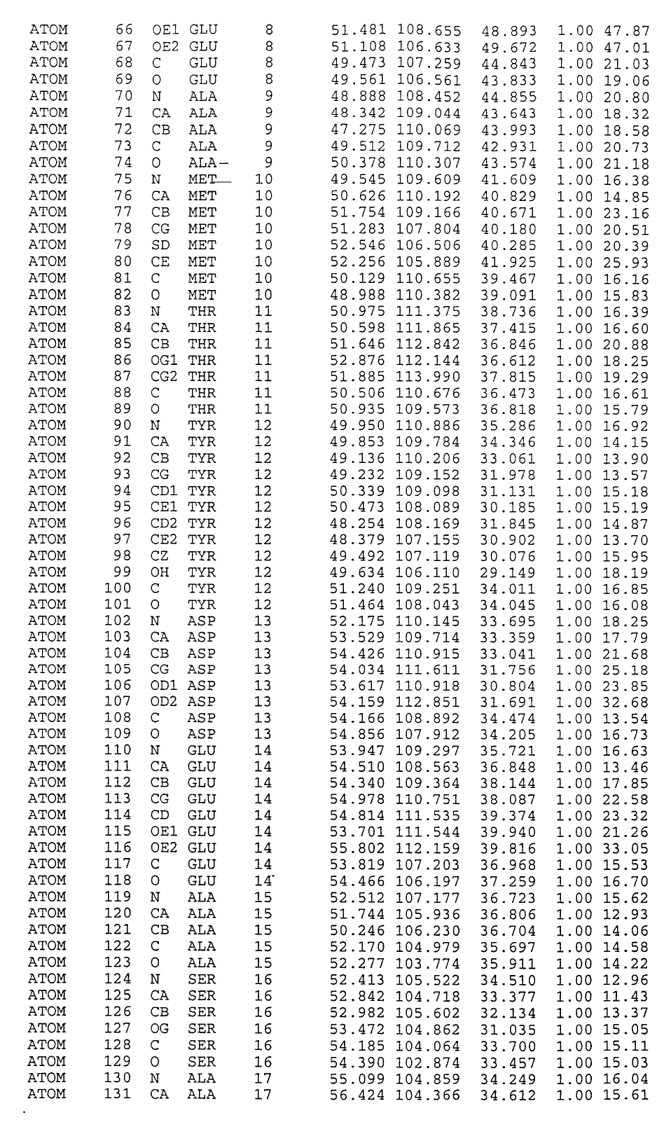

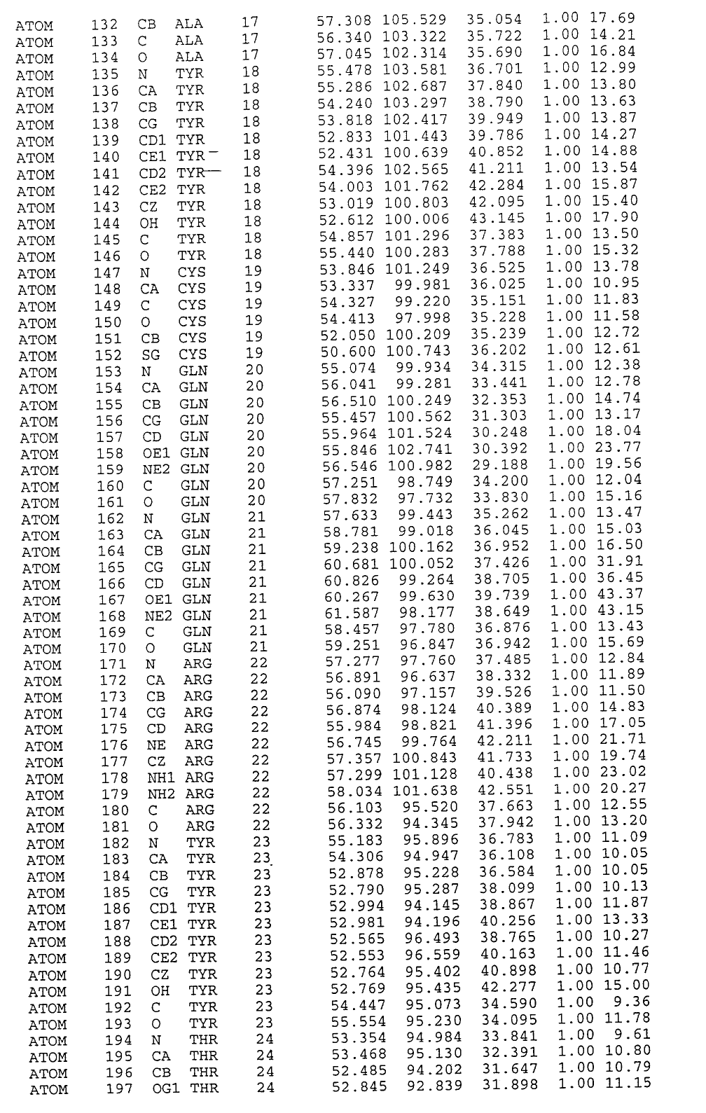

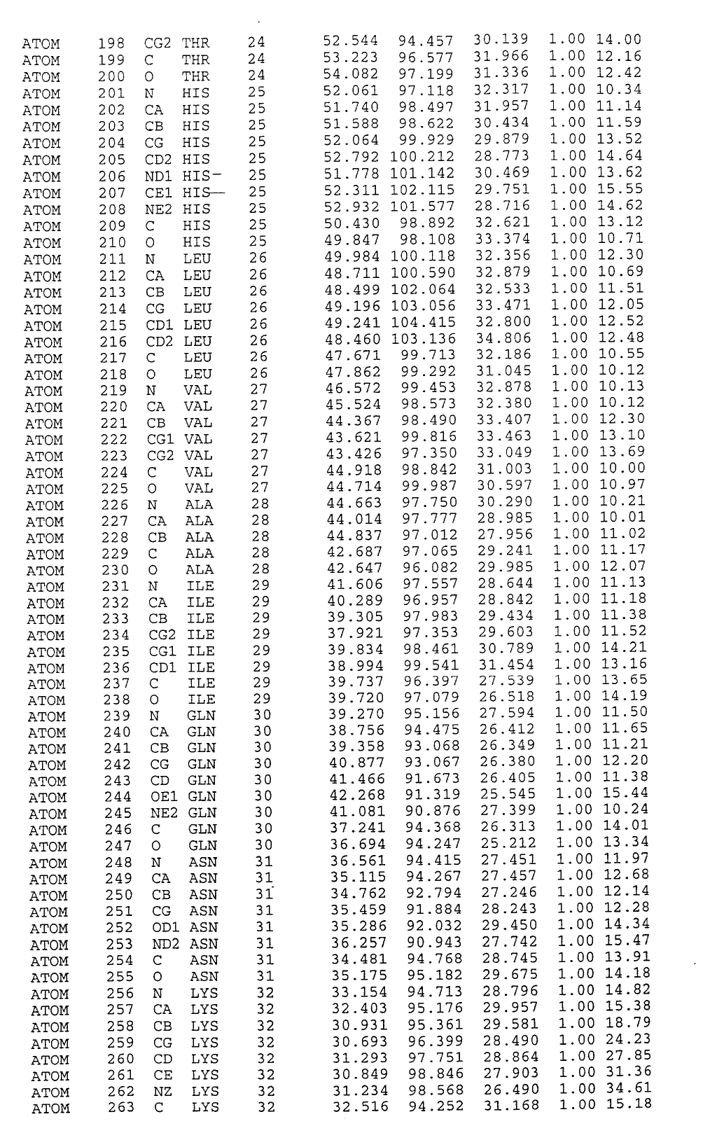

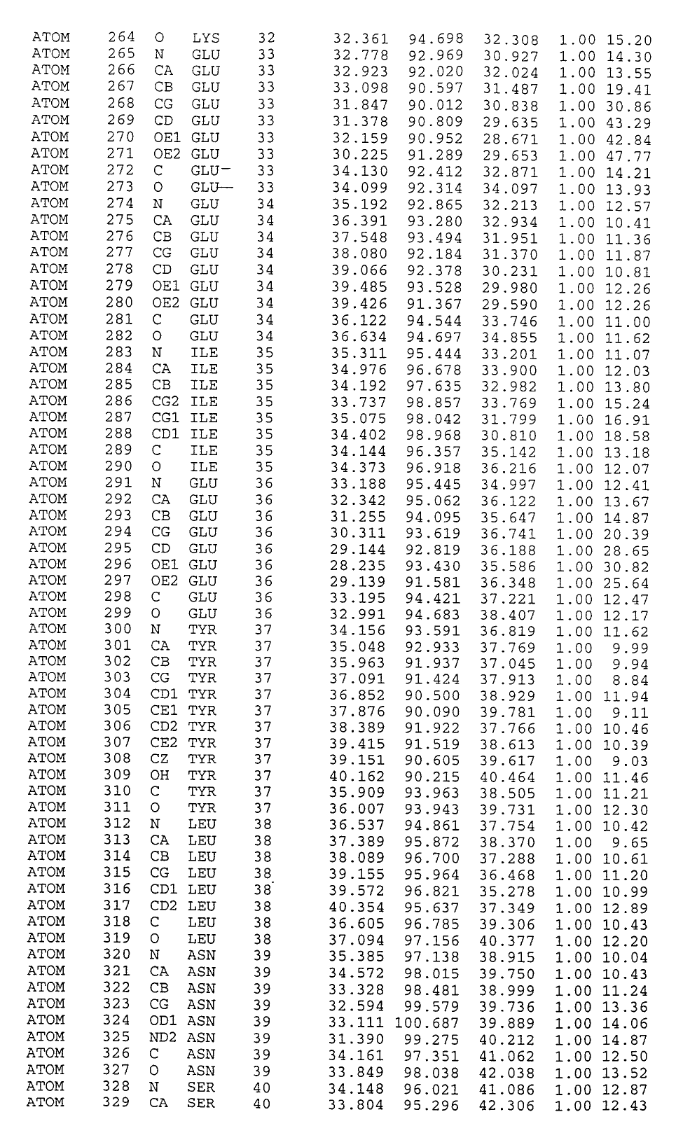

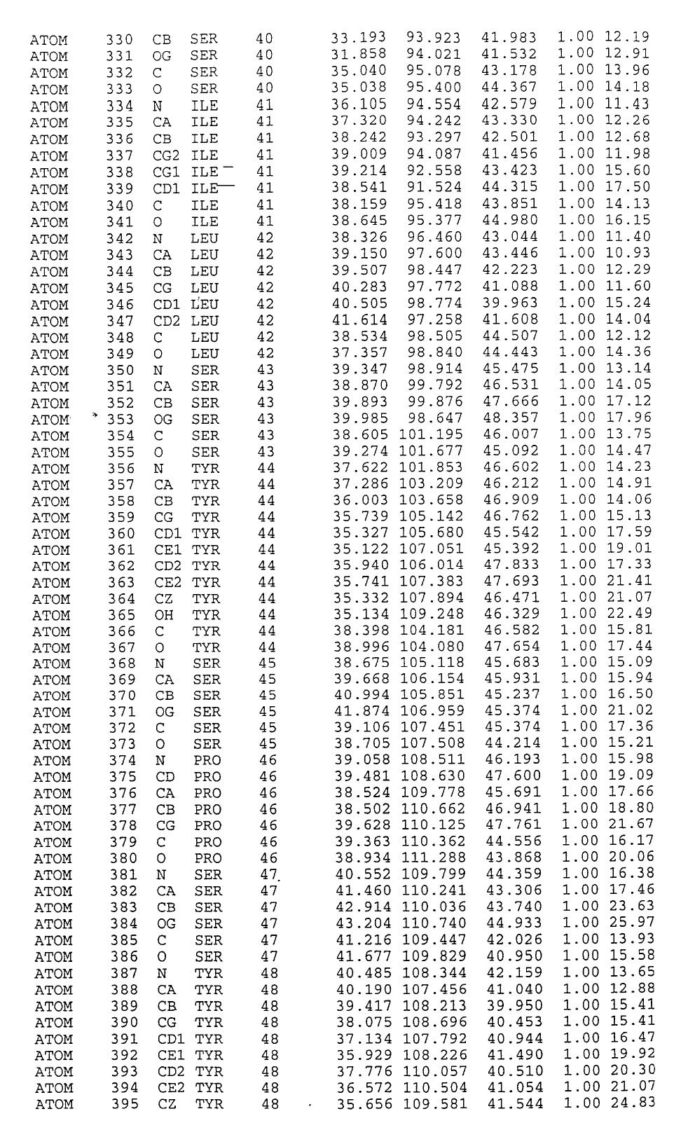

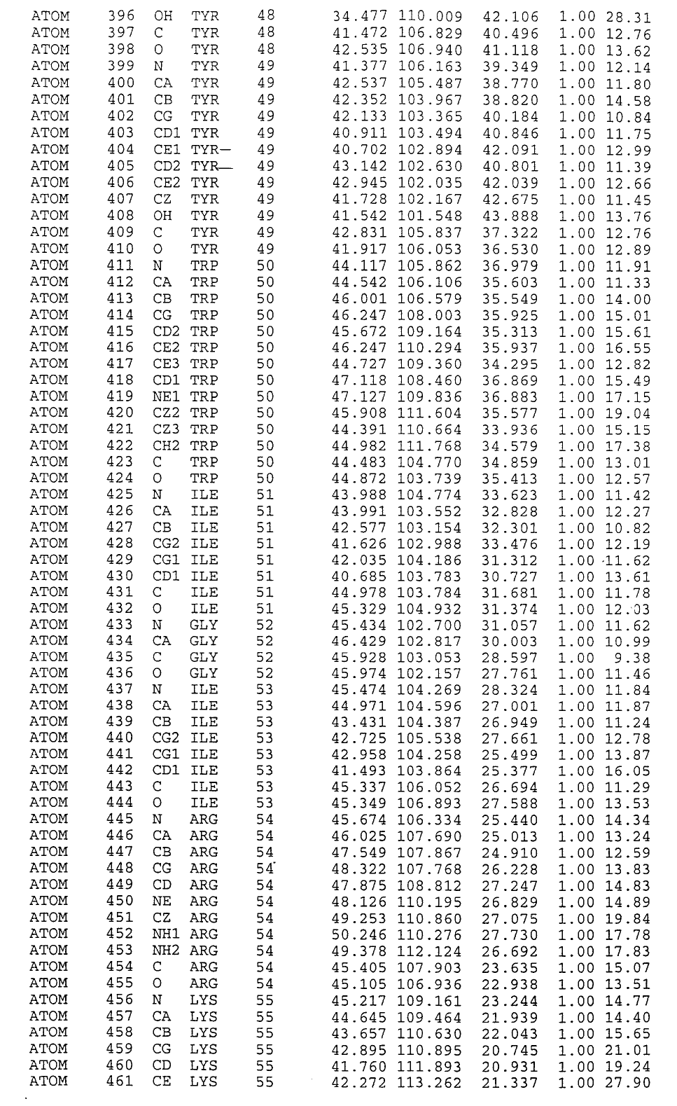

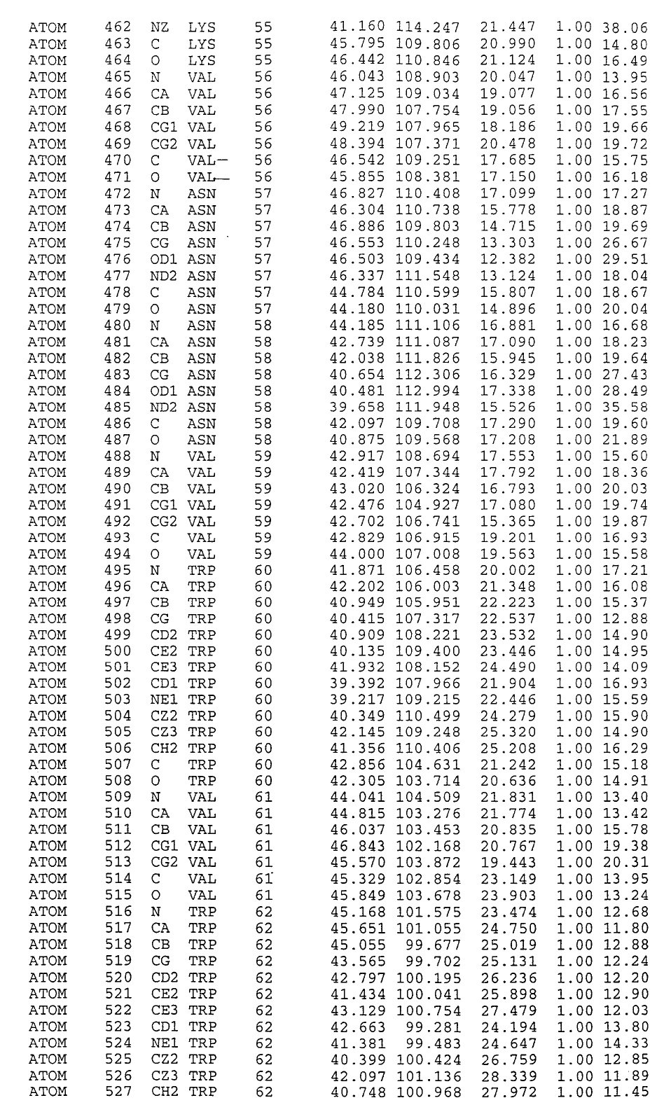

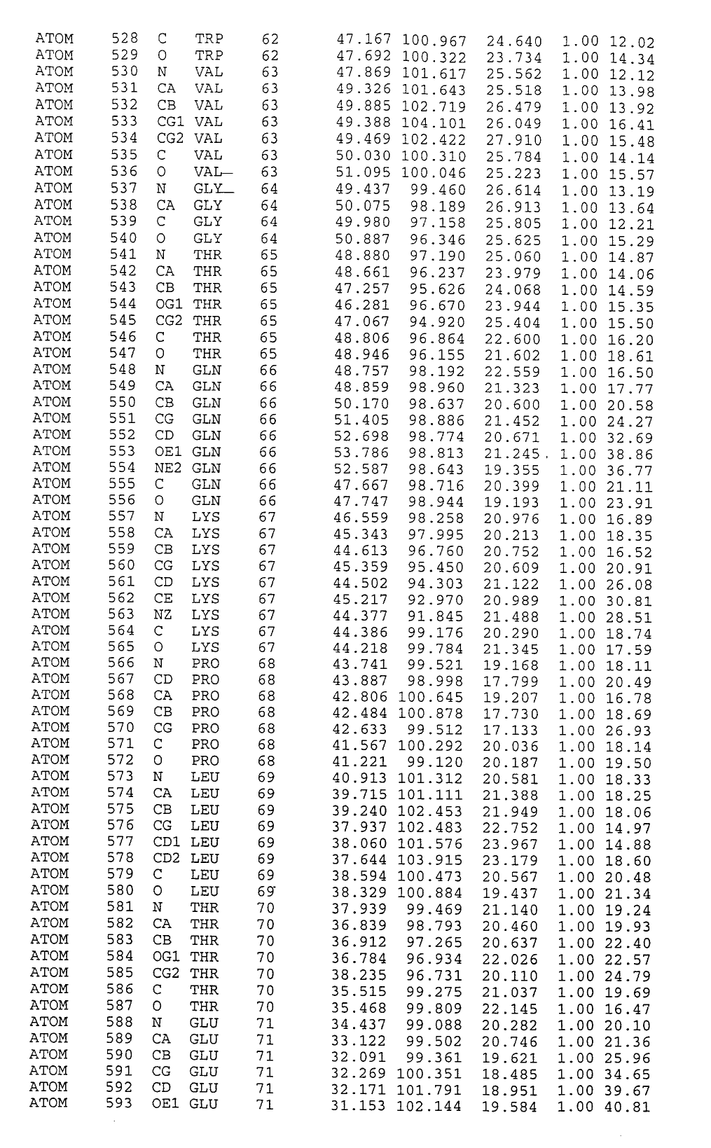

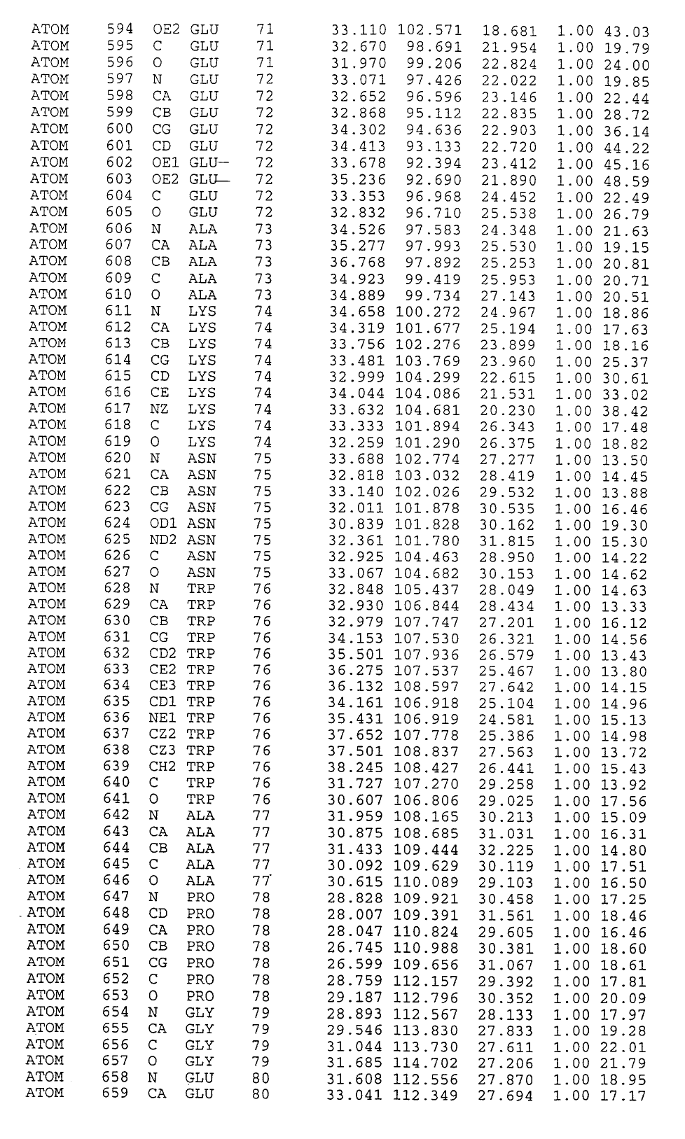

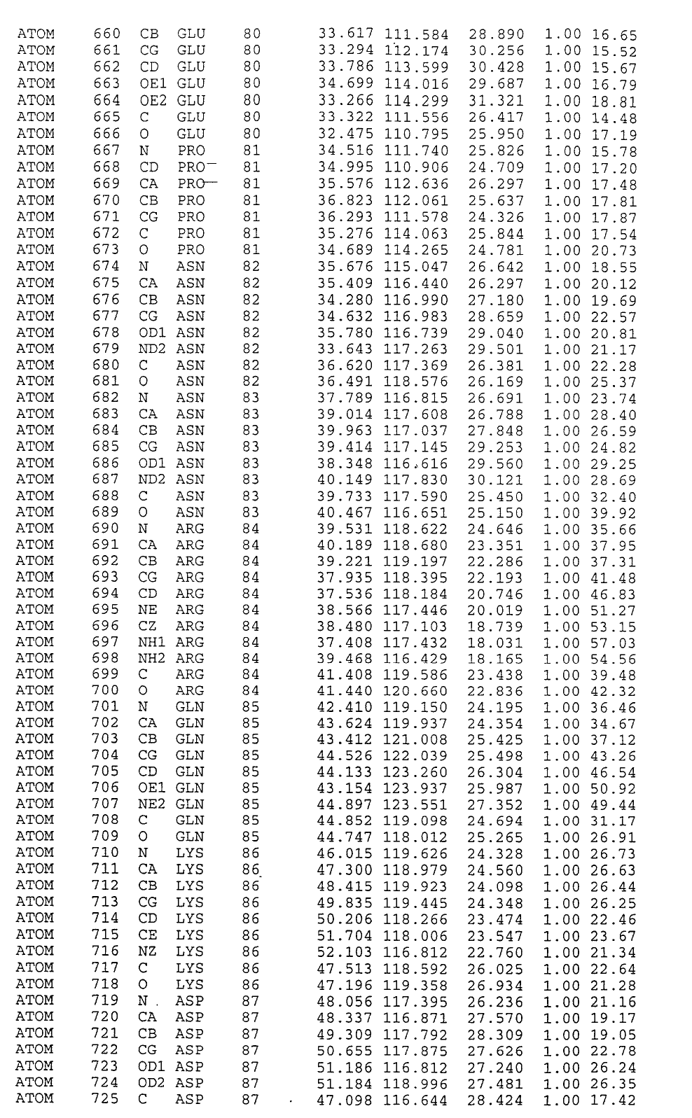

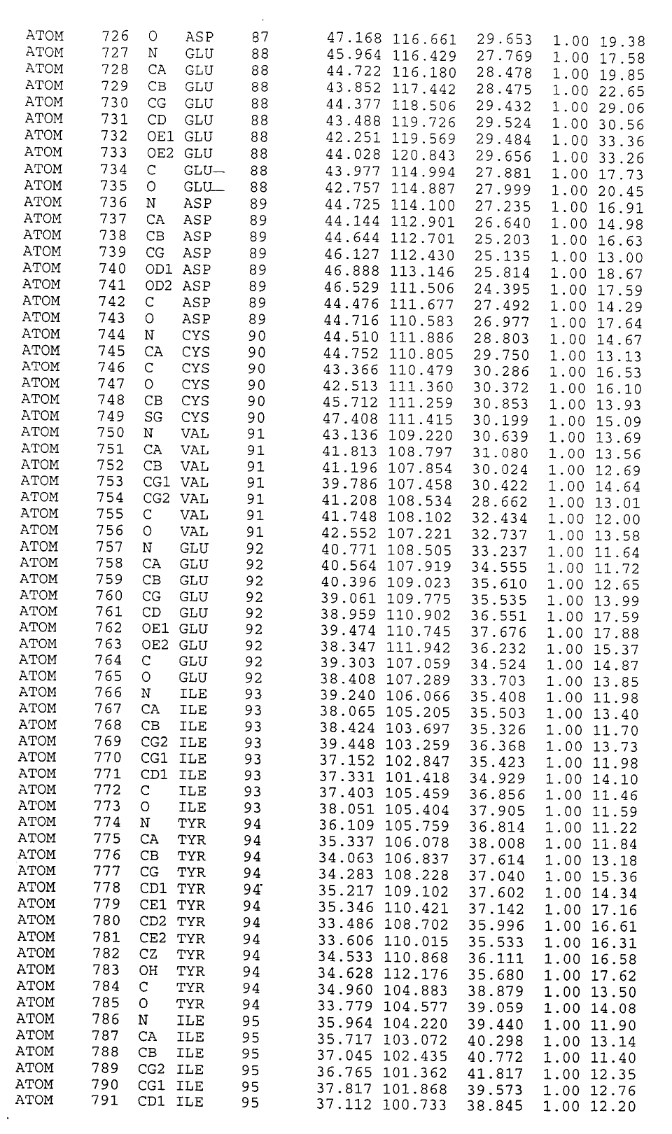

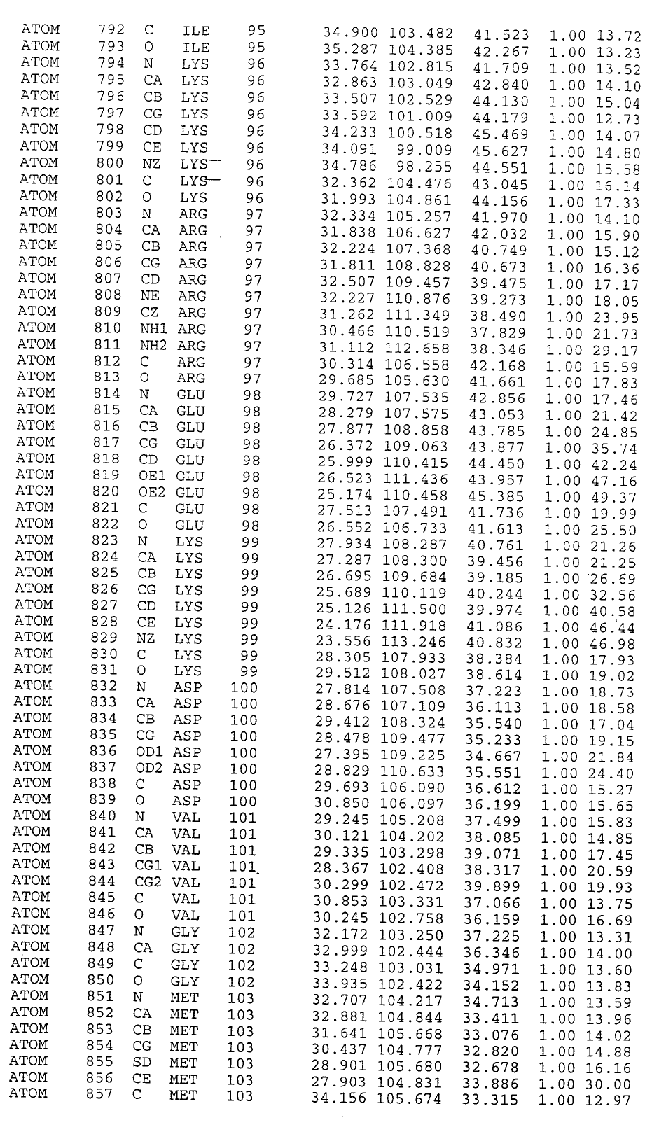

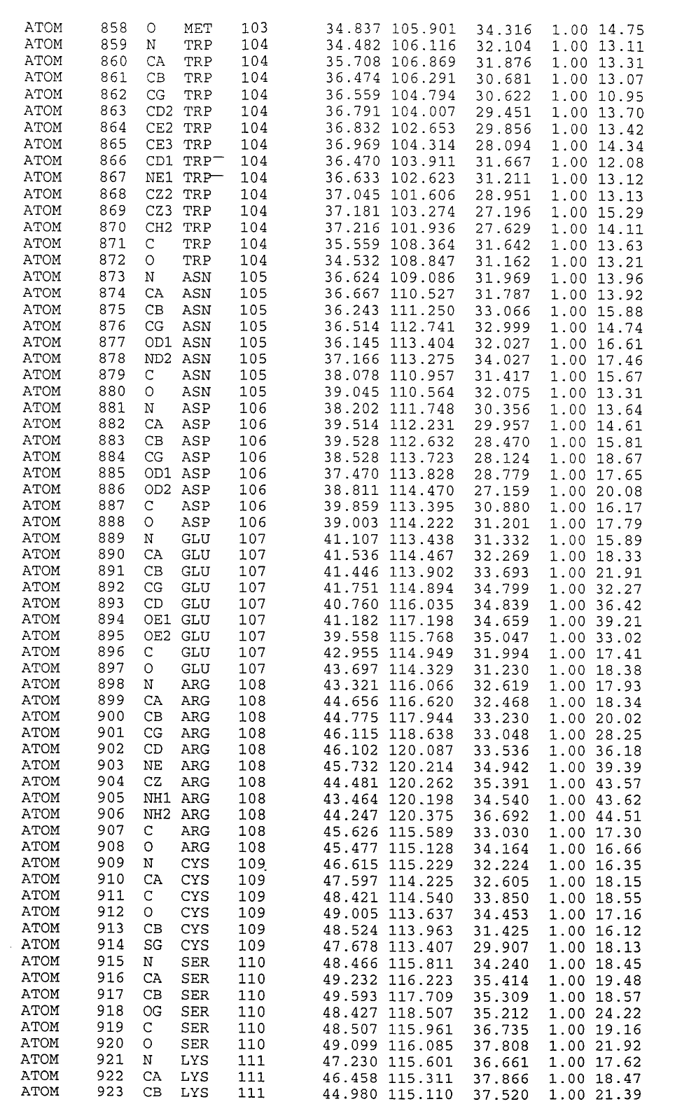

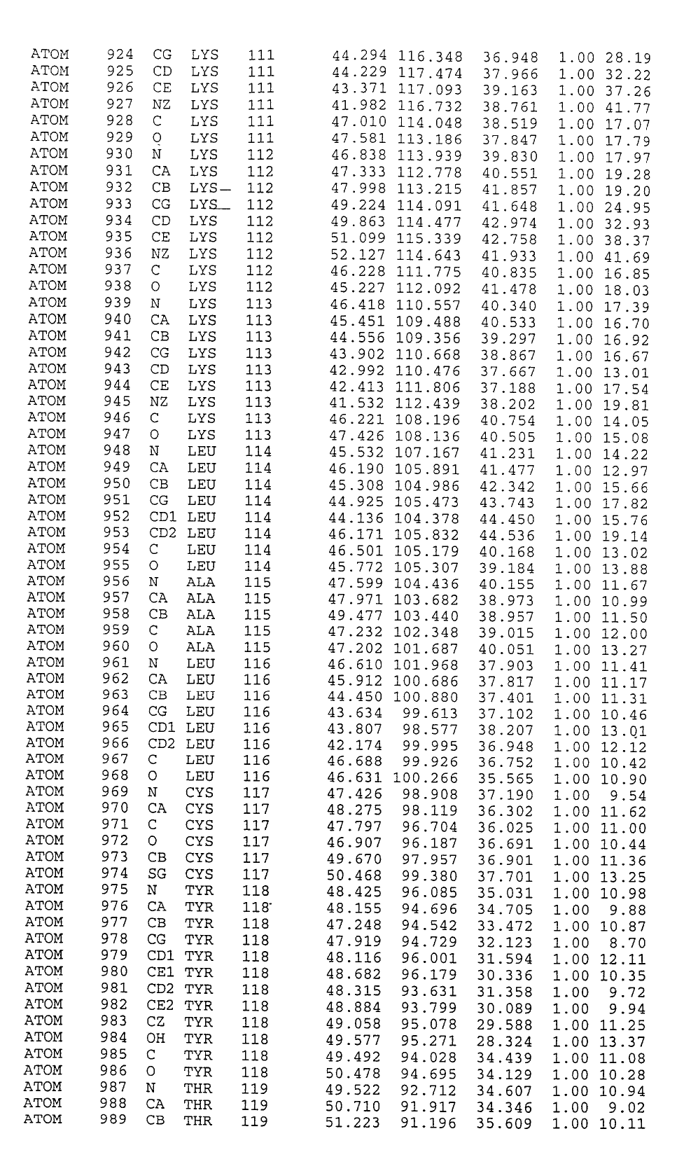

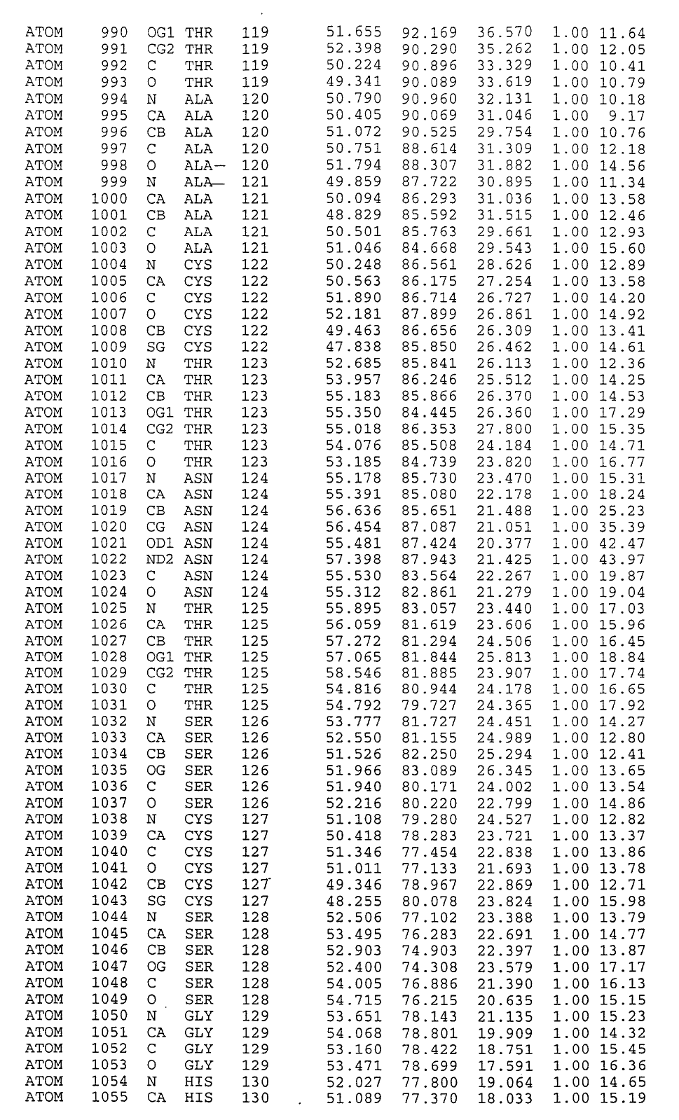

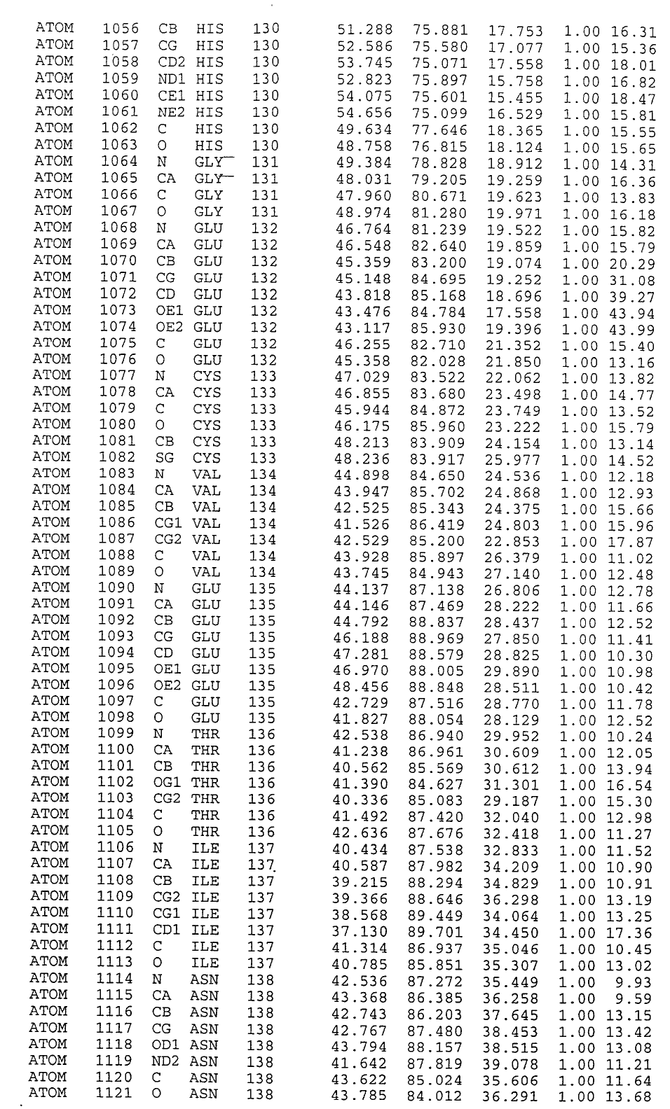

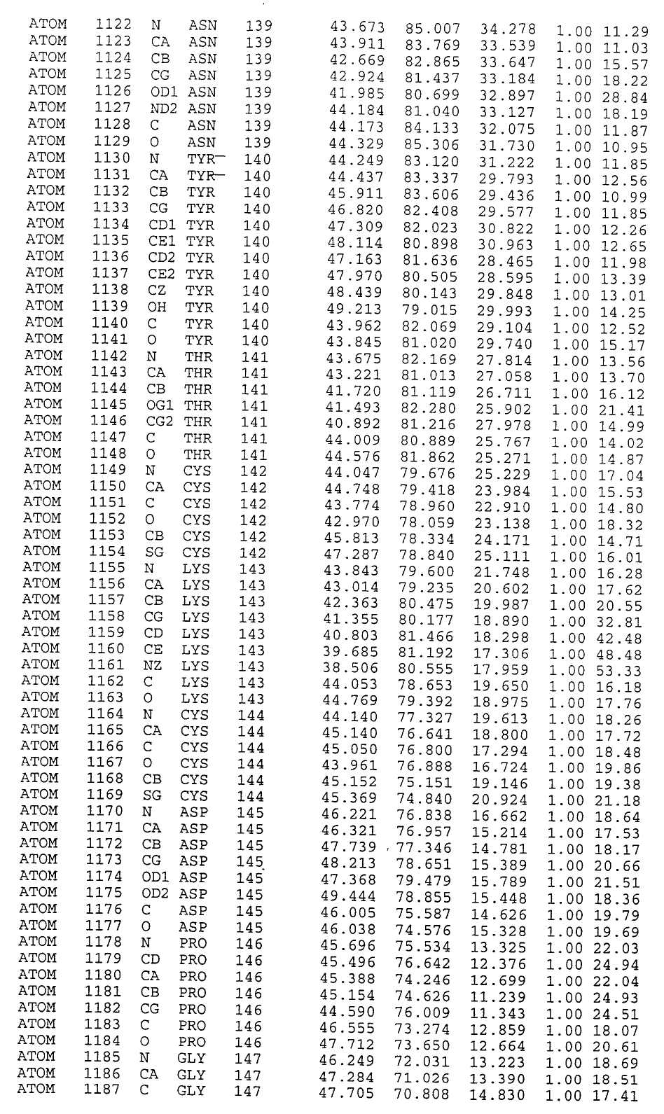

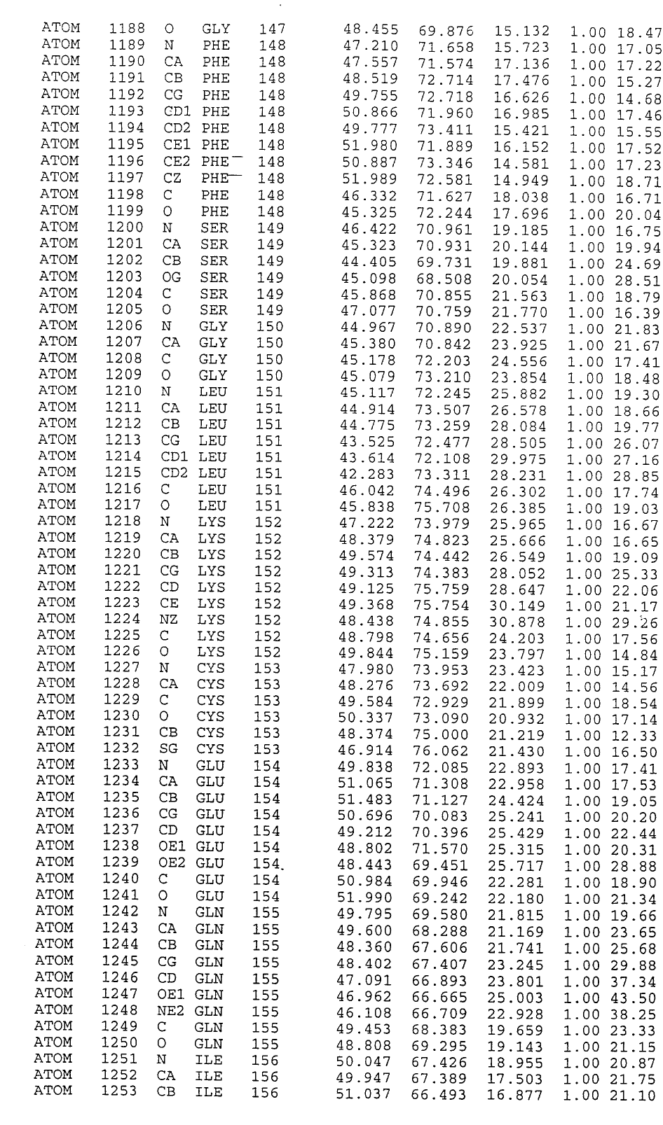

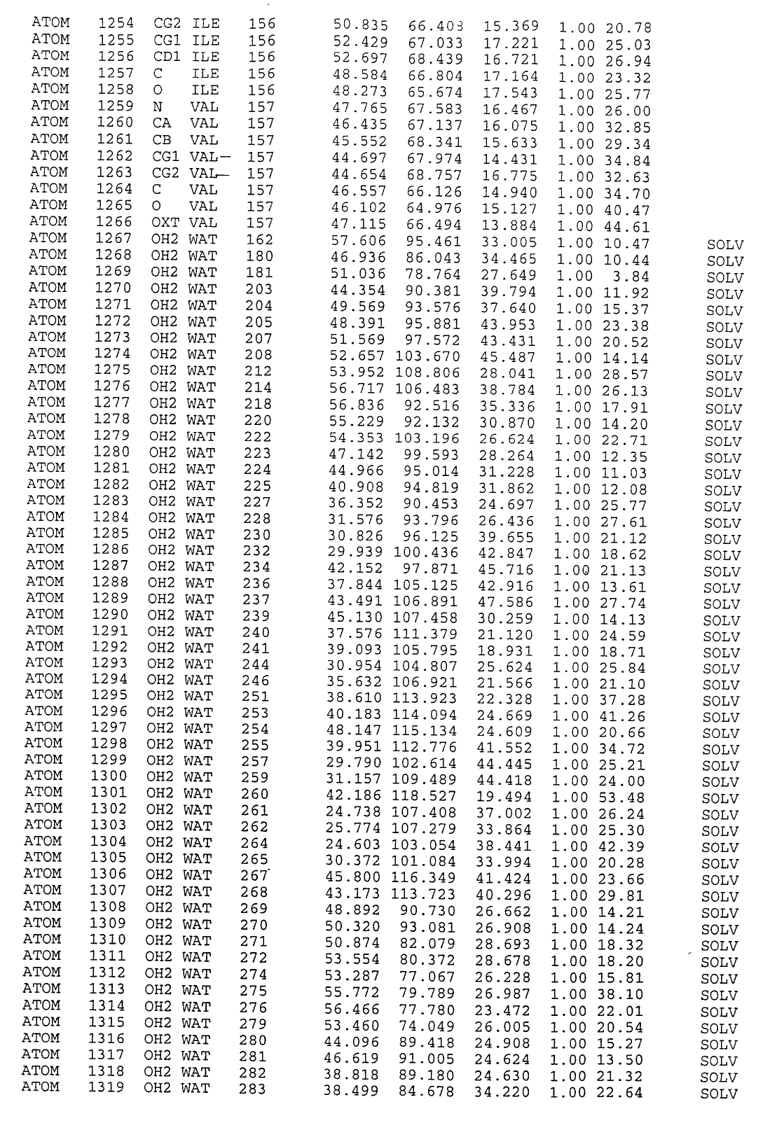

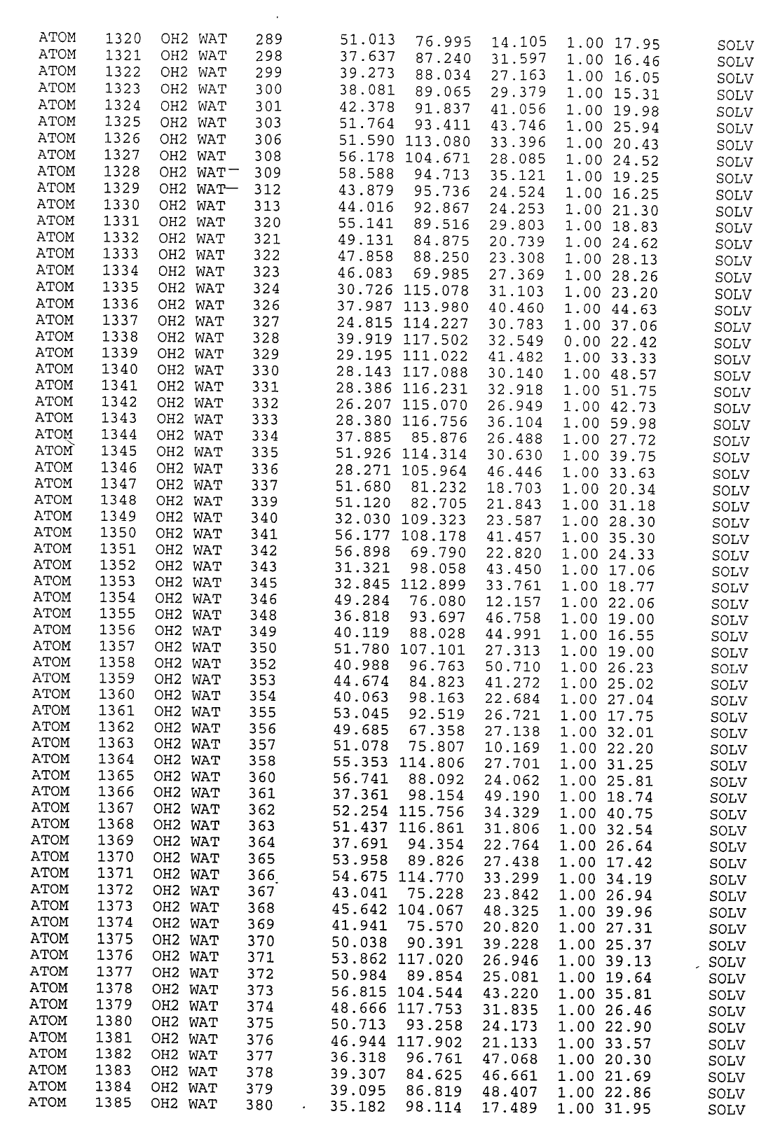

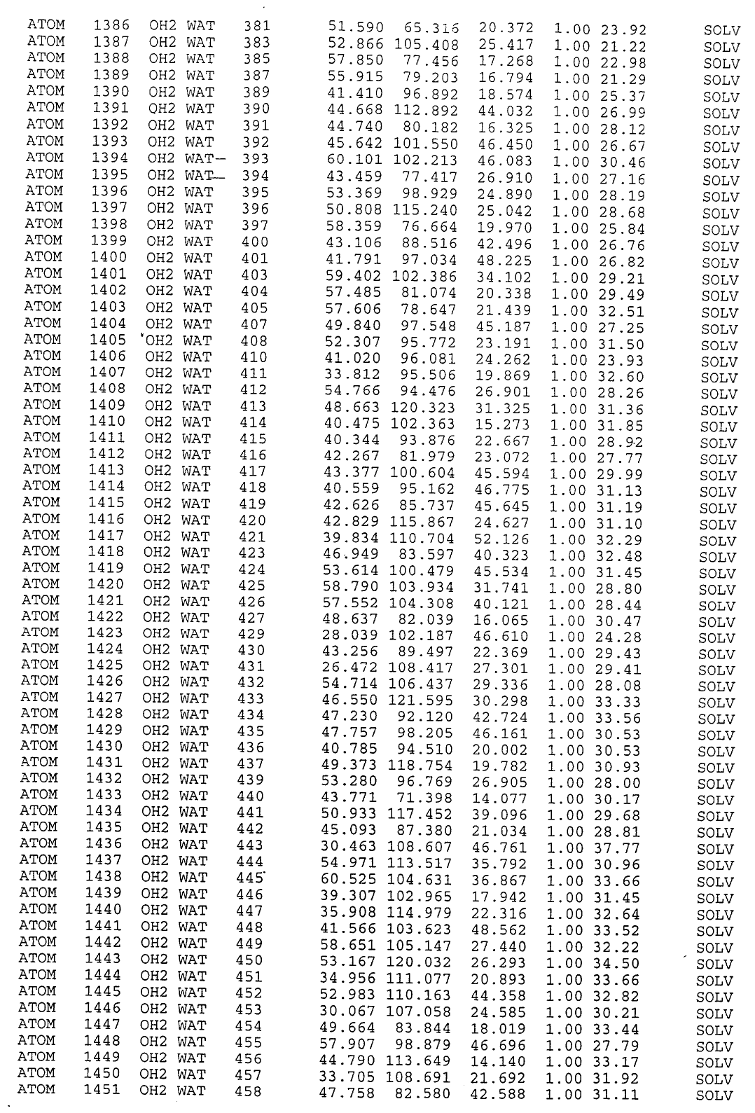

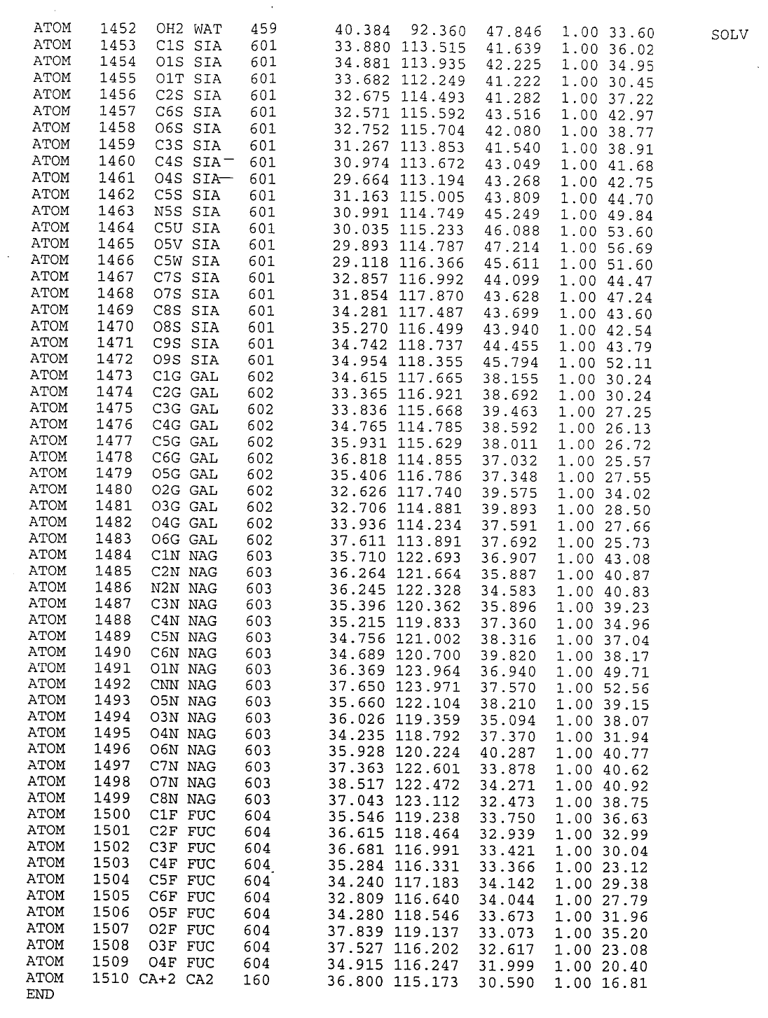

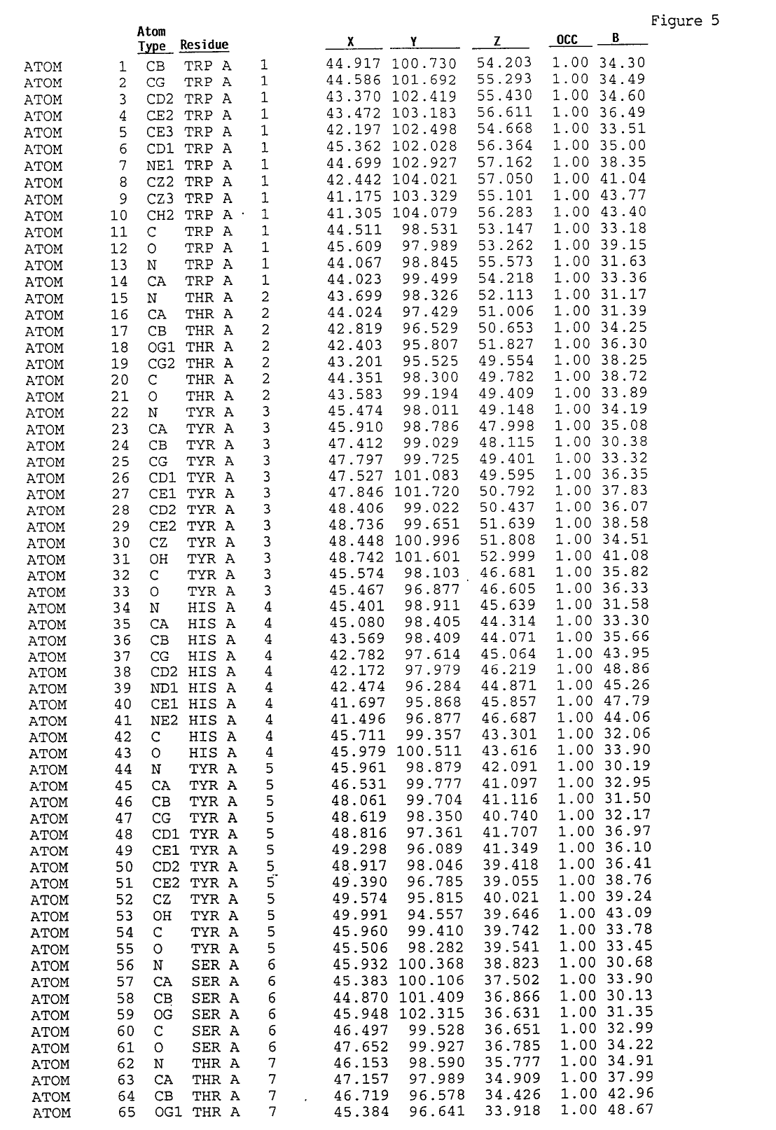

Still further, the present invention provides the three dimensional structures of the P-selectin LE and a PSGL-1 peptide as derived by x-ray diffraction data of the P-selectin LE: PSGL-1 peptide crystal. Specifically, the three dimensional structures of P-selectin LE and the PSGL-1 peptide are defined by the structural coordinates shown in FIG. 5, ± a root mean square deviation from the backbone atoms of the amino acids of not more than 1.5 Å, preferably not more than 1.0 Å, and most preferably not more than 0.5 Å. The structural coordinates P-selectin LE and the PSGL-1 peptide are useful for a number of applications, including, but not limited to, the visualization, identification and characterization of various active sites of P-selectin LE, PSGL-1 and the P-selectin LE:PSGL-1 peptide complex, including PSGL-1 (and SLe^{X}) binding sites. The active site structures may then be used to design agents which interact with P-selectin LE, PSGL-1, as well as P-selectin LE complexed with SLe^{X}, PSGL-1, or related molecules.

The present invention is also directed to an active site of an SLe^{X}binding protein or peptide, and preferably the SLe^{X} binding site of P-selectin LE, comprising the relative structural coordinates of amino acid residues TYR48, GLU80, ASN82, GLU92, TYR94, PRO98, SER99, ASN105, ASP106, GLU107 and bound calcium according to FIG. 3, ± a root mean square deviation from the backbone atoms of said amino acids of not more than 1.5 Å, preferably not more than 1.0 Å, and most preferably not more than 0.5 Å. Alternatively, the active site may include, in addition to the structural coordinates define above, the relative structural coordinates of amino acid residues TYR44, SER46, SER47, ALA77, ASP78, ASN79, PRO81, ASN83, ARG85, GLU88, CYS90, ILE93, LYS96, SER97, ALA100, TRP104, HIS108, LYS111 and LYS113 according to FIG. 3, ± a root mean square deviation from the backbone atoms of the amino acids of not more than 1.5 Å, preferably not more than 1.0 Å, and most preferably not more than 0.5 Å. The SLe^{X} active site may correspond to the configuration of P-selectin LE in its state of association with an agent, preferably, SLe^{X}, or in its unbound state.

The present invention is further directed to an active site of an SLe^{X} binding protein or peptide, and preferably the SLe^{X} binding site of E-selectin LE, comprising the relative structural coordinates of amino acid residues TYR48, GLU80, ASN82, ASN83, GLU92, TYR94, ARG97, GLU98, ASN105, ASP106, GLU107 and bound calcium according to FIG. 4, ± a root mean square deviation from the backbone atoms of said amino acids of not more than 1.5 Å, preferably not more than 1.0 Å, and most preferably not more than 0.5 Å. Alternatively, the active site may include, in addition to the structural coordinates define above, the relative structural coordinates of amino acid residues TYR44, SER45, PRO46, SER47, ALA77, PRO78, GLY79, PRO81, GLU88, CYS90, LYS99, ASP100, TRP104, ARG108, LYS111 and LYS113 according to FIG. 4, ± a root mean square deviation from the backbone atoms of the amino acids of not more than 1.5 Å, preferably not more than 1.0 Å, and most preferably not more than 0.5 Å. The SLe^{X} active site may correspond to the configuration of E-selectin LE in its state of association with an agent, preferably, SLe^{X}, or in its unbound state.

Still further, the present invention provides an active site of a PSGL-1 binding protein or peptide, and preferably the PSGL-1 binding site of P-selectin LE, comprising the relative structural coordinates of amino acid residues ALA9, TYR45, SER46, SER47, TYR48, GLU80, ASN82, LYS84, ARG85, GLU88, GLU92, TYR94, PRO98, SER99, ASN105, ASP106, GLU107, HIS108, LEU110, LYS111, LYS112, LYS113, HIS114 and bound strontium according to FIG. 5, ± a root mean square deviation from the backbone atoms of said amino acids of not more than 1.5 Å, preferably not more than 1.0 Å, and most preferably not more than 0.5 Å. Alternatively, the active site may include, in addition to the structural coordinates define above, the relative structural coordinates of amino acid residues SER6, THR7, LYS8, TYR10, SER11, TYR44, TYR49, TRP50, ALA77, ASP78, ASN79, PRO81, ASN83, ASN86, ASN87, CYS90, ILE93, ILE95, LYS96, SER97, ALA100, TRP104 and CYS109 according to FIG. 5, ± a root mean square deviation from the backbone atoms of the amino acids of not more than 1.5 Å, preferably not more than 1.0 Å, and most preferably not more than 0.5 Å. The PSGL-1 binding site may correspond to the configuration of P-selectin LE in its state of association with an agent, and preferably, PSGL-1 or a PSGL-1 peptide, or in its unbound state. It is also within the confines of the present invention that strontium may be substituted with calcium for purposes of using the structural coordinates for drug design.

Another aspect of the present invention is directed to a method for identifying an agent that interacts with a binding or active site of P-selectin LE. Specifically, the method comprises the steps of: (a) generating a three dimensional model of P-selectin LE using the relative structural coordinates according to FIGS. 2, 3 or 5, ± a root mean square deviation from the backbone atoms of said amino acids of not more than 1.5 Å, preferably not more than 1.0 Å, and most preferably not more than 0.5 Å; and (b) employing said three-dimensional structure to design or select an agent. The agent may be identified using computer fitting analyses utilizing various computer software programs that evaluate the “fit” between the putative active site and the identified agent, by (a) generating a three dimensional model of the putative active site of a molecule or molecular complex using homology modeling or the atomic structural coordinates of the active site, and (b) determining the degree of association between the putative active site and the identified agent. Three dimensional models of the putative active site may be generated using any one of a number of methods known in the art, and include, but are not limited to, homology modeling as well as computer analysis of raw data generated using crystallographic or spectroscopy data. Computer programs used to generate such three dimensional models and/or perform the necessary fitting analyses include, but are not limited to: GRID (Oxford University, Oxford, UK), MCSS (Molecular Simulations, San Diego, Calif.), AUTODOCK (Scripps Research Institute, La Jolla, Calif.), DOCK (University of California, San Francisco, Calif.), Flo99 (Thistlesoft, Morris Township, NJ), Ludi (Molecular Simulations, San Diego, Calif.), QUANTA (Molecular Simulations, San Diego, Calif.), Insight (Molecular Simulations, San Diego, Calif.), SYBYL (TRIPOS, Inc., St. Louis. MO) and LEAPFROG (TRIPOS, Inc., St. Louis, Mo.).

The effect of such an agent identified by computer fitting analyses on P-selectin LE activity may be further evaluated by contacting the identified agent with P-selectin LE and measuring the effect of the agent on P-selectin LE activity. Depending upon the action of the agent on the active site of P-selectin LE, the agent may act either as an inhibitor or activator of P-selectin LE activity. For example, enzymatic assays may be performed and the results analyzed to determine whether the agent is an inhibitor of P-selectin LE and SLe^{X} (i.e., the agent may reduce or prevent binding affinity between P-selectin LE and SLe^{X}) or an activator of P-selectin LE and SLe^{X} (i.e., the agent may increase binding affinity between P-selectin and SLe^{X}). Further tests may be performed to evaluate the potential therapeutic efficacy of the identified agent on conditions associated with selectins such as inflammation.

The present invention is not limited to identifying agents which interact with an active site of P-selectin LE, but also is directed to a method for identifying an activator or inhibitor of any molecule or molecular complex comprising an SLe^{X} binding site or a PSGL-1 binding site, including but not limited to P-selectin, E-selectin, E-selectin LE, P-selectin LE:SLe^{X} complex, and E-selectin LE:SLe^{X} complex.

In one embodiment, the present invention provides a method for identifying an activator or inhibitor of a molecule or molecular complex comprising an SLe^{X} binding site, comprising the steps of: (a) generating a three dimensional model of said molecule or molecular complex comprising an SLe^{X}binding site using (i) the relative structural coordinates according to FIG. 3 of residues TYR48, GLU80, ASN82, GLU92, TYR94, PRO98, SER99, ASN105, ASP106, GLU107 and bound calcium, ± a root mean square deviation from the backbone atoms of said amino acids of not more than 1.5 Å, preferably not more than 1.0 Å, and most preferably not more than 0.5 Å or (ii) the relative structural coordinates according to FIG. 4 of amino acid residues TYR48, GLU80, ASN82, GLU92, TYR94, ARG97, GLU98, ASN105, ASP106, GLU107 and bound calcium, ± a root mean square deviation from the backbone atoms of said amino acids of not more than 1.5 Å, preferably not more than 1.0 Å, and most preferably not more than 0.5 Å; and (b) selecting or designing a candidate activator or inhibitor by performing computer fitting analysis with the three dimensional model generated in step (a). In another embodiment, the relative structural coordinates according to FIG. 3 further comprises amino acid residues TYR44, SER46, SER47, ALA77, ASP78, ASN79, PRO81, ASN83, ARG85, GLU88, CYS90, ILE93, LYS96, SER97, ALA100, TRP104, HIS108, LYS111 and LYS113, ± a root mean square deviation from the backbone atoms of said amino acids of not more than 1.5 Å, preferably not more than 1.0 Å, and most preferably not more than 0.5 Å. In yet another embodiment, the relative structural coordinates according to FIG. 4 further comprises the amino acid residues TYR44, SER45, PRO46, SER47, ALA77, PRO78, GLY79, PRO81, GLU88, CYS90, LYS99, ASP100, TRP104, ARG108, LYS111 and LYS113, ± a root mean square deviation from the backbone atoms of said amino acids of not more than 1.5 Å, preferably not more than 1.0 Å, and most preferably not more than 0.5 Å. Once the candidate activator or inhibitor is obtained or synthesized, the candidate activator or inhibitor may be contacted with the molecule or molecular complex, and the effect the candidate activator or inhibitor has on said molecule or molecular complex may be determined. Preferably, the candidate activator or inhibitor is contacted with the molecule or molecule complex in the presence of SLe^{X} (or a molecule or molecular complex comprising SLe^{X}) in order to determine the effect the candidate activator or inhibitor has on binding of the molecule or molecular complex to SLe^{X}.

In yet another embodiment, the present invention provides a method for identifying an activator or inhibitor of a molecule or molecular complex comprising a PSGL-1 binding site, comprising the steps of: (a) generating a three dimensional model of said molecule or molecular complex comprising a PSGL-1 binding site using the relative structural coordinates according to FIG. 5 of amino acid residues ALA9, TYR45, SER46, SER47, TYR48, GLU80, ASN82, LYS84, ARG85, GLU88, GLU92, TYR94, PRO98, SER99, ASN105, ASP106, GLU107, HIS108, LEU110, LYS111, LYS112, LYS113, HIS114 and bound strontium, ± a root mean square deviation from the backbone atoms of said amino acids of not more than 1.5 Å, preferably not more than 1.0 Å, and most preferably not more than 0.5 Å; and (b) selecting or designing a candidate activator or inhibitor by performing computer fitting analysis with the three dimensional model generated in step (a). In another embodiment, the relative structural coordinates according to FIG. 5 further comprises amino acid residues SER6, THR7, LYS8, TYR10, SER11, TYR44, TYR49, TRP50, ALA77, ASP78, ASN79, PRO81, ASN83, ASN86, ASN87, CYS90, ILE93, ILE95, LYS96, SER97, ALA100, TRP104 and CYS109, ± a root mean square deviation from the backbone atoms of said amino acids of not more than 1.5 Å, preferably not more than 1.0 Å, and most preferably not more than 0.5 Å. Once the candidate activator or inhibitor is obtained or synthesized, the candidate activator or inhibitor may be contacted with the molecule or molecular complex, and the effect the candidate activator or inhibitor has on said molecule or molecular complex may be determined. Preferably, the candidate activator or inhibitor is contacted with the molecule or molecule complex in the presence of PSGL-1 or a PSGL-1 peptide in order to determine the effect the candidate activator or inhibitor has on binding of the molecule or molecular complex to PSGL-1 or the PSGL-1 peptide. Here again, it is also within the confines of the present invention that strontium may be substituted with calcium for purposes of using the structural coordinates for drug design.

In addition, the structural coordinates of SLe^{X} as set forth in FIG. 3 or 4, and the structural coordinates of the PSGL-1 peptide set forth in FIG. 5 can be used for identifying or designing agents which interact with SLe^{X}and PSGL-1, respectively. In this regard, the present invention provides a method for identifying an agent that interacts with SLe^{X}, comprising the steps of: (a) generating a three dimensional model of SLe^{X} using the relative structural coordinates according to FIG. 3 or 4, ± a root mean square deviation from the backbone atoms of said amino acids of not more than 1.5 Å, preferably not more than 1.0 Å, and most preferably not more than 0.5 Å; and (b) employing said three-dimensional structure to design or select an agent that interacts with SLe^{X}. The identified agent can then be synthesized or obtained, and then contacted with SLe^{X} (or a molecule or molecular complex comprising SLe^{X}) to determine the effect the agent has on SLe^{X} activity.

Still further, the present invention provides a method for identifying an agent that interacts with PSGL-1, comprising the steps of: (a) generating a three dimensional model of a PSGL-1 peptide using the relative structural coordinates according to FIG. 5, ± a root mean square deviation from the backbone atoms of said amino acids of not more than 1.5 Å, preferably not more than 1.0 Å, and most preferably not more than 0.5 Å; and (b) employing said three-dimensional structure to design or select an agent that interacts with PSGL-1. The identified agent can then be synthesized or obtained, and then contacted with PSGL-1 or the PSGL-1peptide (or a molecule or molecular complex comprising PSGL-1 or the PSGL-1 peptide) to determine the effect the agent has on PSGL-1 or the PSGL-1 peptide activity.

Various molecular analysis and rational drug design techniques are further disclosed in U.S. Pat. Nos. 5,834,228, 5,939,528 and 5,865,116, as well as in PCT Application No. PCT/US98/16879, published WO 99/09148, the contents of which are hereby incorporated by reference.

The present invention is also directed to the agents, activators or inhibitors identified using the foregoing methods. Such agents, activators or inhibitors may be a protein, polypeptide, peptide, nucleic acid, including DNA or RNA, molecule, compound, or drug. Small molecules or other agents which inhibit or otherwise interfere with the selectin-mediated cellular rolling of leukocytes over vascular tissue may be useful in the treatment of diseases involving abnormal inflammatory responses such as asthma and psoriasis.

In addition, the present invention is directed to a method for determining the three dimensional structure of a molecule or molecular complex whose structure is unknown, comprising the steps of obtaining crystals of the molecule or molecular complex whose structure is unknown and generating X-ray diffraction data from the crystallized molecule or molecular complex. The X-ray diffraction data from the molecule or molecular complex is then compared with the known three dimensional structure determined from any of the aforementioned crystals of the present invention. Then, the known three dimensional structure determined from the crystals of the present invention is “conformed” using molecular replacement analysis to the X-ray diffraction data from the crystallized molecule or molecular complex. Alternatively, spectroscopic data or homology modeling may be used to generate a putative three dimensional structure for the molecule or molecular complex, and the putative structure is refined by conformation to the known three dimensional structure determined from any of the crystals of the present invention.

Finally, the present invention provides a method for obtaining a crystallized complex of an E-selectin type molecule and a compound that coordinates calcium such as SLe^{X}. The method comprises the steps of: (a) contacting a crystallized E-selectin type molecule with a compound that coordinates calcium in the presence of calcium ions and PEG to form a crystallized complex of the E-selectin type molecule and the compound that coordinates calcium; and (b) contacting the crystallized complex in the presence of a reduced concentration of calcium ions, and sufficient concentrations of PEG and an ionic salt to obtain a final crystallized complex, that upon cooling, is suitable for elucidating the three dimensional structures of the E-selectin type molecule and the compound that coordinates calcium by x-ray diffraction of the final crystallized complex.

In the method, the “E-selectin type molecule” may be the entire E-selectin molecule as well as portions thereof, such as the lectin and/or epidermal growth factor (EGF)-like domains, and preferably is E-selectin LE. The compound that coordinates calcium is preferably SLe^{X}. In step (a), the crystallized E-selectin type molecule may be prepared by procedures known in the art. When the crystallized E-selection type molecule is E-selectin LE, the crystallized E-selectin LE is preferably prepared as described in Example 1 below. The source of calcium ions in the method is preferably CaCl₂, while the PEG is preferably PEG-1000 to PEG-20,000, and most preferably PEG-4000. The ionic salt may be a number of ionic salts known in the art, and preferably is NaCl.

An important aspect of the method is the reduction of calcium ions in step (b), or the use of an effective calcium concentration that, prevents or reduces the affect of calcium in inhibiting binding of the compound that coordinates calcium to the E-selectin type molecule, thereby permitting the formation of a final crystal complex that a suitable for elucidating the three dimensional structures of the E-selectin type molecule and the compound that coordinates calcium by x-ray diffraction of the final crystallized complex. That is, the concentration of calcium should be sufficiently low to permit the compound that coordinates calcium to bind to its binding site on the E-selectin type molecule. For example, if the compound that coordinates calcium is SLe^{X}, then it is envisioned that the concentration of calcium ions in step (a) may range from 20 mM to 300 mM, while the concentration of calcium ions in step (b) is lower (i.e. in the range from 100 μM to 20 mM). In steps (a) and (b), PEG should be at a concentration sufficient to maintain the integrity of the crystal in view of the reduced or lower concentration of calcium, and is preferably about 15% to 60% (w/v) PEG. The concentration of the ionic salt in step (b) is about 10 mM to 500 mM. The contacting in step (a) may be affected for about 10-20 hours, and preferably about 15 hours when the compound that coordinates crystal is SLe^{X}. The contacting in step (b) is affected for about 0.5-3 hours, and preferably for about 1 hour.

It is also within the confines of the present invention that steps (a) and (b) can be combined in which case the crystallized E-selectin type molecule is contacted with a compound that coordinates calcium in the presence of sufficient concentrations of calcium ions, PEG and an ionic salt to form a crystallized complex (of the E-selectin type molecule and the compound that coordinates calcium) that is suitable for elucidating the three dimensional structures of the E-selectin type molecule and the compound that coordinates calcium by x-ray diffraction of the final crystallized complex. If steps (a) and (b) are combined, then it is envisioned that the concentrations of calcium ions, PEG and the ionic salt are about 100 μM to 20 mM, 15% to 60% (w/v) and 10 mM to 500 mM, respectively.

The present invention may be better understood by reference to the following non-limiting Example. The following Example is presented in order to more fully illustrate the preferred embodiments of the invention, and should in no way be construed as limiting the scope of the present invention.

### EXAMPLE 1

1. Experimental Procedures

Generation of Constructs and Protein/Peptide Preparation. The lectin-EGF (LE) domains (153 amino acids) of P-selectin (P-LE) and E-selectin (E-LE) fused to the CH2—CH3 region of IgG₁ via an intervening enterokinase cleavage sequence (Asp-Asp-Asp-Asp-Lys) were expressed in CHO cells and recovered from conditioned media by protein A sepharose (Pharmacia) chromatography. Monomeric selectin LE domains were produced by digestion of the dimeric Fc constructs with enterokinase (LaVallie et al., 1993) and the enzyme and residual Fc domains were removed by chromatography over tandem soy bean trypsin inhibitor-agarose (Sigma) and protein A (Perseptive Biosystems) columns. The selectin LE domains were deglycosylated at 37° C. for 48 hrs at a ratio of 25 milliunits N-glycanase/mg protein and purified by anion exchange and hydrophobic interaction chromatography. LE domains were brought to 10-30 mg/ml by vacuum concentration. Both P-LE and E-LE were determined to be correct by mass spectrometry (MS), monomeric by gel filtration HPLC, and functional by surface plasmon resonance (BIAcore) analysis (see below).

A soluble construct (19ek.Fc) containing the N-terminal 19 amino acids of PSGL-1 fused to the Fc region of IgG₁ via a nine amino acid linker containing the enterokinase cleavage sequence was described earlier (Goetz et al., 1997). 19ek.Fc was purified, digested with enterokinase, and the monomeric PSGL-1 19ek peptides were recovered as in the above for the LE constructs. The heterogenous 19ek peptides were purified to individual species by SuperQ anion-exchange chromatography using a gradient of 0-500 mM NaCl. Their structures were determined by MS before and after proteolytic and glycosidic digestions, NMR, and by composition analyses (J. Rouse, D. Tsao, and R. Camphausen, unpublished data).

P-LE/19ek Peptide Binding Studies. Surface plasmon resonance was performed on BIAcore 2000 and 3000 instruments at 25° C. using streptavidin-coated sensor chips (BIAcore) and HBS-P buffer (BIAcore; 10 mM HEPES (pH 7.4), 150 mM NaCl, and 0.005% polysorbate 20 (v/v)) adjusted to 1 mM each CaCl₂ and MgCl₂. The 19ek peptides and sPSGL, a fucosyltransferase-VII modified version of the entire dimeric extracellular domain of PSGL-1 (Croce et al., 1998), were biotinylated at Lys residues with Sulfo-NHS-LC-Biotin (Pierce). Following biotinylation, sPSGL was reacted with immobilized P-selectin in order to isolate functional material (Sako et al., 1995). A synthetic peptide (AnaSpec, Inc.), corresponding to the polypeptide portion of SGP-3 was similarly biotinylated. Biotinylated reagents were coated onto sensor chips using HBS-P buffer. Glycosylated P-LE and E-LE, quantitated by experimentally determined extinction coefficients (280 nm), were injected over the19ek peptide, sPSGL and control surfaces at 40 μL/min. The specificity of binding was validated by control experiments performed in the presence of neutralizing Mabs to P- and E-selectin, 10 mM EDTA, and soluble 19ek peptides (R. Camphausen, unpublished data). Deglycosylated P-LE and E-LE bound comparably to the intact, glycosylated versions.

Crystallization and Data Collection. All diffraction data were collected in-house on Rigaku RU200 generators running at 5.0 KW, with Yale/Molecular Structure Corp. focusing mirrors and RAXIS II or RAXIS IV image plate area detectors except where noted.

Plate shaped crystals of P-LE were grown at 18° C. using vapor diffusion from a solution containing 10 mg/ml protein, 100 mM Tris-HCl (pH 8.5), 150 mM NaCl, 12 mM CaCl₂, 10% (v/v) 2,4 methyl pentane diol (MPD), and 10% (w/v) PEG 6000. These crystals were transferred into 100 mM Tris-HCl (pH 8.5), 75 mM NaCl, 10 mM CaCl₂, 10% (v/v) MPD, and 11% (w/v) PEG 6000, then transferred for two hours to the same buffer diluted by 5% (v/v) with MPD. After a second 5% dilution with MPD the crystal was soaked for a 13 hours prior to flash cooling in liquid propane held at liquid nitrogen temperature. P-LE complexed with SLe^{X} (SLe^{X}-β-O-methyl, Toronto Research Chemicals) was obtained using the same methods but with the addition of 8 mM SLe^{X} to the final soaking solution. The space group of the P-LE crystals was P2₁, with cell parameters a=81.0 Å, b=60.8 Å, c=91.4 Å, and beta=103.6°. Soaking crystals in SLe^{X} reduced the maximum resolution to 3.4 Å, increased the mosaicity to 1.5° and gave cell parameters a=81.1 Å, b=60.5 Å, c=91.4 Å and beta=103.3. Diffraction data were processed and scaled with DENZO/SCALEPACK (HRL Research, Inc.) giving the statistics reported in Table 1.

Large, rod shaped crystals of E-LE were obtained using vapor diffusion at 18° C. from a solution containing 30 mg/ml protein, 100 mM HEPES (pH 7.5), 10 mM Tris-HCl, 200 mM CaCl₂ and 15% (w/v) PEG 4000. Crystals took several weeks to grow and were more reproducible with the use of macroseeding. For complexes with SLe^{X}, E-LE crystals were transferred to a solution containing 100 mM HEPES (pH 7.5), 200 mM CaCl₂, 30% (w/v) PEG 4000, and 15 mM SLe^{X} for 15 hours at 25° C. After this initial incubation, crystals were transferred into 100 mM Tris-HCl (pH 7.4), 300 mM NaCl, 2 mM CaCl₂, 30% (w/v) PEG 4000, and 15 mM SLe^{X} for an additional hour prior to flash cooling as described above. The E-LE/SLe^{X} crystals belonged to space group P2₁2₁2₁, with cell parameters a=34.5 Å, b=72.4 Å, and c=77.6 Å. Diffraction data were processed as in above.

Large crystals of the P-LE/PSGL-1 19ek peptide (SGP-3) complex measuring up to 0.5×0.5×0.3 mm were obtained by repeat macroseeding into vapor diffusion crystallization drops. The crystals grew from 8 mg/ml P-LE, SGP-3 in a 2 fold molar excess, 10 mM Tris-HCl, 100 mM HEPES (pH 7.0), 150 mM NaCl, 4 mM CaCl₂, 50 mM SrCl₂, 5% (w/v) PEG 6000, and 33% (v/v) MPD. Earlier small seed crystals were obtained from a similar buffer containing 50% MPD but with no SrCl₂ or PEG 6000. Crystals were transferred into 100 mM HEPES pH 7.0, 10% PEG 6000, 30% MPD, 100 mM NaCl, and 50 mM SrCl₂ for 15 hours prior to flash cooling. A mercury-derivatized crystal was obtained by adding 0.5 mM mercury acetate to the final soaking buffer for 24 hours prior to flash cooling. The space group of the complex crystals was found to be I222 with cell parameters a=63.4 Å, b=96.8 Å, and c=187.3 Å. The crystals were in bipyramidal form. Native diffraction data were collected at Brookhaven National Labs station X4A using an RAXIS IV to record diffraction data. The mercury derivative data were collected in-house. All data were reduced as described above giving statistics in Table 1.

Structure Determination and Refinement. Crystals of E-LE complexed with SLe^{X}were found to be essentially isomorphous with those reported earlier (Graves, et al., 1994) with a single copy of E-LE in the crystallographic asymmetric unit. Rigid body refinement within CNS (Brunger et al., 1998) was used to obtain initial phases that gave clear electron density for the bound SLe^{X}. These maps were further improved with the use of BUSTER (Bricogne, 1993) allowing a model for bound SLe^{X} to be fitted using QUANTA (Molecular Simulations, Inc.). All further refinement was performed in CNS giving a final model with 86% of residues in the most favored region of the Ramachandran plot and consisting of total 1510 atoms, 186 water molecules, one calcium ion, and one copy of SLe^{X}. Statistics are described in Table 1.

The structure of P-LE was solved with molecular replacement using the published model of the E-selectin lec/EGF construct (PDB accession code 1ESL). The program AMORE (CCP4, 1994) was used to locate all four copies of P-LE. The model was built and refined using the methods described above. One copy of P-LE had poorer electron density than the other three but with the use of non-crystallographic symmetry the refinement progressed well giving the statistics in Table 1. At the end of refinement the fourth copy of P-LE had an average B-factor of 63.7 Å² compared to 44.2 Å² for the other three copies. The final model consists of four copies of P-LE with 81% of the residues in the most favored regions of the Ramachandran plot, 5418 total atoms, 134 water molecules, 2 MPD molecules and 4 calcium ions.

The crystals of P-LE soaked in SLe^{X} were essentially isomorphous with the P-LE structure described above. After rigid body refinement in CNS there was clear density for three bound SLe^{X} molecules. The fourth binding site was partially occluded by crystal contacts, thus explaining the loss of resolution upon soaking in SLe^{X}. QUANTA was used to model bound SLe^{X} and refit protein residues before limited refinement in CNS. This gave a final model with 71% of the residues in the most favored Ramachandran regions, 5455 total atoms, 3 SLe^{X} molecules, 4 calcium ions and 2 MPD molecules.

All attempts to solve the structure of P-LE complexed with the PSGL-1 19ek peptide SGP-3 by molecular replacement failed. The heavy atom site was located with Patterson techniques refined in SHARP (de la Fourtelle and Bricogne, 1997). These phases gave poor maps but of sufficient quality to allow positioning of two lectin domains from the P-LE structure and independent positioning of two EGF domains. These maps also indicated the presence of bound SLe^{X} molecules in the same position as found in the P-LE/SLe^{X}complex and extra density that which could not be interpreted. The heavy atom phases were combined with model phases in BUSTER giving clear maximum entropy maps that allowed fitting of the SGP-3 polypeptide and SLe^{X}moieties in QUANTA. This structure was refined as described above giving statistics in Table 1 and 84% of residues in the most favored regions of the Ramachandran plot. The final model consists of two P-LE/SGP-3 complexes, 3263 atoms, 2 strontium ions, 224 water molecules, 2 sodium ions, and 7 bound MPD molecules.

2. Results and Discussion

X-ray Crystal Structure of the P-selectin Lectin/EGF Domains. We generated a P-selectin construct containing the N-terminal lectin and EGF domains (termed P-LE). While the E-selectin lec/EGF crystal structure (Graves et al., 1994) indicates that there is minimal interaction between the lectin and EGF domains and the putative SLe^{X} binding site is well removed from the lectin-EGF domain junction, we elected to retain the EGF domain because of studies suggesting it may have a functional role (Gibson et al., 1995; Kansas et al., 1994). P-LE was fused to the Fc region of IgG₁ via an intervening enterokinase cleavage sequence. This construct, termed P-LE.Fc, allowed for facile purification from conditioned media and the generation of monomeric P-LE domains following enterokinase digestion. P-LE expressed in CHO cells contains three N-linked glycans, which were enzymatically removed prior to crystallization. P-LE crystals were obtained in space group P2₁, with 4 molecules in the crystallographic asymmetric unit and diffracted to 2.4 Å resolution. The structure was solved by molecular replacement using the E-selectin lec/EGF crystal structure coordinates as a search model.

The P-LE crystal structure adopts an overall conformation essentially identical to that of E-selectin lec/EGF structure. This is consistent with the 62% sequence identity between the selectins in these domains and results in a RMS difference of only 0.7 Å for their C backbones. The lectin and EGF domains of P-LE interact via a small interface making the similarity with the E-selectin construct even more striking since this relationship is also maintained. The movement of several loops in the EGF domain together with a small movement of the interdomain angle is responsible for many of the minor differences between the two structures. The interdomain angle also varies slightly between different copies of P-LE in the crystal and therefore is likely an effect of crystal packing forces.

The putative SLe^{X} binding site, suggested by mutagenesis and structure studies (Kansas, 1996), is remarkably conserved between P-LE and the E-selectin lec/EGF structure. The common feature of this site, and presumably the basis for the metal dependency of selectin function, is a calcium ion coordinated by the side chains of Glu-80, Asn-82, Asn-105 and Asp-106 and the backbone carbonyl of Asp-106. Two water molecules also ligate calcium within P-LE, one of which is stabilized by the side-chain of Asn-83. The similarity of the SLe^{X} binding sites is even more striking considering that the eight bound waters in this area of P-LE are also present (within 0.8 Å) in the E-selectin lec/EGF structure (not shown). The binding sites do however have differences in one area defined largely by the change of Arg-97 in E-selectin to Ser-97 in P-selectin. In the E-selectin lec/EGF structure, Arg-97 stacks on Tyr-94 thereby presenting a positively charged surface in this region whereas in P-LE Tyr-94 is not obstructed by the smaller Ser-97 residue and thereby has the potential to mediate hydrophobic interactions. Arg-97 in the E-selectin lec/EGF construct makes a hydrogen bond with Asp-100, which is an alanine residue in P-LE. Adjacent to this region is Lys-99 which, within E-selectin lec/EGF, points away from the binding site. The equivalent residue in P-LE is a serine residue that faces into the binding site.

Crystal Structures of P- and E-selectin Lec/EGF Domains Complexed with SLe^{X}. We sought to obtain the crystal structure of P-LE complexed with SLe^{X}, which we anticipated could be obtained by soaking SLe^{X} into preformed P-LE crystals. We also wished to understand the structural basis for the approximately 10-fold higher SLe^{X} binding affinity described for E-selectin relative P-selectin (Poppe et al., 1997), which is unexpected given the similarity of their putative SLe^{X}binding sites. Therefore, we produced an E-selectin lectin/EGF construct (E-LE) which was expressed, purified and deglycosylated as for P-LE. E-LE crystallized under conditions similar to those employed earlier for E-selectin lec/EGF (Graves et al., 1994) with one copy of E-LE in the crystallographic asymmetric unit.

P-LE crystals were soaked in a stabilizing solution containing 8 mM SLe^{X} and crystallographic data was collected that extended to 3.4 Å (Table 1). The P-LE/SLe^{X} crystal structure has four copies of P-LE in the asymmetric unit, with one SLe^{X} binding site partially occluded by crystal contacts. It is presumably this crystal contact that causes such a dramatic loss in diffraction quality upon soaking. Despite the low resolution of the data, clear electron density could be seen extending away from the bound calcium in three copies of P-LE. These maps were used to construct the complex structure of SLe^{X} bound to P-LE although these results were later refined after obtaining a high-resolution SLe^{X} complex with E-LE. Initial attempts to soak SLe^{X} into E-LE crystals at concentrations of SLe^{X} up to 20 mM were unsuccessful. We subsequently determined that the high concentration of calcium used for crystallization inhibited E-selectin in SLe^{X} binding assays (not shown). Therefore, a soaking protocol was derived wherein the calcium concentration was reduced to a level which facilitated SLe^{X} binding and still maintained high-resolution diffraction. These conditions resulted in crystals that gave diffraction data to 1.5 Å resolution and clear electron density for bound SLe^{X} (Table 1). The structure of the P-LE/SLe^{X} complex reveals that the interactions are almost entirely electrostatic in nature and the total buried surface area is small (549 Å²) when compared to the size of SLe^{X}. Since the complex with P-LE is low resolution a more detailed description of the conserved interactions will be described below for the E-LE/SLe^{X} complex. The interactions of the Fuc hydroxyls must provide a large amount of the binding energy. The 3- and 4-hydroxyl groups not only coordinate the bound calcium but also form hydrogen bonds with residues that are themselves coordinating the calcium. In addition, Glu-107 is only 3.4 Å away from the Fuc 2-hydroxyl group and may form a weak hydrogen bond. The SLe^{X} Gal residue hydrogen bonds with protein residues using the 4-hydroxyl group (with the hydroxyl group of Tyr-94) and the 6-hydroxyl group (with the carboxylate group of Glu-92). The NeuNAc residue interacts in the region where the two selectins are very different, the Arg-99 (E-selectin) versus Ser-99 (P-selectin) site. In P-LE, the hydroxyl groups of Tyr-48 and Ser-99 form hydrogen bonds to the carboxylate moiety and 4-hydroxyl group, respectively, of NeuNAc. Finally, it appears that C-4 of the NeuNAc ring packs against Pro-98. The positioning of the NeuNAc would make unfavorable contacts with Arg-99 in E-LE and so moves further back (FIG. 2A) to allow for better interactions. The rest of SLe^{X} is essentially superimposible between E-LE and P-LE.

The structure of E-LE complexed with SLe^{X} confirms the interactions seen in the low-resolution structure of P-LE with different contacts to the NeuNAc as a result of the Ser-97:Arg-97 (P-selectin:E-selectin) difference. Fuc 3- and 4-hydroxyl groups coordinate the bound calcium and form a complex network of hydrogen bonds with residues that also coordinate the calcium. The Fuc 4-hydroxyl group replaces exactly a calcium-ligated water molecule observed in the unliganded structure, accepts a hydrogen bond from Asn-82 and donates a hydrogen bond to Glu-80. The Fuc 3-hydroxyl group displaces another calcium coordinated water molecule although its final position is now one Å closer to Asn-105. Upon SLe^{X} binding Asn-83 rotates its 2 torsion angle to 59° so that it can now donate a hydrogen bond to a bound water that in turn hydrogen bonds to both Fuc 2-hydroxyl group and the side chain of Glu-107 (not shown). This rotation now also allows the Asn-83 side chain to coordinate the calcium. The replacement of Ser-97 in P-LE with Arg-97 in E-LE allows the formation of a different set of interactions with the NeuNAc and causes a movement of the sugar away from the side chain to alleviate close contacts that would occur. The Arg-97 donates hydrogen bonds to the glycosidic oxygen and the carboxylate group of NeuNAc. In a similar arrangement to P-LE, the carboxylate group also accepts a hydrogen bond from Tyr-48.

The protein contacts observed for SLe^{X} binding to P-LE and E-LE are in excellent agreement with site directed mutagenesis studies focused on defining residues important for P- and E-selectin recognition (Erbe et al., 1993; Erbe et al., 1992; Graves et al., 1994; Hollenbaugh et al., 1993; Ng and Weis, 1997). Many of these mutations (e.g., substitutions of Tyr-48 and Tyr-94 in E- and P-selectin, and Arg-97 in E-selectin) can now be interpreted to directly affect hydrogen bonding interactions with SLe^{X}. Others mutations likely disrupt function indirectly by altering the orientation of interacting residues. It is this latter explanation which likely accounts for the disruption of function associated with mutations of a triple Lys stretch (residues 111-113) found in both E- and P-selectin. In the structures of P-LE and E-LE complexed with SLe^{X}, Lys-113 does not participate directly in binding. However, this residues does hydrogen bond (via it side-chain amino group) to the carboxylate moiety of Glu-92 which also hydrogen bonds the 6-hydroxyl group of Gal residue within SLe^{X}. Hence, some mutations in this region of the selectins may disrupt SLe^{X} binding by an indirect mechanism.

Comparing the E- and P-LE/SLe^{X} structures to structures of related lectin/glycan complexes reveals important similarities and differences. Examination of the crystal structure of MBP-A bound to oligomannose (Weis et al., 1992) indicates a similar arrangement of calcium-binding interactions also involving the 3- and 4-hydroxyl groups of the ligating mannose residue. However, the Fuc ring in the E- and P-LE/SLe^{X} structures is “flipped” relative to mannose in the MBP-A/oligomannose complex. This results in the swapping of ring positions so that the Fuc 3- and 4-hydroxyl groups of the selectin LE/SLe^{X}complexes occupy the 4- and 3-hydroxyl group positions, respectively, of mannose in the MBP-A/oligomannose complex. Even though different hydroxyl groups are used and their relationship to the sugar ring differs (equatorial-equatorial for the mannose 3- and 4-hydroxyl groups, respectively, versus equatorial-axial in Fuc), the vectors along the sugar ring carbons to hydroxyl groups are maintained. This precise positioning of hydroxyl groups appears to be essential for simultaneous ligation to the calcium ion and hydrogen bond interactions with the protein. The crystal structure of the K3 mutant of MBP-A in which three selectin residues have been introduced (Ng and Weis, 1997) binds SLe^{X} significantly differently than we observe here for the selectin LE/SLe^{X}complexes. While this structure and the structures presented here show Fuc ligation to the bound calcium, different hydroxyl groups are involved. In the K3 mutant/SLe^{X} complex, Fuc 2- and 3-hydroxyl groups ligate calcium and occupy the Fuc 4- and 3-hydroxyl group positions, respectively, found in the selectin LE/SLe^{X} complexes. This results in a 90° rotation of the SLe^{X} orientation within the binding pocket relative to the selectin LE/SLe^{X} complexes and affords a hydrogen bond interaction between the Gal 4-hydroxyl group and the side chain of Lys-111, which we do not observe. This highlights the importance of Glu-92, Tyr-94, and Tyr-48 (and Arg-97 in E-selectin) to the binding of the ligand in the E- and P-LE/SLe^{X} complexes. Finally, models of SLe^{X} molecularly docked into the E-selectin lec/EGF crystal structure (Graves et al., 1994; Kogan et al., 1995; Poppe et al., 1997) compare favorably to our results in terms of the general orientation of SLe^{X} on the binding surface. However all disagree to varying degrees with the structures shown here with regard to the identity of the molecular contacts. With the underlying assumption that ligation of SLe^{X} to the bound calcium mimics that which is observed in the MBP-A/oligomannose complex and in the MBP-A K3 mutant/SLe^{X} complex, all models propose that the Fuc 2- and 3-hydroxyl groups of SLe^{X} ligate the bound calcium. This is in sharp contrast to our observations of two separate selectin LE/SLe^{X} complexes in which Fuc ligation is mediated via the 3- and 4-hydroxyl groups. Other proposed contacts for the E-selectin/SLe^{X} interaction are consistent or inconsistent with our results to varying degrees. The design of SLe^{X} mimetics intended for therapeutic purposes based upon these incorrect structural considerations may ultimately limit the success of these efforts.

Generation of a Functional PSGL-1 Peptide for X-ray Crystallography. We next sought to obtain a crystal structure of P-LE complexed with PSGL-1 to elucidate the structural basis of recognition but anticipated that the physiological or large, extracellular forms of PSGL-1 would prove too heterogeneous for co-crystallization attempts. Mutagenesis and biochemical studies indicate that P-selectin, in contrast to E-selectin, recognizes both polypeptide residues and a SLe^{X}-modified O-glycan within the N-terminus of mature PSGL-1. Candidates for the PSGL-1 determinant include an anionic stretch of amino acids encompassing (numbered from the N-terminus of the mature polypeptide) Tyr-5, Tyr-7, and Tyr-10, one or more of which is sulfated, and a sialylated, fucosylated (presumably SLe^{X}-like) O-glycan localized to Thr-16 by indirect methods (Pouyani and Seed, 1995; Ramachandran et al., 1999; Sako et al., 1995; Wilkins et al., 1995). Sulfation of any one of the three Tyr residues is capable of supporting P-selectin binding, however, the relative role of individual tyrosine sulfates have been inferred by rolling studies (Ramachandran et al., 1999) and not by rigorous affinity determinations. To explore these structural questions and to produce a homogeneously modified form of PSGL-1 suitable for crystallization with P-LE, we expressed a truncated form of PSGL-1 in a CHO cell line previously transfected with fucosyltransferase-VII and core2 N-acetylglucosamine, modification enzymes essential for the generation of functional PSGL-1 in CHO cells (Kumar et al., 1996; Li et al., 1996). We utilized a soluble construct of PSGL-1 (19ek.Fc, (Goetz et al., 1997)) encoding the 19 N-terminal amino acids of PSGL-1 fused to the heavy chain domain of IgG₁ (19.Fc, (Sako et al., 1995)) in which we introduced an enterokinase cleavage sequence that would allow for the isolation of monomeric PSGL-1 peptides following expression and purification. 19ek.Fc coated onto latex microspheres was previously demonstrated to support rolling over CHO cells expressing P- and E-selectin (Goetz et al., 1997).

19ek.Fc was digested with enterokinase in order to produce monomeric PSGL-1 peptides (termed 19ek peptides). Preliminary analysis by MS suggested that the 19ek peptides were structurally heterogeneous (not shown). Therefore, resolution of the mixture was accomplished by anion-exchange HPLC which separated the 19ek peptide mixture into homogenous components dominated by a major species eluting late in the salt gradient (FIG. 1A). The structure of the major 19ek peptide was determined to be the sulfoglycopeptide (termed SGP-3) as shown in FIG. 1A. The PSGL-1 portion of SGP-3 is extensively post-translationally modified and includes sulfates on all three tyrosine residues and a SLe^{X}-containing core2 modified O-glycan at Thr-16 (FIG. 1A, inset). Importantly, the glycan is identical to one of two SLe^{X}-containing O-glycans characterized for PSGL-1 isolated from the HL-60 myeloid cell line (Wilkins et al., 1996). The minor species of 19ek peptides (FIG. 1A) are less modified versions of SGP-3. The structures of 19ek peptides SGP-1 and SGP-2 (FIG. 1A) were determined to be forms of SGP-3 containing one and two tyrosine sulfates, respectively. Preliminary analyses by MS indicated that every permutation of tyrosine sulfation is present within the hyposulfated species but these were not quantitated. Also present within the 19ek peptide pool and resolved by chromatography (FIG. 1A) are forms of SGP-3 containing no tyrosine sulfates (glycopeptide-1, GP-1) or containing no carbohydrate (sulfopeptide-1, SP-1).

We used surface plasmon resonance to determine the binding affinities of P-LE for the individual 19ek peptides in order to select the most functional species for co-crystallization attempts. For comparison, we evaluated a soluble, recombinant form of PSGL-1 (sPSGL) consisting of the entire dimeric, extracellular domain. Consistent with earlier studies, P-LE bound immobilized sPSGL and 19ek peptides with rapid kinetics (FIG. 1B). Binding was determined to be specific based upon control experiments performed with EDTA and neutralizing Mabs (not shown) and with SGP-3 used as soluble inhibitor (FIG. 1B). The highest affinity interaction observed for the individual 19ek peptides was with the fully modified species SGP-3 with a K_{D} of 778 nM. An essentially identical affinity was obtained with sPSGL (FIG. 1C) suggesting that the 19ek peptide SGP-3 functionally mimics the full-length extracellular PSGL-1 construct and that the N-terminal region of PSGL-1 present in the shorter construct constitutes the entire recognition region for P-LE. The binding affinities of P-LE for hyposulfated forms of SGP-3 were slightly weaker (FIG. 1C) exhibiting K_{D} s of 2.88 μM for the disulfated species (SGP-2) and 12.3 μM for monosulfated species (SGP-1). P-LE binding affinities of versions of SGP-3 containing no carbohydrate (SP-1) or sulfotyrosines (GP-1) were weaker yet and are estimated to be 113 μM and 31.1 μM, respectively (FIG. 1C). The affinity of P-LE for a synthetic version of the 19ek peptide containing no carbohydrate or sulfation was considerably lower than any of the modified species and could not be determined at the concentrations of protein employed here. The binding affinity of P-LE binding to sPSGL and to SGP-3 determined here compare reasonably well to values previously determined for the binding of soluble P-selectin and a lectin-EGF construct of P-selectin similar to P-LE to full-length neutrophil PSGL-1 (K_{D} s of 322 nM and 422 nM, respectively) (Mehta et al., 1998). Additionally, the binding affinity of soluble P-selectin to a PSGL-1 glycosulfopeptide modified similarly to SGP-3 was recently reported to have a K_{D} of ˜350 nM (Leppanen et al., 1999).

X-ray Crystal Structure of P-LE Bound to the PSGL-1 Sulfoglycopeptide-3. P-LE was co-crystallized with a two fold molar excess of SGP-3. Repeat macroseeding and replacement of calcium with strontium was required to give large I222 space group crystals that diffracted to 1.9 Å. Phases were generated using mercury data collected in house, revealing two P-LE/SGP-3 complexes in the crystallographic asymmetric unit. The structure was refined using CNS to an R-factor of 20.4% (R-free, 23.5%) (Table 1). Of the 29 amino acids within the SGP-3 polypeptide, residues six through 18, including sulfated Tyr-7 (Tys-7) and sulfated Tyr-10 (Tys-10) are observed. Disordered and absent from the structure are polypeptide residues one through five, containing the most N-terminal sulfated tyrosine (Tys-5), and residues 19-29 containing the enterokinase-linker region. All but one residue of the SLe^{X}-modified O-glycan at Thr-16 is observed. Electron density for the NeuNAc(2,3)-linked to the Gal(1,3)GalNAc branch (FIG. 1A, inset) was not detected but this residue does not appear to be required for binding (Leppanen et al., 1999).

Two different views of the P-LE/SGP-3 complex provide a structural overview of the binding interaction from which aspects of the physiological P-selectin/PSGL-1 interaction may be inferred. SGP-3 binds with 1:1 stoichiometry to the P-LE lectin domain with a large epitope that excludes 1641 Å² from solvent and includes the SLe^{X} binding site described for the P-LE/SLe^{X} complex. Despite the fact that SGP-3 is disordered in solution as determined by transfer NOE studies (D. Tsao, unpublished observations) and likely extended, the bound conformation is compacted by internal folding that produces a hairpin-like structure. While these results do not exclude the possibility that different arms of the PSGL-1 homodimer, each supplying a different component of the binding interaction, might simultaneously engage a single P-selectin lectin domain, they suggest that a single arm of PSGL-1 is capable of providing a complete set of binding determinants. Moreover, this type of binding interaction allows for the possibility that individual monomers of the PSGL-1 homodimer might simultaneously engage two different P-selectin molecules. The co-localization of two parallel binding interactions may be a physiological requirement for P-selectin function in light of the observation that a dimerization mutant of PSGL-1 is incapable of supporting cell binding under the influence of shear (Snapp et al., 1998). Also immediately apparent from examination of the P-LE/SLe^{X} complex is that the binding of SGP-3 occurs on a face of P-LE essentially opposite the lectin domain:EGF domain interface. This provides direct evidence that the EGF domain of P-selectin does not participate in direct binding to PSGL-1, at least to the N-terminal portion of the molecule, but may have an indirect role in P-selectin function (see below).

Despite the fact that calcium was replaced by strontium, interactions seen in the complex of P-LE with SLe^{X} are reproduced in the P-LE/SLe^{X} structure and are consistent with an earlier observation that this metal can effectively substitute for calcium (Asa et al., 1992). In the complex there is a large structural rearrangement of P-LE, which causes a change in the metal ion coordination. In the P-LE/SLe^{X} complex, the 2-hydroxyl of the Fuc makes a weak hydrogen bond with Glu-107 (distance of 3.4 Å). This is not present in the PLE/SGP-3 complex but is replaced by a hydrogen bond with Glu-88 which itself also coordinates the bound strontium. In the structure of P-LE the side chain of Asn-83 is close to but does not coordinate the bound calcium and in E-LE binding of SLe^{X} binding of SLe^{X} causes it to interact with the metal ion and a Fuc hydroxyl. In the P-LE/SGP-3 complex, the position of this residue has been replaced with the side chain of Glu-88. In this respect, the metal ligation now closely mimics that of MBP complexed with oligomannose (Weis et al., 1992) and the K3 mutant of MBP complexed with SLe^{X} (Ng and Weis, 1997) in which the corresponding residue Glu-193 is observed to ligate calcium.

The interactions between the SGP-3 polypeptide and P-LE are a combination of hydrophobic and electrostatic contacts. The most N-terminal residue of the SGP-3 peptide in contact with the protein is Tys-7 followed by three residues in an extended conformation. Residues Tys-10 to Leu-13 form a turn, which changes the direction of the peptide and the remaining residues run to Pro-18 in an extended conformation. The sulfate of Tys-7 makes a series of hydrogen bonds with the side-chain hydroxyl groups of Ser-46 and Ser-47, a backbone amide nitrogen and via an ordered water molecule. In addition, His-114 of P-LE donates a hydrogen bond to the remaining sulfate oxygen completing the hydrogen bonding network. Tys-7 also makes a backbone-backbone hydrogen bond with the amide nitrogen of Lys-112 and several hydrophobic interactions with the side chains of Ser-47 and Lys-113. SGP-3 residue Leu-8 packs against Leu-13 which are both packed against the hydrophobic surface of the protein formed by the side chains of His-108 and Lys-111. These interactions must help stabilize the compact tertiary structure of the peptide. The aromatic ring of the Tys-10 side chain lies against these two leucine residues and places the sulfate in a position where it can hydrogen bond with Arg-85 and the backbone amide of Thr-16. The interactions between Arg-85 and the SGP-3 polypeptide are made possible by a large structural rearrangement of P-LE (see below) and support the findings of an earlier site-directed mutagenesis study targeting this residue within P-selectin (Bajorath et al., 1994). When mutated to Ala, the P-selectin mutant lost high affinity binding to PSGL-1 expressing cells yet retained low affinity (presumably exclusively SLe^{X}-mediated) binding. Similarly, an antibody whose epitope has been mapped to the nearby residues 76-83 has been shown to abolish binding to PSGL-1 but not to SLe^{X} (Hirose et al., 1998). In this P-LE/SGP-3 complex, the side chain of Phe-12 in SGP-3 does not contact the protein but packs against itself in the other copy of the complex related by non-crystallographic symmetry. It seems likely that in solution it would lie against the hydrophobic surface formed by the side chain Leu-110. The only remaining interactions are seen with Pro-14 of SGP-3, which packs against His-108 and accepts a hydrogen bond from Arg-85.

Compared to both unliganded P-LE and the P-LE/SLe^{X} complex, there are several dramatic changes to the conformation of P-LE associated with SGP-3 binding. The most significant conformational change is the movement of a loop of residues from Asn-83 to Asp-89 which brings Arg-85 in contact with Tys-10 of SGP-3 and Glu-88 to a position where it now ligates the bound metal. This movement in turn appears to affect the position of residues Arg-54 to Glu-74 which move to occupy the space vacated by the Asn-83 to Asp-89 loop. In uncomplexed P-LE, Thr-65 packs against the side chain of Trp-1 excluding it from solvent. The movement of loops observed for the P-LE/SGP-3 complex would be expected to expose to Trp-1 to solvent but this residue rearranges to pack between Tyr-118 of the lectin domain and Glu-135 of the EGF domain. In conjunction with the movement of Trp-1 there is a 52 degree movement of the EGF domain relative to the lectin domain and the void created by the movement of the Trp-1 side chain is filled by an MPD molecule that packs against the new position for the side chain. This raises the question as to whether the movement of the EGF domain is caused by the high concentration of MPD used for crystallization or due to binding of SGP-3. While the EGF domain does not appear to play a direct role in binding to PSGL-1, at least to the N-terminal region of the molecule, the change in its relationship to the lectin domain is an interesting observation in light of observations that the EGF domain may modulate ligand recognition (Gibson et al., 1995; Kansas et al., 1994).

Alternatively, these studies may indicate secondary binding interactions between the EGF and CR domains and other regions of PSGL-1 C-terminal to the binding site evaluated here. Additional structural studies are warranted to further explore the potential interaction between the lectin and EGF domains. The internal folding of the SGP-3 polypeptide resulting in the placement of Tys-10 near the O-glycan at Thr-16 suggests that the relationship of the SLe^{X}-modified glycan to tyrosine sulfates within the linear sequence of PSGL-1 may not be absolute above a minimum number of intervening residues. P-selectin may support multiple binding conformations of the PSGL-1 N-terminus, only one of which is shown in this study. The P-LE/SGP-3 structure suggests that Tys-7 and Tys-10 (and not Tys-5) of PSGL-1 are essential for binding, a result consistent with a mutagenesis study indicating a preferential role for these specific tyrosine sulfate residues in P-selectin mediated rolling. However, flexibility of the PSGL-1 N-terminus might allow different permutations of tyrosine sulfates, e.g., Tys-5 and Tys-7 or Tys-5 and Tys-10, to bind in a different register to the basic residues within P-selectin. This hypothesis may explain why the affinity of P-LE for the trisulfated SGP-3 is greater than for disulfated SGP-2. While it is possible that yet a third undefined tyrosine sulfate binding site exists within P-selectin which accounts for this observation, flexibility of the PSGL-1 polypeptide may permit any of the three tyrosine sulfate residues to bind to the two sites defined here. This may explain why mutations of PSGL-1 containing single tyrosine sulfate residues (including Tys-5) support P-selectin binding (Ramachandran et al., 1999). Also supporting this potential flexibility in binding is the observation that the corresponding N-terminal region of murine PSGL-1 is also critical for binding to P-selectin and yet contains two potential tyrosine sulfates only two and four residues removed from a potential glycosylation site corresponding to Thr-16. Murine P-selectin also contains basic residues at positions 85 and 114, and based upon the observations here for the human P-LE/SGP-3 interaction, might be anticipated to make similar ionic interactions with the tyrosine sulfates within murine PSGL-1. A consequence of this interaction would be fewer intervening residues spanning the tyrosine sulfate and SLe^{X} binding sites. Similarly, flexibility of the N-terminal region of PSGL-1 might also permit recognition of other O-glycan structures attached to Thr-16. SLe^{X} and Le^{X} epitopes within a longer poly-N-acetyllactosamine O-glycan have been described for myeloid PSGL-1 and P-selectin may accommodate this in similar fashion.

Concluding Remarks. The crystal structures of P-LE and E-LE complexed with SLe^{X} and of P-LE complexed with the PSGL-1 sulfoglycopeptide shown here contribute significantly to our understanding of the molecular basis of selectin recognition. The structures of P-LE and E-LE complexed with SLe^{X} exhibit both common and dissimilar binding interactions, which explain their differential affinity for this shared ligand. The structure of the P-LE/SGP-3 complex in particular illustrates how the differential recognition of PSGL-1 by E- and P-selectin is achieved. While the primary sequences of E- and P-selectin lectin domains are more than 50% identical, residues critical for the P-LE/SGP-3 interaction are unique to P-selectin and presumably account for the higher affinity of the P-selectin/PSGL-1 interaction. Residues Arg-85 and His-114, important for ionic interactions with tyrosine sulfate residues within SGP-3, are unique to P-selectin (the corresponding residues in human E-selectin are uncharged Gln and Leu, respectively). Interestingly, this trend in differential charge at these positions is observed for P-selectin and E-selectin in the mouse, rat, rabbit and cow suggesting a conserved binding motif for P-selectin interactions with PSGL-1. The structural observations made here offer potential insights into the nature of the L-selectin/PSGL-1 interaction as well. L-selectin also appears to recognize the N-terminal region of PSGL-1 based upon Mab neutralization (Kansas, 1996) and mutagenesis (Ramachandran et al., 1999) studies. While the affinity of this interaction has not yet been determined, it is anticipated that it will be of intermediate affinity relative to the P-selectin and E-selectin interactions with PSGL-1. This is predicated upon the assumption that the highly homologous L-selectin lectin domain adopts a conformation similar to that determined for E- and P-selectin and that recognition of the PSGL-1 N-terminus shares properties with those of P-selectin. As with P-selectin, human L-selectin contains a basic residue at position 85 (a Lys residue) which might be anticipated to make an essential ionic interaction with a sulfated Tyr within the PSGL-1 N-terminus. However, L-selectin does not contain a basic residue at position 114, which could support a second ionic interaction with a tyrosine sulfate within PSGL-1 and hence binding affinity perhaps comparable to P-selectin.

The interactions of the E- and P-selectins with SLe^{X} and the PSGL-1 sulfoglycopeptide shown here, based largely upon a network of hydrogen bonds and selected ionic interactions, are fundamental to the binding affinities and fast binding kinetics described for this class of cell adhesion molecules. The rapid reversibility of relatively low affinity selectin/counterreceptor interactions produces attachment and rolling of leukocytes essential for their subsequent activation, firm adhesion and extravasation into tissues. In the context of inflammation, PSGL-1 appears to play a central role in selectin-mediated processes, mediating the initial attachment of neutrophils to the vascular endothelium as well as promoting neutrophil-neutrophil and neutrophil-platelet interactions likely important for amplification of the inflammatory response. The rational design of small molecule antagonists for the treatment of inflammatory conditions in which E- and P-selectin/SLe^{X} and P-selectin/PSGL-1 interactions are demonstrated will be assisted considerably by the structural information presented herein.

TABLE 1
Data Collection Phasing and Refinement Statistics

| | | | | | |
|---|---|---|---|---|---|
| Structure | P-selection/PSG-1 | P-selection/PSGL-1/Hg | P-selectin | P-selectin/S1EX | P-selectin/S1EX |

Data Collection

| | | | | | |
|---|---|---|---|---|---|
| Resolution Range | 15.0-1.9 (1.97-1.90) | 14.01-3.5 (3.62-3.50) | 15.0-2.4 (2.49-2.4) | 14.0-3.4 (3.46-3.4) | 15.0-1.5 (1.55-1.5) |
| R-sym (%) | 5.8 (27.4) | 5.5 (7.5) | 5.2 (22.5) | 7.2 (32.2) | 4.1 (22.0) |
| % Completness | 98.5 (96.6) | 97.0 (94.0) | 95.8 (74.2) | 97.8 (100.0) | 92.4 (62.0) |
| Total Observations | 207998 | 33998 | 210361 | 44334 | 260283 |
| Unique Reflections | 44923 | 7240 | 32570 | 11667 | 29493 |
| <l/sigl> | 25.5 (3.7) | 27.4 (18.2) | 35.7 (6.6) | 29.0 (5.4) | 47.6 (4.4) |
| Phasing | | | | | |
| Phasing Power | | | | | |
| Centrics Isomorphous | | 0.94 | | | |
| Accentrics Isomorphous | | 1.17 | | | |
| Centrics - Anomalous | | 1.47 | | | |
| Isomorphous Deference on 1(%) | | 32.2 | | | |
| Number of Sites | | 1 | | | |
| I · OM(Centric/Accentric) | | 0.312/0.428 | | | |
| Model Refinement | | | | | |
| Resolution (A) | 14.0-1.9 | | 14.0-2.4 | 14.0-3.4 | 14.0-1.5 |
| R-factor | 20.4 | | 21.3 | 22.7 | 19.6 |
| R-free | 23.5 | | 25.2 | 32.2 | 21.7 |
| RMS deviations from ideal geome- | | | | | |
| try | | | | | |
| Bonds (A) | 0.010 | | 0.010 | 0.011 | 0.009 |
| Angles (0) | 1.54 | | 1.46 | 1.53 | 1.42 |
| <B value> | 12.0 | | 48.8 | 54.1 | 19.9 |
| RMS difference m | | | | | |
| mam cham bonded | | | | | |
| B values | 3.1 | | 3.5 | 3.5 | 18.3 |

TABLE 2
E-selectin SLEX 4Å contacts
TYR48, GLU80, ASN82, ASN83, GLU92, TYR94, ARG97,
GLU98, ASN105, ASP106, GLU107, Bound Calcium
E-selectin SLEX 8Å contacts
TYR44, SER45, PRO46, SER47, TYR48, ALA77, PRO78, GLY79,
GLU80, PRO81, ASN82, ASN83, GLU88, CYS90, GLU92, TYR94,
ARG97, GLU98, LYS99, ASP100, TRP104, ASN105, ASP106,
GLU107, ARG108, LYS111, LYS113, Bound Calcium
P-selectin SLEX 4Å contacts
TYR48, GLU80, ASN82, GLU92, TYR94, PRO98, SER99, ASN105,
ASP106, GLU107, Bound calcium
P-selectin SLEX 8Å contacts
TYR44, SER46, SER47, TYR48, ALA77, ASP78, ASN79, GLU80,
PRO81, ASN82, ASN83, ARG85, GLU88, CYS90, GLU92, ILE93,
TYR94, LYS96, SER97, PRO98, SER99, ALA100, TRP104, ASN105,
ASP106, GLU107, HIS108, LYS111, LYS113, Bound Calcium
P-selectin PSGL-1 4Å contacts
ALA9, TYR45, SER46, SER47, TYR48, GLU80, ASN82, LYS84,
ARG85, GLU88, GLU92, TYR94, PRO98, SER99, ASN105, ASP106,
GLU107, HIS108, LEU110, LYS111, LYS112, LYS113, HIS114,
Bound Strontium
P-selectin PSGL-1 8Å contacts
SER6, THR7, LYS8, ALA9, TYR10, SER11, TYR44, TYR45, SER46,
SER47, TYR48, TYR49, TRP50, ALA77, ASP78, ASN79, GLU80,
PRO81, ASN82, ASN83, LYS84, ARG85, ASN86, ASN87, GLU88,
CYS90, GLU92, ILE93, TYR94, ILE95, LYS96, SER97, PRO98,
SER99, ALA100, TRP104, ASN105, ASP106, GLU107, HIS108,
CYS109, LEU110, LYS111, LYS112, LYS113, HIS114,
Bound Strontium

### REFERENCES

(1) Alon, R., Chen, S., Puri, K. D., Finger, E. B., and Springer, T. A. (1997). The kinetics of L-selectin tethers and the mechanics of selectin-mediated rolling. J. Cell Biol. 138, 1169-1180.

(2) Alon, R., Hammer, D. A., and Springer, T. A. (1995). Lifetime of the P-selectin-carbohydrate bond and its response to tensile force in hydrodynamic flow. Nature 374, 539-542.

(3) Asa, D., Gant, T., Oda, Y., and Brandley, B. K. (1992). Evidence for two classes of carbohydrate binding sites on selecting. Glycobiology 2, 395-399.

(4) Bajorath, J., Hollenbaugh, D., King, G., Harte, W., Eustice, D. C., Darveau, R. P., and Aruffo, A. (1994). CD62/P-selectin binding sites for myeloid cells and sulfatides are overlapping. Biochemistry 33, 1332-1339.

(5) Baumheuter, S., Singer, M. S., Henzel, W., Hemmerich, S., Renz, M., Rosen, S. D., and Lasky, L. A. (1993). Binding of L-selectin to the vascular sialomucin CD34. Science 262, 436-438.

(6) Berg, E. L., McEvoy, L. M., Berlin, C., Bargatze, R. F., and Butcher, E. C. (1993). L-selectin-mediated lymphocyte rolling on MAdCAM-1. Nature 366, 695-698.

(7) Bricogne, G. (1993). Direct phase determination by entropy maximization and likelihood ranking; status report and perspectives. Acta Crystallogr. D49, 37-60.

(8) Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L. (1998). Crystallography and NMR system: A new software suite for macromolecular structure determination. Acta Crystallogr. D54, 905-921.

(9) CCP4 (1994). The CCP-4 suite: programs for X-ray crystallography. Acta Crystallogr. D50, 760-763.

(10) Chen, S., Alon, R., Fuhlbrigge, R. C., and Springer, T. A. (1997). Rolling and transient tethering of leukocytes on antibodies reveal specializations of selecting. Proc. Natl. Acad. Sci. USA 94, 3172-3177.

(11) Croce, K., Freedman, S. J., Furie, B. C., and Furie, B. (1998). Interaction between soluble P-selectin and soluble P-selectin glycoprotein ligand 1: equilibrium binding analysis. Biochemistry 37, 16472-16480.

(12) de la Fourtelle, E., and Bricogne, G. (1997). Maximum-likelihood heavy-atom parameter refinement for multiple isomorphous replacement and multiwavelength anomalous diffraction methods. Methods Enzymol. 276, 472-494.

(13) Drickamer, K. (1988). Two distinct classes of carbohydrate-recognition domains in animal lectins. J. Biol. Chem. 263, 9557-9560.

(14) Erbe, D. V., Watson, S. R., Presta, L. G., Wolitzky, B. A., Foxall, C., Brandley, B. K., and Lasky, L. A. (1993). P- and E-selectin use common sites for carbohydrate ligand recognition and cell adhesion. J. Cell Biol. 120, 1227-1235.

(15) Erbe, D. V., Wolitzky, B. A., Presta, L. G., Norton, C. R., Ramos, R. J., Burns, D. K., Rumberger, J. M., Rao, B. N., Foxall, C., Brandley, B. K., and Lasky, L. A. (1992). Identification of an E-selectin region critical for carbohydrate recognition and cell adhesion. J. Cell Biol. 119, 215-227.

(16) Foxall, C., Watson, S. R., Dowbenko, D., Fennie, C., Lasky, L. A., Kiso, M., Hasegawa, A., Asa, D., and Brandley, B. K. (1992). The three members of the selectin receptor family recognize a common carbohydrate epitope, the sialyl Lewis^{X} oligosaccharide. J. Cell Biol. 117, 895-902.

(17) Gibson, R. M., Kansas, G. S., Tedder, T. F., Furie, B., and Furie, B. C. (1995). Lectin and epidermal growth factor domains of P-selectin at physiologic density are the recognition unit for leukocyte binding. Blood 85, 151-158.

(18) Goetz, D. J., Greif, D. M., Ding, H., Camphausen, R. T., Howes, S., Comess, K. M., Snapp, K. R., Kansas, G. S., and Luscinskas, F. W. (1997). Isolated P-selectin glycoprotein ligand-1 dynamic adhesion to P- and E-selectin. J. Cell Biol. 137, 509-519.

(19) Graves, B. J., Crowther, R. L., Chandran, C., Rumberger, J. M., Li, S., Huang, K. S., Presky, D. H., Familletti, P. C., Wolitzky, B. A., and Burns, D. K. (1994). Insight into E-selectin/ligand interaction from the crystal structure and mutagenesis of the lec/EGF domains. Nature 367, 532-538.

(20) Hirose, M., Kawashima, H., and Miyasaka, M. (1998). A functional epitope on P-selectin that supports binding of P-selectin to P-selectin glycoprotein ligand-1 but not to sialyl Lewis^{X} oligosaccharides. Int. Immunol. 10, 639-649.

(21) Hollenbaugh, D., Bajorath, J., Stenkamp, R., and Aruffo, A. (1993). Interaction of P-selectin (CD62) and its cellular ligand: analysis of critical residues. Biochemistry 32, 2960-2966.

(22) Honig, B., and Nicholls, A. (1995). Classical electrostatics in biology and chemistry. Science 268, 1144-1149.

(23) Kansas, G. S. (1996). Selectins and their ligands: current concepts and controversies. Blood 88, 3259-3287.

(24) Kansas, G. S., Saunders, K. B., Ley, K., Zakrzewicz, A., Gibson, R. M., Furie, B. C., Furie, B., and Tedder, T. F. (1994). A role for the epidermal growth factor-like domain of P-selectin in ligand recognition and cell adhesion. J. Cell Biol. 124, 609-618.

(25) Kogan, T. P., Revelle, B. M., Tapp, S., Scott, D., and Beck, P. J. (1995). A single amino acid residue can determine the ligand specificity of E-selectin. J. Biol. Chem. 270, 14047-14055.

(26) Kraulis, P. J. (1991). MOLSCRIPT: a program to produce both detailed and schematic plots of protein structures. J. Appl. Crystallogr. 24, 946-950.

(27) Kumar, R., Camphausen, R. T., Sullivan, F. X., and Cumming, D. A. (1996). Core2 b-1,6-N-acetylglucosaminyltransferase enzyme activity is critical for P-selectin glycoprotein ligand-1 binding to P-selectin. Blood 88, 3872-3879.

(28) Lasky, L. A., Singer, M. S., Dowbenko, D., Imai, Y., Henzel, W. J., Grimley, C., Fennie, C., Gillett, N., Watson, S. R., and Rosen, S. D. (1992). An endothelial ligand for L-selectin is a novel mucin-like molecule. Cell 69, 927-938.

(29) LaVallie, E. R., Rehemtulla, A., Racie, L. A., DiBlasio, E. A., Ferenz, C., Grant, K. L., Light, A., and McCoy, J. M. (1993). Cloning and functional expression of a cDNA encoding the catalytic subunit of bovine enterokinase. J. Biol. Chem. 268, 23311-23317.

(30) Leppanen, A., Mehta, P., Ouyang, Y. B., Ju, T., Helin, J., Moore, K. L., van Die, I., Canfield, W. M., McEver, R. P., and Cummings, R. D. (1999). A novel glycosulfopeptide binds to P-selectin and inhibits leukocyte adhesion to P-selectin. J. Biol. Chem. 274, 24838-24848.

(31) Levinovitz, A., Muhlhoff, J., Isenmann, S., and Vestweber, D. (1993). Identification of a glycoprotein ligand for E-selectin on mouse myeloid cells. J. Cell Biol. 121, 449-459.

(32) Li, F., Wilkins, P. P., Crawley, S., Weinstein, J., Cummings, R. D., and McEver, R. P. (1996). Post-translational modifications of recombinant P-selectin glycoprotein ligand-1 required for binding to P- and E-selectin. J. Biol. Chem. 271, 3255-3264.

(33) McEver, R. P., and Cummings, R. D. (1997). Role of PSGL-1 binding to selectins in leukocyte recruitment. J. Clin. Invest. 100, S97-103.

(34) Mehta, P., Cummings, R. D., and McEver, R. P. (1998). Affinity and kinetic analysis of P-selectin binding to P-selectin glycoprotein ligand-1. J. Biol. Chem. 273, 32506-32513.

(35) Merrit, E. A., and Murphy, M. E. P. (1994). Raster3D version 2.0: a program for photorealistic molecular graphics. Acta Crystallogr. D50, 869-873.

(36) Moore, K. L., Stults, N. L., Diaz, S., Smith, D. F., Cummings, R. D., Varki, A., and McEver, R. P. (1992). Identification of a specific glycoprotein ligand for P-selectin (CD62) on myeloid cells. J. Cell Biol. 118, 445-456.

(37) Ng, K. K., and Weis, W. I. (1997). Structure of a selectin-like mutant of mannose-binding protein complexed with sialylated and sulfated Lewis^{X} oligosaccharides. Biochemistry 36, 979-988.

(38) Nicholls, A., Sharp, K., and Honig, B. (1991). Protein folding and association: insights from the interfacial and thermodynamic properties of hydrocarbons. Proteins 11, 281-296.

(39) Poppe, L., Brown, G. S., Philo, J. S., Nikrad, P. V., and Shah, B. H. (1997). Conformation of the SLe^{X} tetrasaccharide, free in solution and bound to E-, P-, and L-selectin. J. Am. Chem. Soc. 119, 1727-1736.

(40) Pouyani, T., and Seed, B. (1995). PSGL-1 recognition of P-selectin is controlled by a tyrosine sulfation consensus at the PSGL-1 amino terminus. Cell 83, 333-343.

(41) Puri, K. D., Chen, S., and Springer, T. A. (1998). Modifying the mechanical property and shear threshold of L-selectin adhesion independently of equilibrium properties. Nature 392, 930-933.

(42) Ramachandran, V., Nollert, M. U., Qiu, H., Liu, W. J., Cummings, R. D., Zhu, C., and McEver, R. P. (1999). Tyrosine replacement in P-selectin glycoprotein ligand-1 affects distinct kinetic and mechanical properties of bonds with P- and L-selectin. Proc. Natl. Acad. Sci. USA 96, 13771-13776.

(43) Sako, D., Chang, X. J., Barone, K. M., Vachino, G., White, H. M., Shaw, G., Veldman, G. M., Bean, K. M., Ahern, T. J., Furie, B., Cumming, D. A., and Larsen, G. R. (1993). Expression cloning of a functional glycoprotein ligand for P-selectin. Cell 75, 1179-1186.

(44) Sako, D., Comess, K. M., Barone, K. M., Camphausen, R. T., Cumming, D. A., and Shaw, G. D. (1995). A sulfated peptide segment at the amino terminus of PSGL-1 is critical for P-selectin binding. Cell 83, 323-331.

(45) Snapp, K. R., Craig, R., Herron, M., Nelson, R. D., Stoolman, L. M., and Kansas, G. S. (1998). Dimerization of P-selectin glycoprotein ligand-1 (PSGL-1) required for optimal recognition of P-selectin. J. Cell Biol. 142, 263-270.

(46) Springer, T. A. (1994). Traffic signals for lymphocyte recirculation and leukocyte emigration: the multistep paradigm. Cell 76, 301-314.

(47) Vestweber, D., and Blanks, J. E. (1999). Mechanisms that regulate the function of the selectins and their ligands. Physiol. Rev. 79, 181-213.

(48) Weis, W. I., Drickamer, K., and Hendrickson, W. A. (1992). Structure of a C-type mannose-binding protein complexed with an oligosaccharide. Nature 360, 127-134.

(49) Wilkins, P. P., McEver, R. P., and Cummings, R. D. (1996). Structures of the O-glycans on P-selectin glycoprotein ligand-1 from HL-60 cells. J. Biol. Chem. 271, 18732-18742.

(50) Wilkins, P. P., Moore, K. L., McEver, R. P., and Cummings, R. D. (1995). Tyrosine sulfation of P-selectin glycoprotein ligand-1 is required for high affinity binding to P-selectin. J. Biol. Chem. 270, 22677-22680.

(51) Yang, J., Furie, B. C., and Furie, B. (1999). The biology of P-selectin glycoprotein ligand-1: its role as a selectin counterreceptor in leukocyte-endothelial and leukocyte-platelet interaction. Thromb. Haemost. 81, 1-7.

(52) Yang, J., Hirata, T., Croce, K., Merrill-Skoloff, G., Tchernychev, B., Williams, E., Flaumenhaft, R., Furie, B. C., and Furie, B. (1999). Targeted gene disruption demonstrates that P-selectin glycoprotein ligand 1 (PSGL-1) is required for P-selectin-mediated but not E-selectin-mediated neutrophil rolling and migration. J. Exp. Med. 190, 1769-1782.

All publications mentioned herein above, whether to issued patents, pending applications, published articles, protein structure deposits, or otherwise, are hereby incorporated by reference in their entirety. While the foregoing invention has been described in some detail for purposes of clarity and understanding, it will be appreciated by one skilled in the art from a reading of the disclosure that various changes in form and detail can be made without departing from the true scope of the invention in the appended claims.

What is claimed is:

1. A crystallized P-selectin LE.

2. The crystallized P-selectin LE of claim 1, characterized as being in plate form with space group P2₁, and having unit cell parameters of a=81.0 Å, b=60.8 Å, c=91.4 Å, and beta=103.6°.

3. A crystallized complex of P-selectin LE and SLe^{X}.

4. The crystallized complex of claim 3, characterized as being in plate form with space group P2₁, and having unit cell parameters of a=81.1 Å, b=60.5 Å, c=91.4 Å, and beta=103.3°.

5. A crystallized complex of E-selectin LE and SLe^{X}.

6. The crystallized complex of claim 5, characterized as being in rod form with space group P2₁2₁2₁, and having unit cell parameters of a=34.5 Å, b=72.4 Å, and c=77.6 Å.

7. A crystallized complex of P-selectin LE and a PSGL-1 peptide.

8. The crystallized complex of claim 7, characterized as being in bipyramidal form with space group I222 and having unit cell parameters of a=63.4 Å, b=96.8 Å, and c=187.3 Å.

9. An active site of an SLe^{X} binding protein or peptide, wherein said active site comprises the relative structural coordinates of amino acid residues TYR48, GLU80, ASN82, GLU92, TYR94, PRO98, SER99, ASN105, ASP106, GLU107 and bound calcium according to FIG. 3, ± a root mean square deviation from the backbone atoms of said amino acids of not more than 1.5 Å.

10. The active site of claim 9, wherein said active site further comprises the relative structural coordinates of amino acid residues TYR44, SER46, SER47, ALA77, ASP78, ASN79, PRO81, ASN83, ARG85, GLU88, CYS90, ILE93, LYS96, SER97, ALA100, TRP104, HIS108, LYS111 and LYS113 according to FIG. 3, ± a root mean square deviation from the backbone atoms of said amino acids of not more than 1.5 Å.

11. An active site of an SLe^{X} binding protein or peptide, wherein said active site comprises the relative structural coordinates of amino acid residues TYR48, GLU80, ASN82, ASN83, GLU92, TYR94, ARG97, GLU98, ASN105, ASP106, GLU107 and bound calcium according to FIG. 4, ± a root mean square deviation from the backbone atoms of said amino acids of not more than 1.5 Å.

12. The active site of claim 11, wherein said active site further comprises the relative structural coordinates of amino acid residues TYR44, SER45, PRO46, SER47, ALA77, PRO78, GLY79, PRO81, GLU88, CYS90, LYS99, ASP100, TRP104, ARG108, LYS111 and LYS113 according to FIG. 4, ± a root mean square deviation from the backbone atoms of said amino acids of not more than 1.5 Å.

13. An active site of a PSGL-1 binding protein or peptide, wherein said active site comprises the relative structural coordinates of amino acid residues ALA9, TYR45, SER46, SER47, TYR48, GLU80, ASN82, LYS84, ARG85, GLU88, GLU92, TYR94, PRO98, SER99, ASN105, ASP106, GLU107, HIS108, LEU110, LYS111, LYS112, LYS113, HIS114 and bound strontium according to FIG. 5, ± a root mean square deviation from the backbone atoms of said amino acids of not more than 1.5 Å.

14. The active site of claim 13, wherein said active site further comprises the relative structural coordinates of amino acid residues SER6, THR7, LYS8, TYR10, SER11, TYR44, TYR49, TRP50, ALA77, ASP78, ASN79, PRO81, ASN83, ASN86, ASN87, CYS90, ILE93, ILE95, LYS96, SER97, ALA100, TRP104 and CYS109 according to FIG. 5, ± a root mean square deviation from the backbone atoms of said amino acids of not more than 1.5 Å.

15. A method for identifying an agent that interacts with P-selectin LE, comprising the steps of:
(a) generating a three dimensional model of P-selectin LE using the relative structural coordinates according to FIGS. 2, 3 or 5, ± a root mean square deviation from the backbone atoms of said amino acids of not more than 1.5 Å; and
(b) employing said three-dimensional model to design or select an agent that interacts with P-selectin LE.

16. The method of claim 15, further comprising the steps of: (c) obtaining the identified agent; and (d) contacting the identified agent with P-selectin LE in order to determine the effect the agent has on P-selectin LE activity.

17. A method for identifying an activator or inhibitor of a molecule or molecular complex comprising an SLe^{X} binding site, comprising the steps of:
(a) generating a three dimensional model of said molecule or molecular complex comprising an SLe^{X} binding site using (i) the relative structural coordinates according to FIG. 3 of residues TYR48, GLU80, ASN82, GLU92, TYR94, PRO98, SER99, ASN105, ASP106, GLU107 and bound calcium, ± a root mean square deviation from the backbone atoms of said amino acids of not more than 1.5 Å, or (ii) the relative structural coordinates according to FIG. 4 of amino acid residues TYR48, GLU80, ASN82, GLU92, TYR94, ARG97, GLU98, ASN105, ASP106, GLU107 and bound calcium, ± a root mean square deviation from the backbone atoms of said amino acids of not more than 1.5 Å; and
(b) selecting or designing a candidate activator or inhibitor by performing computer fitting analysis of the candidate activator or inhibitor with the three dimensional model generated in step (a).

18. The method of claim 17, wherein the relative structural coordinates according to FIG. 3 further comprises amino acid residues TYR44, SER46, SER47, ALA77, ASP78, ASN79, PRO81, ASN83, ARG85, GLU88, CYS90, ILE93, LYS96, SER97, ALA100, TRP104, HIS108, LYS111 and LYS113, ± a root mean square deviation from the backbone atoms of said amino acids of not more than 1.5 Å.

19. The method of claim 17, wherein the relative structural coordinates according to FIG. 4 further comprises the amino acid residues TYR44, SER45, PRO46, SER47, ALA77, PRO78, GLY79, PRO81, GLU88, CYS90, LYS99, ASP100, TRP104, ARG108, LYS111 and LYS113, ± a root mean square deviation from the backbone atoms of said amino acids of not more than 1.5 Å.

20. The method of claim 17, which further comprises the steps of: (c) obtaining the candidate activator or inhibitor; and (d) contacting the candidate activator or inhibitor with the molecule or molecular complex and determining the effect the candidate activator or inhibitor has on the molecule or molecular complex.

21. The method of claim 20, wherein the candidate activator or inhibitor is contacted with the molecule or molecule complex in the presence of SLe^{X} in order to determine the effect the candidate activator or inhibitor has on binding of the molecule or molecular complex to SLe^{X}.

22. A method for identifying an activator or inhibitor of a molecule or molecular complex comprising a PSGL-1 binding site, comprising the steps of:
(a) generating a three dimensional model of said molecule or molecular complex comprising a PSGL-1 binding site using the relative structural coordinates according to FIG. 5 of amino acid residues ALA9, TYR45, SER46, SER47, TYR48, GLU80, ASN82, LYS84, ARG85, GLU88, GLU92, TYR94, PRO98, SER99, ASN105, ASP106, GLU107, HIS108, LEU110, LYS111, LYS112, LYS113, HIS114 and bound strontium, ± a root mean square deviation from the backbone atoms of said amino acids of not more than 1.5 Å; and
(b) selecting or designing a candidate activator or inhibitor by performing computer fitting analysis of the candidate activator or inhibitor with the three dimensional model generated in step (a).

23. The method of claim 22, wherein the relative structural coordinates according to FIG. 5 further comprises amino acid residues SER6, THR7, LYS8, TYR10, SER11, TYR44, TYR49, TRP50, ALA77, ASP78, ASN79, PRO81, ASN83, ASN86, ASN87, CYS90, ILE93, ILE95, LYS96, SER97, ALA100, TRP104 and CYS109, ± a root mean square deviation from the backbone atoms of said amino acids of not more than 1.5 Å.

24. The method of claim 22, which further comprises the steps of: (c) obtaining the candidate activator or inhibitor; and (d) contacting the candidate activator or inhibitor with the molecule or molecular complex and determining the effect the candidate activator or inhibitor has on the molecule or molecular complex.

25. The method of claim 24, wherein the candidate activator or inhibitor is contacted with the molecule or molecule complex in the presence of PSGL-1 or a PSGL-1 peptide in order to determine the effect the candidate activator or inhibitor has on binding of the molecule or molecular complex to PSGL-1 or a PSGL-1 peptide.

26. A method for identifying an agent that interacts with SLe^{X}, comprising the steps of:
(a) generating a three dimensional model of SLe^{X} using the relative structural coordinates according to FIG. 3 or 4, ± a root mean square deviation from the backbone atoms of said amino acids of not more than 1.5 Å; and
(b) employing said three-dimensional structure to design or select an agent that interacts with SLe^{X}.

27. The method of claim 26, further comprising the steps of: (c) obtaining the identified agent; and (d) contacting the identified agent with SLe^{X} in order to determine the effect the agent has on SLe^{X} activity.

28. A method for identifying an agent that interacts with PSGL-1, comprising the steps of:
(a) generating a three dimensional model of a PSGL-1 peptide using the relative structural coordinates according to FIG. 5, ± a root mean square deviation from the backbone atoms of said amino acids of not more than 1.5 Å; and
(b) employing said three-dimensional structure to design or select an agent that interacts with PSGL-1.

29. The method of claim 28, further comprising the steps of: (c) obtaining the identified agent; and (d) contacting the identified agent with PSGL-1 or a PSGL-1 peptide in order to determine the effect the agent has on PSGL-1 or the PSGL-1 peptide activity.

30. An agent identified by the method of claim 15.

31. An inhibitor or activator identified by the method of claim 17.

32. An inhibitor or activator identified by the method of claim 22.

33. An agent identified by the method of claim 26.

34. An agent identified by the method of claim 28.

35. A method for obtaining a crystallized complex of an E-selectin type molecule and a compound that coordinates calcium, said method comprising the steps of:
(a) contacting a crystallized E-selectin type molecule with a compound that coordinates calcium in the presence of calcium ions and PEG to form a crystallized complex of the E-selectin type molecule and said compound that coordinates calcium; and
(b) contacting said crystallized complex in the presence of a reduced concentration of calcium ions, and sufficient concentrations of PEG and an ionic salt to obtain a final crystallized complex, that upon cooling, is suitable for elucidating the three dimensional structures of the E-selectin type molecule and said compound that coordinates calcium by x-ray diffraction of said final crystallized complex.
